(19) United States
(12) Patent Application Publication
(10) Pub. No.: US 2006/0172011 A1
(43) Pub. Date: Aug. 3, 2006

(54) Medical treatment

(76) Inventors: Brian Robert Champion, Cambridge (GB); Silvia Ragno, Cambridge (GB)

Correspondence Address: FROMMER LAWRENCE & HAUG, 745 FIFTH AVENUE- 10TH FL., NEW YORK, NY, 10151, US

(21) Appl. No.: 11/232,404

(22) Filed: Sep. 21, 2005

Related U.S. Application Data
(63) Continuation-in-part of application No. PCT/GB04/01229, filed on Mar. 22, 2004.

(30) Foreign Application Priority Data
Mar. 21, 2003 (GB) 0306583.6
Mar. 21, 2003 (GB) 0306582.8
Mar. 22, 2003 (GB) 0306621.4
Mar. 22, 2003 (GB) 0306622.2
Mar. 22, 2003 (GB) 0306626.3
Mar. 22, 2003 (GB) 0306624.8
Mar. 22, 2003 (GB) 0306640.4
Mar. 22, 2003 (GB) 0306644.6
Mar. 22, 2003 (GB) 0306650.3
Mar. 22, 2003 (GB) 0306651.1
Mar. 22, 2003 (GB) 0306654.5

(51) Int. Cl. A61K 48/00; A61K 38/17; C12N 15/87; A61K 9/14

(52) U.S. Cl. 424/489; 514/44; 435/459; 514/12

(57) ABSTRACT

Provided is a particle capable of being inserted into or taken up by a cell comprising i) a polynucleotide coding for a modulator of Notch signalling; and ii) a polynucleotide coding for an antigen or antigenic determinant thereof. Methods for using the particles are also described.

### CROSS-REFERENCE TO RELATED APPLICATIONS

This application is a continuation-in-part of International Application No. PCT/GB2004/001229, filed Mar. 22, 2004, published as WO 2004/083372 on Sep. 30, 2004, and claiming priority to GB Application Serial Nos. 0306583.6 and 0306582.8, filed Mar. 21, 2004; 0306621.4, 0306622.2, 0306626.3, 0306624.8, 0306640.4, 0306644.6, 0306650.3, 0306651.1, and 0306654.5, all filed Mar. 22, 2004.

Reference is made to U.S. application Ser. No. 09/310,685, filed May 4, 1999; Ser. No. 09/870,902, filed May 31, 2001; Ser. No. 10/013,310, filed Dec. 7, 2001; Ser. No. 10/147,354, filed May 16, 2002; Ser. No. 10/357,321, filed Feb. 3, 2002; Ser. No. 10/682,230, filed Oct. 9, 2003; Ser. No. 10/720,896, filed Nov. 24, 2003; Ser. Nos. 10/763,362, 10/764,415 and 10/765,727, all filed Jan. 23, 2004; Ser. No. 10/812,144, filed Mar. 29, 2004; Ser. Nos. 10/845,834 and 10/846,989, both filed May 14, 2004; Ser. No. 10/877,563, filed Jun. 25, 2004; Ser. No. 10/899,422, filed Jul. 26, 2004; Ser. No. 10/958,784, filed Oct. 5, 2004; Ser. No. 11/050,328, filed Feb. 3, 2005; Ser. No. 11/058,066, filed Feb. 14, 2005; Ser. No. 11/071,796, filed Mar. 3, 2005; Ser. No. 11/078,735, filed Mar. 10, 2005; Ser. No. 11/103,077, filed Apr. 11, 2005; Ser. No. 11/178,724, filed Jul. 11, 2005; and Ser. No. 11/188,417, filed Jul. 25, 2005.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to modulation of the Notch signalling pathway in therapy.

### BACKGROUND OF THE INVENTION

International Patent Publication No WO 98/20142 describes how manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted.

It has also been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins.

A description of the Notch signalling pathway and conditions affected by it may be found, for example, in our published PCT Applications as follows:

PCT/GB97/03058 (filed on 6 Nov. 1997 and published as WO 98/20142; claiming priority from GB 9623236.8 filed on 7 Nov. 1996, GB 9715674.9 filed on 24 Jul. 1997 and GB 9719350.2 filed on 11 Sep. 1997);

PCT/GB99/04233 (filed on 15 Dec. 1999 and published as WO 00/36089; claiming priority from GB 9827604.1 filed on 15 Dec. 1999);

PCT/GB00/04391 (filed on 17 Nov. 2000 and published as WO 0135990; claiming priority from GB 9927328.6 filed on 18 Nov. 1999);

PCT/GB01/03503 (filed on 3 Aug. 2001 and published as WO 02/12890; claiming priority from GB 0019242.7 filed on 4 Aug. 2000);

PCT/GB02/02438 (filed on 24 May 2002 and published as WO 02/096952; claiming priority from GB 0112818.0 filed on 25 May 2001);

PCT/GB02/03381 (filed on 25 Jul. 2002 and published as WO 03/012111; claiming priority from GB 0118155.1 filed on 25 Jul. 2001); PCT/GB02/03397 (filed on 25 Jul. 2002 and published as WO 03/012441; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);

PCT/GB02/03426 (filed on 25 Jul. 2002 and published as WO 03/011317; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);

PCT/GB02/04390 (filed on 27 Sep. 2002 and published as WO 03/029293; claiming priority from GB 0123379.0 filed on 28 Sep. 2001);

PCT/GB02/05137 (filed on 13 Nov. 2002 and published as WO 03/041735; claiming priority from GB 0127267.3 filed on 14 Nov. 2001, PCT/GB02/03426 filed on 25 Jul. 2002, GB 0220849.4 filed on 7 Sep. 2002, GB 0220913.8 filed on 10 Sep. 2002 and PCT/GB02/004390 filed on 27 Sep. 2002);

PCT/GB02/05133 (filed on 13 Nov. 2002 and published as WO 03/042246; claiming priority from GB 0127271.5 filed on 14 Nov. 2001 and GB 0220913.8 filed on 10 Sep. 2002). All of the foregoing are hereby incorporated herein by reference.

Reference is made also to Hoyne G. F. et al (1999) Int Arch Allergy Immunol 118:122-124; Hoyne et al. (2000) Immunology 100:281-288; Hoyne G. F. et al (2000) Intl Immunol 12:177-185; Hoyne, G. et al. (2001) Immunological Reviews 182:215-227), also incorporated herein by reference.

The present invention seeks to provide further methods of modulating the Notch signalling pathway, and, in particular, for modulating immune responses.

### SUMMARY OF THE INVENTION

According to a first aspect of the invention there is provided a polynucleotide delivery agent capable of being inserted into or taken up by a cell comprising:

i) a polynucleotide coding for a modulator of Notch signalling; and

ii) a polynucleotide coding for an antigen or antigenic determinant thereof.

Suitably the delivery agent is in the form of a particle. Thus, according to a preferred aspect of the invention there is provided a particle capable of being inserted into or taken up by a cell comprising:

i) a polynucleotide coding for a modulator of Notch signalling; and

ii) a polynucleotide coding for an antigen or antigenic determinant thereof.

According to a further aspect of the invention there is provided a product comprising:

i) a polynucleotide coding for a modulator of Notch signalling; and

ii) a polynucleotide coding for an antigen or antigenic determinant;

as a combined preparation for simultaneous, contemporaneous, separate or sequential use for modulation of the immune response to said antigen or antigenic determinant.

According to a further aspect of the invention there is provided a method for modulating an immune response to an antigen in a subject by administering a particle as claimed described herein to a subject in need thereof.

According to a further aspect of the invention there is provided a method for modifying an immune response to an antigen or antigenic determinant by causing to be inserted into or taken up by a cell:

i) a polynucleotide coding for a modulator of Notch signalling; and

ii) a polynucleotide coding for the antigen or antigenic determinant thereof.

Suitably the particle is a microparticle. Suitably the particle comprises a matrix, carrier or substrate.

Suitably the particle is capable of being inserted into or taken up by an immune cell, such as an antigen presenting cell, for example a dendritic cell or Langerhans cell.

Suitably the particle is capable of being inserted into a cell, for example by a ballistic/biolistic delivery method.

Alternatively the particle may be capable of being taken up by a cell, for example by endocytosis or phagocytosis.

Suitably one or both polynucleotides are borne on a surface of a matrix, carrier or substrate. Alternatively or in addition one or both polynucleotides may be borne within a matrix, carrier or substrate.

In one embodiment the polynucleotide coding for a modulator of Notch signalling codes for an activator of Notch signalling, such as a Notch receptor agonist. In an alternative embodiment the polynucleotide coding for a modulator of Notch signalling codes for an inhibitor of Notch signalling, such as a Notch receptor antagonist.

Suitably the polynucleotide coding for the modulator of Notch signalling codes for a protein or polypeptide comprising a Notch ligand or an active fragment, derivative, homologue, analogue or allelic variant thereof, such as a Delta or Serrate/Jagged protein or polypeptide or a fragment, derivative, homologue, analogue or allelic variant thereof.

For example, the polynucleotide coding for the modulator of Notch signalling may codes for a fusion protein comprising a segment of a Notch ligand extracellular domain and an immunoglobulin F_{c} segment.

Preferably the polynucleotide coding for the modulator of Notch signalling codes for a protein or polypeptide comprising a Notch ligand DSL domain and at least one EGF-like domain.

Preferably the polynucleotide coding for the modulator of Notch signalling codes for a protein or polypeptide comprising a Notch ligand DSL domain and at least two EGF-like domains.

Preferably the polynucleotide coding for the modulator of Notch signalling codes for a protein or polypeptide comprising a Notch ligand DSL domain and at least three EGF-like domains.

Suitably the polynucleotide coding for the modulator of Notch signalling codes for a protein or polypeptide comprising a Notch ligand DSL domain, at least one EGF-like domain and a membrane binding or transmembrane domain.

Alternatively the polynucleotide coding for the modulator of Notch signalling may code for Notch intracellular domain (Notch IC) or a fragment, derivative, homologue, analogue or allelic variant thereof.

In a further embodiment the polynucleotide coding for the modulator of Notch signalling may code for a dominant negative version of a Notch signalling repressor or a polypeptide capable of upregulating the expression or activity of a Notch ligand or a downstream component of the Notch signalling pathway.

Suitably the polynucleotide sequence coding for a modulator of Notch signalling may code for a protein or polypeptide comprising:

i) a Notch ligand DSL domain;

ii) 1-16 or more Notch ligand EGF domains;

iii) preferably all or part of a transmembrane domain;

iv) preferably all or part of a Notch ligand N-terminal domain;

iv) preferably all or part of a Notch ligand intracellular domain; and

v) optionally one or more heterologous amino acid sequences.

Suitably a polynucleotide sequence coding for a modulator of Notch signalling may code for a protein or polypeptide comprising:

i) a Notch ligand DSL domain;

ii) 2-12 or more Notch ligand EGF domains;

iii) preferably all or part of a transmembrane domain;

iv) preferably all or part of a Notch ligand N-terminal domain;

iv) preferably all or part of a Notch ligand intracellular domain; and

v) optionally one or more heterologous amino acid sequences.

Suitably a polynucleotide sequence coding for a modulator of Notch signalling may code for a protein or polypeptide comprising:

i) a Notch ligand DSL domain;

ii) 3-8 or more Notch ligand EGF domains;

iii) preferably all or part of a transmembrane domain;

iv) preferably all or part of a Notch ligand N-terminal domain;

iv) preferably all or part of a Notch ligand intracellular domain; and

v) optionally one or more heterologous amino acid sequences.

Suitably the Notch ligand domains are from Delta1, Delta3, Delta4, Jagged1 or Jagged2, preferably human Delta1, human Delta3, human Delta4, human Jagged1 or human Jagged2.

Preferably the protein or polypeptide will be expressed on the surface of a cell in use (e.g. as a membrane protein), preferably on the surface of an immune cell such as a T-cell, B-cell or APC.

Suitably for example the protein or polypeptide which the polynucleotide sequence codes for may have at least a region which has at least 50%, preferably at least 70%, preferably at least 90%, for example at least 95% amino acid sequence similarity (or preferably sequence identity) to the following sequence (SEQ ID NO: 1), preferably along the entire length of the latter:

GVFELKLQEFVNKKGLLGNRNCCRGGAGPPPCACRTFFRVCLKHYQASVS
PEPPCTYGSAVTPVLGVDSFSLPDGGGADSAFSNPIRFPFGFTWPGTFSL
IIEALHTDSPDDLATENPERLISRLATQRHLTVGEEWSQDLHSSGRTDLK
YSYRFVCDEHYYGEGCSVFCRPRDDAFGHFTCGERGEKVCNPGWKGPYCT
EPICLPGCDEQHGFCDKPGECKCRVGWQGRYCDECIRYPGCLHGTCQQPW
QCNCQEGWGGLFCNQDLNYCTHHKPCKNGATCTNTGQGSYTCSCRPGYTG
ATCELGIDEC

Where reference is made herein to % homology, similarity or identity, this preferably means that the relevant % homology, similarity or identity occurs over over a region of at least 50 nucleic acid bases or amino acids, preferably over a region of at least 100 nucleic acid bases or amino acids, and preferably over the entire length of the reference sequence.

The invention further provides a pharmaceutical composition comprising i) a particle comprising a polynucleotide coding for a modulator of Notch signalling; and

ii) a same or different particle comprising a polynucleotide coding for an antigen or antigenic determinant thereof. Suitably the composition may be for biolistic delivery.

Suitably the antigen or antigenic determinant may be an allergen or antigenic determinant thereof; an autoantigen or antigenic determinant thereof; an MHC (transplant) antigen or antigenic determinant thereof; a pathogen antigen or antigenic determinant thereof; or a tumour (cancer) antigen or antigenic determinant thereof.

Ine one embodiment the particle may comprise a liposomal structure.

In an embodiment the particle is suitable for transdermal, intradermal or mucosal delivery to a subject.

Suitably the particle has a size of from about 0.005 to about 500 micrometres, preferably from about 0.05 to about 50 micrometres, preferably from about 0.5 to about 5 micrometres.

Suitably the particle comprises a carrier particle, such as a metal particle, for example selected from tungsten, gold, platinum and iridium particles.

In one embodiment the particle is suitable for administration to a subject by means of a needleless syringe or ballistic/biolistic delivery device.

In one embodiment the particle comprises a polymeric matrix or carrier.

Alternatively or in addition the particle may comprise a lipid matrix or carrier such as a cationic lipid, an anionic lipid, and/or a zwitterionic lipid, for example cetyltrimethylammonium or a phospholipid (such as phosphatidylcholine).

Preferably one or both of the polynucleotides comprises an expression control sequence operatively linked to a coding sequence. Preferably one or both of the polynucleotides is present in an expression vector and suitably one or both of the polynucleotides is circular.

In one embodiment the polynucleotide is in the form of a plasmid.

Suitably the particle further comprises a targeting molecule and/or a stabilizer.

Suitably one or both of the polynucleotides codes for a trafficking sequence selected from the group consisting of a sequence which trafficks to endoplasmic reticulum, a sequence which trafficks to a lysosome, a sequence which trafficks to an endosome, a sequence which trafficks to an intracellular vesicle, and a sequence which trafficks to the nucleus.

Suitably the particle comprises a biodegradable polymeric matrix such as a synthetic, biodegradable copolymer, such as polylactic-co-glycolic acid (PLGA). In such particles the ratio of lactic acid to glycolic acid in the copolymer is suitably within the range of about 1:2 to about 4:1 by weight for example about 65:35 by weight.

Suitably, where the particle is for phagocytosis, at least about 10%, suitably at least 50% of the nucleic acid, by weight, comprises supercoiled DNA molecules.

According to a further aspect of the invention there is provided a preparation of particles comprising a plurality of particles as described above.

According to a further aspect of the invention there is provided a pharmaceutical composition comprising a particle as described above.

According to a further aspect of the invention there is provided a method for preparing a particle as described above by combining (in any order):

i) a polynucleotide coding for a modulator of Notch signalling;

ii) a polynucleotide coding for an antigen or antigenic determinant thereof; and optionally a substrate, matrix or carrier.

Suitably the particle may be administered intradermally, transdermally or mucosally. Suitably the antigen or antigenic determinant is an allergen, autoantigen, tumour antigen Suitably the polynucleotides are administered in or on a particle or particles.

Suitably in such a method the polynucleotide coding for a modulator of Notch signalling and the polynucleotide coding for the antigen or antigenic determinant thereof are administered in or on separate particles. Alternatively the polynucleotide coding for a modulator of Notch signalling and the polynucleotide coding for the antigen or antigenic determinant thereof are administered in or on the same particle.

Preferably the particle is administered directly into skin or muscle tissue or mucosal tissue. For example, the particle may be administered topically or by inhalation.

According to a further aspect of the invention there is provided a particle acceleration device suitable for use for biolistic delivery, wherein said device is loaded with particles as described above.

According to a further aspect of the invention there is provided a polynucleotide conjugate comprising first and second polynucleotide sequences, wherein the first sequence codes for an antigen or antigenic determinant, and the second sequence codes for a polypeptide or polynucleotide for Notch signalling modulation.

Suitably the conjugate may be in the form of a vector comprising a first polynucleotide sequence coding for a modulator of the Notch signalling pathway and a second polynucleotide sequence coding for an antigen or antigenic determinant.

Suitably the conjugate may take the form of an expression vector or plasmid.

Preferably the first and second sequences are operably linked to one or more promoters.

Suitably the first and second sequences are operably linked to one or more enhancers.

Suitably the first sequence is operably linked to a first promoter and the second sequence is operably linked to a second promoter.

Preferably the first and second sequences are operably linked to one or more polyadenylation sequences.

Preferably the conjugate is suitable for expression in mammalian cells.

Suitably the conjugate comprises a selection marker.

In one embodiment, the Notch modulator and antigen (or antigenic determinant) sequences may be present in a single “dual expression” or “multiple expression” vector (as shown schematically, for example, in the Figures). This vector may be incorporated within and/or coated onto the particles as described herein.

Alternatively or in addition, the sequences may be present in separate (individual) expression vectors which may be co-incorporated within and/or co-coated onto the particles in similar manner.

### BRIEF DESCRIPTION OF THE DRAWINGS

Various preferred features and embodiments of the present invention will now be described in more detail by way of non-limiting example and with reference to the accompanying Figures, in which:

FIG. 1 shows a schematic representation of the Notch signalling pathway;

FIG. 2 shows schematic representations of the Notch ligands Jagged and Delta;

FIG. 3 shows a schematic representation of a dual expression vector useful in some embodiments of the invention;

FIG. 4 shows aligned amino acid sequences of DSL domains from various Drosophila and mammalian Notch ligands (SEQ ID NOs: 2-17);

FIG. 5 is a schematic representation showing one preferred embodiment of the invention, wherein a dual expression vector is coated onto a particle and administered to APCs which then express Notch ligand and antigen which is duly presented. Without being bound by any theory of mode of action, it is believed that the interaction of the resulting APCs with T-cells results in modified immune responses;

FIG. 6 shows a modification of the embodiment shown in FIG. 5, wherein separate vectors coding for Notch ligand and antigen are combined on or in a particle and then administered;

FIG. 7A shows a nucleic acid sequence of human Delta-1 (SEQ ID NO: 18);

FIG. 7B shows a nucleic acid sequence of human Delta-4 (SEQ ID NO: 19);

FIGS. 8A and 8B show a nucleic acid sequence of human Jagged-1 (SEQ ID NO: 20);

FIG. 9 shows plasmid maps of pBUD.OVA vectors from Example 1;

FIG. 10 shows plasmid maps of pBUD.Derp1 vectors from Example 1;

FIG. 11 shows results from Example 4;

FIG. 12 shows further results from Example 4;

FIG. 13 shows results from Example 5;

FIG. 14 shows further results from Example 5;

FIG. 15 shows results from Example 7 (a);

FIG. 16 shows results from Example 7 (b);

FIG. 17 shows results from Example 7 (c);

FIG. 18 shows results from Example 8 (a);

FIG. 19 shows results from Example 8 (b);

FIG. 20 shows results from Example 8 (c);

FIG. 21 shows results from Example 9 (a);

FIG. 22 shows results from Example 9 (b);

FIG. 23 shows results from Example 9 (c);

FIG. 24 shows results from Example 10;

FIG. 25 shows results from Example 10;

FIG. 26 shows results from Example 11;

FIG. 27 shows results from Example 12;

FIG. 28 shows results from Example 12;

FIG. 29 shows results from Example 13;

FIG. 30 shows results from Example 14;

FIG. 31 shows results from Example 14;

FIG. 32 shows results from Example 14;

FIG. 33 shows results from Example 14;

FIG. 34 shows results from Example 14;

FIG. 35 shows results from Example 15;

FIG. 36 shows results from Example 15;

FIG. 37 shows results from Example 15;

FIG. 38 shows results from Example 16;

FIG. 39 shows results from Example 16;

FIG. 40 shows results from Example 16;

FIG. 41 shows results from Example 17;

FIG. 42 shows results from Example 17;

FIG. 43 shows results from Example 18;

FIG. 44 shows results from Example 18;

FIG. 45 shows results from Example 18;

FIG. 46 shows results from Example 19; and

FIG. 47 shows results from Example 20.

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and J. E. Coligan, A. M. Kruisbeek, D. H. Margulies, E. M. Shevach and W. Strober (1992 and periodic supplements; Current Protocols in Immunology, John Wiley & Sons, New York, N.Y.). Each of these general texts is hereby incorporated herein by reference.

For the avoidance of doubt, Drosophila and vertebrate names are used interchangeably and all homologues are included within the scope of the invention.

Notch Signalling

As used herein, the expression “Notch signalling” is synonymous with the expression “the Notch signalling pathway” and refers to any one or more of the upstream or downstream events that result in, or from, (and including) activation of the Notch receptor.

Preferably, by “Notch signalling” we refer to any event directly upstream or downstream of Notch receptor activation or inhibition including activation or inhibition of Notch/Notch ligand interactions, upregulation or downregulation of Notch or Notch ligand expression or activity and activation or inhibition of Notch signalling transduction including, for example, proteolytic cleavage of Notch and upregulation or downregulation of the Ras-Jnk signalling pathway.

Thus, by “Notch signalling” we refer to the Notch signalling pathway as a signal tranducing pathway comprising elements which interact, genetically and/or molecularly, with the Notch receptor protein. For example, elements which interact with the Notch protein on both a molecular and genetic basis are, by way of example only, Delta, Serrate and Deltex. Elements which interact with the Notch protein genetically are, by way of example only, Mastermind, Hairless, Su(H) and Presenilin.

In one aspect, Notch signalling includes signalling events taking place extracellularly or at the cell membrane. In a further aspect, it includes signalling events taking place intracellularly, for example within the cell cytoplasm or within the cell nucleus.

Modulators of Notch Signalling

The term “modulate” as used herein refers to a change or alteration in the biological activity of the Notch signalling pathway or a target signalling pathway thereof. The term “modulator” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator” may refer to agonists of Notch signalling, i.e. compounds which stimulate or upregulate, at least to some extent, the normal biological activity of the Notch signalling pathway. Conveniently such compounds may be referred to as upregulators or agonists. Preferably the modulator is an agonist of Notch signalling, and preferably an agonist of the Notch receptor (e.g. an agonist of the Notch1, Notch2, Notch3 and/or Notch4 receptor).

The polynucleotide may also code for an antibody.

The term “antibody” includes intact molecules as well as fragments thereof, such as Fab, F(ab′)2, Fv and scFv which are capable of binding the epitopic determinant. These antibody fragments retain some ability to selectively bind with its antigen or receptor and include, for example:

(i) Fab, the fragment which contains a monovalent antigen-binding fragment of an antibody molecule can be produced by digestion of whole antibody with the enzyme papain to yield an intact light chain and a portion of one heavy chain;

(ii) Fab′, the fragment of an antibody molecule can be obtained by treating whole antibody with pepsin, followed by reduction, to yield an intact light chain and a portion of the heavy chain; two Fab′ fragments are obtained per antibody molecule;

(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;

(iv) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference).

Modulators may be synthetic compounds or natural isolated compounds.

In one form the agent for modulation of the Notch signalling pathway may code for a protein for Notch signalling transduction. By a protein which is for Notch signalling transduction is meant a molecule which participates in signalling through Notch receptors including activation of Notch, the downstream events of the Notch signalling pathway, transcriptional regulation of downstream target genes and other non-transcriptional downstream events (e.g. post-translational modification of existing proteins). More particularly, the protein is a domain that allows activation of target genes of the Notch signalling pathway, or a polynucleotide sequence which codes therefor.

A very important component of the Notch signalling pathway is Notch receptor/Notch ligand interaction. Thus Notch signalling may involve changes in expression, nature, amount or activity of Notch signalling pathway membrane proteins or G-proteins or Notch signalling pathway enzymes such as proteases, kinases (e.g. serine/threonine kinases), phosphatases, ligases (e.g. ubiquitin ligases) or glycosyltransferases. Alternatively the signalling may involve changes in expression, nature, amount or activity of DNA binding elements such as transcription factors.

In the present invention Notch signalling preferably means specific signalling, meaning that the signalling results substantially or at least predominantly from the Notch signalling pathway, and preferably from Notch/Notch ligand interaction, rather than any other significant interfering or competing cause, such as cytokine signalling. Thus, in a preferred embodiment, Notch signalling excludes cytokine signalling.

The Notch signalling pathway is described in more detail below.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H) to contribute an activation domain that allows Su(H) to activate the transcription of E(spl) as well as other target genes. It should also be noted that Su(H) is not required for all Notch-dependent decisions, indicating that Notch mediates some cell fate choices by associating with other DNA-binding transcription factors or be employing other mechanisms to transduce extracellular signals.

According to one aspect of the present invention the active agent may code for Notch or a fragment thereof which retains the signalling transduction ability of Notch or an analogue of Notch which has the signalling transduction ability of Notch.

As used herein the term “analogue of Notch” includes variants thereof which retain the signalling transduction ability of Notch. By “analogue” we include a protein which has Notch signalling transduction ability, but generally has a different evolutionary origin to Notch. Analogues of Notch include proteins from the Epstein Barr virus (EBV), such as EBNA2, BARF0 or LMP2A.

By a protein which is for Notch signalling activation we mean a molecule which is capable of activating Notch, the Notch signalling pathway or any one or more of the components of the Notch signalling pathway.

In one embodiment, the active agent may code for a Notch ligand. Notch ligands of use in the present invention include endogenous Notch ligands which are typically capable of binding to a Notch receptor polypeptide present in the membrane of a variety of mammalian cells, for example hemapoietic stem cells.

The term “Notch ligand” as used herein means an agent capable of interacting with a Notch receptor to cause a biological effect. The term as used herein therefore includes naturally occurring protein ligands such as Delta and Serrate/Jagged as well as antibodies to the Notch receptor, and polypeptides which have corresponding biological effects to the natural ligands. Preferably the Notch ligand interacts with the Notch receptor by binding.

Particular examples of mammalian Notch ligands identified to date include the Delta family, for example Delta or Delta-like 1 (Genbank Accession No. AF003522—Homo sapiens), Delta-3 (Genbank Accession No. AF084576—Rattus norvegicus) and Delta-like 3 (Mus musculus) (Genbank Accession No. NM_{—}016941—Homo sapiens) and U.S. Pat. No. 6,121,045 (Millennium), Delta-4 (Genbank Accession Nos. AB043894 and AF 253468—Homo sapiens) and the Serrate family, for example Serrate-1 and Serrate-2 (WO97/01571, WO96/27610 and WO92/19734), Jagged-1 (Genbank Accession No. U73936—Homo sapiens) and Jagged-2 (Genbank Accession No. AF029778—Homo sapiens), and LAG-2. Homology between family members is extensive.

In one embodiment, an activator may code for a constitutively active Notch receptor or Notch intracellular domain.

In an alternative embodiment, an activator of Notch signalling will act downstream of the Notch receptor. Thus, for example, the activator of Notch signalling may be a constitutively active Deltex polypeptide or a polynucleotide encoding such a polypeptide. Other downstream components of the Notch signalling pathway of use in the present invention include the polypeptides involved in the Ras/MAPK cascade catalysed by Deltex, polypeptides involved in the proteolytic cleavage of Notch such as Presenilin and polypeptides involved in the transcriptional regulation of Notch target genes, preferably in a constitutively active form.

By polypeptide for Notch signalling activation is also meant any polypeptides expressed as a result of Notch activation and any polypeptides involved in the expression of such polypeptides, or polynucleotides coding for such polypeptides.

Any one or more of appropriate targets—such as an amino acid sequence and/or nucleotide sequence—may be used for identifying a compound capable of modulating the Notch signalling pathway and/or a targeting molecule in any of a variety of drug screening techniques. The target employed in such a test may be free in solution, affixed to a solid support, borne on a cell surface, or located intracellularly.

An agent may be considered to modulate Notch-Notch ligand interactions if it is capable of promoting the interaction of Notch with its ligands, preferably to an extent sufficient to provide therapeutic efficacy.

The expression “Notch-Notch ligand” as used herein means the interaction between a Notch family member and a ligand capable of binding to one or more such member. Thus by the expression “upregulating interaction of Notch or a Notch-ligand” we mean promoting the interaction of Notch in a lymphocyte or APC with a Notch ligand or promoting the interaction of a Notch ligand in a lymphocyte or APC with Notch. Preferably the lymphocyte is a T cell.

Polypeptides and Polynucleotides for Notch Signalling Transduction

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299—Homo sapiens).

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs in the Golgi apparatus and is mediated by a furin-like convertase.

Notch receptors are inserted into the membrane as heterodimeric molecules consisting of an extracellular domain containing up to 36 epidermal growth factor (EGF)-like repeats [Notch 1/2=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura K, et al. (1995) Curr. Biol. 5:1416-1423 (Tamura)). The Notch ligands also display multiple EGF-like repeats in their extracellular domains together with a cysteine-rich DSL (Delta-Serrate Lag2) domain that is characteristic of all Notch ligands (Artavanis-Tsakomas et al. (1995) Science 268:225-232, Artavanis-Tsakomas et al. (1999) Science 284:770-776).

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber et al. (1993) Genes Dev 7(10):1949-65 (Lieber)). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter, E. H. et al. (1998) Nature 393(6683):382-6 (Schroeter)). It is thought that the proteolytic cleavage step that releases the cdc10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl, G. et al. (1998) Cell 93(4):649-60 (Struhl)). The NotchIC-CBF1 complexes then activate target genes, such as the bHLH proteins HES (hairy-enhancer of split like) 1 and 5 (Weinmaster G. (2000) Curr. Opin. Genet. Dev. 10:363-369 (Weinmaster)). This nuclear function of Notch has also been shown for the mammalian Notch homologue (Lu, F. M. et al. (1996) Proc Natl Acad Sci 93(11):5663-7 (Lu)).

S3 processing occurs only in response to binding of Notch ligands Delta or Serrate/Jagged. The post-translational modification of the nascent Notch receptor in the Golgi (Munro S, Freeman M. (2000) Curr. Biol. 10:813-820 (Munro); Ju B J, et al. (2000) Nature 405:191-195 (Ju)) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney D J, et al. (2000) Nature 406:369-375 (Moloney), Brucker K, et al. (2000) Nature 406:411-415 (Brucker)). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin V M, et al. (1997) Nature 387:908-912 (Panin), Hicks C, et al. (2000) Nat. Cell. Biol. 2:515-520 (Hicks)). Although Drosophila has a single Fringe gene, vertebrates are known to express multiple genes (Radical, Manic and Lunatic Fringes) (Irvine K D (1999) Curr. Opin. Genet. Devel. 9:434-441 (Irvine)).

In an alternative embodiment, the activator of Notch signalling may act downstream of the Notch receptor. Thus, for example, the activator of Notch signalling may be a constitutively active Deltex polypeptide or a polynucleotide encoding such a polypeptide. Other downstream components of the Notch signalling pathway of use in the present invention include Deltex-1, Deltex-2, Deltex-3, Suppressor of Deltex (SuDx), Numb and isoforms thereof, Numb associated Kinase (NAK), Notchless, Dishevelled (Dsh), emb5, Fringe genes (such as Radical, Lunatic and Manic), PON, LNX, Disabled, Numblike, Nur77, NFkB2, Mirror, Warthog, Engrailed-1 and Engrailed-2, Lip-1 and homologues thereof, the polypeptides involved in the Ras/MAPK cascade modulated by Deltex, polypeptides involved in the proteolytic cleavage of Notch such as Presenilin and polypeptides involved in the transcriptional regulation of Notch target genes, preferably in a constitutively active form, and analogues, derivatives, variants and fragments thereof.

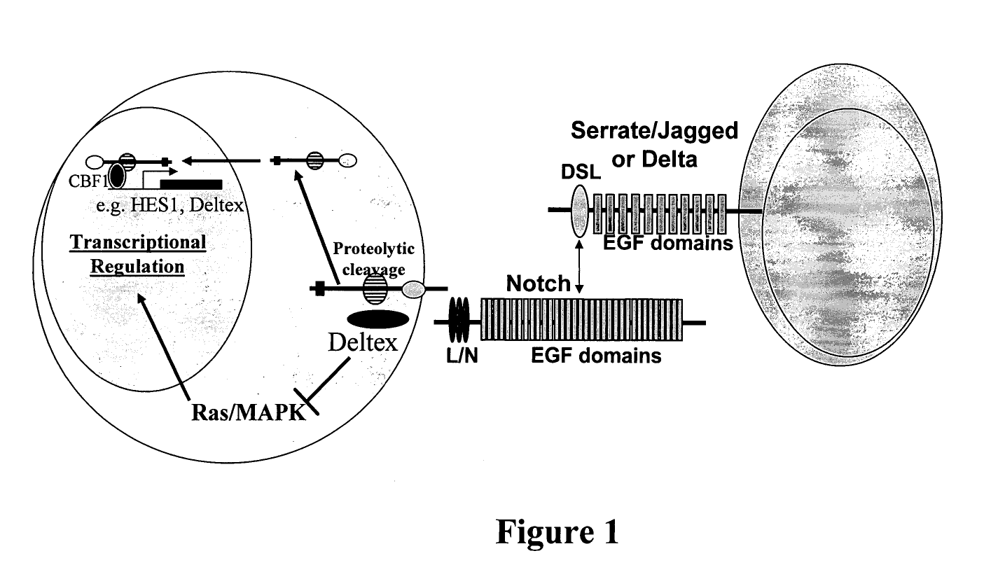

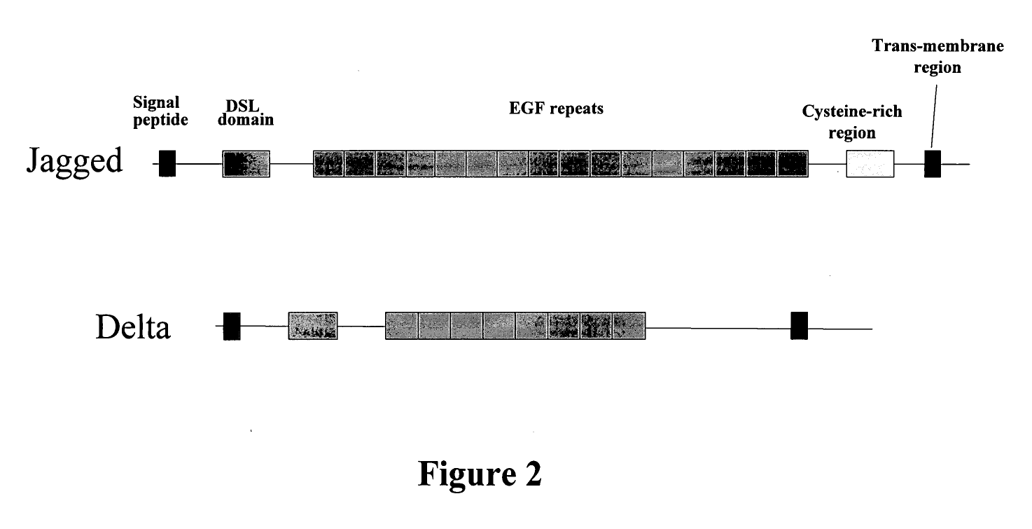

Signal transduction from the Notch receptor can occur via two different pathways (FIG. 1). The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex. Unlike CBF1, Deltex does not move to the nucleus following Notch activation but instead can interact with Grb2 and modulate the Ras-JNK signalling pathway.

Thus, signal transduction from the Notch receptor can occur via two different pathways both of which are illustrated in FIG. 1. Target genes of the Notch signalling pathway include Deltex, genes of the Hes family (Hes-1 in particular), Enhancer of Split [E(spl)] complex genes, IL-10, CD-23, CD-4 and Dll-1.

Deltex, an intracellular docking protein, replaces Su(H) as it leaves its site of interaction with the intracellular tail of Notch. Deltex is a cytoplasmic protein containing a zinc-finger (Artavanis-Tsakomas et al. (1995) Science 268:225-232; Artavanis-Tsakomas et al. (1999) Science 284:770-776; Osborne B, Miele L. (1999) Immunity 11:653-663 (Osborne)). It interacts with the ankyrin repeats of the Notch intracellular domain. Studies indicate that Deltex promotes Notch pathway activation by interacting with Grb2 and modulating the Ras-JNK signalling pathway (Matsuno et al. (1995) Development 121(8):2633-44; Matsuno K, et al. (1998) Nat. Genet. 19:74-78). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich et al. (1998) Mol. Cell. Biol. 18:2230-2239 (Ordentlich)). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi K. et al. (1994) J Biol Chem 269(7):150-6 (Takebayashi)) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister C. et al. (1999) Mech Dev 85(1-2):173-7 (Leimeister)) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

Interleukin-10 (IL-10) was first characterised in the mouse as a factor produced by Th2 cells which was able to suppress cytokine production by Th1 cells. It was then shown that IL-10 was produced by many other cell types including macrophages, keratinocytes, B cells, Th0 and Th1 cells. It shows extensive homology with the Epstein-Barr bcrf1 gene which is now designated viral IL-10. Although a few immunostimulatory effects have been reported, it is mainly considered as an immunosuppressive cytokine. Inhibition of T cell responses by IL-10 is mainly mediated through a reduction of accessory functions of antigen presenting cells. IL-10 has notably been reported to suppress the production of numerous pro-inflammatory cytokines by macrophages and to inhibit co-stimulatory molecules and MHC class II expression. IL-10 also exerts anti-inflammatory effects on other myeloid cells such as neutrophils and eosinophils. On B cells, IL-10 influences isotype switching and proliferation. More recently, IL-10 was reported to play a role in the induction of regulatory T cells and as a possible mediator of their suppressive effect. Although it is not clear whether it is a direct downstream target of the Notch signalling pathway, its expression has been found to be strongly up-regulated coincident with Notch activation. The mRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. The sequence for CD-23 may be found in GenBank ref. No. GI1783344.

CTLA4 (cytotoxic T-lymphocyte activated protein 4) is an accessory molecule found on the surface of T-cells which is thought to play a role in the regulation of airway inflammatory cell recruitment and T-helper cell differentiation after allergen inhalation. The promoter region of the gene encoding CTLA4 has CBF1 response elements and its expression is upregulated as a result of Notch activation. The sequence of CTLA4 can be found in GenBank Accession No. L15006.

Dlx-1 (distalless-1) (McGuinness T. Et al (1996) Genomics 35(3):473-85 (McGuiness)) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

As described above the Notch receptor family participates in cell-cell signalling events that influence T cell fate decisions. In this signalling NotchIC localises to the nucleus and functions as an activated receptor. Mammalian NotchIC interacts with the transcriptional repressor CBF1. It has been proposed that the NotchIC cdc10/ankyrin repeats are essential for this interaction. Hsieh et al (Hsieh et al. (1996) Molecular & Cell Biology 16(3):952-959) suggests rather that the N-terminal 114 amino acid region of mouse NotchIC contains the CBF1 interactive domain. It is also proposed that NotchIC acts by targeting DNA-bound CBF1 within the nucleus and abolishing CBF1-mediated repression through masking of the repression domain. It is known that Epstein Barr virus (EBV) immortalizing protein EBNA” also utilises CBF1 tethering and masking of repression to upregulate expression of CBF1-repressed B-cell genes. Thus, mimicry of Notch signal transduction is involved in EBV-driven immortalization. Strobl et al. (Strobl et al. (2000) J Virol 74(4):1727-35) similarly reports that “EBNA2 may hence be regarded as a functional equivalent of an activated Notch receptor”. Other EBV proteins which fall in this category include BARF0 (Kusano and Raab-Truab (2001) J Virol 75(1):384-395 (Kusano and Raab-Traub)) and LMP2A.

Polypeptides and Polynucleotides for Notch Signalling Activation

Examples of mammalian Notch ligands identified to date include the Delta family, for example Delta-1 (Genbank Accession No. AF003522—Homo sapiens), Delta-3 (Genbank Accession No. AF084576—Rattus norvegicus) and Delta-like 3 (Mus musculus), the Serrate family, for example Serrate-1 and Serrate-2 (WO97/01571, WO96/27610 and WO92/19734), Jagged-1 and Jagged-2 (Genbank Accession No. AF029778—Homo sapiens), and LAG-2. Homology between family members is extensive.

For example, an exemplary human Delta 4 is contained in a plasmid which was deposited with the American Type Culture Collection (ATCC) on Mar. 5, 1997, and has been assigned ATCC accession number 98348 (e.g. see U.S. Pat. No. 6,121,045; Millennium)

By a “homologue” is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequnce of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example the National Center for Biotechnology Information website and Ausubel et al., Current Protocols in Molecular Biology (1995), John Wiley & Sons, Inc.)

Notch ligands identified to date have a diagnostic DSL domain (D. Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain at the N-terminus and up to 14 or more EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the target cell.

The activating molecule of the present invention may, in an alternative embodiment, be capable of modifying Notch-protein expression or presentation on the cell membrane or signalling pathways. Agents that enhance the presentation of a fully functional Notch-protein on the target cell surface include matrix metalloproteinases such as the product of the Kuzbanian gene of Drosophila (Dkuz et al. (1997) Cell 90: 271-280 (Dkuz)) and other ADAMALYSIN gene family members.

Notch Ligand Domains

As discussed above, Notch ligands typically comprise a number of distinctive domains. Some predicted/potential domain locations for various naturally occurring human Notch ligands (based on amino acid numbering in the precursor proteins) are shown below:

| | | |
|---|---|---|
| Component | Amino acids | Proposed function/domain |

Human Delta 1

| | | |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| | | |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| | | |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| | | |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| | | |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence (SEQ ID NO: 21):

Cys Xaa Xaa Xaa Xaa Xaa Xaa Xaa Xaa Cys Xaa Xaa Xaa Cys Xaa Xaa
Xaa Xaa Xaa Xaa Xaa Xaa Xaa Xaa Xaa Cys Xaa Xaa Xaa Xaa Xaa Xaa
Xaa Cys Xaa Xaa Xaa Xaa Xaa Xaa Xaa Xaa Cys

Preferably the DSL domain may include most or all of the following consensus amino acid sequence (SEQ ID NO: 22):

Cys Xaa Xaa Xaa ARO ARO Xaa Xaa Xaa Cys Xaa Xaa Xaa Cys BAS NOP
BAS ACM ACM Xaa ARO NOP ARO Xaa Xaa Cys Xaa Xaa Xaa NOP Xaa Xaa
Xaa Cys Xaa Xaa NOP ARO Xaa NOP Xaa Xaa Cys

wherein:

ARO is an aromatic amino acid residue, such as tyrosine, phenylalanine, tryptophan or histidine;

NOP is a non-polar amino acid residue such as glycine, alanine, proline, leucine, isoleucine or valine;

BAS is a basic amino acid residue such as arginine or lysine; and

ACM is an acid or amide amino acid residue such as aspartic acid, glutamic acid, asparagine or glutamine.

Preferably the DSL domain may include most or all of the following consensus amino acid sequence (SEQ ID NO: 23):

Cys Xaa Xaa Xaa Tyr Tyr Xaa Xaa Xaa Cys Xaa Xaa Xaa Cys Arg Pro
Arg Asx Asp Xaa Phe Gly His Xaa Xaa Cys Xaa Xaa Xaa Gly Xaa Xaa
Xaa Cys Xaa Xaa Gly Trp Xaa Gly Xaa Xaa Cys

(wherein Xaa may be any amino acid and Asx is either aspartic acid or asparagine).

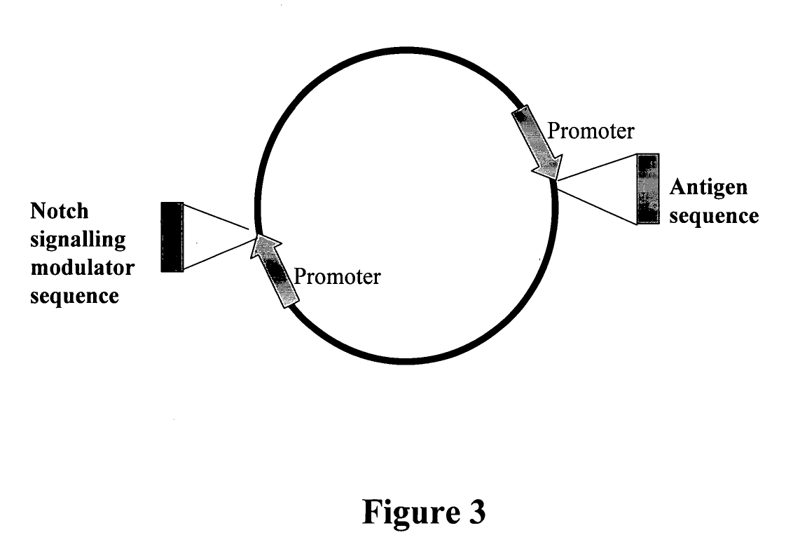

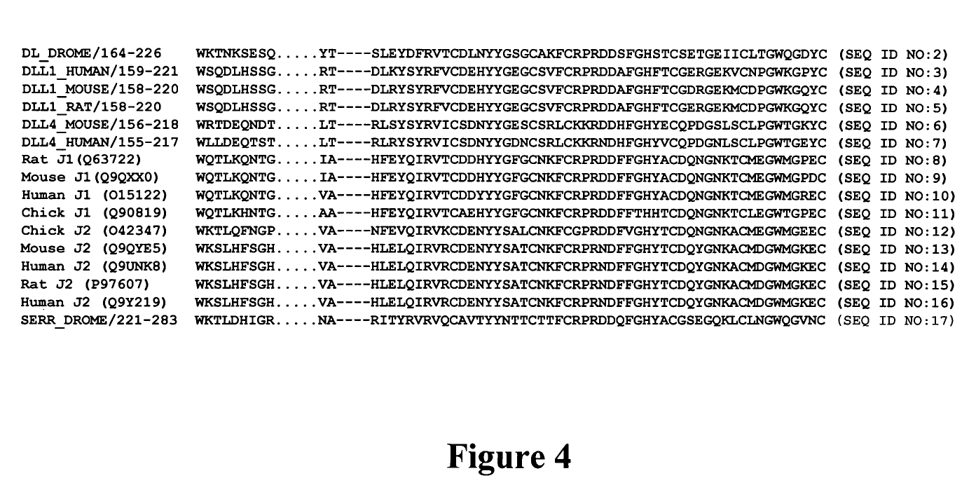

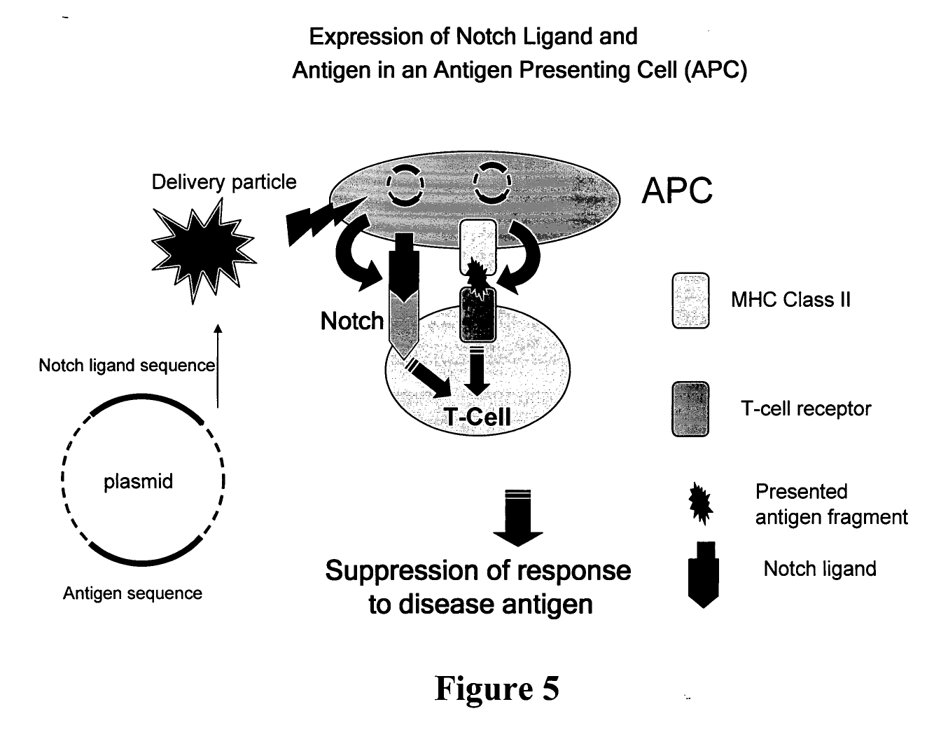

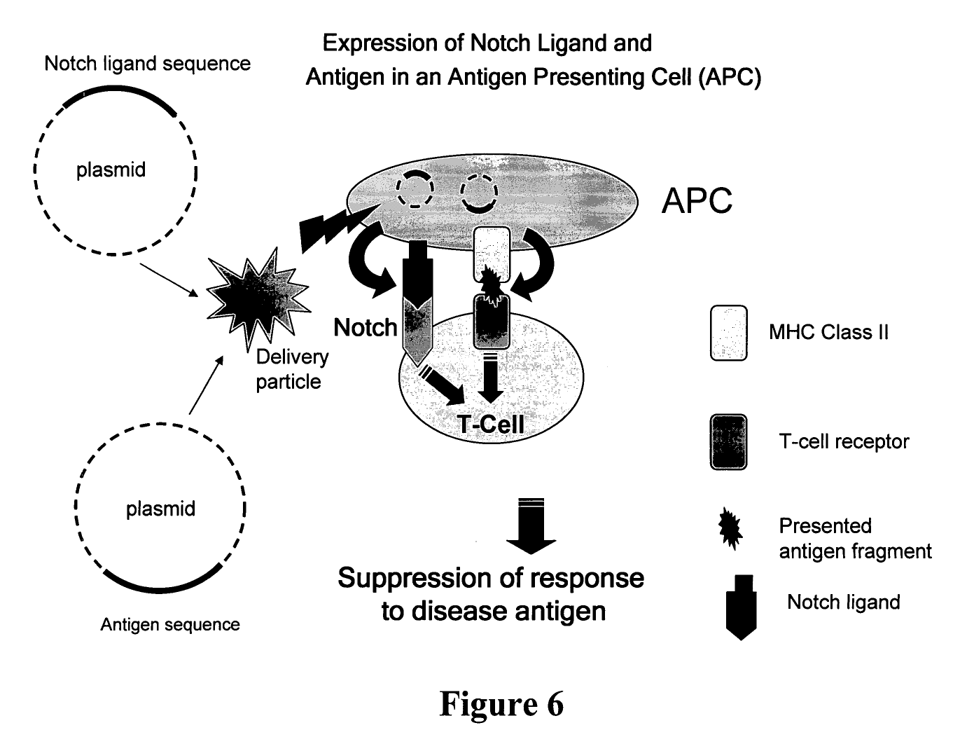

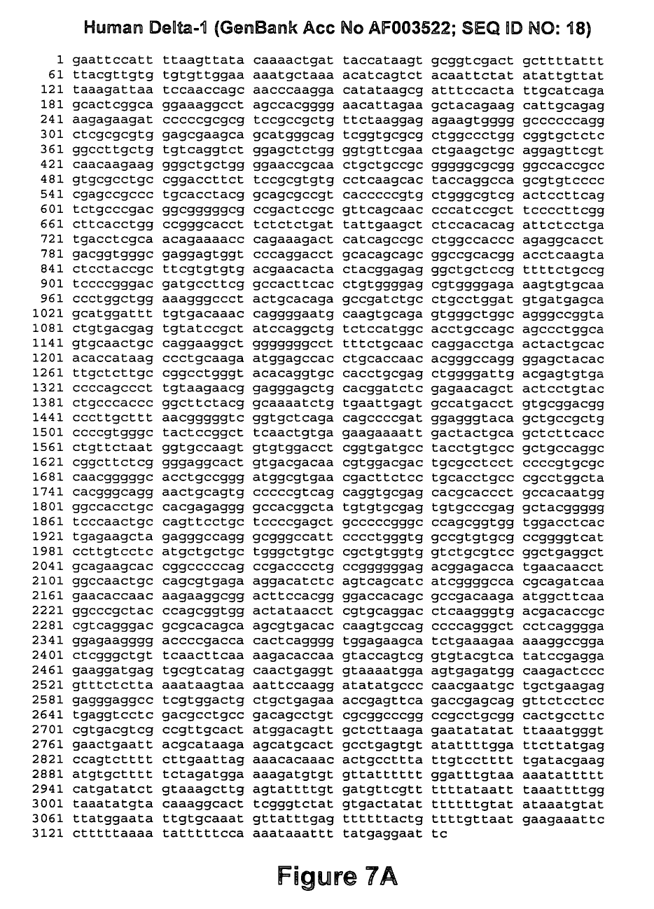

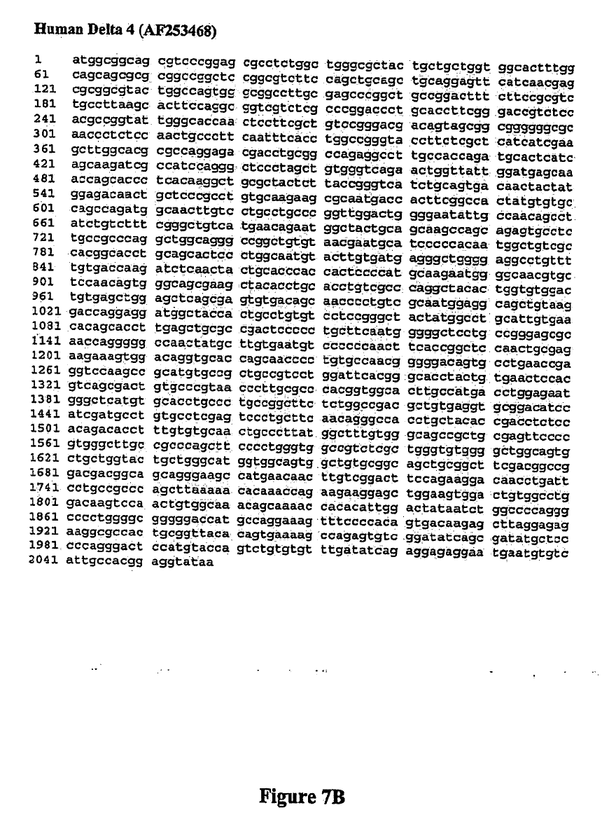

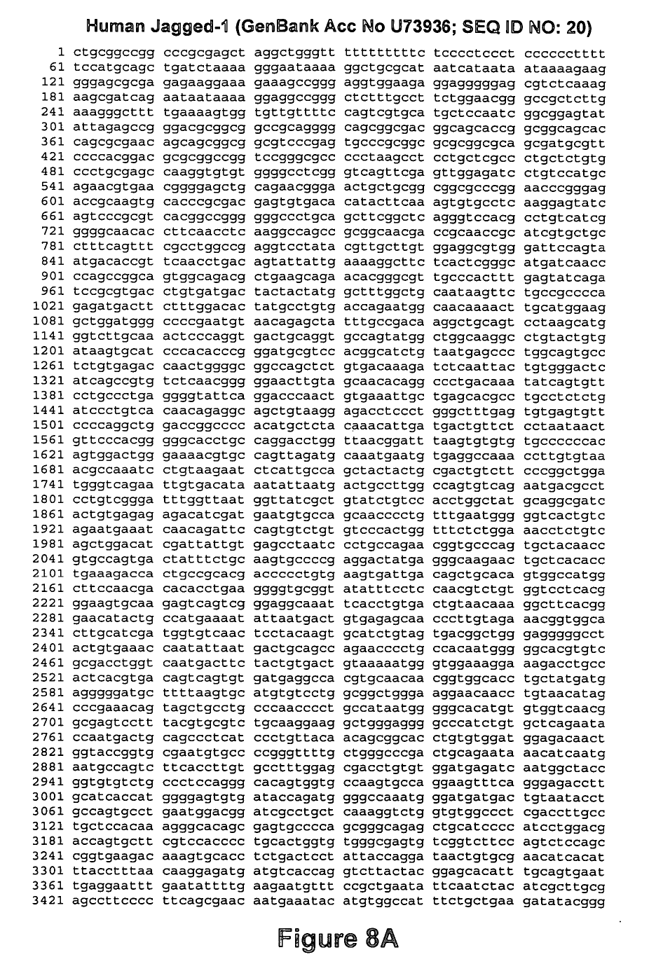

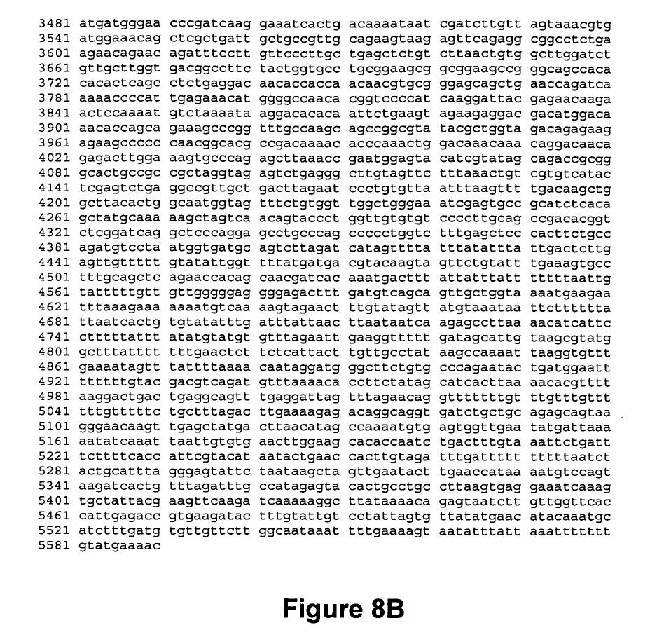

An alignment of DSL domains from Notch ligands from various sources is shown in FIG. 3.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

It will be appreciated that the term “DSL domain” as used herein includes sequence variants, fragments, derivatives and mimetics having activity corresponding to naturally occurring domains.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors IX and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE a typical EGF domain may include six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain (SEQ ID NO: 24): 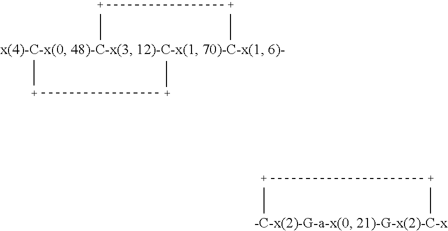
wherein:
‘C’: conserved cysteine involved in a disulfide bond.
‘G’: often conserved glycine
‘a’: often conserved aromatic amino acid
‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

It will be appreciated that the term “EGF domain” as used herein includes sequence variants, fragments, derivatives and mimetics having activity corresponding to naturally occurring domains.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X % identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

The term “transmembrane domain” includes a domain which is retained within a cell membrane, which preferably anchors the protein or polypeptide to the membrane when expressed.

The term “membrane binding domain” includes a domain which binds to a cell membrane without necessarily passing through it, or passing entirely through it.

The term “heterologous amino acid sequence” or “heterologous nucleotide sequence” as used herein means a sequence which is not found in the native Notch ligand or its coding sequence.

Whether an agent can be used for activating Notch may be determined using suitable screening assays, for example, as described in our co-pending International Patent Application claiming priority from GB 0118153.6 (WO 03/012441, Lorantis, e.g. Example 8) and the examples herein.

Activation of Notch signalling may also be achieved by repressing inhibitors of the Notch signalling pathway. As such, polypeptides for Notch signalling activation will include molecules capable of repressing any Notch signalling inhibitors. Preferably the molecule will be a polypeptide, or a polynucleotide encoding such a polypeptide, that decreases or interferes with the production or activity of compounds that are capable of producing an decrease in the expression or activity of Notch, Notch ligands, or any downstream components of the Notch signalling pathway. In a preferred embodiment, the molecules will be capable of repressing polypeptides of the Toll-like receptor protein family and growth factors such as the bone morphogenetic protein (BMP), BMP receptors and activins, derivatives, fragments, variants and homologues thereof.

Substances Capable of Upregulating Endogenous Notch or Notch Ligand Expression

Substances that may be used to upregulate Notch ligand expression include polypeptides that bind to and reduce or neutralise the activity of bone morphogenetic proteins (BMPs). Binding of extracellular BMPs (Wilson and Hemmati-Brivanlou (1997) Neuron 18:699-710; Hemmati-Brivanlou and Melton (1997) Cell 88:13-17) to their receptors leads to down-regulated Delta transcription due to the inhibition of the expression of transcription factors of the achaete/scute complex. This complex is believed to be directly involved in the regulation of Delta expression. Thus, any substance that inhibits BMP expression and/or inhibits the binding of BMPs to their receptors may be capable of producing an increase in the expression of Notch ligands such as Delta and/or Serrate. Particular examples of such inhibitors include Noggin (Valenzuela et al. (1995) J. Neurosci. 15:6077-6084 (Valenzuela)), Chordin (Sasai et al. (1994) Cell 79:779-790 (Sasai)), Follistatin (Iemura et al. (1998) PNAS 95:9337-9345 (Iemura)), Xnr3, and derivatives and variants thereof. Noggin and Chordin bind to BMPs thereby preventing activation of their signalling cascade which leads to decreased Delta transcription. Consequently, increasing Noggin and Chordin levels may lead to increase Notch ligand, in particular Delta, expression.

Furthermore, any substance that upregulates expression of transcription factors of the achaete/scute complex may also upregulate Notch ligand expression.

Other suitable substances that may be used to upregulate Notch ligand expression include transforming growth factors such as members of the fibroblast growth factor (FGF) family. The FGF may be a mammalian basic FGF, acidic FGF or another member of the FGF family such as an FGF-1, FGF-2, FGF-3, FGF-4, FGF-5, FGF-6, FGF-7. Preferably the FGF is not acidic FGF (FGF-1; Zhao et al. (1995) J. Immunol. 155:3904-3911 (Zhao))). Most preferably, the FGF is a member of the FGF family which acts by stimulating the upregulation of expression of a Serrate polypeptide on APCs. The inventors have shown that members of the FGF family can upregulate Serrate-1 gene expression in APCs.

Immunosuppressive cytokines may also be used to upregulate Notch ligand expression. Examples include members of the TGF-β family such as TGF-β-1 and TGF-β-2, and interleukins such as IL-4, IL-10 and IL-13, and FLT3 ligand.

The substance capable of upregulating expression of Notch or a Notch ligand may be selected from polypeptides and fragments thereof, linear peptides, cyclic peptides, synthetic and natural compounds including low molecular weight organic or inorganic compounds. The substances capable of upregulating expression of a Notch ligand may be derived from a biological material such as a component of extracellular matrix. Suitable extracellular matrix components are derived from immunologically privileged sites such as the eye. For example aqueous humour or components thereof may be used.

Polypeptide substances such as Noggin, FGFs and TGF-β may be purified from mammalian cells, obtained by recombinant expression in suitable host cells or obtained commercially. Alternatively, nucleic acid constructs encoding the polypeptides may be introduced into APCs and/or lymphocytes (T cells) by transfection using standard techniques or viral infection/transduction. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the APC or lymphocte (T cell).

It is particularly preferred to use combinations of substances, for example a combination of at least two substances. In a preferred embodiment, an immunosuppressive cytokine is used in combination with another substance capable of upregulating Notch ligand expression. Other examples of preferred combinations include at least one substance capable of upregulating Serrate expression (such as FGF), preferably in an APC, together with at least one substance capable of upregulating Delta expression (such as Noggin or Chordin), preferably in a T cell. Alternatively, a preferred combination comprises at least one substance which acts via inhibition of binding of BMPs to their receptors together with at least one substance which has a different mode of action.

Preferably, the composition, preferably a nucleic acid sequence, for use in the present invention is capable of upregulating Serrate and Delta, preferably Serrate 1 and Serrate 2 as well as Delta 1 and Delta 3 expression in APCs such as dendritic cells.

Preferably, the substance for use in the present invention is capable of upregulating Serrate expression in APCs such as dendritic cells. In particular, the substance may be capable of upregulating Serrate expression but not Delta expression in APCs. Alternatively, the substance for use in the present invention is capable of upregulating Delta expression in T cells such as CD4⁺ helper T cells or other cells of the immune system that express Delta (for example in response to stimulation of cell surface receptors). In particular, the substance may be capable of upregulating Delta expression but not Serrate expression in T cells. In a particularly preferred embodiment, the substance is capable of upregulating Notch ligand expression in both T cells and APC, for example Serrate expression in APCs and Delta expression in T cells.

Suitable substances for use according to the present invention may be conveniently identified using a simple screening procedure. In one such assay procedure; lymphocytes, such as T cells, or APCs in culture may be contacted with a candidate substance and the effect on expression of an endogenous Notch ligand, such as Delta or Serrate, determined, for example by (i) measuring transcription initiated from the gene encoding the Notch ligand as described in the Examples or by quantitative-reverse transcriptase-polymerase chain reaction (RT-PCR); (ii) detecting Notch ligand protein by techniques such as Western blotting of cell extracts, immunohistochemistry or flow cytometry; and/or (iii) functional assays such as cell adhesion assays.

The present invention also relates to modification of Notch-protein expression or presentation on the cell membrane or signalling pathways. Agents that enhance the presentation of a fully functional Notch-protein on the lymphocyte or APC surface include matrix metalloproteinases such as the product of the Kuzbanian gene of Drosophila (Dkuz et al (1997) Cell 90: 271-280) and other ADAMALYSIN gene family members.

In more detail, whether a substance can be used for modulating Notch-Notch ligand expression may be determined using suitable screening assays.

Screening assays for the detection of increased Notch, Notch ligand expression and/or processing include:

Notch-Notch ligand expression may be assessed following exposure of isolated cells to test compounds in culture using for example:

(a) at the protein level by specific antibody staining using immunohistochemistry or flow cytometry.

(b) at the RNA level by quantitative—reverse transcriptase-polymerase chain reaction (RT-PCR). RT-PCR may be performed using a control plasmid with in-built standards for measuring endogenous gene expression with primers specific for Notch 1 and Notch 2, Serrate 1 and Serrate 2, Delta 1 and Delta 2 and Delta 3. This construct may be modified as new ligand members are identified.

(c) at the functional level in cell adhesion assays.

Increased Notch ligand or Notch expression should lead to increased adhesion between cells expressing Notch and its ligands. Test cells will be exposed to a particular treatment in culture and radiolabelled or flourescein labelled target cells (transfected with Notch/Notch ligand protein) will be overlayed. Cell mixtures will be incubated at 37° C. for 2 hours. Nonadherent cells will be washed away and the level of adherence measured by the level of radioactivity/immunofluorescence at the plate surface.

Using such methods it is possible to detect compounds or Notch-ligands that affect the expression or processing of a Notch-protein or Notch-ligand. The invention also relates to compounds, or Notch-ligands detectable by these assays methods, and also to their use in the methods of the present invention.

These procedures may also be used to identify particularly effective combinations of substances for use according to the present invention.

Polypeptides and Polynucleotides for Notch Signalling Inhibition

Substances that may be used to inhibit Notch ligand expression include nucleic acid sequences encoding polypeptides that affect the expression of genes encoding Notch ligands. For instance, for Delta expression, binding of extracellular BMPs (bone morphogenetic proteins; Wilson and Hemmati-Brivanlou (1997) Neuron 18: 699-710; Hemmati-Brivanlou and Melton (1997) Cell 88: 13-17) to their receptors leads to down-regulated Delta transcription due to the inhibition of the expression of transcription factors of the achaete/scute complex. This complex is believed to be directly involved in the regulation of Delta expression. Thus, any polypeptide that upregulates BMP expression and/or stimulates the binding of BMPs to their receptors may be capable of producing a decrease in the expression of Notch ligands such as Delta and/or Serrate. Examples may include nucleic acids encoding BMPs themselves. Furthermore, any substance that inhibits expression of transcription factors of the achaete/scute complex may also downregulate Notch ligand expression.

Members of the BMP family include BMP1 to BMP6, BMP7 also called OP1, OP2 (BMP8) and others. BMPs belong to the transforming growth factor beta (TGF-beta) superfamily, which includes, in addition to the TGF-betas, activins/inhibins (e.g., alpha-inhibin), mullerian inhibiting substance, and glial cell line-derived neurotrophic factor.

Other examples of polypeptides that inhibit the expression of Delta and/or Serrate include the Toll-like receptor (Medhzhitov et al. (1997) Nature 388:394-397 (Medhzhitov)) or any other receptors linked to the innate immune system (for example CD14, complement receptors, scavenger receptors or defensin proteins), and other polypeptides that decrease or interfere with the production of Noggin (Valenzuela et al. (1995) J. Neurosci 15: 6077-6084), Chordin (Sasai et al. (1994) Cell 79:779-790), Follistatin (Iemura et al. (1998) PNAS 95:9337-9345), Xnr3, and derivatives and variants thereof. Noggin and Chordin bind to BMPs thereby preventing activation of their signalling cascade which leads to decreased Delta transcription. Consequently, reducing Noggin and Chordin levels may lead to decreased Notch ligand, in particular Delta, expression (Hoyne et al, 2000).

In more detail, in Drosophila, the Toll transmembrane receptor plays a central role in the signalling pathways that control amongst other things the innate nonspecific immune response. This Toll-mediated immune response reflects an ancestral conserved signalling system that has homologous components in a wide range of organisms. Human Toll homologues have been identified amongst the Toll-like receptor (TLR) genes and Toll/interleukin-1 receptor-like (TIL) genes and contain the characteristic Toll motifs: an extracellular leucine-rich repeat domain and a cytoplasmic interleukin-1 receptor-like region. The Toll-like receptor genes (including TIL genes) now include TLR4, TIL3, TIL4, and 4 other identified TLR genes.

Other suitable sequences that may be used to downregulate Notch ligand expression include those encoding immune costimulatory molecules (for example CD80, CD86, ICOS, SLAM) and other accessory molecules that are associated with immune potentiation (for example CD2, LFA-1).

Other suitable substances that may be used to downregulate Notch ligand expression include nucleic acids that inhibit the effect of transforming growth factors such as members of the fibroblast growth factor (FGF) family. The FGF may be a mammalian basic FGF, acidic FGF or another member of the FGF family such as an FGF-1, FGF-2, FGF-3, FGF-4, FGF-5, FGF-6, FGF-7. Preferably the FGF is not acidic FGF (FGF-1; Zhao et al. (1995) J. Immunol 155:3904-3911). Most preferably, the FGF is a member of the FGF family which acts by stimulating the upregulation of expression of a Serrate polypeptide on APCs. The inventors have shown that members of the FGF family can upregulate Serrate-1 gene expression in APCs.

Suitable nucleic acid sequences may include anti-sense constructs, for example nucleic acid sequences encoding antisense Notch ligand constructs as well as antisense constructs designed to reduce or inhibit the expression of upregulators of Notch ligand expression (see above). The antisense nucleic acid may be an oligonucleotide such as a synthetic single-stranded DNA. However, more preferably, the antisense is an antisense RNA produced in the patient's own cells as a result of introduction of a genetic vector. The vector is responsible for production of antisense RNA of the desired specificity on introduction of the vector into a host cell.

Preferably, the nucleic acid sequence for use in the present invention is capable of inhibiting Serrate and Delta, preferably Serrate 1 and Serrate 2 as well as Delta 1, Delta 3 and Delta 4 expression in APCs such as dendritic cells. In particular, the nucleic acid sequence may be capable of inhibiting Serrate expression but not Delta expression in APCs. Alternatively, the nucleic acid sequence for use in the present invention is capable of inhibiting Delta expression in T cells such as CD4⁺ helper T cells or other cells of the immune system that express Delta (for example in response to stimulation of cell surface receptors). In particular, the nucleic acid sequence may be capable of inhibiting Delta expression but not Serrate expression in T cells. In a particularly preferred embodiment, the nucleic acid sequence is capable of inhibiting Notch ligand expression in both T cells and APC, for example Serrate expression in APCs and Delta expression in T cells.

Preferred suitable substances that may be used to downregulate Notch ligand expression include growth factors and cytokines. More preferably soluble protein growth factors may be used to inhibit Notch or Notch ligand expression. For instance, Notch ligand expression may be reduced or inhibited by the addition of BMPs or activins (a member of the TGF-β superfamily). In addition, T cells, APCs or tumour cells could be cultured in the presence of inflammatory type cytokines including IL-112, IFN-γ, IL-18, TNF-α, either alone or in combination with BMPs.

Molecules for inhibition of Notch signalling will also include polypeptides, or polynucleotides which encode therefor, capable of modifying Notch-protein expression or presentation on the cell membrane or signalling pathways. Molecules that reduce or interfere with its presentation as a fully functional cell membrane protein may include P inhibitors such as hydroxymate-based inhibitors.

Other substances which may be used to reduce interaction between Notch and Notch ligands are exogenous Notch or Notch ligands or functional derivatives thereof. Such Notch ligand derivatives would preferably have the DSL domain at the N-terminus and between 3 to 8 EGF-like repeats on the extracellular surface. A peptide corresponding to the Delta/Serrate/LAG-2 domain of hJagged1 and supernatants from COS cells expressing a soluble form of the extracellular portion of hJagged1 was found to mimic the effect of Jagged1 in inhibiting Notch1 (L1 et al (1998) Immunity 8(1):43-55 (L1)).

Whether a substance can be used for modulating Notch-Notch ligand expression may be determined using suitable screening assays.

Polypeptides, Proteins and Amino Acid Sequences

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

“Peptide” usually refers to a short amino acid sequence that is 10 to 40 amino acids long, preferably 10 to 35 amino acids.

The amino acid sequence may be prepared and isolated from a suitable source, or it may be made synthetically or it may be prepared by use of recombinant DNA techniques.

Nucleotide Sequences

As used herein, the term “nucleotide sequence” is synonymous with the term “polynucleotide”.

The nucleotide sequence may be DNA or RNA of genomic or synthetic or of recombinant origin. They may also be cloned by standard techniques. The nucleotide sequence may be double-stranded or single-stranded whether representing the sense or antisense strand or combinations thereof.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector. In general, primers will be produced by synthetic means, involving a step wise manufacture of the desired nucleic acid sequence one nucleotide at a time. Techniques for accomplishing this using automated techniques are readily available in the art.

“Polynucleotide” refers to a polymeric form of nucleotides of at least 10 bases in length and up to 5,000 or 10,000 bases or even more, either ribonucleotides or deoxyribonucleotides or a modified form of either type of nucleotide. The term includes single and double stranded forms of RNA and DNA.

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art.

For some applications, preferably, the nucleotide sequence is DNA. For some applications, preferably, the nucleotide sequence is prepared by use of recombinant DNA techniques (e.g. recombinant DNA). For some applications, preferably, the nucleotide sequence is cDNA. For some applications, preferably, the nucleotide sequence may be the same as the naturally occurring form.

Variants, Derivatives, Analogues, Homologues and Fragments

In addition to the specific nucleotide sequences mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be modified by addition, deletion, substitution modification replacement and/or variation of at least one residue present in the naturally-occurring nucleic acid/protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Within the definitions of “proteins” useful in the present invention, the specific amino acid residues may be modified in such a manner that the protein in question retains at least one of its endogenous functions, such modified proteins are referred to as “variants”. A variant protein can be modified by addition, deletion and/or substitution of at least one amino acid present in the naturally-occurring protein.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | 3- | | |
|---|---|---|---|
| Symbol | letter | Meaning | Codons |
| A | Ala | Alanine | GCT, GCC, GCA, GCG |
| B | Asp, | Aspartic, | GAT, GAC, AAT, AAC |
| | Asn | Asparagine | |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, |
| | | | CTC, CTA, CTG |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, |
| | | | CGG, AGA, AGG |
| S | Ser | Serine | TCT, TCC, TCA, |
| | | | TCG, AGT, AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, | Glutamic, | GAA, GAG, CAA, CAG |
| | Gln | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Polynucleotide variants will preferably comprise codon optimised sequences. Codon optimisation is known in the art as a method of enhancing RNA stability and therefor gene expression. The redundancy of the genetic code means that several different codons may encode the same amino-acid. For example, Leucine, Arginine and Serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms. Preferably, at least part of the sequence is codon optimised. Even more preferably, the sequence is codon optimised in its entirety.

As used herein, the term “homology” can be equated with “identity”. An homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (University of Wisconsin, U.S.A.; Devereux). Examples of other software than can perform sequence comparisons include, but are not limited to, the BLAST package, FASTA (Atschul et al. (1990) J. Mol. Biol. 403-410 (Atschul)) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

The five BLAST programs available at the National Center for Biotechnology Information website (maintained by the National Institutes of Health) perform the following tasks:

blastp—compares an amino acid query sequence against a protein sequence database.

blastn—compares a nucleotide query sequence against a nucleotide sequence database.

blastx—compares the six-frame conceptual translation products of a nucleotide query sequence (both strands) against a protein sequence database.

tblastn—compares a protein query sequence against a nucleotide sequence database dynamically translated in all six reading frames (both strands).

tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Clayerie & States (1993) Computers and Chemistry 17:191-201, or, for BLASTN, by the DUST program of Tatusov and Lipman (see the National Center for Biotechnology Information website, maintained by the National Institutes of Health). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at the National Center for Biotechnology Information website (maintained by the National Institutes of Health).

In some aspects of the present invention, no gap penalties are used when determining sequence identity.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleotide sequences which are homologous to or variants of sequences of use in the present invention can be obtained in a number of ways, for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

PCR technology as described e.g. in section 14 of Sambrook et al., 1989, requires the use of oligonucleotide probes that will hybridise to nucleic acid. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating α³²P dATP with the Klenow fragment of DNA polymerase in a random priming reaction, as is well known in the art. Oligonucleotides are usually end-labelled with γ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

Preferred are such sequences, probes which hybridise under high-stringency conditions.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the polynucleotide or encoded polypeptide.

In general, the terms “variant”, “homologue” or “derivative” in relation to the nucleotide sequence used in the present invention includes any substitution of, variation of, modification of, replacement of, deletion of or addition of one (or more) nucleic acid from or to the sequence providing the resultant nucleotide sequence codes for a target protein or protein for T cell signalling modulation.

As indicated above, with respect to sequence homology, preferably there is at least 75%, more preferably at least 85%, more preferably at least 90% homology to the reference sequences. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

Hybridisation

The present invention also encompasses nucleotide sequences that are capable of hybridising selectively to the reference sequences, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction (PCR) technologies.

Nucleotide sequences useful in the invention capable of selectively hybridising to the nucleotide sequences presented herein, or to their complement, will be generally at least 75%, preferably at least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to the nucleotide sequence, preferably at least 80 or 90% and more preferably at least 95% homologous to the nucleotide sequence.

The term “selectively hybridizable” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC (1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency preferably refers to conditions that permit hybridisation of only those nucleic acid sequences that form stable hybrids in 1 M Na+ at 65-68° C. High stringency conditions can be provided, for example, by hybridisation in an aqueous solution containing 6×SSC, 5× Denhardt's, 1% SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Cloning and Expression

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the target protein or protein for T cell signalling modulation encoded by the nucleotide sequences.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

The present invention also relates to vectors which comprise a polynucleotide useful in the present invention, host cells which are genetically engineered with vectors of the invention and the production of polypeptides useful in the present invention by such techniques.

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transfection, microinjection, cationic lipid-mediated transfection, electroporation, transduction, scrape loading, ballistic introduction and infection. It will be appreciated that such methods can be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

Anti-Sense

Suitable nucleic acid sequences may include anti-sense constructs, for example nucleic acid sequences encoding antisense Notch ligand constructs as well as antisense constructs designed to reduce or inhibit the expression of upregulators of Notch ligand expression (see above). The antisense nucleic acid may be an oligonucleotide such as a synthetic single-stranded DNA. However, more preferably, the antisense is an antisense RNA produced in the patient's own cells as a result of introduction of a genetic vector. The vector is responsible for production of antisense RNA of the desired specificity on introduction of the vector into a host cell.

Antisense nucleic acids can be oligonucleotides that are double-stranded or single-stranded, RNA or DNA or a modification or derivative thereof, which can be directly administered to a cell, or which can be produced intracellularly by transcription of exogenous, introduced sequences.

For example, as described in U.S. Pat. No. 2,002,0119540 inhibitory antisense or double stranded oligonucleotides can additionally comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluraci-1, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyamoinomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

An antisense oligonucleotide may also comprise one or more modified sugar moieties such as, for example, arabinose, 2-fluoroarabinose, xylulose, or hexose.

In yet another embodiment, the antisense oligonucleotide may if desired comprise at least one modified phosphate backbone such as, for example, a phosphorothioate, a phosphorodithioate, a phosphoramidothioate, a phosphoramidate, a phosphordiamidate, a methylphosphonate, an alkyl phosphotriester, or a formacetal or analog thereof. Alternatively another polymeric backbone such as a modified polypeptide backbone may be used (e.g. protein nucleic acid: PNA).

In yet another embodiment, the antisense oligonucleotide may be an alpha-anomeric oligonucleotide. An alpha-anomeric oligonucleotide forms specific double-stranded hybrids with complementary RNA in which, contrary to the usual beta-units, the strands run parallel to each other (Gautier et al., 1987, Nucl. Acids Res. 15:6625-6641). The oligonucleotide may for example be a 2′-O-methylribonucleotide (Inoue et al., 1987, Nucl. Acids Res. 15:6131-6148), or a chimeric RNA-DNA analogue (Inoue et al., 1987, FEBS Lett. 215:327-330). Oligonucleotides may be synthesized by standard methods known in the art, e.g. by use of an automated DNA synthesizer (such as are commercially available from Biosearch, Applied Biosystems, etc.). Merely as examples, phosphorothioate oligonucleotides can be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. U.S.A. 85:7448-7451), etc.

Assays

Whether a substance can be used for modulating Notch-Notch ligand expression may be determined using suitable screening assays, for example, as described in PCT Patent Publication WO 03/012441, Lorantis, or for example as described in the Examples herein.

For example, Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or IL-10 mRNA may, for instance, indicate induced anergy while an increase in levels of Dll-1 or IFN-γ mRNA, or in the levels of mRNA encoding cytokines such as IL-2, IL-5 and IL-13, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989) supra.

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee M. et al. (1996) Science 274:601-614 (Chee)). These high density arrays are particularly useful for diagnostic and prognostic purposes. Use may also be made of In vivo Expression Technology (IVET) (Camilli et al. (1994) Proc Natl Acad Sci USA 91:2634-2638 (Camilli)). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

As described above the modulator of Notch signalling may also be an immune cell which has been treated to modulate expression or interaction of Notch, a Notch ligand or the Notch signalling pathway. Such cells may readily be prepared, for example, as described in WO 00/36089 in the name of Lorantis Ltd, the text of which is herein incorporated by reference.

Particles and Particle Delivery

A variety of particles and delivery systems may be used in the present invention, including but not limited to, the following:

(i) Ballistic Particle Delivery

In one embodiment, particles according to the present invention may be administered by a needleless or “ballistic” (biolistic) delivery mechanism. A range of such delivery systems are known in the art. One system, developed by Powderject Vaccines, is particularly useful and a variety of suitable forms and embodiments are described, for example, in the following publications, which are incorporated herein by reference:

WO03011380 Silencing Device And Method For Needleless Syringe; WO03011379 Particle Cassette, Method And Kit Therefor; WO02101412 Spray Freeze-Dried Compositions; WO02100380 Production Of Hard, Dense Particles; WO02055139 Needleless Syringe; WO0243774 Nucleic Acid Immunization; WO0219989 Alginate Particle Formulation; WO0207803 Needleless Syringe; WO0193829 Powder Compositions; WO0183528 Nucleic Acid Immunization; WO0168167 Apparatus And Method For Adjusting The Characteristics Of A Needleless Syringe; WO0134185 Induction Of Mucosal Immunity By Vaccination Via The Skin Route; WO0133176 Apparatus And Method For Dispensing Small Quantities Of Particles; WO0105455 Needleless Syringe; WO0063385 Nucleic Acid Immunization; WO0062846 Needleless Syringe; WO0054827 Needleless Syringe; WO0053160 Delivery Of Microparticle Formulations Using Needleless Syringe Device For Sustained-Release Of Bioactive Compounds; WO0044421 Particle Delivery Device; WO0026385 Nucleic Acid Constructs For Genetic Immunization; WO0023592 Minimal Promoters And Uses Thereof; WO0019982 Spray Coated Microparticles For Use In Needleless Syringes; WO9927961 Transdermal Delivery Of Particulate Vaccine Compositions; WO9908689 Mucosal Immunization Using Particle-Mediated Delivery Techniques; WO9901169 Syringe And Capsule Therefor; WO9901168 Drug Particle Delivery; WO9821364 Method And Apparatus For Preparing Sample Cartridges For A Particle Acceleration Device; WO9813470 Gas-Driven Particle Delivery Device; WO9810750 Nucleic Acid Particle Delivery; WO9748485 Method For Providing Dense Particle Compositions For Use In Transdermal Particle Delivery; WO9734652 Needleless Syringe With Therapeutic Agent Particles Entrained In Supersonic Gas Flow.

As described, for example, in 20020165176 A1, particle-mediated methods for delivering such nucleic acid preparations are known in the art. Thus, once prepared and suitably purified, the nucleic acid molecules can be coated onto carrier particles (e.g., core carriers) using a variety of techniques known in the art. Carrier particles are selected from materials which have a suitable density in the range of particle sizes typically used for intracellular delivery from a particle-mediated delivery device. The optimum carrier particle size will, of course, depend on the diameter of the target cells. Alternatively, colloidal gold particles can be used wherein the coated colloidal gold is administered (e.g., injected) into tissue (e.g., skin or muscle) and subsequently taken-up by immune-competent cells.

Suitable particles include metal particles such as, tungsten, gold, platinum and iridium carrier particles. Tungsten and gold particles are preferred. Tungsten particles are readily available in average sizes of 0.5 to 2.0 um in diameter. Gold particles or microcrystalline gold (e.g., gold powder A1570, available from Engelhard Corp., East Newark, N.J.) may also be used. Gold particles provide uniformity in size (available from Alpha Chemicals in particle sizes of 1-3 um, or available from Degussa, South Plainfield, N.J. in a range of particle sizes including 0.95 um) and low toxicity. Microcrystalline gold provides a diverse particle size distribution, typically in the range of 0.1-5 um. The irregular surface area of microcrystalline gold provides for highly efficient coating with nucleic acids.

A large number of methods are known and have been described for coating or precipitating polynucleotides such as DNA or RNA onto articles such as gold or tungsten particles. Typically such methods combine a predetermined amount of gold or tungsten with plasmid DNA, CaCl₂ and spermidine. The resulting solution is suitably vortexed continually during the coating procedure to ensure uniformity of the reaction mixture. After precipitation of the nucleic acid, the coated particles can for example be transferred to suitable membranes and allowed to dry prior to use, coated onto surfaces of a sample module or cassette, or loaded into a delivery cassette for use in particular particle-mediated delivery instruments.

Following their formation, carrier particles coated with the nucleic acid preparations can be delivered to a subject using particle-mediated delivery techniques.

Various particle acceleration devices suitable for particle-mediated delivery are known in the art, and are all suited for use in the practice of the invention. Current device designs employ an explosive, electric or gaseous discharge to propel coated carrier particles toward target cells. The coated carrier particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is described in U.S. Pat. No. 5,120,657. Another electric discharge apparatus suitable for use herein is described in U.S. Pat. No. 5,149,655. The disclosure of all of these patents is incorporated herein by reference in their entireties.

If desired, these particle acceleration devices can be provided in a preloaded condition containing a suitable dosage of the coated carrier particles comprising the polynucleotide vaccine composition, with or without additional influenza vaccine compositions and/or a selected adjuvant component. The loaded syringe can be packaged in a hermetically sealed container.

The coated particles are administered to the subject to be treated in a manner compatible with the dosage formulation, and in an amount that will be effective to bring about a desired immune response. The amount of the composition to be delivered which, in the case of nucleic acid molecules is generally in the range of from 0.001 to 1000 ug, more preferably 0.01 to 10.0 ug of nucleic acid molecule per dose, depends on the subject to be treated. The exact amount necessary will vary depending on the age and general condition of the individual being immunized and the particular nucleotide sequence or peptide selected, as well as other factors. An appropriate effective amount can be readily determined by one of skill in the art.

The formulated compositions may suitably be prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in International Publication No. WO 97/48485, incorporated herein by reference.

These methods can be used to obtain nucleic acid particles having a size ranging from about 0.01 to about 250 um, preferably about 10 to about 150 um, and most preferably about 20 to about 60 um; and a particle density ranging from about 0.1 to about 25 g/cm³, and a bulk density of about 0.5 to about 3.0 g/cm³, or greater.

Single unit dosages or multidose containers, in which the particles may be packaged prior to use, may suitably comprise a hermetically sealed container enclosing a suitable amount of the particles. The particulate compositions can be packaged as a sterile formulation, and the hermetically sealed container can thus be designed to preserve sterility of the formulation until use in the methods of the invention. If desired, the containers can be adapted for direct use in a needleless syringe system. Such containers can take the form of capsules, foil pouches, sachets, cassettes, and the like. Appropriate needleless syringes are described herein above.

The container in which the particles are packaged can further be labeled to identify the composition and provide relevant dosage information. In addition, the container can be labeled with a notice in the form prescribed by a governmental agency, for example the Food and Drug Administration, wherein the notice indicates approval by the agency under Federal law of the manufacture, use or sale of the composition contained therein for human administration.

Following their formation, the particulate composition (e.g., powder) can be delivered transdermally to the subject's tissue using a suitable transdermal delivery technique. Various particle acceleration devices suitable for transdermal delivery of the substance of interest are known in the art, and will find use in the practice of the invention. A particularly preferred transdermal delivery system employs a needleless syringe to fire solid drug-containing particles in controlled doses into and through intact skin and tissue. See, e.g., U.S. Pat. No. 5,630,796 to Bellhouse et al. which describes a needleless syringe (also known as “the PowderJect® needleless syringe device”). Other needleless syringe configurations are known in the art and are described herein.

Suitably, the particulate compositions will be delivered via a powder injection method, e.g., delivered from a needleless syringe system such as those described in commonly owned International Publication Nos. WO 94/24263, WO 96/04947, WO 96/12513, and WO 96/20022, all of which are incorporated herein by reference. Delivery of particles from such needleless syringe systems is typically practised with particles having an approximate size generally ranging from 0.1 to 250 um, preferably ranging from about 1-70 um. Particles larger than about 250 um can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec, or greater. With appropriate gas pressure, particles having an average diameter of 1-70 um can be accelerated through the nozzle at velocities approaching the supersonic speeds of a driving gas flow.

If desired, these needleless syringe systems can be provided in a preloaded condition containing a suitable dosage of the particles comprising the antigen of interest and/or the selected adjuvant. The loaded syringe can be packaged in a hermetically sealed container, which may further be labeled as described above.

Compositions containing a therapeutically effective amount of the powdered molecules described herein can be delivered to any suitable target tissue via the above-described needleless syringes. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues. For nucleic acid molecules, delivery is preferably to, and the molecules expressed in, terminally differentiated cells; however, the molecules can also be delivered to non-differentiated, or partially differentiated cells such as stem cells of blood and skin fibroblasts.

The powdered compositions are administered to the subject to be treated in a manner compatible with the dosage formulation, and in an amount that will be prophylactically and/or therapeutically effective. The amount of the composition to be delivered, generally in the range of from 0.5 ug/kg to 100 ug/kg of nucleic acid molecule per dose, depends on the subject to be treated. Doses for other pharmaceuticals, such as physiological active peptides and proteins, generally range from about 0.1 ug to about 20 mg, preferably 10 ug to about 3 mg. The exact amount necessary will vary depending on the age and general condition of the individual to be treated, the severity of the condition being treated, the particular preparation delivered, the site of administration, as well as other factors. An appropriate effective amount can be readily determined by one of skill in the art.

(ii) Liposome Particle Delivery

In an alternative embodiment, particles may take the form of lipid complexes and/or liposomes.

For example, lipid-nucleic acid formulations can be formed by combining the nucleic acid with a preformed cationic liposome (see, U.S. Pat. Nos. 4,897,355, 5,264,618, 5,279,833 and 5,283,185). In such methods, the nucleic acid is attracted to the cationic surface charge of the liposome and the resulting complexes are thought to be of the liposome-covered “sandwich-type.”

Liposome-based delivery of polynucleotides is also described, for example, in N.J. Caplen, et al., Liposome-mediated CFTR gene transfer to the nasal epithelium of patients with cystic fibrosis, Nature Medicine, 1(1995) 39; M. Cotten and E. Wagner, Non-viral approaches to gene therapy, Current opinion in biotechnology, (1993) 705-710; A. Singhal and L. Huang, Gene transfer in mammalian cells using liposomes as carriers, in Gene Therapeutics: Methods and Applications of Direct Gene Transfer, J. A. Wolff, Editor. 1994, Birkhauser: Boston; and J. P. Schonfield and C. T. Caskey, Non-viral approaches to gene therapy, Brit. Med. J., 51(1995) 56.

(iii) Delivery of Particles for Uptake by Cells

In an alternative embodiment, particles may be administered for active uptake by cells, for example by phagocytosis, as described for example in U.S. Pat. No. 5,783,567 (Pangaea), which is herein incorporated by reference.

As described, for example, in U.S. Pat. No. 5,783,567, phagocytosis of microparticles by macrophages and other antigen presenting cells (APCs) is an effective means for introducing the nucleic acid into these cells. Phagocytosis by these cells can be increased by maintaining a particle size preferably below about 20 um, and preferably below about 11 um. The type of polymer used in the microparticle can also affect the efficiency of uptake by phagocytic cells, as discussed below.

The microparticles can be delivered directly into the bloodstream (i.e., by intravenous or intraarterial injection or infusion) if uptake by the phagocytic cells of the reticuloendothelial system (RES) is desired. Alternatively, one can target, via subcutaneous injection, take-up by the phagocytic cells of the draining lymph nodes. The microparticles can also be introduced intradermally (i.e., to the APCs of the skin, such as dendritic cells and Langerhans cells). Another useful route of delivery (particularly for DNAs encoding tolerance-inducing polypeptides) is via the gastrointestinal tract, e.g., orally. Alternatively, the microparticles can be introduced into organs such as the lung (e.g., by inhalation of powdered microparticles or of a nebulized or aerosolized solution containing the microparticles), where the particles are picked up by the alveolar macrophages, or may be administered intranasally or buccally.

Once a phagocytic cell phagocytoses the microparticle, the nucleic acid is released into the interior of the cell. Upon release, it can perform its intended function: for example, expression by normal cellular transcription/translation machinery.

Because these microparticles are passively targeted to dendritic cells, macrophages and other types of phagocytic cells, they represent a means for modulating immune function. Macrophages serve as professional APCs, expressing both MHC class I and class II molecules.

Suitable polymeric material may be obtained from commercial sources or can be prepared by known methods. For example, polymers of lactic and glycolic acid can be generated as described in U.S. Pat. No. 4,293,539 or purchased from Aldrich.

Alternatively, or in addition, the polymeric matrix can include, for example, polylactide, polyglycolide, poly(lactide-co-glycolide), polyanhydride, polyorthoester, polycaprolactone, polyphosphazene, proteinaceous polymer, polypeptide, polyester, or polyorthoester.

Polymeric particles containing nucleic acids are suitably prepared using a double emulsion technique, for example, as follows: First, the polymer is dissolved in an organic solvent. A preferred polymer is polylactic-co-glycolic acid (PLGA), with a lactic/glycolic acid weight ratio of 65:35, 50:50, or 75:25. Next, a sample of nucleic acid suspended in aqueous solution is added to the polymer solution and the two solutions are mixed to form a first emulsion. The solutions can be mixed by vortexing or shaking, and in a preferred method, the mixture can be sonicated. Most preferable is any method by which the nucleic acid receives the least amount of damage in the form of nicking, shearing, or degradation, while still allowing the formation of an appropriate emulsion. For example, acceptable results can be obtained with a Vibra-cell model VC-250 sonicator with a ⅛″ microtip probe, at setting #3.

During this process, the polymer forms into minute “microparticles,” each of which contains some of the nucleic acid-containing solution. If desired, one can isolate a small amount of the nucleic acid at this point in order to assess integrity, e.g., by gel electrophoresis.

The first emulsion is then added to an organic solution. The solution can be comprised of, for example, methylene chloride, ethyl acetate, or acetone, preferably containing polyvinyl alcohol (PVA), and most preferably having a 1:100 ratio of the weight of PVA to the volume of the solution. The first emulsion is generally added to the organic solution with stirring in a homogenizer or sonicator. For example, one can use a Silverson Model L4RT homogenizer (⅝″ probe) set at 7000 RPM for about 12 seconds. A 60 second homogenization time would be too harsh at this homogenization speed.

This process forms a second emulsion which is subsequently added to another organic solution with stirring (e.g., in a homogenizer). In a preferred method, the latter solution is 0.05% w/v PVA. The resultant microparticles are washed several times with water to remove the organic compounds. Particles can be passed through sizing screens to selectively remove those larger than the desired size. If the size of the microparticles is not crucial, one can dispense with the sizing step. After washing, the particles can either be used immediately or be lyophilized for storage.

The size distribution of the microparticles prepared by the above method can be determined with a COULTERM™ counter. This instrument provides a size distribution profile and statistical analysis of the particles. Alternatively, the average size of the particles can be determined by visualization under a microscope fitted with a sizing slide or eyepiece.

If desired, the nucleic acid can be extracted from the microparticles for analysis by the following procedure. Microparticles are dissolved in an organic solvent such as chloroform or methylene chloride in the presence of an aqueous solution. The polymer stays in the organic phase, while the DNA goes to the aqueous phase. The interface between the phases can be made more distinct by centrifugation. Isolation of the aqueous phase allows recovery of the nucleic acid. To test for degradation, the extracted nucleic acid can be analyzed by HPLC or gel electrophoresis.

To increase the recovery of nucleic acid, additional organic solvents, such as phenol and chloroform, can be added to the dissolved microparticles, prior to the addition of the aqueous solution. Following addition of the aqueous solution, the nucleic acid enters the aqueous phase, which can easily be partitioned from the organic phase after mixing. For a clean interface between the organic and aqueous phases, the samples should be centrifuged. The nucleic acid is retrieved from the aqueous phase by precipitation with salt and ethanol in accordance with standard methods.

Microparticles containing nucleic acid can be injected into mammals intramuscularly, intravenously, intraarterially, intradermally, intraperitoneally, or subcutaneously, or they can be introduced into the gastrointestinal tract or the respiratory tract, e.g., by inhalation of a solution or powder containing the microparticles. Expression of the nucleic acid may be monitored by an appropriate method.

Vectors for Introduction and Expression of Polynucleotides in Cells

An important aspect of the present invention is the use of delivery agents to introduce selected polynucleotide sequences into cells in vitro and in vivo, followed by expression of the selected gene in the host cell. Thus, the nucleic acids in the particles are typically in the form of vectors that are capable of being expressed in the desired subject host cell. Promoter, enhancer, stress or chemically-regulated promoters, antibiotic-sensitive or nutrient-sensitive regions, as well as therapeutic protein encoding sequences, may be included as required.

As described, for example, in U.S. Pat. No. 5,976,567 (Inex), the expression of natural or synthetic nucleic acids is typically achieved by operably linking a nucleic acid of interest to a promoter (which may be either constitutive or inducible), preferably incorporating the construct into an expression vector, and introducing the vector into a suitable host cell. Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors may be suitable for replication and integration in prokaryotes, eukaryotes, or preferably both. See, Giliman and Smith (1979), Gene, 8: 81-97; Roberts et al. (1987), Nature, 328: 731-734; Berger and Kimmel, Guide to Molecular Cloning Techniques, Methods in Enzymology, volume 152, Academic Press, Inc., San Diego, Calif. (Berger); Sambrook et al. (1989), MOLECULAR CLONING—A LABORATORY MANUAL (2nd ed.) Vol. 1-3, Cold Spring Harbor Laboratory, Cold Spring Harbor Press, N.Y., (Sambrook); and F. M. Ausubel et al., CURRENT PROTOCOLS IN MOLECULAR BIOLOGY, eds., Current Protocols, a joint venture between Greene Publishing Associates, Inc. and John Wiley & Sons, Inc., (1994 Supplement) (Ausubel). Product information from manufacturers of biological reagents and experimental equipment also provide information useful in known biological methods. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.), as well as many other commercial sources.

Vectors to which foreign nucleic acids are operably linked may be used to introduce these nucleic acids into host cells and mediate their replication and/or expression. “Cloning vectors” are useful for replicating and amplifying the foreign nucleic acids and obtaining clones of specific foreign nucleic acid-containing vectors. “Expression vectors” mediate the expression of the foreign nucleic acid. Some vectors are both cloning and expression vectors.

In general, the particular vector used to transport a foreign gene into the cell is not particularly critical. Any of the conventional vectors used for expression in the chosen host cell may be used.

An expression vector typically comprises a eukaryotic transcription unit or “expression cassette” that contains all the elements required for the expression of exogenous genes in eukaryotic cells. A typical expression cassette contains a promoter operably linked to the DNA sequence encoding a desired protein and signals required for efficient polyadenylation of the transcript.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated. Suitable promoters include the immediate early promoter from human cytomegalovirus (hCMV) and its associated intron A sequence (see e.g. WO0023592 for a suitable minimal promoter).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Another suitable enhancer element is the HBV 3′-enhancer and HBV preS2 5′-UTR (see for example GenBank Accession No AF462041). Other enhancer/promoter combinations that are suitable for the present invention include those drived from polyoma virus, human or murine cytomegalovirus, the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In addition to a promoter sequence, the expression cassette should also contain a transcription termination region downstream of the structural gene to provide for efficient termination. The termination region may be obtained from the same source as the promoter sequence or may be obtained from a different source.

If the mRNA encoded by the selected structural gene is to be efficiently translated, polyadenylation sequences are also commonly added to the vector construct (e.g. Rabbit B-globin pA: GenBank Accession No V00882). Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40, or a partial genomic copy of a gene already resident on the expression vector.

In addition to the elements already described, the expression vector of the present invention may typically contain other specialized elements intended to increase the level of expression of cloned nucleic acids or to facilitate the identification of cells that carry the transduced DNA. For instance, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The expression vectors of the present invention will typically contain both prokaryotic sequences that facilitate the cloning of the vector in bacteria as well as one or more eukaryotic transcription units that are expressed only in eukaryotic cells, such as mammalian cells. The prokaryotic sequences are preferably chosen such that they do not interfere with the replication of the DNA in eukaryotic cells.

Selected genes are normally be expressed when the DNA sequence is functionally inserted into a vector. “Functionally inserted” means that it is inserted in proper reading frame and orientation and operably linked to proper regrulatory elements. Typically, a gene will be inserted downstream from a promoter and will be followed by a stop codon, although production as a hybrid protein followed by cleavage may be used, if desired.

Expression vectors containing regulatory elements from eukaryotic viruses such as retroviruses are typically used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

While a variety of vectors may be used, it should be noted that viral vectors such as retroviral vectors are useful for modifying eukaryotic cells because of the high efficiency with which the retroviral vectors transfect target cells and integrate into the target cell genome. Additionally, the retroviruses harboring the retroviral vector are capable of infecting cells from a wide variety of tissues.

In addition to the retroviral vectors mentioned above, cells may be lipofected with adeno-associated viral vectors. See, e.g., Methods in Enzymology, Vol. 185, Academic Press, Inc., San Diego, Calif. (D. V. Goeddel, ed.) (1990) or M. Krieger (1990), Gene Transfer and Expression—A Laboratory Manual, Stockton Press, New York, N.Y., and the references cited therein. Adeno associated viruses (AAVs) require helper viruses such as adenovirus or herpes virus to achieve productive infection. In the absence of helper virus functions, AAV integrates (site-specifically) into a host cell's genome, but the integrated AAV genome has no pathogenic effect. The integration step allows the AAV genome to remain genetically intact until the host is exposed to the appropriate environmental conditions (e.g., a lytic helper virus), whereupon it re-enters the lytic life-cycle. Samulski (1993), Current Opinion in Genetic and Development, 3: 74-80, and the references cited therein provides an overview of the AAV life cycle. See also West et al. (1987), Virology, 160: 38-47; Carter et al. (1989), U.S. Pat. No. 4,797,368; Carter et al. (1993), WO 93/24641; Kotin (1994), Human Gene Therapy, 5: 793-801; Muzyczka (1994), J. Clin. Invest., 94: 1351 and Samulski, supra, for an overview of AAV vectors.

Plasmids designed for producing recombinant vaccinia, such as pGS62, (Langford, C. L. et al. (1986), Mol. Cell. Biol., 6: 3191-3199) may also be used. This plasmid consists of a cloning site for insertion of foreign nucleic acids, the P7.5 promoter of vaccinia to direct synthesis of the inserted nucleic acid, and the vaccinia TK gene flanking both ends of the foreign nucleic acid.

Whatever the vector is used, generally the vector is genetically engineered to contain, in expressible form, a gene of interest. The particular gene selected will depend on the intended tretment. Examples of such genes of interest are described below at Section D.3. Insertion of Functional Copy of a Gene, and throughout the specification.

The vectors further usually comprise selectable markers which result in nucleic acid amplification such as the sodium, potassium ATPase, thymidine kinase, aminoglycoside phosphotransferase, hygromycin B phosphotransferase, xanthine-guanine phosphoribosyl transferase, CAD (carbamyl phosphate synthetase, aspartate transcarbamylase, and dihydroorotase), adenosine deaminase, dihydro folate reductase, and asparagine synthetase and ouabain selection. Alternatively, high yield expression systems not involving nucleic acid amplification are also suitable, such as using a bacculovirus vector in insect cells, with the encoding sequence under the direction of the polyhedrin promoter or other strong baculovirus promoters.

Vectors

It will be appreciated that any suitable expression vector may be used according to the present invention. Some particular examples include for example: pUMVC1, pUMVC2, pUMVC3, pUMVC4a, pUMVC4b, pUMVC6a, pUMVC7, pUMVC5 from Aldevron (Fargo, N. Dak.); pVAC1-mcs (with signal sequence) and pVAC2-mcs from Invitrogen (Carlsbad, Calif.); and RapidVACC™ and pDNAVACC™ vectors from Nature Technology Corporation (Licoln, Nebr.).

Notch Ligand Polynucleotide Sequences

An exemplary human Delta 4 is contained in a plasmid which was deposited with the American Type Culture Collection (ATCC) on Mar. 5, 1997, and has been assigned ATCC accession number 98348 (e.g. see U.S. Pat. No. 6,121,045; Millennium)

A transformant in which vector pUCDL-1F, which reportedly contains cDNA coding total amino acid sequence of human Delta-1, is transformed into E. coli JM109, has been deposited in the National Institute of Bioscience and Human-Technology, Agency of Industrial Science and Technology, MITI, of 1-1-3, Higasi, Tsukuba-shi, Ibaragi-ken, Japan, as E. coli: JM109-pUCDL-1F. Date of deposit was Oct. 28, 1996, and Deposition No. is FBRM BP-5728. A transformant in which vector pUCSR-1, which reportedly contains cDNA coding total amino acid sequence of human Serrate-1, is transformed into E. coli JM109, has been deposited in the National Institute of Bioscience and Human-Technology, Agency of industrial Science and Technology, MITI, of 1-1-3, Higasi, Tsukuba-shi, Ibaragi-ken, Japan, as E. coli: JM109-pUCSR-1. Date of deposit was Oct. 28, 1996, and Deposition No. is FBRPM BP-5726 (See U.S. Pat. No. 6,337,387).

When nucleic acids other than plasmids are used the nucleic acids can contain nucleic acid analogs, for example, the antisense derivatives described in a review by Stein, et al., Science 261:1004-1011 (1993) and in U.S. Pat. Nos. 5,264,423 and 5,276,019, the disclosures of which are incorporated herein by reference.

Antigen Presenting Cells

Where required, antigen-presenting cells (APCs) may be “professional” antigen presenting cells or may be another cell that may be induced to present antigen to T cells. Alternatively a APC precursor may be used which differentiates or is activated under the conditions of culture to produce an APC. An APC for use in the ex vivo methods of the invention is typically isolated from a tumour or peripheral blood found within the body of a patient. Preferably the APC or precursor is of human origin. However, where APCs are used in preliminary in vitro screening procedures to identify and test suitable nucleic acid sequences, APCs from any suitable source, such as a healthy patient, may be used.

APCs include dendritic cells (DCs) such as interdigitating DCs or follicular DCs, Langerhans cells, PBMCs, macrophages, B-lymphocytes, or other cell types such as epithelial cells, fibroblasts or endothelial cells, activated or engineered by transfection to express a MHC molecule (Class I or II) on their surfaces. Precursors of APCs include CD34⁺ cells, monocytes, fibroblasts and endothelial cells. The APCs or precursors may be modified by the culture conditions or may be genetically modified, for instance by transfection of one or more genes encoding proteins which play a role in antigen presentation and/or in combination of selected cytokine genes which would promote to immune potentiation (for example IL-2, IL-12, IFN-γ, TNF-α, IL-18 etc.). Such proteins include MHC molecules (Class I or Class II), CD80, CD86, or CD40. Most preferably DCs or DC-precursors are included as a source of APCs.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba K, et al. (1992) J. Exp. Med. 175: 1157-1167 (Inaba)), or from bone marrow, non-adherent CD34⁺ cells can be treated with GM-CSF and TNF-a (Caux C, et al. (1992) Nature 360: 258-261 (Caux)). DCs can also be routinely prepared from the peripheral blood of human volunteers, similarly to the method of Sallusto and Lanzavecchia (Sallusto F and Lanzavecchia A (1994) J. Exp. Med. 179: 1109-1118) using purified peripheral blood mononucleocytes (PBMCs) and treating 2 hour adherent cells with GM-CSF and IL-4. If required, these may be depleted of CD19⁺ B cells and CD3⁺, CD2⁺ T cells using magnetic beads (Coffin R S, et al. (1998) Gene Therapy 5: 718-722 (Coffin)). Culture conditions may include other cytokines such as GM-CSF or IL-4 for the maintenance and, or activity of the dendritic cells or other antigen presenting cells.

Thus, it will be understood that the term “antigen presenting cell or the like” are used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

T Cells

Where required, T cells from any suitable source, such as a healthy patient, may be used and may be obtained from blood or another source (such as lymph nodes, spleen, or bone marrow). They may optionally be enriched or purified by standard procedures. The T cells may be used in combination with other immune cells, obtained from the same or a different individual. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used. It may also be convenient to use cell lines such as T cell hybridomas.

Exposure of Agent to APCs and T Cells

T cells/APCs may be cultured as described above. The APCs/T cells may be incubated/exposed to substances which are capable of interferring with or down-regulating Notch or Notch ligand expression. For example, they may be prepared for administration to a patient or incubated with T cells in vitro (ex vivo).

Where treated ex-vivo, modified cells of the present invention are preferably administered to a host by direct injection into the lymph nodes of the patient. Typically from 10⁴ to 10⁸ treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. Immunol. Meth., 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibody coated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

It will be appreciated that in one embodiment the therapeutic agents used in the present invention may be administered directly to patients in vivo. Alternatively or in addition, the agents may be administered to cells such as T cells and/or APCs in an ex vivo manner. For example, leukocytes such as T cells or APCs may be obtained from a patient or donor in known manner, treated/incubated ex vivo in the manner of the present invention, and then administered to a patient. In addition, it will be appreciated that a combination of routes of administration may be employed if desired. For example, where appropriate one component (such as the modulator of Notch signalling) may be administered ex-vivo and the other may be administered in vivo, or vice versa.

Introduction of Nucleic Acid Sequences into APCs and T-Cells

T-cells and APCs as described above are cultured in a suitable culture medium such as DMEM or other defined media, optionally in the presence of fetal calf serum.

Polypeptide substances may be administered to T-cells and/or APCs by introducing nucleic acid constructs/viral vectors encoding the polypeptide into cells under conditions that allow for expression of the polypeptide in the T-cell and/or APC. Similarly, nucleic acid constructs encoding antisense constructs may be introduced into the T-cells and/or APCs by transfection, viral infection or viral transduction.

In a preferred embodiment, nucleotide sequences will be operably linked to control sequences, including promoters/enhancers and other expression regulation signals. The term “operably linked” means that the components described are in a relationship permitting them to function in their intended manner. A regulatory sequence “operably linked” to a coding sequence is peferably ligated in such a way that expression of the coding sequence is achieved under condition compatible with the control sequences.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for lymphocytes, dendritic cells, skin, brain cells and epithelial cells within the eye are particularly preferred, for example the CD2, CD11c, keratin 14, Wnt-1 and Rhodopsin promoters respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) I.E. promoter.

It may also be advantageous for the promoters to be inducible so that the levels of expression of the heterologous gene can be regulated during the life-time of the cell. Inducible means that the levels of expression obtained using the promoter can be regulated.

Any of the above promoters may be modified by the addition of further regulatory sequences, for example enhancer sequences. Chimeric promoters may also be used comprising sequence elements from two or more different promoters.

Alternatively (or in addition), the regulatory sequences may be cell specific such that the gene of interest is only expressed in cells of use in the present invention. Such cells include, for example, APCs and T-cells.

If required, a small aliquot of cells may be tested for up-regulation of Notch signalling activity as described above. The cells may be prepared for administration to a patient or incubated with T-cells in vitro (ex vivo).

Tolerisation Assays

Any of the assays described above (see “Assays”) can be adapted to monitor or to detect reduced reactivity and tolerisation in immune cells for use in clinical applications. Such assays will involve, for example, detecting increased Notch-ligand expression or activity in host cells or monitoring Notch cleavage in donor cells. Further methods of monitoring immune cell activity are set out below.

Immune cell activity may be monitored by any suitable method known to those skilled in the art. For example, cytotoxic activity may be monitored. Natural killer (NK) cells will demonstrate enhanced cytotoxic activity after activation. Therefore any drop in or stabilisation of cytotoxicity will be an indication of reduced reactivity.

Once activated, leukocytes express a variety of new cell surface antigens. NK cells, for example, will express transferrin receptor, HLA-DR and the CD25 IL-2 receptor after activation. Reduced reactivity may therefore be assayed by monitoring expression of these antigens.

Hara et al. Human T-cell Activation: III, Rapid Induction of a Phosphorylated 28 kD/32 kD Disulfide linked Early Activation Antigen (EA-1) by 12-0-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and can therefore be used to monitor immune cell reactivity.

Additionally, leukocyte reactivity may be monitored as described in EP 0325489, which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognises a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the reactivity of leukocytes.

Further details of techniques for the monitoring of immune cell reactivity may be found in: ‘The Natural Killer Cell’ Lewis C. E. and J. O'D. McGee 1992. Oxford University Press; Trinchieri G. ‘Biology of Natural Killer Cells’ Adv. Immunol. 1989 vol 47 pp 187-376; ‘Cytokines of the Immune Response’ Chapter 7 in “Handbook of Immune Response Genes”. Mak T. W. and J. J. L. Simard 1998, which are incorporated herein by reference.

Preparation of Primed APCs and Lymphocytes

According to one aspect of the invention immune cells may be used to present antigens or allergens and/or may be treated to modulate expression or interaction of Notch, a Notch ligand or the Notch signalling pathway. Thus, for example, Antigen Presenting Cells (APCs) may be cultured in a suitable culture medium such as DMEM or other defined media, optionally in the presence of a serum such as fetal calf serum. Optimum cytokine concentrations may be determined by titration. One or more substances capable of up-regulating or down-regulating the Notch signalling pathway are then typically added to the culture medium together with the antigen of interest. The antigen may be added before, after or at substantially the same time as the substance(s). Cells are typically incubated with the substance(s) and antigen for at least one hour, preferably at least 3 hours, suitably at least 9, 12, 24, 48 or 36 or more hours at 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction. The resulting APCs that show increased levels of a Notch signalling are now ready for use.

Preparation of Regulatory T Cells (and B Cells) Ex Vivo

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s), then the T cell will not be brought into contact with the different substance(s) used with the APC. Once primed APCs have been prepared, it is not always necessary to administer any substances to the T cell since the primed APC is itself capable of inducing immunotolerance leading to increased Notch or Notch ligand expression in the T cell, presumably via Notch/Notch ligand interactions between the primed APC and T cell.

Incubations will typically be for at least 1 hour, preferably at least 3, 6, 12, 24, 48 or 36 or more hours, in suitable culture medium at 37° C. The progress of Notch signalling may be determined for a small aliquot of cells using the methods described above. T cells transfected with a nucleic acid construct directing the expression of, for example Delta, may be used as a control. Induction of immunotolerance may be determined, for example, by subsequently challenging T cells with antigen and measuring IL-2 production compared with control cells not exposed to APCs.

Primed T cells or B cells may also be used to induce immunotolerance in other T cells or B cells in the absence of APCs using similar culture techniques and incubation times.

Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Induction of immunotolerance may be determined by subsequently challenging T cells with antigen and measuring IL-2 production compared with control cells not exposed to APCs.

T cells or B cells which have been primed in this way may be used according to the invention to promote or increase immunotolerance in other T cells or B cells.

Therapeutic Uses

Immunological Indications

In the preferred embodiment the therapeutic effect results from a protein for Notch signalling. A detailed description of the Notch signalling pathway and conditions affected by it may be found in our WO98/20142, WO00/36089 and WO01/35990.

Diseased or infectious states that may be described as being mediated by T cells include, but are not limited to, any one or more of asthma, allergy, graft rejection, autoimmunity, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple schlerosis, rheumatoid arthritis and diabetes. The present invention may also be used in organ transplantation or bone marrow transplantation.

As indicated above, the present invention is useful in treating immune disorders such as autoimmune diseases or graft rejection such as allograft rejection.

Autoimmune Disease

Examples of disorders that may be treated include a group commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail: Organ-specific autoimmune diseases include multiple sclerosis, insulin dependent diabetes mellitus, several forms of anemia (aplastic, hemolytic), autoimmune hepatitis, thyroiditis, insulitis, iridocyclitis, scleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The present invention is also useful in cancer therapy. The present invention is especially useful in relation to adenocarcinomas such as: small cell lung cancer, and cancer of the kidney, uterus, prostrate, bladder, ovary, colon and breast.

Transplant Rejection

The present invention may be used, for example, for the treatment of organ transplants (e.g. kidney, heart, lung, liver or pancreas transplants), tissue transplants (e.g. skin grafts) or cell transplants (e.g. bone marrow transplants or blood transfusions).

A brief overview of the most common types of organ and tissue transplants is set out below.

1. Kidney Transplants:

Kidneys are the most commonly transplanted organs. Kidneys can be donated by both cadavers and living donors and kidney transplants can be used to treat numerous clinical indications (including diabetes, various types of nephritis and kidney failure). Surgical procedure for kidney transplantation is relatively simple. However, matching blood types and histocompatibility groups is desirable to avoid graft rejection. It is indeed important that a graft is accepted as many patients can become “sensitised” after rejecting a first transplant. Sensitisation results in the formation of antibodies and the activation of cellular mechanisms directed against kidney antigens. Thus, any subsequent graft containing antigens in common with the first is likely to be rejected. As a result, many kidney transplant patients must remain on some form of immunosuppressive treatment for the rest of their lives, giving rise to complications such as infection and metabolic bone disease.

2. Heart Transplantation

Heart transplantation is a very complex and high-risk procedure. Donor hearts must be maintained in such a manner that they will begin beating when they are placed in the recipient and can therefore only be kept viable for a limited period under very specific conditions. They can also only be taken from brain-dead donors. Heart transplants can be used to treat various types of heart disease and/or damage. HLA matching is obviously desirable but often impossible because of the limited supply of hearts and the urgency of the procedure.

3. Lung Transplantation

Lung transplantation is used (either by itself or in combination with heart transplantation) to treat diseases such as cystic fibrosis and acute damage to the lungs (e.g. caused by smoke inhalation). Lungs for use in transplants are normally recovered from brain-dead donors.

4. Pancreas Transplantation

Pancreas transplantation is mainly used to treat diabetes mellitus, a disease caused by malfunction of insulin-producing islet cells in the pancreas. Organs for transplantation can only be recovered from cadavers although it should be noted that transplantation of the complete pancreas is not necessary to restore the function needed to produce insulin in a controlled fashion. Indeed, transplantation of the islet cells alone could be sufficient. Because kidney failure is a frequent complication of advanced diabetes, kidney and pancreas transplants are often carried out simultaneously.

5. Skin Grafting

Most skin transplants are done with autologous tissue. However, in cases of severe burning (for example), grafts of foreign tissue may be required (although it should be noted that these grafts are generally used as biological dressings as the graft will not grow on the host and will have to be replaced at regular intervals). In cases of true allogenic skin grafting, rejection may be prevented by the use of immunosuppressive therapy. However, this leads to an increased risk of infection and is therefore a major drawback in burn victims.

6. Liver Transplantation

Liver transplants are used to treat organ damage caused by viral diseases such as hepititis, or by exposure to harmful chemicals (e.g. by chronic alcoholism). Liver transplants are also used to treat congenital abnormalities. The liver is a large and complicated organ meaning that transplantation initially posed a technical problem. However, most transplants (65%) now survive for more than a year and it has been found that a liver from a single donor may be split and given to two recipients. Although there is a relatively low rate of graft rejection by liver transplant patients, leukocytes within the donor organ together with anti-blood group antibodies can mediate antibody-dependent hemolysis of recipient red blood cells if there is a mismatch of blood groups. In addition, manifestations of GVHD have occurred in liver transplants even when donor and recipient are blood-group compatible.

Administration

Suitably the active agents are administered in combination with a pharmaceutically acceptable carrier or diluent. The pharmaceutically acceptable carrier or diluent may be, for example, sterile isotonic saline solutions, or other isotonic solutions such as phosphate-buffered saline. The conjugates of the present invention may be admixed with any suitable binder(s), lubricant(s), suspending agent(s), coating agent(s), solubilising agent(s). It is also preferred to formulate the compound in an orally active form.

Pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

For some applications, active agents may be administered orally in the form of tablets containing excipients such as starch or lactose, or in capsules or ovules either alone or in admixture with excipients, or in the form of elixirs, solutions or suspensions containing flavouring or colouring agents.

Alternatively or in addition, active agents may be administered by inhalation, intranasally or in the form of aerosol, or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilisers and preservatives as may be required.

Active agents such as polynucleotides and proteins/polypeptides may also be administered by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes. Active agents may be adminstered by conventional DNA delivery techniques, such as DNA vaccination etc., or injected or otherwise delivered with needleless systems, such as ballistic delivery on particles coated with the DNA for delivery to the epidermis or other sites such as mucosal surfaces.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

In general, a therapeutically effective oral or intravenous dose is likely to range from 0.01 to 50 mg/kg body weight of the subject to be treated, preferably 0.1 to 20 mg/kg. The conjugate may also be administered by intravenous infusion, at a dose which is likely to range from 0.001-10 mg/kg/hr.

Tablets or capsules of the conjugates may be administered singly or two or more at a time, as appropriate. It is also possible to administer the conjugates in sustained release formulations.

Active agents may also be injected parenterally, for example intracavemosally, intravenously, intramuscularly or subcutaneously

For parenteral administration, active agents may be used in the form of a sterile aqueous solution which may contain other substances, for example enough salts or monosaccharides to make the solution isotonic with blood.

For buccal or sublingual administration, agents may be administered in the form of tablets or lozenges which can be formulated in a conventional manner.

For oral, parenteral, buccal and sublingual administration to subjects (such as patients), the dosage level of active agents and their pharmaceutically acceptable salts and solvates may typically be from 10 to 500 mg (in single or divided doses). Thus, and by way of example, tablets or capsules may contain from 5 to 100 mg of active agent for administration singly, or two or more at a time, as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

The routes of administration and dosages described are intended only as a guide since a skilled practitioner will be able to determine readily the optimum route of administration and dosage for any particular patient depending on, for example, the age, weight and condition of the patient.

The term treatment or therapy as used herein should be taken to encompass diagnostic and prophylatic applications.

The treatment of the present invention includes both human and veterinary applications.

Where treated ex-vivo, modified cells of the present invention are preferably administered to a host by direct injection into the lymph nodes of the patient. Typically from 10⁴ to 10⁸ treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. Immunol. Meth., 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibody coated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

Combination Treatments

By “simultaneously” is meant that the active agents are administered at substantially the same time, and preferably together in the same formulation.

By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours, and preferably within less than about one to about four hours. When administered contemporaneously, the agents are preferably administered at the same site on the animal. The term “same site” includes the exact location, but can be within about 0.5 to about 15 centimeters, preferably from within about 0.5 to about 5 centimeters.

The term “separately” as used herein means that the agents are administered at an interval, for example at an interval of about a day to several weeks or months. The active agents may be administered in either order.

The term “sequentially” as used herein means that the agents are administered in sequence, for example at an interval or intervals of minutes, hours, days or weeks. If appropriate the active agents may be administered in a regular repeating cycle.

It will be appreciated that in one embodiment the therapeutic agents used in the present invention may be administered directly to patients in vivo. Alternatively or in addition, the agents may be administered to cells such as T cells and/or APCs in an ex vivo manner. For example, leukocytes such as T cells or APCs may be obtained from a patient or donor in known manner, treated/incubated ex vivo in the manner of the present invention, and then administered to a patient. In addition, it will be appreciated that a combination of routes of administration may be employed if desired. For example, where appropriate one component (such as the modulator of Notch signalling) may be administered ex-vivo and the other may be administered in vivo, or vice versa.

Chemical Cross-Linking

Chemically coupled sequences can be prepared from individual sequences and coupled using known chemically coupling techniques. The conjugate can be assembled using conventional solution- or solid-phase synthesis methods, affording a fully protected precursor with only the terminal amino group in deprotected reactive form. This function can then be reacted directly with a protein for T cell signalling modulation or a suitable reactive derivative thereof. Alternatively, this amino group may be converted into a different functional group suitable for reaction with a cargo moiety or a linker. Cross-linking reagents which can be utilized are discussed, for example, in Neans, G. E. and Feeney, R. E., Chemical Modification of Proteins, Holden-Day, 1974, pp. 39-43.

As discussed above the target protein and protein for T cell signalling modulation may be linked directly or indirectly via a cleavable linker moiety. Direct linkage may occur through any convenient functional group on the protein for T cell signalling modulation such as a hydroxy, carboxy or amino group. Indirect linkage which is preferable, will occur through a linking moiety. Suitable linking moieties include bi- and multi-functional alkyl, aryl, aralkyl or peptidic moieties, alkyl, aryl or aralkyl aldehydes acids esters and anyhdrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like. The functional groups on the linker moiety used to form covalent bonds between linker and protein for T cell signalling modulation on the one hand, as well as linker and target protein on the other hand, may be two or more of, e.g., amino, hydrazino, hydroxyl, thiol, maleimido, carbonyl, and carboxyl groups, etc. The linker moiety may include a short sequence of from 1 to 4 amino acid residues that optionally includes a cysteine residue through which the linker moiety bonds to the target protein.

Antigens

An antigen suitable for use in the present invention may be any substance that can be recognised by the immune system, and is generally recognised by an antigen receptor. Preferably the antigen used in the present invention is an immunogen. An allergic response occurs when the host is re-exposed to an antigen that it has encountered previously.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNγ but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen or allergen used in the present invention may be a peptide or polypeptide, glycoprotein, or more complex material containing multiple antigenic epitopes such as a protein complex, cell-membrane preparation, or virus/viral component. In particular, it is preferred to use antigens known to be associated with auto-immune diseases such as myelin basic protein (associated with multiple sclerosis), collagen (associated with rheumatoid arthritis), and insulin (diabetes), or antigens associated with rejection of non-self tissue such as MHC antigens. Where primed the APCs and/or T cells of the present invention are to be used in tissue transplantation procedures, antigens may be obtained from the tissue donor.

Autoantigens

In one embodiment, a polynucleotide coding for an activator of Notch signalling, such as a Notch receptor agonist (e.g. a sequence coding for all or part of a Notch ligand), may be administered with a polynucleotide coding for an autoantigen or antigenic determinant, to downregulate the immune response to the autoantigen or antigenic determinant. Preferably in this case the polynucleotide coding for the modulator of Notch signalling codes for a protein or polypeptide comprising a Notch ligand DSL domain, at least one EGF-like domain (and preferably 2 or 3 or more such domains) and a membrane binding or transmembrane domain.

Sequences encoding autoantigens may be derived from tissues, proteins etc. associated with the disease which give rise to the relevant autoimmune response. For example:

| Autoimmune Condition | Source of Autoantigens |
|---|---|
| Addison's disease | adrenal cell antigens; 21- |
| | hydroxylase; 17-hydroxylase |
| Alopecia | hair follicle antigens |
| Autoimmune hepatitis | liver cell antigens |
| Autoimmune parotitis | parotid gland antigens |
| Autoimmune haemolytic anemia | red cell membrane proteins; |
| | 95-110 kD membrane protein |
| Chronic active hepatitis | liver cell antigens |
| Goodpasture's syndrome | renal and lung basement |
| | membrane antigens; collagens |
| Guillain-Barre syndrome | nerve cell antigens |
| Hypophysial insufficiency | Hypophyseal antigens |
| Biermer's gastritis | Parietal cell of the stomach; |
| | intrinsic Factor |
| Idiopathic leukopenia | granulocyte antigens |
| Idiopathic thrombocytopenia | platelet membrane proteins; |
| | Glycoprotein IIa/IIIb |
| Isaac's syndrome | voltage-gated potassium channels |
| Lambert-Eaton myasthenic | synaptogamin in voltage-gated |
| syndrome (LEMS) | calcium channels |
| Myocardial infarction | heart cell antigens |
| Paraneoplastic encephalitis | RNA-binding protein (HuD) |
| Pemphigus vulgaris | “PeV antigen complex”; desmoglein |
| | (DG) (see e.g. Eur. J. |
| | Cell Biol. 55: 200 (91)) |
| Primary biliary cirrhosis | mitochondrial antigens; |
| | dihydrolipoamide |
| | acetyltransferase; pyruvate |
| | dehydrogenase complex 2 |
| | (PDC-E2) |
| Progressive systemic sclerosis | DNA topoisomerase; RNA |
| | polymerase |
| Spontaneous infertility | Sperm antigens (e.g. post- |
| | acrosomal sperm protein |
| | (PASP)) (see e.g. Biol. |
| | Reprod. 43: 559 (90)) |
| Uveitis | Ocular antigen, S-antigen, |
| | interphotoreceptor retinoid |
| | binding protein (see e.g. Exp. |
| | Eye Res. 56: 463 (93)) |
| Vitiligo | melanocyte antigens |

It will be appreciated that combinations of such autoimmune antigens and autoimmune antigenic determinants and/or polynucleotide sequences coding for them may also be used as appropriate.

An antigen suitable for use in the present invention may be any substance that can be recognised by the immune system, and is generally recognised by an antigen (T-cell) receptor. Preferably the antigen used in the present invention is an immunogen.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNγ but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component.

The antigen moiety may be, for example, a synthetic MHC-peptide complex i.e. a fragment of the MHC molecule bearing the antigen groove bearing an element of the antigen. Such complexes have been described in Altman et al. (1996) Science 274: 94-96.

Goodpasture's Autoantigens and Bystander Antigens

In one embodiment of the present invention the autoantigen or bystander antigen may be a Goodpasture's autoantigen or bystander antigen.

The term “Goodpasture's autoantigen” as used herein includes any substance or a component thereof normally found within a mammal that, in Goodpasture's disease, becomes a target of attack by the immune system, preferably the primary (or a primary) target of attack. The term also includes antigenic substances that induce conditions having the characteristics of Goodpasture's disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Goodpasture's bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Goodpasture's disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of Goodpasture's autoantigens and Goodpasture's bystander antigens include, but are not limited to collagens in particular, type IV, alpha 3 collagens.

An amino acid sequence for a human collagen, type IV, alpha 3 (Goodpasture antigen) is provided under GenBank Accession No NM_{—}001723. (See also Turner et al, Molecular cloning of the human Goodpasture antigen demonstrates it to be the alpha 3 chain of type IV collagen, J. Clin. Invest. 89 (2), 592-601 (1992)) Further sequences are provided, for example, under GenBank Accession Nos NM_{—}031366.1, NM_{—}031364.1, NM_{—}031363.1, NM_{—}031362.1 and NM_{—}000091.2 (collagen, type IV, alpha 3 (Goodpasture antigen) (COL4A3)) and NM_{—}130778.1 and NM_{—}000494.2 (collagen, type XVII, alpha 1 (COL17A1)).

Renal Autoantigens and Bystander Antigens

In one embodiment the autoantigen or bystander antigen may be a renal autoantigen or renal bystander antigen.

The term “autoimmune disease of the kidney” as used herein includes any disease in the kidney or a component thereof comes under autoimmune attack.

The term “renal autoantigen” as used herein includes any substance or a component thereof normally found within a mammal that, in autoimmune disease of the kidney, becomes a target of attack by the immune system, preferably the primary (or a primary) target of attack. The term also includes antigenic substances that induce conditions having the characteristics of an autoimmune disease of the kidney when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “renal bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the kidney under autoimmune attack in an autoimmune disease of the kidney. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of renal autoantigens and renal bystander antigens include, but are not limited to glomerular basement membrane (GBM) antigens (Goodpasture's antigens as described further above) and tubular basement membrane (TBM) antigens associated with tubulointerstitial nephritis (TIN).

Pemphigus Autoantigens and Bystander Antigens

In an alternative embodiment of the present invention the autoantigen or bystander antigen may be a Pemphigus autoantigen or bystander antigen.

The term “Pemphigus autoantigen” as used herein includes any substance or a component thereof normally found within a mammal that, in Pemphigus, becomes a target of attack by the immune system, preferably the primary (or a primary) target of attack. The term also includes antigenic substances that induce conditions having the characteristics of Pemphigus when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Pemphigus bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Pemphigus. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Pemphigus includes, for example, pemphigus vulgaris, pemphigus foliaceus and bullous pemphigoid.

Examples of Pemphigus autoantigens and Pemphigus bystander antigens include, but are not limited to desmoglein 1 and desmoglein 3.

A sequence for a human desmoglein 1 (DSG1) autoantigen is provided under GenBank Accession No AF097935. (See also Nilles et al, Structural analysis and expression of human desmoglein: a cadherin-like component of the desmosome, J. Cell. Sci. 99 (Pt 4), 809-821 (1991))

A sequence for a human bullous pemphigoid antigen 1, 230/240 kDa (BPAG1) is provided under GenBank Accession No NM_{—}001723 (see also, for example Sawamura et al, Bullous pemphigoid antigen (BPAG1): cDNA cloning and mapping of the gene to the short arm of human chromosome 6, Genomics 8 (4), 722-726 (1990)).

Further sequences are provided, for example, under GenBank Accession Nos

NM_{—}015548.1, NM_{—}020388.2 and NM_{—}001723.2 (Bullous pemphigoid antigen 1 ( 230/240 kD) (BPAG1)), M91669.1 (Bullous pemphigoid autoantigen BP180), NM_{—}001942.1 (desmoglein 1 (DSG1)) and NM_{—}001944.1 (desmoglein 3 (pemphigus vulgaris antigen; DSG3))

In one embodiment one or more antigenic determinants may be used in place of a full antigen. For example, some specific class II MHC-associated autoantigen peptide sequences are as follows (see U.S. Pat. No. 5,783,567):

| | |
|---|---|
| Peptide Sequence | Source |

| | |
|---|---|
| LNSKIAFKIVSQEPA (SEQ ID NO: 25) | desmoglein 3 (aa 190-204) |
| TPMFLLSRNTGEVRT (SEQ ID NO: 26) | desmoglein 3 (aa 206-220) |

Wegener's Autoantigens and Bystander Antigens

In an alternative embodiment of the present invention the autoantigen or bystander antigen may be a Wegener's autoantigen or bystander antigen.

The term “Wegener's autoantigen” as used herein includes any substance or a component thereof normally found within a mammal that, in Wegener's disease, becomes a target of attack by the immune system, preferably the primary (or a primary) target of attack. The term also includes antigenic substances that induce conditions having the characteristics of Wegener's disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Wegener's bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Wegener's disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of Wegener's autoantigens and Wegener's bystander antigens include, but are not limited to myeloblastin/proteinase 3.

A sequence for a Wegener's autoantigen/myeloblastin/proteinase 3 autoantigen is provided under GenBank Accession No M75154 (see also Labbaye et al, Wegener autoantigen and myeloblastin are encoded by a single mRNA, Proc. Natl. Acad. Sci. U.S.A. 88 (20), 9253-9256 (1991))

Autoimmune Anemia Autoantigens and Bystander Antigens

In an alternative embodiment of the present invention the autoantigen or bystander antigen may be an autoimmune anemia autoantigen or bystander antigen.

The term “autoimmune anemia” as used herein includes any disease in which red blood cells (RBCs) or a component thereof come under autoimmune attack. The term includes, for example, autoimmune haemolytic anemia, including both “warm autoantibody type” and “cold autoantibody type”.

The term “autoimmune anemia autoantigen” as used herein includes any substance or a component thereof normally found within a mammal that, in autoimmune anemia, becomes a target of attack by the immune system, preferably the primary (or a primary) target of attack. The term also includes antigenic substances that induce conditions having the characteristics of autoimmune anemia when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune anemia bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the red blood cells (RBCs) under autoimmune attack in autoimmune anemia. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Autoimmune anemia includes, in particular, autoimmune hemolytic anemia. Examples of autoimmune hemolytic anemia autoantigens and bystander antigens include, but are not limited to Rhesus (Rh) antigens such as E, e or C, red cell proteins and glycoproteins such as red cell protein band 4.1 and red cell membrane band 3 glycoprotein. Further examples include Wr^{b}, En^{a}, Ge, A, B and antigens within the Kidd and Kell blood group systems.

Autoimmune Thrombocytopenia Autoantigens and Bystander Antigens

In an alternative embodiment of the present invention the autoantigen or bystander antigen may be an autoimmune thrombocytopenia autoantigen or bystander antigen.

The term “autoimmune thrombocytopenia autoantigen” as used herein includes any substance or a component thereof normally found within a mammal that, in autoimmune thrombocytopenia, becomes a target of attack by the immune system, preferably the primary (or a primary) target of attack. The term also includes antigenic substances that induce conditions having the characteristics of autoimmune thrombocytopenia when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune thrombocytopenia bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the platelets under autoimmune attack in autoimmune thrombocytopenia. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Autoimmune thrombocytopenia includes, in particular, autoimmune thrombocytopenia purpura. Examples of autoimmune thrombocytopenia purpura autoantigens and bystander antigens include, but are not limited to platelet glycoproteins such as GPIIb/IIIa and/or GPIb/IX.

For example, a sequence for a human platelet glycoprotein IIb (GPIIb) is provided under GenBank Accession No M34480.

A sequence for a human platelet glycoprotein IIIa (GPIIIa) is provided under GenBank Accession No M35999.

Autoimmune Gastritis Autoantigens and Bystander Antigens

In an alternative embodiment of the present invention the autoantigen or bystander antigen may be an autoimmune gastritis autoantigen or bystander antigen.

The term “autoimmune gastritis” as used herein includes any disease in which gastric tissue or a component thereof comes under autoimmune attack. The term includes, for example, pernicious anemia.

The term “autoimmune gastritis autoantigen” as used herein includes any substance or a component thereof normally found within a mammal that, in autoimmune gastritis, becomes a target of attack by the immune system, preferably the primary (or a primary) target of attack. The term also includes antigenic substances that induce conditions having the characteristics of autoimmune gastritis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune gastritis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the gastric tissue under autoimmune attack in autoimmune gastritis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Autoimmune gastritis includes, in particular, pernicious anemia. Examples of autoimmune gastritis autoantigens and bystander antigens include, but are not limited to parietal cell antigens such as gastric H+/K+ ATPase, (100 kDa alpha subunit and 60-90 kDa beta subunit; especially the beta subunit) and intrinsic factor.

For example a sequence for a human H,K-ATPase beta subunit is provided under GenBank Accession No M75110. (See also GenBank Accession No J05451; human gastric (H+/K+)-ATPase gene and GenBank Accession No M63962; human gastric H,K-ATPase catalytic subunit gene).

Autoimmune Hepatitis Autoantigens and Bystander Antigens

In an alternative embodiment of the present invention the autoantigen or bystander antigen may be an autoimmune hepatitis autoantigen or bystander antigen.

The term “autoimmune hepatitis” as used herein includes any disease in which the liver or a component of the liver comes under autoimmune attack. The term thus includes, for example, primary biliary cirrhosis (PBC) and primary sclerosing cholangitis.

The term “autoimmune hepatitis autoantigen” as used herein includes any substance or a component thereof normally found within a mammal that, in autoimmune hepatitis, becomes a target of attack by the immune system, preferably the primary (or a primary) target of attack. The term also includes antigenic substances that induce conditions having the characteristics of autoimmune hepatitis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune hepatitis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in autoimmune gastritis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of autoimmune hepatitis autoantigens and bystander antigens include, but are not limited to cytochrome P450s such as cytochrome P450 2D6, cytochrome P450 2C9 and cytochrome P450 1A2, the asialoglycoprotein receptor (ASGP R) and UDP-glucuronosyltransferases (UGTs).

For example, cDNA encoding human cytochrome P450-2d6 (coding for antigen for AIH Type2a LKM1 antibody) is provided under GenBank Accession No E15820).

A sequence for a human cytochrome P450-1A2 (CYP1A2) is provided under GenBank Accession No AF182274.

Examples of primary biliary cirrhosis (PBC) autoantigens and bystander antigens include, but are not limited to mitochondrial antigens such as pyruvate dehydrogenase (E1-alpha decarboxylase, E1-beta decarboxylase and E2 acetyltransferase), branched-chain 2-oxo-acid dehydrogenases and 2-oxoglutarate dehydrogenases.

Autoimmune Vasculitis Autoantigens and Bystander Antigens

In an alternative embodiment of the present invention the autoantigen or bystander antigen may be an autoimmune vasculitis autoantigen or bystander antigen.

The term “autoimmune vasculitis” as used herein includes any disease in which blood vessels or a component thereof come under autoimmune attack and includes, for example, large vessel vasculitis such as giant cell arteritis and Takayasu's disease, medium-sized vessel vasculitis such as polyarteritis nodosa and Kawasaki disease and small vessel vasculitis such as Wegener's granulomatosis, Churg-Strauss syndrome, microscopic polyangiitis, Henoch Schonlein purpura, essential cryoglobulinaemic vasculitis and cutaneous leukocytoclastic angiitis.

The term “autoimmune vasculitis autoantigen” as used herein includes any substance or a component thereof normally found within a mammal that, in autoimmune vasculitis, becomes a target of attack by the immune system, preferably the primary (or a primary) target of attack. The term also includes antigenic substances that induce conditions having the characteristics of autoimmune vasculitis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune vasculitis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the blood vessel tissue under autoimmune attack in autoimmune vasculitis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of vasculitis autoantigens and bystander antigens include, but are not limited to basement membrane antigens (especially the noncollagenous domain of the alpha 3 chain of type IV collagen) and endothelial cell antigens.

Ocular Autoantigens and Bystander Antigens

In an alternative embodiment of the present invention the autoantigen or bystander antigen may be an ocular autoantigen or bystander antigen.

The term “autoimmune disease of the eye” includes any disease in which the eye or a component thereof comes under autoimmune attack. The term thus includes, for example, cicatricial pemphigoid, uveitis, Mooren's ulcer, Reiter's syndrome, Behcet's syndrome, Vogt-Koyanagi-Harada Syndrome, scleritis, lens-induced uveitis, optic neuritis and giant-cell arteritis.

The term “ocular autoantigen” as used herein includes any substance or a component thereof normally found within the eye of a mammal that, in an autoimmune disease of the eye, becomes a target of attack by the immune system, preferably the primary (or a primary) target of attack. The term also includes antigenic substances that induce conditions having the characteristics of autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “ocular bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the eye under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of ocular autoantigens and bystander antigens include, but are not limited to retinal antigens such as ocular antigen, S-antigen, interphotoreceptor retinoid binding protein (see e.g. Exp. Eye Res. 56:463 (93)) in uveitis and alpha crystallin in lens-induced uveitis.

A sequence for a human retinal S-antigen (48 KDa protein) is provided under GenBank Accession No X12453.

A sequence for a human alpha crystallin is provided under GenBank Accession No U05569.

Adrenal Autoantigens and Bystander Antigens

In an alternative embodiment of the present invention the autoantigen or bystander antigen may be an adrenal autoantigen or bystander antigen.

The term “adrenal autoimmune disease” as used herein includes any disease in which the adrenal gland or a component thereof comes under autoimmune attack. The term includes, for example, Addison's disease.

The term “adrenal autoantigen” as used herein includes any substance or a component thereof normally found within a mammal that, in adrenal autoimmune disease, becomes a target of attack by the immune system, preferably the primary (or a primary) target of attack.

The term also includes antigenic substances that induce conditions having the characteristics of adrenal autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “adrenal bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the adrenal gland under autoimmune attack in adrenal autoimmune disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of adrenal autoantigens and bystander antigens include, but are not limited to adrenal cell antigens such as the adrenocorticotropic hormone receptor (ACTH receptor) and enzymes such as 21-hydroxylase and 17-hydroxylase.

For example, an amino acid sequence for a human steroid 17-alpha-hydroxylase is provided under GenBank Accession No NM_{—}000102. (See also Krohn et al: Identification by molecular cloning of an autoantigen associated with Addison's disease as steroid 17 alpha-hydroxylase, Lancet 339 (8796), 770-773 (1992))

Cardiovascular Autoantigens and Bystander Antigens

In an alternative embodiment of the present invention the autoantigen or bystander antigen may be a cardiac autoantigen or bystander antigen.

The term “cardiac autoimmune disease” as used herein includes any disease in which the heart or a component thereof comes under autoimmune attack. The term includes, for example, autoimmune myocarditis, dilated cardiomyopathy, autoimmune rheumatic fever and Chagas' disease.

The term “cardiac autoantigen” as used herein includes any substance or a component thereof normally found within a mammal that, in cardiac autoimmune disease, becomes a target of attack by the immune system, preferably the primary (or a primary) target of attack. The term also includes antigenic substances that induce conditions having the characteristics of cardiac autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “cardiac bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the heart tissue under autoimmune attack in cardiac autoimmune disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of cardiac autoantigens and bystander antigens include, but are not limited to heart muscle cell antigens such as mysosin, laminin, beta-1 adrenergic receptors, adenine nucleotide translocator (ANT) protein and branched-chain ketodehydrogenase (BCKD).

Scleroderma/Polymyositis Autoantigens and Bystander Antigens

In an alternative embodiment of the present invention the autoantigen or bystander antigen may be a scleroderma or myositis autoantigen or bystander antigen.

The term “myositis/scleroderma autoantigen” as used herein includes any substance or a component thereof normally found within a mammal that, in myositis (particularly in dermatomyositis or polymyositis) or scleroderma, becomes a target of attack by the immune system, preferably the primary (or a primary) target of attack. The term also includes antigenic substances that induce conditions having the characteristics of myositis (particularly in dermatomyositis or polymyositis) or scleroderma when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “myositis/scleroderma bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in myositis (particularly in dermatomyositis or polymyositis) or scleroderma. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

As described, for example, in U.S. Pat. No. 5,862,360, scleroderma, or systemic sclerosis, is characterized by deposition of fibrous connective tissue in the skin, and often in many other organ systems. It may be accompanied by vascular lesions, especially in the skin, lungs, and kidneys. The course of this disease is variable, but it is usually slowly progressive. Scleroderma may be limited in scope and compatible with a normal life span. Systemic involvement, however, can be fatal.

Scleroderma may be classified as either diffuse or limited, on the basis of the extent of skin and internal organ involvement. The diffuse form is characterized by thickening and fibrosis of skin over the proximal extremities and trunk. The heart, lungs, kidneys, and gastrointestinal tract below the esophagus are often involved. Limited scleroderma is characterized by cutaneous involvement of the hands and face. Visceral involvement occurs less commonly. The limited form has a better prognosis than the diffuse form, except when pulmonary hypertension is present.

Antinuclear antibodies are found in over 95 percent of patients with scleroderma. Specific antinuclear antibodies have been shown to be directed to topoisomerase I, centromere proteins, RNA polymerases, or nucleolar components. Different antibodies are associated with particular clinical patterns of scleroderma. For example, antibodies to topoisomerase I (Scl-70) and to RNA polymerases (usually RNA polymerase III) are seen in patients with diffuse scleroderma. Antibodies to nuclear ribonucleoprotein (nRNP) are associated with diffuse and limited scleroderma.

Patients with scleroderma typically show autoreactivity against centrosomes (Tuffanelli, et al., Arch. Dermatol., 119:560-566, 1983). Centrosomes are essential structures that are highly conserved, from plants to mammals, and are important for various cellular processes. Centrosomes play a crucial role in cell division and its regulation. Centrosomes organize the mitotic spindle for separating chromosomes during cell division, thus ensuring genetic fidelity. In most cells, the centrosome includes a pair of centrioles that lie at the center of a dense, partially filamentous matrix, the pericentriolar material (PCM). The microtubule cytoskeleton is anchored to the centrosome or some other form of microtubule organizing center (MTOC), which is thought to serve as a site of microtubule nucleation.

As discussed in U.S. Pat. No. 6,160,107 the idiopathic inflammatory myopathies polymyositis, dermatomyositis and the related overlap syndromes disorder, such as polymyositis-scleroderma overlap, are inflammatory myopathies that are characterized by chronic muscle inflammation and proximal muscle weakness. The muscle inflammation causes muscle tenderness, muscle weakness, and ultimately muscle atrophy and fibrosis (see, for example, Plotz, et al. Annals of Internal Med. 111: 143-157(1989)). Also associated with the muscle inflammation are elevated serum levels of aldolase, creatine kinase, transaminases, such as alanine aminotransferase and aspartate aminotransferase, and lactic dehydrogenase. Other systems besides muscle can be affected by these conditions, resulting in arthritis, Raynaud's phenomenon, and interstitial lung disease. Clinically, polymyositis and dermatomyositis are distinguished by the presence of a characteristic rash in patients with dermatomyositis. Differences in the myositis of these conditions can be distinguished in some studies of muscle pathology.

Autoantibodies can be detected in about 90% of patients with polymyositis and dermatomyositis (Reichlin and Arnett, Arthritis and Rheum. 27: 1150-1156 (1984)). Sera from about 60% of these patients form precipitates with bovine thymus extracts on Ouchterlony immunodiffusion (ID), while sera from other patients stain tissue culture substrates, such as HEp-2 cells, by indirect immunofluorescence (IIF) (see, e.g., Targoff and Reichlin Arthritis and Rheum. 28: 796-803 (1985); Nishikai and Reichlin Arthritis and Rheum. 23: 881-888 (1980); Reichlin, et al., J. Clin. Immunol. 4:40-44 (1984)). There are numerous precipitating autoantibody specificities in myositis patients, but each individual antibody specificity occurs in only a fraction of the patients.

Many autoantibodies associated with myositis or myositis-overlap syndromes have been defined, and, in some cases, the antibodies have been identified. These include antibodies that are present in other disorders and also disease-specific antibodies (see, e.g., (Targoff and Reichlin Mt. Sinai J. of Med. 55: 487-493 (1988)). For example, a group of myositis-associated autoantibodies have been identified which are directed at cytoplasmic proteins that are related to tRNA and protein synthesis, particularly aminoacyl-tRNA synthetases. These include anti-Jo-1, which is the most common autoantibody associated with myositis autoimmune disorders (about 20% of such patients (Nishikai, et al. Arthritis Rheum. 23: 881-888 (1980)) and which is directed against histidyl-tRNA synthetase; anti-PL-7, which is directed against threonyl-tRNA synthetase; and anti-PL12, which is directed against alanyl-tRNA synthetase. Anti-U1 RNP, which is frequently found in patients with SLE, may also be found in mixed connective tissue disease, overlap syndromes involving myositis, or in some cases of myositis alone. This antibody reacts with proteins that are uniquely present on the U1 small nuclear ribonucleoprotein, which is one of the nuclear RNPs that are involved in splicing mRNA. Autoantibodies such as anti-Sm, anti-Ro/SSA, and anti-La/SSB, that are usually associated with other conditions, are sometimes found in patients with overlap syndromes. Anti-Ku has been found in myositis-scleroderma overlap syndrome and in SLE. The Ku antigen is a DNA binding protein complex with two polypeptide components, both of which have been cloned.

Anti Jo-1 and other anti-synthetases are disease specific. Other myositis-associated antibodies are anti-PM-Scl, which is present in about 5-10% of myositis patients, many of whom have polymyositis-scleroderma overlap, and anti-Mi-2, which is present in about 8% of myositis patients, almost exclusively in dermatomyositis. Mi-2 is found in high titer in about 20% of all dermatomyositis patients and in low titer in less than 5% of polymyositis patients (see, e.g., Targoff and Reichlin, Mt. Sinai J. of Med. 55: 487-493 (1988)).

Anti-Mi was first described by Reichlin and Mattioli, Clin. Immunol. and Immunopathol. 5: 12-20 (1976)). A complement-fixation reaction was used to detect it and, in that study, patients with dermatomyositis, polymyositis and polymyositis overlap syndromes had positive reactions. The prototype or reference serum, from patient Mi, forms two precipitin lines on immunodiffusion (ID) with calf thymus antigens, Mi-1 and Mi-2. Mi-1, which has been purified from bovine thymus nuclear extracts (Nishikai, et al. Mol. Immunol. 17: 1129-141 (1980)) is rarely found in other sera and is not myositis specific (Targoff, et al., Clin. Exp. Immunol. 53: 76-82 (1983)).

Anti-Mi-2 was found to be a myositis-specific autoantibody by Targoff, et al. Arthritis and Rheum. 28: 796-803 (1985). Furthermore, all patients with the antibody have the dermatomyositis rash.

Bovine thymus Mi-2 antigen was originally found to be a nuclear protein that separates in SDS polyacrylamide (SDS-PAGE) gels into two bands with apparent molecular weights of 53 kilodaltons (hereinafter kDa) and 61 KDa, respectively. Recently, additional higher molecular weight bands have been found. The bovine thymus antigenic activity is destroyed by SDS-PAGE and is trypsin sensitive, but not RNAse sensitive (Targroff et al. Arthritis and Rheum. 28: 796-803 (1985)).

Anti-PM-1 was first identified as an antibody found in 61% of dermatomyositis/polymyositis patients, including patients; with polymyositis-scleroderma overlap (Wolfe, et al. J. Clin. Invest. 59: 176-178 (1977)). PM-1 was subsequently shown to be more than one antibody. The unique specificity component of PM-1 was later named PM-Scl (Reichlin, et al. J. Clin. Immunol. 4: 40-44 (1984)). Anti-PM-Scl is found in the sera of about 5-10% of myositis patients, but is most commonly associated with polymyositis-scleroderma overlap syndrome. It also occurs in patients with polymyositis or dermatomyositis alone or in patients with scleroderma without myositis.

Anti-PM-Scl antibody immunoprecipitates a complex from HeLa cell extracts of at least eleven polypeptides that have molecular weights ranging from about 20 to 110 kDa (see, Reimer, et al., J. Immunol. 137:3802-3808 (1986). The antigen is trypsin-sensitive, occurs in nucleoli (see, e.g., Targoff and Reichlin Arthritis Rheum. 28: 226-230 (1985)) and is believed to be a preribosomal particle.

In an abstract, Bluthner, et al., First Int. Workshop on the Mol. and Cell Biology of Autoantibodies and Autoimmunity in Heidelberg (Springer-Verlag Jul. 27-29, 1989) report that sera from patients suffering from polymyositis/scleroderma-overlap syndrome (PM/Scl) recognize two major nucleolar proteins of 95 and 75 kDa molecular weight in Western blots of a Hela cell extract. They also report that cDNA that encodes a 20 kDa protein reactive with autoantibodies eluting from the 95 kDa PM-Scl HeLa antigen subunit has been cloned from a HeLa cDNA library. The sequence of the cloned DNA has not as yet been reported.

It will be appreciated that combinations of myositis/scleroderma autoimmune/bystander antigens and myositis/scleroderma autoimmune/bystander antigenic determinants and/or polynucleotide sequences coding for them may also be used as appropriate.

Examples of myositis/scleroderma autoantigens and myositis/scleroderma bystander antigens include, but are not limited to, Jo-1 (his-tRNA synthetase), PM-Scl, Mi-2, Ku, PL-7 (thr-tRNA synthetase), PL-12 (ala-tRNA-synthetase), SRP (signal recognition particle), Anti-nRNP (U1 small nuclear RNP), Ro/SS-A, and La/SS-B.

For example, a sequence for a human 100 kD Pm-Scl autoantigen protein (PM/Scl-100a) is provided under GenBank Accession No L01457. (See also Gee et al, Cloning of a complementary DNA coding for the 100-kD antigenic protein of the PM-Scl autoantigen, J. Clin. Invest. 90 (2), 559-570 (1992)) A sequence for a human 100 kD Pm-Scl autoantigen (PM/Scl-100b) is provided under GenBank Accession No X66113. (See also Bluthner and Bautz, Cloning and characterization of the cDNA coding for a polymyositis-scleroderma overlap syndrome-related nucleolar 100-kD protein, J. Exp. Med. 176 (4), 973-980 (1992)).

A sequence for a human 75 kD Pm-Scl autoantigen protein (PM/Scl-75a) is provided under GenBank Accession No M58460. (See also Alderuccio et al, Molecular characterization of an autoantigen of PM-Scl in the polymyositis/scleroderma overlap syndrome: a unique and complete human cDNA encoding an apparent 75-kD acidic protein of the nucleolar complex, J. Exp. Med. 173 (4), 941-952 (1991)).

A sequence for a human 75 kD Pm-Scl autoantigen protein (PM/Scl-75b) is provided under GenBank Accession No U09215.

A sequence for a Jo-1 (histidyl-tRNA synthetase) autoantigen protein is provided under GenBank Accession No Z11518. (See also Raben et al, Human histidyl-tRNA synthetase: recognition of amino acid signature regions in class 2a aminoacyl-tRNA synthetases, Nucleic Acids Res. 20 (5), 1075-1081 (1992)).

A sequence for a PL-7 (threonyl-tRNA synthetase) autoantigen protein is provided under GenBank Accession No M63180. (See also Cruzen et al, Nucleotide and deduced amino acid sequence of human threonyl-tRNA synthetase reveals extensive homology to the Escherichia coli and yeast enzymes, J. Biol. Chem. 266 (15), 9919-9923 (1991)) A sequence for a PL-12 (alanyl-tRNA synthetase) autoantigen protein is provided under GenBank Accession No D32050.

A sequence for an EJ (glycyl-tRNA synthetase) autoantigen protein is provided under GenBank Accession No U09587.

Further sequences are provided, for example, under GenBank Accession Nos AF241268.1, AF353396.1 (scleroderma-associated autoantigen); NM_{—}005033.1 (polymyositis/scleroderma autoantigen 1 (75 kDa) (PMSCL1)); XM_{—}001527.4, NM_{—}002685.1 (polymyositis/scleroderma autoantigen 2 (100 kDa) (PMSCL2)).

Nervous System Autoantigens and Bystander Antigens

In an alternative embodiment of the present invention the autoantigen or bystander antigen may be a nervous system autoantigen or bystander antigen for use to treat an autoimmune disease of the nervous system.

The term “autoimmune disease of the nervous system” includes any disease in which nervous tissue or a component thereof comes under autoimmune attack. The term includes, for example central nervous system diseases having an autoimmune etiology such as multiple sclerosis (MS), perivenous encephalomyelitis, autoimmune myelopathies, paraneoplastic cerebellar degeneration, paraneoplastic limbic (cortical) degeneration, stiff man syndrome, choreas (such as Sydenham's chorea), stroke, focal epilepsy and migraine; and peripheral nervous system diseases having an autoimmune etiology such as Guillain-Barre syndrome, Miller Fisher syndrome, chronic inflammatory demyelinating neuropathy, multifocal motor neuropathy with conduction block, demyelinating neuropathy associated with anti-myelin-associated glycoprotein antibodies, paraneoplastyic sensory neuropathy, POEMS, dorsal root ganglion neuronitis, acute panautonomic neuropathy and brachial neutritis.

The term “nervous system autoantigen” as used herein includes any nervous system substance or a component thereof normally found within a mammal that, in an autoimmune disease of the nervous system, becomes a target of attack by the immune system, preferably the primary (or a primary) target of attack. The term also includes antigenic substances that induce conditions having the characteristics of an autoimmune disease of the nervous system when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “nervous system bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in an autoimmune disease of the nervous system. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Preferably the nervous system autoantigen or nervous system bystander antigen is an MS autoantigen or MS bystander antigen.

The term “MS autoantigen” as used herein includes any nervous system substance or a component thereof normally found within a mammal that, in multiple sclerosis (MS), becomes a target of attack by the immune system, preferably the primary (or a primary) target of attack. The term also includes antigenic substances that induce conditions having the characteristics of MS when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “MS bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of nervous tissue under autoimmune attack in MS. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of nervous system autoimmune/bystander antigens and nervous system autoimmune/bystander antigenic determinants and/or polynucleotide sequences coding for them may also be used as appropriate.

Examples of nervous system autoantigens and nervous system bystander antigens include, but are not limited to, myelin basic proteins (MBPs), DM20, central nervous system white matter; proteolipid proteins (PLPs); myelin oligodendrocyte-associated proteins (MOGs), myelin associated glycoproteins (MAGs), alpha B-crystallins (see e.g. J. Chromatog. Biomed. Appl. 526:535 (90)).

The protein components of myelin proteins, including myelin basic protein (MBP) I proteolipid protein (PLP), myelin-associated glycoprotein (MAG) and myelin oligodendrocyte glycoprotein (MOG), are of particular interest. The suppression of T cell responsiveness to these antigens may be used to prevent or treat demyelinating diseases.

Proteolipid is a major constituent of myelin, and is known to be involved in demyelinating diseases (see, for example Greer et al. (1992) J. Immunol. 149: 783-788 and Nicholson (1997) Proc. Natl. Acad. Sci. USA 94: 9279-9284).

The integral membrane protein PLP is a dominant autoantigen of myelin.

Determinants of PLP antigenicity have been identified in several mouse strains, and includes residues 139-151 (Tuohy et al. (1989) J. Immunol. 142: 1523-1527), residues 103-116 (Tuohy et al. (1988) J. Immunol. 141: 1126-1130), residues 215-232 (Endoh et al. (1990) Int. Arch. Allerqv Appl. Immunol. 92: 433-438), residues 43-64 (Whitham et al (1991) J. Immunol. 147: 3803-3808) and residues 178-191 (Greer, et al. (1992) J. Immunol. 149: 783-788). Immunization with native PLP or with synthetic peptides corresponding to PLP epitopes induces experimental allergic encephalomyelitis (EAE). Analogues of PLP peptides generated by amino acid substitution can prevent EAE induction and progression (Kuchroo et al. (1994) J. Immunol. 153: 3326-3336, Nicholson et al. (1997) Proc. Natal. Acad. Sci. USA 94:9279-9284).

An amino acid sequence for a human proteolipid protein is reported as follows (GenBank Accession No M27110; SEQ ID NO: 27):

MGLLECCARCLVGAPFASLVATGLCFFGVALFCGCGHEALTGTEKLIETY
FSKNYQDYEYLINVIHAFQYVIYGTASFFFLYGALLLAEGFYTTGAVRQI
FGDYKTTICGKGLSATVTGGQKGRGSRGQHQAHSLERVCTCLGKWLGHPD
KFVGITYALTVVWLLVFACSAVPVYIYFNTWTTCQSIAFPSKTSASIGSL
CADARMYGVLPWNAFPGKVCGSNLLSICKTAEFQMTFHLFIAAFVGAAAT
LVSLLTFMIAATYNFAVLKLMGRGTKF

MBP is an extrinsic myelin protein that has been studied extensively. At least 26 MBP epitopes have been reported (Meinl et al (1993) J. Clin. Invest. 92: 2633-2643). Of particular interest are residues 1-11, 59-76 and 87-99. Analogues of MBP peptides generated by truncation have been shown to reverse EAE (Karin et al (1998) J. Immunol. 160: 5188-5194). DNA encoding polypeptide fragments may comprise coding sequences for immunogenic epitopes, e.g. myelin basic protein p84-102, more particularly myelin basic protein p87-99, VHFFKNIVTPRTP (p87-99), or the truncated 7-mer peptide FKNIVTP. The sequences of myelin basic protein exon 2, including the immunodominant epitope bordered by amino acids 59-85, are also of interest. For examples, see Sakai et al. (1988) J Neuroimmunol 19: 21-32; Baxevanis et al (1989) J Neuroimmunol 22: 23-30; Ota et al (1990) Nature 346: 183-187; Martin et al (1992) J Immunol. 148: 1350-1366, Valli et al (1993) J Clin In 91: 616. The immunodominant MBP (84102) peptide has been found to bind with high affinity to DRB1*1501 and DRB5*0101 molecules of the disease-associated DR2 haplotype. Overlapping but distinct peptide segments were important for binding to these molecules; hydrophobic residues (Val189 and Phe92) in the MBP (88-95) segment for peptide binding to DRB1*1501 molecules; hydrophobic and charged residues (Phe92, Lys93) in the MBP (89-101/102) sequence contributed to DRB5*0101 binding.

An amino acid sequence for a human myelin basic protein (MBP) is reported as follows (GenBank Accession No M13577; SEQ ID NO: 28):

MASQKRPSQRHGSKYLATASTMDHARHGFLPRHRDTGILDSIGRFFGGDR
GAPKRGSGKDSHHPARTAHYGSLPQKSHGRTQDENPVVHFFKNIVTPRTP
PPSQGKGRGLSLSRFSWGAEGQRPGFGYGGPASDYKSAHKGFKGVDAQGT
LSKIFKLGGRDSRSGSPMARR

The transmembrane glycoprotein MOG is a minor component of myelin that has been shown to induce EAE. Immunodominant MOG epitopes that have been identified in several mouse strains include residues 1-22, 35-55, 64-96 (deRosbo et al. (1998) J. Autoimmunity 11: 287-299, deRosbo et al. (1995) Eur J Immunol. 25: 985-993) and 41-60 (Leadbetter et al (1998) J Immunol 161: 504-512).

An amino acid sequence for a human myelin/oligodendrocyte glycoprotein (MOG) protein (25.1 kD) is reported as follows (GenBank Accession No U64564; SEQ ID NO: 29):

MASLSRPSLPSCLCSFLLLLLLQVSSSYAGQFRVIGPRHPIRALVGDEVE
LPCRISPGKNATGMEVGWYRPPFSRVVHLYRNGKDQDGDQAPEYRGRTEL
LKDAIGEGKVTLRIRNVRFSDEGGFTCFFRDHSYQEEAAMELKVEDPFYW
VSPGVLVLLAVLPVLLLQITVGLVFLCLQYRLRGKLRAEIENLHRTFDPH
FLRVPCWKITLFVIVPVLGPLVALIICYNWLHRRLAGQFLEELRNPF

An amino acid sequence for a human myelin-associated glycoprotein (MAG) is reported as follows (GenBank Accession No M29273; SEQ ID NO: 30):

MIFLTALPLFWIMISASRGGHWGAWMPSSISAFEGTCVSIPCRFDFPDEL
RPAVVHGVWYFNSPYPKNYPPVVFKSRTQVVHESFQGRSRLLGDLGLRNC
TLLLSNVSPELGGKYYFRGDLGGYNQYTFSEHSVLDIVNTPNIVVPPEVV
AGTEVEVSCMVPDNCPELRPELSWLGHEGLGEPAVLGRLREDEGTWVQVS
LLHFVPTREANGHRLGCQASFPNTTLQFEGYASMDVKYPPVIVEMNSSVE
AIEGSHVSLLCGADSNPPPLLTWMRDGTVLREAVAESLLLELEEVTPAED
GVYACLAENAYGQDNRTVGLSVMYAPWKPTVNGTMVAVEGETVSILCSTQ
SNPDPILTIFKEKQILSTVIYESELQLELPAVSPEDDGEYWCVAENQYGQ
RATAFNLSVEFAPVLLLESHCAAARDTVQCLCVVKSNPEPSVAFELPSRN
VTVNESEREFVYSERSGLVLTSILTLRGQAQAPPRVICTARNLYGAKSLE
LPFQGAHRLMWAKIGPVGAVVAFAILIAIVCYITQTRRKKNVTESPSFSA
GDNPPVLFSSDFRISGAPEKYESERRLGSERRLLGLRGEPPELDLSYSHS
DLGKRPTKDSYTLTEELAEYAEIRVK

In one embodiment one or more antigenic determinants may be used in place of a full antigen. For example, some specific class II MHC-associated autoantigen peptide sequences are as follows (see U.S. Pat. No. 5,783,567):

| | |
|---|---|
| Peptide Sequence | Source |

| | | |
|---|---|---|
| GRTQDENPVVHFFKNIVTPRTPP | (SEQ ID NO: 31) | MBP (aa 80-102) |
| AVYVYIYFNTWTTCQFIAFPFK | (SEQ ID NO: 32) | PLP (aa 170-191) |
| SQRHGSKYLATASTMDHARHG | (SEQ ID NO: 33) | MBP (aa 7-27) |
| RDTGILDSIGRFFGGDRGAP | (SEQ ID NO: 34) | MBP (aa 33-52) |
| QKSHGRTQDENPVVNFFKNI | (SEQ ID NO: 35) | MBP (aa 74-93) |
| DENPVVHFFKNIVT | (SEQ ID NO: 36) | MBP (aa 84-97) |
| ENPVVHFFKNIVTPR | (SEQ ID NO: 37) | MBP (aa 85-99) |
| HFFKNIVTPRTPP | (SEQ ID NO: 38) | MBP (aa 90-102) |
| KGFKGVDAQGTLSK | (SEQ ID NO: 39) | MBP (aa 139-152) |
| VDAQGTLSKIFKLGGRDSRS | (SEQ ID NO: 40) | MBP (aa 144-163) |

Autoimmune Arthritis Autoantigens and Bystander Antigens

In an alternative embodiment of the present invention the autoantigen or bystander antigen may be autoimmune arthritis autoantigen or bystander antigen for use to treat autoimmune arthritis.

The term “autoimmune arthritis autoantigen” as used herein includes any substance or a component thereof normally found within a mammal that, in autoimmune arthritis (especially rheumatoid arthritis (RA)), becomes a target of attack by the immune system, preferably the primary (or a primary) target of attack. The term also includes antigenic substances that induce conditions having the characteristics of autoimmune arthritis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune arthritis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in autoimmune arthritis, especially rheumatoid arthritis (RA). The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

The term “autoimmune arthritis” includes rheumatoid arthritis, juvenile arthritis, psoriatic arthritis, spondylo arthritis, relapsing polychondritis and other connective tissue diseases having an autoimmune disease component.

It will be appreciated that combinations of RA autoimmune/bystander antigens and RA autoimmune/bystander antigenic determinants and/or polynucleotide sequences coding for them may also be used as appropriate.

Some examples of RA autoantigens and RA bystander antigens include, but are not limited to, antigens from connective tissue, collagen (especially types I, II, III, IX, and XI), heat shock proteins and immunoglobulin Fc domains (see, e.g. J. Immunol. Methods 121:21 9 (89) and 151:177 (92)).

Collagen is a family of fibrous proteins that have been classified into a number of structurally and genetically distinct types (Stryer, L. Biochemistry, 2nd Edition, W. H. Freeman & Co., 1981, pp. 184-199). Type I collagen is the most prevalent form and is found inter alia, in skin, tendons, cornea and bones and consists of two subunits of alpha1(I) collagen and one subunit of a different sequence termed alpha2. Other types of collagen, including type II collagen, have three identical subunits or chains, each consisting of about 1,000 amino acids. Type II collagen (“CII”) is the type of collagen found inter alia, in cartilage, the interverbebral disc and the vitreous body. Type II collagen contains three alpha1(II) chains (alpha1(II)₃). Type III collagen is found inter alia, in blood vessels, the cardiovascular system and fetal skin and contains three alpha1(III) chains (alpha1(III)₃). Type IV collagen is localized, inter alia, in basement membranes and contains three alpha 1 (IV) chains (alpha1(IV)₃).

Diabetes Autoantigens and Bystander Antigens

In an alternative embodiment of the present invention the autoantigen or bystander antigen may be a diabetes autoantigen or bystander antigen for use to treat autoimmune diabetes.

The term “autoimmune diabetes” as used herein includes all forms of diabetes having an autoimmune component, and, in particular, Type I diabetes (also known as juvenile diabetes or insulin-dependent diabetes mellitus; IDDM). Type I diabetes is a disease that affects mainly children and young adults. The clinical features of the disease are caused by an insufficiency in the body's own insulin production due to a significant or even total reduction in of insulin production. It has been found that this type of diabetes is an autoimmune disease (cf. Castano, L. and G. S. Eisenbirth (1990) Type I diabetes: A chronic autoimmune disease of human, mouse and rat. Annu. Rev. Immunol. 8:647-679).

All cells of the immune system play a more or less important role. The B lymphocytes produce autoantibodies, whereas the monocytes/macrophages are probably involved in the induction of autoimmunity as antigen presenting cells. It is understood that T lymphocytes play a major role as effector cells in the destruction reaction. Like most autoimmune diseases type I diabetes arises because the tolerance of the T cells towards the body's own tissue (“self”) is lost. In particular, loss of tolerance towards pancreatic beta cells will result in the destruction thereof and diabetes will arise.

It is reported that about 30% to 40% of diabetic children will eventually develop nephropathy requiring dialysis and transplantation (see U.S. Pat. No. 5,624,895). Other significant complications include cardiovascular disease, stroke, blindness and gangrene. Moreover, diabetes mellitus accounts for a significant proportion of morbidity and mortality among dialysis and transplant patients.

Onset of Type I diabetes mellitus normally results from a well-characterized insulitis. During this condition, the inflammatory cells are typically directed against the beta cells of the pancreatic islets. It has been demonstrated that a large proportion of the infiltrating T lymphocytes produced during Type I diabetes mellitus are CD8-positive cytotoxic cells, which confirms the cytotoxic activity of the cellular infiltrate. CD4-positive lymphocytes are also present, the majority of which are helper T cells (Bottazzo et at., 1985, New England Journal of Medicine, 313, 353-359). The infiltrating cells also include lymphocytes or B cells that produce immunoglobulin-G (IgG) which suggest that these antibody-producing cells infiltrate the pancreatic islets (Glerchmann et at., 1987, Immunology Today, 8, 167-170).

The term “diabetes autoantigen” as used herein includes any substance or a component thereof normally found within a mammal that, in autoimmune diabetes, becomes a target of attack by the immune system, preferably the primary (or a primary) target of attack. The term also includes antigenic substances that induce conditions having the characteristics of autoimmune diabetes when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “diabetes bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue (usually the pancreas) under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of diabetes autoimmune/bystander antigens and diabetes autoimmune/bystander antigenic determinants and/or polynucleotide sequences coding for them may also be used as appropriate.

Examples of diabetes autoantigens and bystander antigens include, but are not limited to, pancreatic beta cell (Type I) antigens, insulins, insulin receptors, insulin associated antigens (IA-w), glucagons, amylins, gamma amino decarboxylases (GADs) and heat shock proteins (HSPs), carboxypeptidases, peripherins and gangliosides. Some of these are discussed in more detail below.

a) Preproinsulin

Human insulin mRNA is translated as a 110 amino acid single chain precursor called preproinsulin, and removal of its signal peptide during insertion into the endoplasmic reticulum generates proinsulin. Proinsulin consists of three domains: an amino-terminal B chain, a carboxy-terminal A chain and a connecting peptide in the middle known as the C peptide. Within the endoplasmic reticulum, proinsulin is exposed to several specific endopeptidases which excise the C peptide, thereby generating the mature form of insulin which consists of the A and B chain. Insulin and free C peptide are packaged in the Golgi into secretory granules which accumulate in the cytoplasm. The preproinsulin peptide sequence is reported as follows (SEQ ID NO: 41):

MALWMRLLPL LALLALWGPD PAAAFVNQHL CGSHLVEALY LVCGERGFFY TPKTRREAED
LQVGQVELGG GPGAGSLQPL ALEGSLQKRG IVEQCCTSIC SLYQLENYCN

The insulin A chain includes amino acids 90-110 of this sequence. The B chain includes amino acids 25-54. The connecting sequence (amino acids 55-89) includes a pair of basic amino acids at either end. Proteolytic cleavage of proinsulin at these dibasic sequences liberates the insulin molecule and free C peptide, which includes amino acids 57-87. The human preproinsulin or an immunologically active fragment thereof, e.g., B chain or an immunogenic fragment thereof, e.g., amino acids 33-47 (corresponding to residues 9-23 of the B-chain), are useful as autoantigens in the methods and compositions described herein.

b) GAD65

Gad65 is a primary beta-cell antigen involved in the autoimmune response leading to insulin dependent diabetes mellitus (Christgau et al. (1991) J Biol Chem. 266 (31): 21257-64). The presence of autoantibodies to GAD65 is used as a method of diagnosis of type 1 diabetes. Gad65 is a 585 amino acid protein with a sequence reported as follows (SEQ ID NO: 42):

MASPGSGFWS FGSEDGSGDS ENPGTARAWC QVAQKFTGGI GNKLCALLYG DAEKPAESGG
SQPPPAAARK AACACDQKPC SCSKVDVNYA FLHATDLLPA CDGERPTLAF LQDVMNILLQ
YVVKSFDRST KVIDFHYPNE LLQEYNWELA DQPQNLEEIL MHCQTTLKYA IKTGHPRYFN
QLSTGLDMVG LAADWLTSTA NTNMFTYEIA PVFVLLEYVT LKKMREIIGW PGGSGDGIFS
PGGAISNMYA MMIARFKMFP EVKEKGMAAL PRLIAFTSEH SHFSLKKGAA ALGIGTDSVI
LIKCDERGKM IPSDLERRIL EAKQKGFVPF LVSATAGTTV YGAFDPLLAV ADICKKYKIW
MHVDAAWGGG LLMSRKHKWK LSGVERANSV TWNPHKMMGV PLQCSALLVR EEGLMQNCNQ
MHASYLFQQD KHYDLSYDTG DKALQCGRHV DVFKLWLMWR AKGTTGFEAH VDKCLELAEY
LYNIIKNREG YEMVFDGKPQ HTNVCFWYIP PSLRTLEDNE ERMSRLSKVA PVIKARMMEY
GTTMVSYQPL GDKVNFFRMV ISNPAATHQD IDFLIEEIER LGQDL

c) Islet Tyrosine Phosphatase IA-2

IA-2/ICA512, a member of the protein tyrosine phosphatase family, is another major autoantigen in type 1 diabetes (Lan et al. DNA Cell Biol 13: 505-514, 1994).

It is reported that 70% of diabetic patients have autoantibodies to IA-2, which may appear years before the development of clinical disease. The IA-2 molecule is 979 amino acids in length and consists of an intracellular, transmembrane, and extracellular domain (Rabin et al. (1994) J. Immunol. 152 (6), 3183-3188). Autoantibodies are typically directed to the intracellular domain, e.g., amino acids 600-979 and fragments thereof (Zhang et al. (1997) Diabetes 46: 40-43; Xie et al. (1997) J Immunol 159: 3662-3667). The amino acid sequence of IA-2 is reported as follows (SEQ ID NO: 43):

MRRPRRPGGL GGSGGLRLLL CLLLLSSRPG GCSAVSAHGC LFDRRLCSHL EVCIQDGLFG
QCQVGVGQAR PLLQVTSPVL QRLQGVLRQL MSQGLSWHDD LTQYVISQEM ERIPRLRPPE
PRPRDRSGLA PKRPGPAGEL LLQDIPTGSA PAAQHRLPQP PVGKGGAGAS SSLSPLQAEL
LPPLLEHLLL PPQPPHPSLS YEPALLQPYL FHQFGSRDGS RVSEGSPGMV SVGPLPKAEA
PALFSRTASK GIFGDHPGHS YGDLPGPSPA QLFQDSGLLY LAQELPAPSR ARVPRLPEQG
SSSRAEDSPE GYEKEGLGDR GEKPASPAVQ PDAALQRLAA VLAGYGVELR QLTPEQLSTL
LTLLQLLPKG AGRNPGGVVN VGADIKKTME GPVEGRDTAE LPARTSPMPG HPTASPTSSE
VQQVPSPVSS EPPKAARPPV TPVLLEKKSP LGQSQPTVAG QPSARPAAEE YGYIVTDQKP
LSLAAGVKLL EILAEHVHMS SGSFINISVV GPALTFRIRH NEQNLSLADV TQQAGLVKSE
LEAQTGLQIL QTGVGQREEA AAVLPQTAHS TSPMRSVLLT LVALAGVAGL LVALAVALCV
RQHARQQDKE RLAALGPEGA HGDTTFEYQD LCRQHMATKS LFNPAEGPPE PSRVSSVSSQ
FSDAAQASPS SHSSTPSWCE EPAQANMDIS TGHMILAYME DHLRNRDRLA KEWQALCAYQ
AEPNTCATAQ GEGNIKKNRH PDFLPYDHAR IKLKVESSPS RSDYINASPI IEHDPRMPAY
IATQGPLSHT IADFWQMVWE SGCTVIVMLT PLVEDGVKQC DRYWPDEGAS LYHVYEVNLV
SEHIWCEDFL VRSFYLKNVQ TQETRTLTQF HFLSWPAEGT PASTRPLLDF RRKVNKCYRG
RSCPIIVHCS DGAGRTGTYI LIDMVLNRMA KGVKEIDIAA TLEHVRDQRP GLVRSKDQFE
FALTAVAEEV NAILKALPQ

d) ICA12

ICA12 (Kasimiotis et al. (2000) Diabetes 49 (4): 555-61; GenBank Accession No. AAD16237) is one of a number of islet cell autoantigens associated with diabetes. The amino acid sequence of ICA12 is reported as follows (SEQ ID NO: 44):

MSMRSPISAQ LALDGVGTMV NCTIKSEEKK EPCHEAPQGS ATAAEPQPGD PARASQDSAD
PQAPAQGNFR GSWDCSSPEG NGSPEPKRPG ASEAASGSQE KLDFNRNLKE VVPAIEKLLS
SDWKERFLGR NSMEAKDVKG TQESLAEKEL QLLVMIHQLS TLRDQLLTAH SEQKNMAAML
FEKQQQQMEL ARQQQEQIAK QQQQLIQQQH KINLLQQQIQ QVNMPYVMIP AFPPSHQPLP
VTPDSQLALP IQPIPCKPVE YPLQLLHSPP APVVKRPGAM ATHHPLQEPS QPLNLTAKPK
APELPNTSSS PSLKMSSCVP RPPSHGGPTR DLQSSPPSLP LGFLGEGDAV TKAIQDARQL
LHSHSGALDG SPNTPFRKDL ISLDSSPAKE RLEDGCVHPL EEAMLSCDMD GSRHFPESRN
SSHIKRPMNA FMVWAKDERR KILQAFPDMH NSSISKILGS RWKSMTNQEK QPYYEEQARL
SRQHLEKYPD YKYKPRPKRT CIVEGKRLRV GEYKALMRTR RQDARQSYVI PPQAGQVQMS
SSDVLYPRAA GMPLAQPLVE HYVPRSLDPN MPVIVNTCSL REEGEGTDDR HSVADGEMYR
YSEDEDSEGE EKSDGELVVL TD

e) ICA69

ICA69 is another autoantigen associated with type 1 diabetes (Pietropaolo et al. J Clin Invest 1993; 92: 359-371). An amino acid sequence of ICA69 is reported as follows (SEQ ID NO: 45):

MSGHKCSYPW DLQDRYAQDK SVVNKMQQRY WETKQAFIKA TGKKEDEHVV ASDADLDAKL
ELFHSIQRTC LDLSKAIVLY QKRICFLSQE ENELGKFLRS QGFQDKTRAG KMMQATGKAL
CFSSQQRLAL RNPLCRFHQE VETFRHRAIS DTWLTVNRME QCRTEYRGAL LWMKDVSQEL
DPDLYKQMEK FRKVQTQVRL AKKNFDKLKM DVCQKVDLLG ASRCNLLSHM LATYQTTLLH
FWEKTSHTMA AIHESFKGYQ PYEFTTLKSL QDPMKKLVEK EEKKKINQQE STDAAVQEPS
QLISLEEENQ RKESSSFKTE DGKSILSALD KGSTHTACSG PIDELLDMKS EEGACLGPVA
GTPEPEGADK DDLLLLSEIF NASSLEEGEF SKEWAAVFGD GQVKEPVPTM ALGEPDPKAQ
TGSGFLPSQL LDQNMKDLQA SLQEPAKAAS DLTAWFSLFA DLDPLSNPDA VGKTDKEHEL
LNA

f) Glima 38

Glima 38 is a 38 kDa islet cell membrane autoantigen which is specifically immunoprecipitated with sera from a subset of prediabetic individuals and newly diagnosed type 1 diabetic patients. Glima 38 is an amphiphilic membrane glycoprotein, specifically expressed in islet and neuronal cell lines, and thus shares the neuroendocrine expression patterns of GAD65 and IA2 (Aanstoot et al. J Clin Invest. 1996 Jun. 15; 97 (12): 2772-2783).

g) Heat Shock Protein 60 (HSP60)

HSP60, e.g., an immunologically active fragment of HSP60, e.g., p277 (see Elias et al., Eur J Immunol 1995 25 (10): 2851-7), can also be used as an autoantigen in the methods and compositions described herein. Other useful epitopes of HSP 60 are described, for example, in U.S. Pat. No. 6,110,746.

h) Carboxypeptidase H

Carboxypeptidase H has been identified as an autoantigen, e.g., in pre-type 1 diabetes patients (Castano et al. (1991) J Clin Endocrinol Metab 73 (6): 1197-201; Alcalde et al. J Autoimmun. 1996 August; 9 (4): 525-8.). Therefore, carboxypeptidase H or immunologically reactive fragments thereof (e.g., the 136-amino acid fragment of carboxypeptidase-H described in Castano, supra) can be used in the methods and compositions described herein.

i) Peripherin

Peripherin is a 58 KDa diabetes autoantigen identified in nod mice (Boitard et al. (1992) Proc Natl Acad Sci USA 89 (1): 172-6). A human peripherin sequence is reported as follows (SEQ ID NO: 46):

MSHHPSGLRA GFSSTSYRRT FGPPPSLSPG AFSYSSSSRF SSSRLLGSAS PSSSVRLGSF
RSPRAGAGAL LRLPSERLDF SMAEALNQEF LATRSNEKQE LQELNDRFAN FIEKVRFLEQ
QNAALRGELS QARGQEPARA DQLCQQELRE LRRELELLGR ERDRVQVERD GLAEDLAALK
QRLEEETRKR EDAEHNLVLF RKDVDDATLS RLELERKIES LMDEIEFLKK LHEEELRDLQ
VSVESQQVQQ VEVEATVKPE LTAALRDIRA QYESIAAKNL QEAEEWYKSK YADLSDAANR
NHEALRQAKQ EMNESRRQIQ SLTCEVDGLR GTNEALLRQL RELEEQFALE AGGYQAGAAR
LEEELRQLKE EMARHLREYQ ELLNVKMALD IEIATYRKLL EGEESRISVP VHSFASLNIK
TTVPEVEPPQ DSHSRKTVLI KTIETRNGEQ VVTESQKEQR SELDKSSAHS Y

i) Gangliosides

Gangliosides can also be useful autoantigens in the methods and compositions described herein. Gangliosides are sialic acid-containing glycolipids which are formed by a hydrophobic portion, the ceramide, and a hydrophilic part, i.e. the oligosaccharide chain. Gangliosides are expressed, inter alia, in cytosol membranes of secretory granules of pancreatic islets. Auto-antibodies to gangliosides have been described in type 1 diabetes, e.g., GM1-2 ganglioside is an islet autoantigen in diabetes autoimmunity and is expressed by human native (3 cells (Dotta et al. Diabetes. 1996 September; 45 (9): 1193-6). Gangliosides GT3, GD3 and GM-1 are also the target of autoantibodies associated with autoimmune diabetes (reviewed in Dionisi et al. Aim Ist Super Sanita 1997; 33 (3): 433-5). Ganglioside GM3 participates in the pathological conditions of insulin resistance (Tagami et al. J Biol Chem 2001 Nov. 13; online publication ahead of print).

Further sequences are provided, for example, under GenBank Accession Nos U26593.1, BC008640.1, NM_{—}022308.1, NM_{—}022307.1, NM_{—}004968.1, AF146363.1, AF147807.1, AH008870.1, U37183.1, U38260.1, AH005787.1, U71264.1, U71263.1, U71262.1, U71261.1, U71260.1, U71259.1, U71258.1, U71257.1, U71256.1, U71255.1, U71254.1, U71253.1, U71252.1, U01882.1, U17989.1 (diabetes mellitus type I autoantigen (ICAp69)), X62899.2 (islet cell antigen 512), A28076.1 (islet GAD sequence (HIGAD-FL)) and AF098915.1 (type 1 diabetes autoantigen ICA12).

Myastbenia Gravis Autoaontigens and Bystander Antigens

In an alternative embodiment of the present invention the autoantigen or bystander antigen may be a Myasthenia Gravis autoantigen or bystander antigen for use to treat Myasthenia Gravis.

The term “Myasthenia Gravis autoantigen” as used herein includes any substance or a component thereof normally found within a mammal that, in Myasthenia Gravis, becomes a target of attack by the immune system, preferably the primary (or a primary) target of attack. The term also includes antigenic substances that induce conditions having the characteristics of Myasthenia Gravis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Myasthenia Gravis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Myasthenia Gravis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of Myasthenia Gravis autoimmune/bystander antigens and Myasthenia Gravis autoimmune/bystander antigenic determinants and/or polynucleotide sequences coding for them may also be used as appropriate. Some examples of Myasthenia Gravis autoantigens and Myasthenia Gravis bystander antigens include, but are not limited to, acetyl choline receptors and components thereof, preferably human acetyl choline receptors and components thereof (see e.g. Eur. J. Pharm. 172:231(89)).

An amino acid sequence for a human gravin (A kinase (PRKA) anchor protein) autoantigen is reported as follows (GenBank Accession No M96322; SEQ ID NO: 47):

DCQAKSTPVIVSATTKKGLSSDLEGEKTTSLKWKSDEVDEQVACQEVKVS
VAIEEDLEPENGILELETKSSKLVQNIIQTAVDQFVRTEETATEMLTSEL
QTQAHMIKADSQDAGQETEKEGEEPQASAQDETPITSAKEESESTAVGQA
HSDISKDMSEASEKTMTVEVEGSTVNDQQLEEVVLPSEEEGGGAGTKSVP
EDDGHALLAERIEKSLVEPKEDEKGDDVDDPENQNSALADTDASGGLTKE
SPDTNGPKQKEKEDAQEVELQEGKVHSESDKAITPQAQEELQKQERESAK
SELTES

An amino acid sequence for a human cholinergic receptor (gamma subunit) autoantigen is reported as follows (GenBank Accession No NM_{—}005199; SEQ ID NO: 48):

MHGGQGPLLLLLLLAVCLGGTQRNLRNQEERLLADLMQNYDPNLRPAERD
SDVVNVSLKLTLTNLISLNEREEALTTNVWIEMQWCDYRLRWDPRDYEGL
WVLRVPSTMVWRPDIVLENNVDGVFEVALYCNVLVSPDGCIYWLPPAIFR
SACSISVTYFPFDWQNCSLIFQSQTYSTNEIDLQLSQEDGQTIEWIFIDP
EAFTENGEWAIQHRPAKMLLDPAAPAQEAGHQKVVFYLLIQRKPLFYVIN
IIAPCVLISSVAILIHFLPAKAGGQKCTVAINVLLAQTVFLFLVAKKVPE
TSQAVPLISKYLTFLLVVTILIVVNAVVVLNVSLRSPHTHSMARGVRKVF
LRLLPQLLRMHVRPLAPAAVQDTQSRLQNGSSGWSITTGEEVALCLPRSE
LLFQQWQRQGLVAAALEKLEKGPELGLSQFCGSLKQAAPAIQACVEACNL
IACARHQQSHFDNGNEEWFLVGRVLDRVCFLAMLSLFICGTAGIFLMAHY
NRVPALPFPGDPRPYLPS

An amino acid sequence for a human cholinergic receptor (alpha subunit) autoantigen is reported as follows (GenBank Accession No S77094; SEQ ID NO: 49):

MEPWPLLLLFSLCSAGLVLGSEHETRLVAKLFKDYSSVVRPVEDHRQVVE
VTVGLQLIQLINVDEVNQIVTTNVRLKQQWVDYNLKWNPDDYGGVKKIHI
PSEKIWRPDLVLYNNADGDFAIVKFTKVLLQYTGHITWTPPAIFKSYCEI
IVTHFPFDEQNCSMKLGTWTYDGSVVAINPESDQPDLSNFMESGEWVIKE
SRGWKHSVTYSCCPDTPYLDITYHFVMQRLPLYFIVNVIIPCLLFSFLTG
LVFYLPTDSGEKMTLSISVLLSLTVFLLVIVELIPSTSSAVPLIGKYMLF
TMVFVIASIIITVIVINTHHRSPSTHVMPNWVRKVFIDTIPNIMFFSTMK
RPSREKQDKKIFTEDIDISDISGKPGPPPMGFHSPLIKHFEVKSAIEGIK
YIAETMKSDQESNNAAAEWKYVAMVMDHILLGVFMLVCIIGTLAVFAGRL
IELNQQG

(See also Gattenlohner et al, Cloning of a cDNA coding for the acetylcholine receptor alpha-subunit from a thymoma associated with myasthenia gravis, Thymus 23 (2), 103-113 (1994))

Purified acetylcholine receptor can be isolated, for example, by the method of Mcintosh et al. J Neuroimmunol. 25: 75, 1989.

In an alternative embodiment one or more antigenic determinants may be used in place of a full antigen. For example, some specific class II MHC-associated autoantigen peptide sequences are as follows (see U.S. Pat. No. 5,783,567):

| | |
|---|---|
| Peptide Sequence | Source |

| | | |
|---|---|---|
| TVGLQLIQLINVDEVNQIVTTNVRLKQQ | (SEQ ID NO: 50) | AChR alpha (aa 32-67) |
| WVDYNLKW | | |
| QIVTTNVRLKQQWVDYNLKW | (SEQ ID NO: 51) | AChR alpha (aa 48-67) |
| QWVDYNL | (SEQ ID NO: 52) | AChR alpha (aa 59-65) |
| GGVKKIHIPSEKIWRPDL | (SEQ ID NO: 53) | AChR alpha (aa 73-90) |
| AIVKFTKVLLQY | (SEQ ID NO: 54) | AChR alpha (aa 101-112) |
| WTPPAIFKSYCEIIVTHFPF | (SEQ ID NO: 55) | AChR alpha (aa 118-137) |
| MKLGTWTYDGSVV | (SEQ ID NO: 56) | AChR alpha (aa 144-156) |
| MKLGIWTYDGSVV | (SEQ ID NO: 57) | AChR alpha (aa 144-157) |
| | | analog (I-148) |
| WTYDGSVVA | (SEQ ID NO: 58) | AChR alpha (aa 149-157) |
| SCCPDTPYLDITYHFVM | (SEQ ID NO: 59) | AChR alpha (aa 191-207) |
| DTPYLDITYHFVMQRLPL | (SEQ ID NO: 60) | AChR alpha (aa 195-212) |
| FIVNVIIPCLLFSFLTGLVFY | (SEQ ID NO: 61) | AChR alpha (aa 214-234) |
| LLVIVELIPSTSS | (SEQ ID NO: 62) | AChR alpha (aa 257-269) |
| STHVMPNWVRKVFIDTIPN | (SEQ ID NO: 63) | AChR alpha (aa 304-322) |
| NWVRKVFIDTIPNIMFFS | (SEQ ID NO: 64) | AChR alpha (aa 310-327) |
| IPNIMFFSTMKRPSREKQ | (SEQ ID NO: 65) | AChR alpha (aa 320-337) |
| AAAEWKYVAMVMDHIL | (SEQ ID NO: 66) | AChR alpha (aa 395-410) |
| IIGTLAVFAGRLIELNQQG | (SEQ ID NO: 67) | AChR alpha (aa 419-437) |
| GQTIEWIFIDPEAFTENGEW | (SEQ ID NO: 68) | AChR gamma (aa 165-184) |
| MAHYNRVPALPFPGDPRPYL | (SEQ ID NO: 69) | AChR gamma (aa 476-495) |

SLE Autoantigens and SLE Bystander Antigens

In an alternative embodiment of the present invention the autoantigen or bystander antigen may be a Systemic Lupus Erythematosus (SLE) autoantigen or bystander antigen for use to treat SLE.

The term “SLE autoantigen” as used herein includes any substance or a component thereof normally found within a mammal that, in Systemic Lupus Erythematosus (SLE), becomes a target of attack by the immune system, preferably the primary (or a primary) target of attack. The term also includes antigenic substances that induce conditions having the characteristics of an autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “SLE bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in SLE. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction, such as heatshock proteins (HSP), which although not necessarily specific to a particular tissue are normally shielded from the immune system.

It will be appreciated that combinations of SLE autoimmune/bystander antigens and SLE autoimmune/bystander antigenic determinants and/or polynucleotide sequences coding for them may also be used as appropriate.

Some examples of SLE autoantigens and SLE bystander antigens include, but are not limited to, ds-DNA, chromatins, histones, nucleolar antigens, soluble RNA protein particles (such as U1RNP, Sm, Ro/SSA and La/SSB) erythrocyte antigens and platelet antigens. Examples of proteins include, for example, the human Ku and La antigens.

For example, an amino acid sequence for a human lupus p70 (Ku) autoantigen protein is reported as follows (GenBank Accession No J04611; SEQ ID NO: 70):

MSGWESYYKTEGDEEAEEEQEENLEASGDYKYSGRDSLIFLVDASKAMFE
SQSEDELTPFDMSIQCIQSVYISKIISSDRDLLAVVFYGTEKDKNSVNFK
NIYVLQELDNPGAKRILELDQFKGQQGQKRFQDMMGHGSDYSLSEVLWVC
ANLFSDVQFKMSHKRIMLFTNEDNPHGNDSAKASRARTKAGDLRDTGIFL
DLMHLKKPGGFDISLFYRDIISIAEDEDLRVHFEESSKLEDLLRKVRAKE
TRKRALSRLKLKLNKDIVISVGIYNLVQKALKPPPIKLYRETNEPVKTKT
RTFNTSTGGLLLPSDTKRSQIYGSRQIILEKEETEELKRFDDPGLMLMGF
KPLVLLKKHHYLRPSLFVYPEESLVIGSSTLFSALLIKCLEKEVAALCRY
TPRRNIPPYFVALVPQEEELDDQKIQVTPPGFQLVFLPFADDKRKMPFTE
KIMATPEQVGKMKAIVEKLRFTYRSDSFENPVLQQHFRNLEALALDLMEP
EQAVDLTLPKVEAMNKRLGSLVDEFKELVYPPDYNPEGKVTKRKHDNEGS
GSKRPKVEYSEEELKTHISKGTLGKFTVPMLKEACRAYGLKSGLKKQELL
EALTKHFQD

(See also Reeves, W. H. and Sthoeger, Z. M., Molecular cloning of cDNA encoding the p70 (Ku) lupus autoantigen, J. Biol. Chem. 264 (9), 5047-5052 (1989).)

An amino acid sequence for a human lupus p80 (Ku) autoantigen protein is reported as follows (GenBank Accession No J04977; SEQ ID NO: 71):

MVRSGNKAAVVLCMDVGFTMSNSIPGIESPFEQAKKVITMFVQRQVFAEN
KDEIALVLFGTDGTDNPLSGGDQYQNITVHRHLMLPDFDLLEDIESKIQP
GSQQADFLDALIVSMDVIQHETIGKKFEKRHIEIFTDLSSRFSKSQLDII
IHSLKKCDISLQFFLPFSLGKEDGSGDRGDGPFRLGGHGPSFPLKGITEQ
QKEGLEIVKMVMISLEGEDGLDEIYSFSESLRKLCVFKKIERHSIHWPCR
LTIGSNLSIRIAAYKSILQERVKKTWTVVDAKTLKKEDIQKETVYCLNDD
DETEVLKEDIIQGFRYGSDIVPFSKVDEEQMKYKSEGKCFSVLGFCKSSQ
VQRRFFMGNQVLKVFAARDDEAAAVALSSLIHALDDLDMVAIVRYAYDKR
ANPQVGVAFPHIKHNYECLVYVQLPFMEDLRQYMFSSLKNSKKYAPTEAQ
LNAVDALIDSMSLAKKDEKTDTLEDLFPTTKIPNPRFQRLFQCLLHRALH
PREPLPPIQQHIWNMLNPPAEVTTKSQIPLSKIKTLFPLIEAKKKDQVTA
QEIFQDNHEDGPTAKKLKTEQGGAHFSVSSLAEGSVTSVGSVNPAENFRV
LVKQKKASFEEASNQLINHIEQFLDTNETPYFMKSIDCIRAFREEAIKFS
EEQRFNNFLKALQEKVEIKQLNHFWEIVVQDGITLITKEEASGSSVTAEE
AKKFLAPKDKPSGDTAAVFEEGGDVDDLLDMI

(See also Yaneva, M., Wen, J., Ayala, A. and Cook, R., cDNA-derived amino acid sequence of the 86-kDa subunit of the Ku antigen, J. Biol. Chem. 264 (23), 13407-13411 (1989).)

An amino acid sequence for a human La protein/SS-B antigen is reported as follows (GenBank Accession No J04205 M11108; SEQ ID NO: 72):

MAENGDNEKMAALEAKICHQIEYYFGDFNLPRDKFLKEQIKLDEGWVPLE
IMIKFNRLNRLTTDFNVIVEALSKSKAELMEISEDKTKIRRSPSKPLPEV
TDEYKNDVKNRSVYIKGFPTDATLDDIKEWLEDKGQVLNIQMRRTLHKAF
KGSIFVVFDSIESAKKFVETPGQKYKETDLLILFKDDYFAKKNEERKQNK
VEAKLRAKQEQEAKQKLEEDAEMKSLEEKIGCLLKFSGDLDDQTCREDLH
ILFSNHGEIKWIDFVRGAKEGIILFKEKAKEALGKAKDANNGNLQLRNKE
VTWEVLEGEVEKEALKKIIEDQQESLNKWKSKGRRFKGKGKGNKAAQPGS
GKGKVQFQGKKTKFASDDEHDEHDENGATGPVKRAREETDKEEPASKQQK
TENGAGDQ

(See also Chambers et al, Genomic structure and amino acid sequence domains of the human La autoantigen, J. Biol. Chem. 263 (34), 18043-18051 (1988).)
Bowel Autoantigens and Bystander Antigens

In an alternative embodiment of the present invention the autoantigen or bystander antigen may be a bowel autoantigen or bystander antigen for use to treat an autoimmune disease of the bowel.

The term “autoimmune disease of the bowel” as used herein includes any disease in which the bowel or a component of the bowel comes under autoimmune attack. The main autoimmune diseases of the bowel are inflammatory bowel disease (IBD) and celiac (also known as coeliac) disease.

Inflammatory bowel disease (IBD) is the term generally applied to four diseases of the bowel, namely Crohn's disease, ulcerative colitis, indeterminate colitis, and infectious colitis.

Ulcerative colitis is a chronic inflammatory disease mainly affecting the large intestine. The course of the disease may be continuous or relapsing, mild or severe. The earliest lesion is typically an inflammatory infiltration with abscess formation at the base of the crypts of Lieberkuhn. Coalescence of these distended and ruptured crypts tends to separate the overlying mucosa from its blood supply, leading to ulceration. Signs and symptoms of the disease include cramping, lower abdominal pain, rectal bleeding, and frequent, loose discharges consisting mainly of blood, pus, and mucus with scanty fecal particles. A total colectomy may be required for acute severe or chronic, unremitting ulcerative colitis.

Crohn's disease (also known as regional enteritis or ulcerative ileitis) is also a chronic inflammatory disease of unknown etiology but, unlike ulcerative colitis, it can affect any part of the bowel. The most prominent feature of the disease is the granular, reddish-purple edematous thickening of the bowel wall. With the development of inflammation, these granulomas often lose their circumscribed borders and integrate with the surrounding tissue. Diarrhea and obstruction of the bowel are the predominant clinical features. As with ulcerative colitis, the course of the disease may be continuous or relapsing, mild or severe but, unlike ulcerative colitis, it is not curable by resection of the involved segment of bowel. Many patients with Crohn's disease require surgery at some point, but subsequent relapse is common and continuous medical treatment is usual.

Celiac disease (CD) is a disease of the intestinal mucosa and is usually identified in infants and children. Celiac disease is associated with an inflammation of the mucosa, which causes malabsorption. Individuals with celiac disease are intolerant to the protein gluten, which is present in foods such as wheat, rye and barley. When exposed to gluten, the immune system of an individual with celiac disease responds by attacking the lining of the small intestine.

The term “bowel autoantigen” as used herein includes any substance or a component thereof normally found within a mammal that, in an autoimmune disease of the bowel, becomes a target of attack by the immune system, preferably the primary (or a primary) target of attack. The term also includes antigenic substances that induce conditions having the characteristics of an autoimmune disease of the gut when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “bowel bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the bowel under autoimmune attack in an autoimmune disease of the bowel. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of bowel autoimmune/bystander antigens and bowel autoimmune/bystander antigenic determinants and/or polynucleotide sequences coding for them may also be used as appropriate.

Examples of bowel autoantigens and bystander antigens include, but are not limited to, gliadins and tissue transglutaminases (tTG) (associated with celiac disease; see Marsh, Nature Medicine 1997;7:725-6) and tropomyosins, in particular tropomyosin isoform 5, (associated with ulcerative colitis).

Sjogren's Syndrome Autoantigens and Bystander Antigens

In an alternative embodiment of the present invention the autoantigen or bystander antigen may be a Sjogren's syndrome autoantigen or bystander antigen or antigenic determinant thereof, for use to treat an autoimmune disease of the bowel.

The term “Sjogren's syndrome autoantigen” as used herein includes any substance or a component thereof normally found within a mammal that, in Sjogren's syndrome, becomes a target of attack by the immune system, preferably the primary (or a primary) target of attack. The term also includes antigenic substances that induce conditions having the characteristics of Sjogren's syndrome when administered to mammals. Additionally, the term includes fragments comprising antigenic determinants (epitopes; preferably immunodominant epitopes) or epitope regions (preferably immunodominant epitope regions) of Sjogren's syndrome autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Sjogren's syndrome bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Sjogren's syndrome. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Some examples of Sjogren's syndrome autoantigens and Sjogren's syndrome bystander antigens include, but are not limited to, the following:

For example, an amino acid sequence for a human 52-kD SS-A/Ro autoantigen is reported as follows (GenBank Accession No M62800 M35041; SEQ ID NO: 73):

MASAARLTMMWEEVTCPICLDPFVEPVSIECGHSFCQECISQVGKGGGSV
CAVCRQRFLLKNLRPNRQLANMVNNLKEISQEAREGTQGERCAVHGERLH
LFCEKDGKALCWVCAQSRKHRDHAMVPLEEAAQEYQEKLQVALGELRRKQ
ELAEKLEVEIAIKRADWKKTVETQKSRIHAEFVQQKNFLVEEEQRQLQEL
EKDEREQLRILGEKEAKLAQQSQALQELISELDRRCHSSALELLQEVIIV
LERSESWNLKDLDITSPELRSVCHVPGLKKMLRTCAVHITLDPDTANPWL
ILSEDRRQVRLGDTQQSIPGNEERFDSYPMVLGAQHFHSGKHYWEVDVTG
KEAWDLGVCRDSVRRKGHFLLSSKSGFWTIWLWNKQKYEAGTYPQTPLHL
QVPPCQVGIFLDYEAGMVSFYNITDHGSLIYSFSESAFTGPLRPFFSPGF
NDGGKNTAPLTLCPLNIGSQGSTDY

(See also Chan et al, Molecular definition and sequence motifs of the 52-kD component of human SS-A/Ro autoantigen, J. Clin. Invest. 87 (1), 68-76 (1991).)

An amino acid sequence for Human SS-A/Ro ribonucleoprotein autoantigen 60 kd subunit is reported as follows (GenBank Accession No. M25077; SEQ ID NO: 74):

MEESVNQMQPLNEKQIANSQDGYVWQVTDMNRLHRFLCFGSEGGTYYIKE
QKLGLENAEALIRLIEDGRGCEVIQEIKSFSQEGRTTKQEPMLFALAICS
QCSDISTKQAAFKAVSEVCRIPTHLFTFIQFKKDLKESMKCGMWGRALRK
AIADWYNEKGGMALALAVTKYKQRNGWSHKDLLRLSHLKPSSEGLAIVTK
YITKGWKEVHELYKEKALSVETEKLLKYLEAVEKVKRTRDELEVIHLIEE
HRLVREHLLTNHLKSKEVWKALLQEMPLTALLRNLGKMTANSVLEPGNSE
VSLVCEKLCNEKLLKKARIHPFHILIALETYKTGHGLRGKLKWRPDEEIL
KALDAAFYKTFKTVEPTGKRFLLAVDVSASMNQRVLGSILNASTVAAAMC
MVVTRTEKDSYVVAFSDEMVPCPVTTDMTLQQVLMAMSQIPAGGTDCSLP
MIWAQKTNTPADVFIVFTDNETFAGGVHPAIALREYRKKMDIPAKLIVCG
MTSNGFTIADPDDRALQNTLLNKSF

(See also Ben-Chetrit et al, Isolation and characterization of a cDNA clone encoding the 60-kD component of the human SS-A/Ro ribonucleoprotein autoantigen, J. Clin. Invest. 83 (4), 1284-1292 (1989).)

Further sequences are provided, for example, under GenBank Accession Nos. NM_{—}003141.2 (Sjogren syndrome antigen A1 (52 kDa, ribonucleoprotein autoantigen SS-A/Ro) (SSA1)); NM_{—}004600.1 (Sjogren syndrome antigen A2 (60 kDa, ribonucleoprotein autoantigen SS-A/Ro) (SSA2)); NM_{—}003142.1, BC001289.1, BC020818.1 (Sjogren syndrome antigen B (autoantigen La) (SSB)); NM_{—}003731.1, BC000864.1 (Sjogren's syndrome nuclear autoantigen 1 (SSNA1)); NM_{—}006396.1, BC014791.1 (Sjogren's syndrome/scleroderma autoantigen 1 (SSSCA1)); AJ277541.1, AF282065.1 (SLA/LP autoantigen).

Thyroid Autoantigens and Bystander Antigens

In an alternative embodiment of the present invention the autoantigen or bystander antigen may be a thyroid autoantigen or bystander antigen or antigenic determinant thereof, for use to treat an autoimmune disease of the thyroid.

The term “thyroid autoimmune disease” as used herein includes any condition in which there is an autoimmune reaction to the thyroid or a component thereof. The best known autoimmune diseases of the thyroid include Graves' disease (also known as thyrotoxicosis), Hashimoto's thyroiditis and primary hypothyroidism. Further examples include atrophic autoimmune thyroiditis, primary myxoedema, asymptomatic thyroiditis, postpartal thyroiditis and neonatal hypothyroidism.

Diagnosis is typically based on the detection of autoantibodies in the patient. The three main thyroid autoantigens are the TSH receptor, thyroperoxidase (TPO, also known as microsomal antigen) and thyroglobulin (Tg) (Dawe, K., Hutchings, P., Champion, B., Cooke, A., Roitt, I., “Autoantigens in Thyroid diseases”, Springer Semin. Immunopathol. 14, 285-307, 1993).

The term “thyroid autoantigen” as used herein includes any substance or a component thereof normally found within a mammal that, in a thyroid autoimmune disease, becomes a target of attack by the immune system, preferably the primary (or a primary) target of attack. The term also includes antigenic substances that induce conditions having the characteristics of a thyroid autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ (usually the thyroid gland) under autoimmune attack and recognized by a substantial percentage (e.g. a majority though not necessarily an absolute majority) of autoimmune attack T-cells.

The term “thyroid bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the thyroid gland under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of thyroid autoantigens and thyroid bystander antigens include, but are not limited to, the thyroid stimulatory hormone (TSH) receptor (associated in particular with Grave's disease), thyroperoxidases (TPO; associated with Hashimoto's thyroiditis) and thyroglobulins (Tg).

For example, an amino acid sequence for a human thyroid stimulatory hormone receptor (TSHR) is reported as follows (GenBank Accession No. M32215; SEQ ID NO: 75):

MRPADLLQLVLLLDLPRDLGGMGCSSPPCECHQEEDFRVTCKDIQRIPSL
PPSTQTLKLIETHLRTIPSHAFSNLPNISRIYVSIDVTLQQLESHSFYNL
SKVTHIEIRNTRNLTYIDPDALKELPLLKFLGIFNTGLKMFPDLTKVYST
DIFFILEITDNPYMTSIPVNAFQGLCNETLTLKLYNNGFTSVQGYAFNGT
KLDAVYLNKNKYLTVIDKDAFGGVYSGPSLLDVSQTSVTALPSKGLEHLK
ELIARNTWTLKKLPLSLSFLHLTRADLSYFSHCCAFKNQKKIRGILESLM
CNESSMQSLRQRKSVNALNSPLHQEYEENLGDSIVGYKEKSKFQDTHNNA
HYYVFFEEQEDEIIGFGQELKNPQEETLQAFDSHYDYTICGDSEDMVCTP
KSDEFNPCEDIMGYKFLRIVVWFVSLLALLGNVFVLLILLTSHYKLNVPR
FLMCNLAFADFCMGMYLLLIASVDLYTHSEYYNHAIDWQTGPGCNTAGFF
TVFASELSVYTLTVITLERWYAITFAMRLDRKIRLRHACAIMVGGWVCCF
LLALLPLVGISSYAKVSICLPMDTETPLALAYIVFVLTLNIVAFVIVCCC
YVKIYITVRNPQYNPGDKDTKIAKRMAVLIFTDFICMAPISFYALSAILN
KPLITVSNSKILLVLFYPLNSCANPFLYAIFTKAFQRDVFILLSKFGICK
RQAQAYRGQRVPPKNSTDIQVQKVTHEMRQGLHNMEDVYELIEKSHLTPK
KQGQISEEYMQTVL

An amino acid sequence for a human thyroperoxidase (described as the primary autoantigen in human autoimmune thyroiditis (Hashimoto's thyroiditis) is reported as follows (GenBank Accession No. M17755; SEQ ID NO: 76):

MRALAVLSVTLVMACTEAFFPFISRGKELLWGKPEESRVSSVLEESKRLV
DTAMYATMQRNLKKRGILSGAQLLSFSKLPEPTSGVIARAAEIMETSIQA
MKRKVNLKTQQSQHPTDALSEDLLSIIANMSGCLPYMLPPKCPNTCLANK
YRPITGACNNRDHPRWGASNTALARWLPPVYEDGFSQPRGWNPGFLYNGF
PLPPVREVTRHVIQVSNEVVTDDDRYSDLLMAWGQYIDHDIAFTPQSTSK
AAFGGGSDCQMTCENQNPCFPIQLPEEARPAAGTACLPFYRSSAACGTGD
QGALFGNLSTANPRQQMNGLTSFLDASTVYGSSPALERQLRNWTSAEGLL
RVHGRLRDSGRAYLPFVPPRAPAACAPEPGNPGETRGPCFLAGDGRASEV
PSLTALHTLWLREHNRLAAALKALNAHWSADAVYQEARKVVGALHQIITL
RDYIPRILGPEAFQQYVGPYEGYDSTANPTVSNVFSTAAFRFGHATIHPL
VRRLDASFQEHPDLPGLWLHQAFFSPWTLLRGGGLDPLIRGLLARPAKLQ
VQDQLMNEELTERLFVLSNSSTLDLASINLQRGRDHGLPGYNEWREFCGL
PRLETPADLSTAIASRSVADKILDLYKHPDNIDVWLGGLAENFLPRARTG
PLFACLIGKQMKALRDGDWFWWENSHVFTDAQRRELEKHSLSRVICDNTG
LTRVPMDAFQVGKFPEDFESCDSITGMNLEAWRETFPQDDKCGFPESVEN
GDFVHCEESGRRVLVYSCRHGYELQGREQLTCTQEGWDFQPPLCKDVNEC
ADGAHPPCHASARCRNTKGGFQCLCADPYELGDDGRTCVDSGRLPRVTWI
SMSLAALLIGGFAGLTSTVICRWTRTGTKSTLPISETGGGTPELRCGKHQ
AVGTSPQRAAAQDSEQESAGMEGRDTHRLPRAL

Skin Autoantigens and Bystander Antigens

In an alternative embodiment of the present invention the autoantigen or bystander antigen may be a skin autoantigen or bystander antigen or antigenic determinant thereof, for use to treat an autoimmune disease of the skin.

The term “skin autoantigen” as used herein includes any substance or a component thereof normally found within a mammal that, in an autoimmune disease of the skin, such as Psoriasis or Vitiligo (or e.g. Pemphigus as mentioned above), becomes a target of attack by the immune system, preferably the primary (or a primary) target of attack. The term also includes antigenic substances that induce conditions having the characteristics of an autoimmune disease of the skin when administered to mammals. Additionally, the term includes fragments comprising antigenic determinants (epitopes; preferably immunodominant epitopes) or epitope regions (preferably immunodominant epitope regions) of skin autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “skin bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in an autoimmune disease of the skin. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Endocrine Autoantigens and Bystander Antigens

In an alternative embodiment of the present invention the autoantigen or bystander antigen may be an enocrine autoantigen or bystander antigen or antigenic determinant thereof, for use to treat an autoimmune disease of an endocrine gland.

The term “endocrine autoantigen” as used herein includes any substance or a component thereof normally found within a mammal that, in an autoimmune disease of an endocrine gland, such as Autoimmune oophoritis (or e.g. Grave's disease or diabetes as mentioned above), becomes a target of attack by the immune system, preferably the primary (or a primary) target of attack. The term also includes antigenic substances that induce conditions having the characteristics of an autoimmune disease of the skin when administered to mammals. Additionally, the term includes fragments comprising antigenic determinants (epitopes; preferably immunodominant epitopes) or epitope regions (preferably immunodominant epitope regions) of endocrine autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “endocrine bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in an autoimmune disease of an endocrine gland. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Allergens and Antigenic Determinants Thereof

In an alternative embodiment of the present invention the antigen or bystander antigen may be an allergen or bystander antigen for use to treat an allergic condition.

The term “allergen” as used herein means any substance which can induce an allergic response, especially a type I hypersensitive response. Typical allergens include, but are not limited to, pollens, molds, foods, animal danders or their excretions, smuts and insects, their venoms or their excretions. Allergens may, for example, be natural or synthetic organic molecules such as peptides/proteins, polysaccharides or lipids. They may be administered singly or as a mixture. Allergens may be chemically or physically modified. Such modified allergens, or allergen derivatives, are known in the art. Examples include, but are not limited to, peptide fragments, conjugates or polymerized allergen derivatives. Thus, the term “allergen” as used herein includes naturally occurring (native) allergens as well as any biologically active fragment, derivative, homologue or variant thereof or any antigenic determinant or epitope (especially immunodominant epitope) thereof or any polynucleotide coding for an allergen (including any biologically active fragment, derivative, homologue or variant) or antigenic determinant or epitope (especially immunodominant epitope) thereof.

The amount of allergen to be administered can be determined empirically and depends on the sensitivity of the individual as well as the desired clinical result. Generally, a regimen of desensitization initially involves the periodic administration of smaller amounts of allergen, which level is increased over the course of the regimen until a predetermined (planned) upper limit is reached or the individual can tolerate exposure to such allergen without a significant adverse allergic response. The particular regimen often is tailored to individual patient needs. The embodiment and potential advantage of the present invention is that it may be possible to meaningfully decrease the level of allergens administered and/or the number of injections and, thereby, the length of the desensitization regimen. Further, with a meaningful decrease of the level (dose) of allergen administered to particularly sensitive individuals, there is a possible diminished risk of severe allergic reaction to the administration of the allergen.

The progress of immunotherapy can be monitored by any clinically acceptable diagnostic tests. Such tests are well known in the art and include symptom levels and requirement levels for ancillary therapy recorded in a daily diary, as well as skin testing and in vitro serological tests for specific IgE antibody and/or specific IgG antibody.

The present invention may be used for preventing and treating all forms of allergy and allergic disorder, including without limitation: ophthalmic allergic disorders, including allergic conjunctivitis, vernal conjunctivitis, vernal keratoconjunctivitis, and giant papillary conjunctivitis; nasal allergic disorders, including allergic rhinitis and sinusitis; otic allergic disorders, including eustachian tube itching; allergic disorders of the upper and lower airways, including intrinsic and extrinsic asthma; allergic disorders of the skin, including dermatitis, eczema and urticaria; and allergic disorders of the gastrointestinal tract.

Any form of allergen (including any biologically active fragment, derivative, homologue or variant) or antigenic determinant or epitope (especially immunodominant epitope) thereof or any polynucleotide coding for an allergen (including any biologically active fragment, derivative, homologue or variant) or antigenic determinant or epitope (especially immunodominant epitope) thereof may be used, including but not limited to pollen allergens, mite allergens, animal dander allergens, latexes, food allergens, insect allergens (e.g. mite or cockroach allergens), fungal allergens, drug allergens and venom allergens and antigenic determinants or epitopes (especially immunodominant epitopes) thereof, for example:

Pollen Allergens and Antigenic Determinants Thereof

1. Grass Pollen Antigens and Antigenic Determinants

A. Ryegrass Pollen Antigens and Antigenic Determinants

For example, Lol p 1 (e.g. GenBank Accession No M57474), Lol p 1b (e.g. GenBank Accession No M59163), Lol p 2 (e.g. GenBank Accession No X73363), Lol p 2a (e.g. SwissProt Accession No P14947), Lol p 2b (e.g. PIR Accession No A48595), Lol p 3 (e.g. SwissProt Accession No P14948), Lol p 4 (e.g. PIR Accession No A60737), Lol p 5 (e.g. PIR Accession No S38288), Lol p 9 (e.g. GenBank Accession No L13083) or Lol p 11 (e.g. PIR Accession No A54002); and antigenic determinants thereof.

For example, an amino acid sequence for Lol p 1 is reported as follows (GenBank Accession No M57474; SEQ ID NO: 77):

MASSSSVLLVVALFAVFLGSAHGIAKVPPGPNITAEYGDKWLDAKSTWYG
KPTGAGPKDNGGACGYKNVDKAPFNGMTGCGNTPIFKDGRGCGSCFEIKC
TKPESCSGEAVTVTITDDNEEPIAPYHFDLSGHAFGSMAKKGEEQNVRSA
GELELQFRRVKCKYPDDTKPTFHVEKASNPNYLAILVKYVDGDGDVVAVD
IKEKGKDKWTELKESWGAVWRIDTPDKLTGPFTVRYTTEGGTKSEFEDVI
PEGWKADTSYSAK

b. Timothy Grass Pollen Antigens and Antigenic Determinants

For example, Phl p 1 (e.g. GenBank Accession No X78813), Phl p 2 (e.g. GenBank Accession No X75925), Phl p 5 (e.g. GenBank Accession No Z27083), Phl p 5a (e.g. GenBank Accession No X70942), Phl p 5b (e.g. GenBank Accession No Z27083), Phl p 6 (e.g. GenBank Accession No Z27082), Phl p 11 (e.g. GenBank Accession No X77583), Phl p 32K (e.g. PIR Accession No S38294) and Phl p 38K (e.g. PIR Accession No S38293); and antigenic determinants thereof.

For example, an amino acid sequence for Phl p 1 is reported as follows (GenBank Accession No X78813; SEQ ID NO: 78):

MASSSSVLLVVVLFAVFLGSAYGIPKVPPGPNITATYGDKWLDAKSTWYG
KPTGAGPKDNGGACGYKDVDKPPFSGMTGCGNTPIFKSGRGCGSCFEIKC
TKPEACSGEPVVVHITDDNEEPIAPYHFDLSGHAFGAMAKKGDEQKLRSA
GELELQFRRVKCKYPEGTKVTFHVEKGSNPNYLALLVKYVNGDGDVVAVD
IKEKGKDKWIELKESWGAIWRIDTPDKLTGPFTVRYTTEGGTKTEAEDVI
PEGWKADTSYESK

c. Bent Grass Pollen Antigens and Antigenic Determinants

For example, Agr a 1 (e.g. PIR Accession No E37396); and antigenic determinants thereof.

d. Bermuda Grass Pollen Antigens and Antigenic Determinants

For example, Cyn d 1 (e.g. PIR Accession No A61226) and Cyn d 2 (e.g. GenBank Accession No AJ131335); and antigenic determinants thereof.

e. Blue Grass Pollen Antigens and Antigenic Determinants

For example, Poa p 1 (e.g. PIR Accession No F37396), Poa p 2 (e.g. GenBank Accession No AJ131337) and Poa p 9 (e.g. GenBank Accession No M38342); and antigenic determinants thereof.

f. Canary Grass Pollen Antigens and Antigenic Determinants

For example, Pha a 1 (e.g. SwissProt Accession No Q41260) Pha a 5.1 (e.g. SwissProt Accession No P56154) Pha a 5.2 (e.g. SwissProt Accession No P56165) Pha a 5.3 (e.g. SwissProt Accession No P56166) and Pha a 5.4 (e.g. SwissProt Accession No P56167); and antigenic determinants thereof.

g. Orchard Grass Pollen Antigens and Antigenic Determinants

For example, Dac g 1 (e.g. PIR Accession No D58493) Dac g 2 (e.g. GenBank Accession No S45354) Dac g 3 (e.g. PIR Accession No A60359); and antigenic determinants thereof.

2. Tree Pollen Antigens and Antigenic Determinants

A. Birch Pollen Antigens and Antigenic Determinants

For example, Bet v 1a (e.g. GenBank Accession No X15877), Bet v 1b (e.g. GenBank Accession No X77200), Bet v 1c (e.g. GenBank Accession No X77265), Bet v 1d (e.g. GenBank Accession No X77266), Bet v 1e (e.g. GenBank Accession No X77267), Bet v If (e.g. GenBank Accession No X77268), Bet v 1g (e.g. GenBank Accession No X77269), Bet v 1j (e.g. GenBank Accession No X77271), Bet v 1k (e.g. GenBank Accession No X77272), Bet v 1L (e.g. GenBank Accession No X77273), Bet v 1m (e.g. GenBank Accession No X81972), Bet v 2 (e.g. Genbank Accession No M65179), Bet v 3 (e.g. GenBank Accession No X79267), or Bet v 4 (e.g. GenBank Accession No Y12560); and antigenic determinants thereof.

For example, an amino acid sequence for Bet v 1a is reported as follows (GenBank Accession No X15877; SEQ ID NO: 79):

MGVFNYETETTSVIPAARLFKAFILDGDNLFPKVAPQAISSVENIEGNGG
PGTIKKISFPEGFPFKYVKDRVDEVDHTNFKYNYSVTEGGPIGDTLEKIS
NEIKIVATPDGGSILKISNKYHTKGDHEVKAEQVKASKEMGETLLRAVES
YLLAHSDAYN

b. Chestnut Tree Pollen Antigens and Antigenic Determinants

For example, Cas s 1 (e.g. PIR Accession No PC2001); and antigenic determinants thereof.

c. Hornbeam Tree Pollen Antigens and Antigenic Determinants

For example, Car b 1 (e.g. GenBank Accession No X66932); and antigenic determinants thereof.

d. Oak Tree Pollen Antigens and Antigenic Determinants

For example, Que a 1 (e.g. PIR Accession No D53288); and antigenic determinants thereof.

e. Olive Tree Pollen Antigens and Antigenic Determinants

For example, Ole e 1 (e.g. GenBank Accession No S75766), Ole e 3 (e.g. GenBank Accession No AF015810), Ole e 4 (e.g. SwissProt Accession No P80741) Ole e 5 (e.g. SwissProt Accession No P80740) Ole e 6 (e.g. GenBank Accession No U86342); and antigenic determinants thereof.

3. Weed Pollen Antigens and Antigenic Determinants

a. Ragweed (A. artemisiifloria)

For example, Amb a 1.1 (e.g. GenBank Accession No M80558), Amb a 1.2 (e.g. GenBank Accession No M80559), Amb a 1.3 (e.g. GenBank Accession No M62961), Amb a 1.4 (e.g. GenBank Accession No. M80562), Amb a 2 (e.g. GenBank Accession No. M80561) Amb a 3 (e.g. GenBank Accession No. P00304) and Amb a 5 (e.g. SwissProt Accession No. P02878); and antigenic determinants thereof.

For example, an amino acid sequence for Amb a 1.1 is reported as follows (GenBank Accession No. M80558; SEQ ID NO: 80):

MGIKHCCYILYFTLALVTLLQPVRSAEDLQEILPVNETRRLTTSGAYNII
DGCWRGKADWAENRKALADCAQGFGKGTVGGKDGDIYTVTSELDDDVANP
KEGTLRFGAAQNRPLWIIFERDMVIRLDKEMVVNSDKTIDGRGAKVEIIN
AGFTLNGVKNVIIHNINMHDVKVNPGGLIKSNDGPAAPRAGSDGDAISIS
GSSQIWIDHCSLSKSVDGLVDAKLGTTRLTVSNSLFTQHQFVLLFGAGDE
NIEDRGMLATVAFNTFTDNVDQRMPRCRHGFFQVVNNNYDKWGSYAIGGS
ASPTILSQGNRFCAPDERSKKNVLGRHGEAAAESMKWNWRTNKDVLENGA
IFVASGVDPVLTPEQSAGMIPAEPGESALSLTSSAGVLSCQPGAPC

b. Ragweed (A. psilostachya)

For example, Amb p 5 (e.g. GenBank Accession No. L24465); and antigenic determinants thereof.

c. Ragweed (A. trifida)

For example, Amb t 5 (e.g. GenBank Accession No. M38782); and antigenic determinants thereof.

4. Crop Pollen Antigens and Antigenic Determinants

a. Brassica (Rape)

For example, Bra n 1 (e.g. GenBank Accession No D63151) and Bra n 2 (e.g. GenBank Accession No D63152); and antigenic determinants thereof.

For example, an amino acid sequence for Bra n 1 is reported as follows (GenBank Accession No D63151; SEQ ID NO: 81):

MADAEHERIFKKFDTDGDGKISAAELEEALKKLGSVTPDDVTRMMAKIDT
DGDGNISFQEFTEFASANPGLMKDVAKVF

b. Maize Pollens

For example, Zea m 1 (e.g. GenBank Accession No L14271); and antigenic determinants thereof.

c. Rice Pollens

For example, Ory s 1 (e.g. GenBank Accession No U31771); and antigenic determinants thereof.

Mite Allergens

Mite allergens include all types of allergens found in mites. Common types of mite allergen include, for example, enzymes such as proteases (e.g. trypsin, chymotrypsin) amylase, and glutathione transferase or structural proteins such as tropomyosin. Suitably the mite allergen is a dust mite allergen.

Any form of mite antigen or antigenic determinant or any polynucleotide coding for a mite antigen or antigenic determinant (including any biologically active fragment, derivative, homologue or variant) may be used, including but not limited to epitopic polypeptide or polynucleotide sequences of the following:

antigens or antigenic determinants from dust mites such as Dermatophagoides farinae such as Der f 1 (e.g. SwissProt Accession Nos P16311, Q9GYY0), Der f 2 (e.g. SwissProt Accession Nos Q00855, Q9BIX2), Der f 3 (e.g. SwissProt Accession Nos P49275+, Q94508, Q9TWV8), Der f 6 (e.g. SwissProt Accession No P49276), Der f 7 (e.g. SwissProt Accession No Q26456), Der f mag (e.g. SwissProt Accession No P39673), Der f mag29 (e.g. SwissProt Accession No P39674), Der f mag3 (e.g. SwissProt Accession No Q94507),

Der f 15 (e.g. SwissProt Accession No Q9U6R7);

antigens or antigenic determinants from mites such as Glycyphagus domesticus, such as Gly d 2.02 (e.g. SwissProt Accession No Q9NFQ4);

antigens or antigenic determinants from dust mites such as Dermatophagoides pteronyssinus such as Der p 1 (e.g. SwissProt Accession No P08176), Der p 2 (e.g. SwissProt Accession No P49278), Der p 3 (e.g. SwissProt Accession No P39675),

Der p 4 (e.g. SwissProt Accession Nos P49274, Q9Y197), Der p 5 (e.g. SwissProt Accession No P14004) Der p 6 (e.g. SwissProt Accession No P49277), Der p 7 (e.g. SwissProt Accession No P49273), Der p 10 (e.g. SwissProt Accession No O18416), Der p 8 (e.g. SwissProt Accession No P46419);

antigens or antigenic determinants from dust mites such as Dermatophagoides microceras, such as Der m 1 (e.g. SwissProt Accession No P16312);

antigens or antigenic determinants from mites such as Euroglyphus, such as Eur m 1 (e.g. SwissProt Accession No P25780), Eur m 2.0101 (e.g. SwissProt Accession No Q9TZZ2) or Eur m 3.0101 (e.g. SwissProt Accession No O97370);

antigens or antigenic determinants from mites such as Lepidoglyphus, such as Lep d 1 (e.g. SwissProt Accession No P80384+), Lep d 5 (e.g. SwissProt Accession No Q9U5P2), Lep d 7 (e.g. SwissProt Accession No Q9U1G2), Lep d 10 (e.g. SwissProt Accession No Q9NFZ4), Lep d 13 (e.g. SwissProt Accession No Q9U5P1);

For example, an amino acid sequence for Der p I is reported as follows (SwissProt Accession No P08176; SEQ ID NO: 82):

| | |
|---|---|
| 1 | mkivialasl lalsavyarp ssiktfeeyk kafnksyatf edeeaarknf lesvkyvqsn |
| 61 | ggainhlsdl sldefknrfl msaeafehlk tqfdlnaetn acsingnapa eidlrqmrtv |
| 121 | tpirmqggcg scwafsgvaa tesaylayrn qsldlaeqel vdcasqhgch gdtiprgiey |
| 181 | iqhngvvqes yyryvareqs crrpnaqrfg isnycqiypp nvnkireala qthsaiavii |
| 241 | gikdldafrh ydgrtiiqrd ngyqpnyhav nivgysnaqg vdywivrnsw dtnwgdngyg |
| 301 | yfaanidlmm ieeypyvvil |

For example, an amino acid sequence for Der f I is reported as follows (e.g. SwissProt Accession No P 16311; SEQ ID NO: 83):

| | |
|---|---|
| 1 | mkfvlaiasl lvlstvyarp asiktfeefk kafnknyatv eeeevarknf leslkyvean |
| 61 | kgainhlsdl sldefknryl msaeafeqlk tqfdlnaets acrinsvnvp seldlrslrt |
| 121 | vtpirmqggc gscwafsgva atesaylayr ntsldlseqe lvdcasqhgc hgdtiprgie |
| 181 | yiqqngvvee rsypyvareq rcrrpnsqhy gisnycqiyp pdvkqireal tqthtaiavi |
| 241 | igikdlrafq hydgrtiiqh dngyqpnyha vnivgygstq gddywivrns wdttwgdsgy |
| 301 | gyfqagnnlm mieqypyvvi m |

Animal Food Allergens

Animal food allergens include all types of allergens found in foods originating with animals, such as milk, eggs and fish/seafoods. Common types of animal food allergen include, for example antigens or antigenic determinants from tropomyosins, parvalbumins, ovomucoids, ovalbumins, ovotransferrins, lysozymes, vitellogenins, apovitellenins, serum albumins (such as Bovine Serum Albumin; BSA), beta-lactoglobulins, alpha-lactalbumins and caseins (such as Casein, alpha-S1 Casein and Alpha-S2 Casein).

Any form of animal food antigen or antigenic determinant or any polynucleotide coding for an animal food antigen or antigenic determinant (including any biologically active fragment, derivative, homologue or variant) may be used, including but not limited to polypeptide or polynucleotide sequences of the following:

Fish allergens such as those from Carp (e.g. Cyc p 1.02 and Cyc p 1.01), Cod (e.g. Gad c1; e.g. SwissProt Accession No P02622), Mackerel (e.g. Tra j 1; e.g. SwissProt Accession No Q91482) and Salmon (e.g. Sal s 1; e.g. SwissProt Accession No Q91482);

Marine mollusc allergens such as those from Crab (e.g. Cha f 1; e.g. SwissProt Accession No Q9N2R3), Lobster (e.g. Hom a 1; e.g. SwissProt Accession No O44119), Shrimp (e.g. Met e1; e.g. SwissProt Accession No Q25456) and Spiny Lobster (e.g. Pan s 1; e.g. SwissProt Accession No O61379);

Egg allergens such as ovomucoids (e.g. Gal d1; e.g. SwissProt Accession No. P01005), ovalbumins (e.g. Gal d2; e.g. SwissProt Accession No P01014), ovotransferrins (e.g. Gal d3; e.g. SwissProt Accession No P02789), lysozymes (e.g. Gal d4; e.g. SwissProt Accession No P00698), vitellogenins, apovitellenins and tropomyosins (e.g. Hom a 1);

Milk allergens such as those from cow milk, such as BSA (e.g. Bos d 6; e.g. SwissProt Accession No P02769), beta-lacto globulins, alpha-lactalbumins, alpha-S1 caseins and alpha-S2 caseins.

Plant Food Allergens

Plant food allergens include all types of allergens found in plant matter used as food. Common examples include, for example plant enzymes such as papains, pectate lyases, superoxide dismutases, glyoxalases, beta-fructofuranosidases and phosphate isomerases; plant enzyme inhibitors such as amylase inhibitors and trypsin inhibitors; plant profilins, patatins, actinidins, glycinins, beta-conglycinins, agglutinins and gliadins

Any form of plant food allergen or antigenic determinant or any polynucleotide coding for a plant food allergen or antigenic determinant (including any biologically active fragment, derivative, homologue or variant) may be used in the present invention, including but not limited to polypeptide or polynucleotide sequences of the following:

Avocado allergens and antigenic determinants such as those from Prs a 1 (e.g. SwissProt Accession No P93680);

Apple allergens and antigenic determinants such as those from Mal d 1 (e.g. SwissProt Accession No P43211), Mal d 4 (e.g. SwissProt Accession No Q9XF42), Mal d 3 (e.g. SwissProt Accession No Q9M5×7);

Apricot allergens and antigenic determinants such as those from Pru ar 3 (e.g. SwissProt Accession No P81651), Pru ar 1 (e.g. SwissProt Accession No O50001);

Barley allergens and antigenic determinants such as those from Hor v 1 (e.g. SwissProt Accession No P16968) and Hor v 9 (e.g. SwissProt Accession No Q9S8H1);

Buckwheat allergens and antigenic determinants such as those from Fag ag 1 (e.g. SwissProt Accession No Q9XFM4);

Carrot allergens and antigenic determinants such as those from Dau c 1 (e.g. SwissProt Accession No O04298);

Castor Bean allergens and antigenic determinants such as those from Ric c 1 (e.g. SwissProt Accession No P01089);

Celery allergens and antigenic determinants such as those from Api g 1 (e.g. SwissProt Accession No P49372) Api g 5 (e.g. SwissProt Accession No P81943) and Api g 1.0201 (e.g. SwissProt Accession No P92918), Api g 3 (e.g. SwissProt Accession No P92919) Api g 4 (e.g. SwissProt Accession No Q9XF37);

Cherry allergens and antigenic determinants such as those from Pru a 1 (e.g. SwissProt Accession No 024248), Pru a 2 (e.g. SwissProt Accession No P50694), Pru av 3 (e.g. SwissProt Accession No Q9M5×8), Pru av 4 (e.g. SwissProt Accession No Q9XF39);

Kidney Bean allergens and antigenic determinants such as those from PR Protein (e.g. SwissProt Accession Nos P25985+ and P25986);

Kiwi allergens and antigenic determinants such as those from Act c 1 (e.g. SwissProt Accession No P00785);

Maize allergens and antigenic determinants such as those from Zea m 14 (e.g. SwissProt Accession No P19656), Profilin (e.g. SwissProt Accession No O22655), Zea m 1 (e.g. SwissProt Accession No Q07154);

Mustard leaf allergens and antigenic determinants such as those from Bra j 1 L (e.g. SwissProt Accession No P80215);

Mustard white allergens and antigenic determinants such as those from Sin a 1 (e.g. SwissProt Accession No Q41196);

Olive allergens and antigenic determinants such as those from Ole e 1 (e.g. SwissProt Accession No P19963), Ole e 3 (e.g. SwissProt Accession No O81092), Ole e 4 (e.g. SwissProt Accession No P80741), Ole e 5 (e.g. SwissProt Accession No P80740), Ole e 6 (e.g. SwissProt Accession No O24172), Ole e 7 (e.g. SwissProt Accession No P81430), Ole e 8 (e.g. SwissProt Accession No Q9M7R0), Ole e 9 (e.g. Entez Accession No AAK58515), Ole e 2 (e.g. SwissProt Accession No O24169);

Papaya allergens and antigenic determinants such as those from papain (e.g. SwissProt Accession No P00784);

Peach allergens and antigenic determinants such as those from Pru p 1 (e.g. SwissProt Accession No P81402);

Pear allergens and antigenic determinants such as those from Pyr c 1 (e.g. SwissProt Accession No O65200), Pyr c 3 (e.g. SwissProt Accession No Q9M5×6), Pyr c 4 (e.g. SwissProt Accession No Q9XF38);

Pineapple allergens and antigenic determinants such as those from pineapple profilin (e.g. Entrez Accession No AAK54835);

Plantain allergens and antigenic determinants such as those from Pla 11 (e.g. SwissProt Accession No P82242), Pla 1 1.0101 (e.g. SwissProt Accession No CAC41633), Pla 1 1.0102 (e.g. SwissProt Accession No CAC41634), Pla 1 1.0103 (e.g. SwissProt Accession No CAC41635);

Plum allergens and antigenic determinants such as those from Pru d 3 (e.g. SwissProt Accession No P82534);

Potato allergens and antigenic determinants such as those from patatins (e.g. SwissProt Accession Nos P07745, P15476, P15477, P11768 and P15478) Sol a t 2 (e.g. SwissProt Accession No P16348) and Sol a t 4 (e.g. SwissProt Accession No P30941);

Rice allergens and antigenic determinants such as those from RA 1 (e.g. SwissProt Accession No Q01884), RA 2 (e.g. SwissProt Accession No Q01885), RA 5 (e.g. SwissProt Accession No Q01881), RA 14 (e.g. SwissProt Accession No Q01882), RA 17 (e.g. SwissProt Accession No Q01883) Glyoxalase (e.g. SwissProt Accession No Q9ZWJ2), Ory s 1 (e.g. SwissProt Accession No Q40638);

Soybean allergens and antigenic determinants such as those from A1aBx (e.g. SwissProt Accession No P04776), A2B1a (e.g. SwissProt Accession No P04405), A3B4 (e.g. SwissProt Accession No P04347) A5A4B3 (e.g. SwissProt Accession No P02858), Gy3 (e.g. SwissProt Accession No P11828), Gy4 (e.g. SwissProt Accession No Q43452), A5A4B3 (e.g. SwissProt Accession No Q39921), Beta-Conglycinin (e.g. SwissProt Accession No P13916), Lectin (e.g. SwissProt Accession No P05046), Trypsin Inhibitor (e.g. SwissProt Accession Nos Q39869, Q39898 and Q39899), Gly m 1 (e.g. SwissProt Accession No P22895), Gly m 1A (e.g. SwissProt Accession No Q9S8F3), Gly m 2 (e.g. SwissProt Accession No Q39802), Gly m 3 (e.g. SwissProt Accession No 065809), Gly m 3 (e.g. SwissProt Accession No 065810), Gly m bd (e.g. SwissProt Accession No Q9AVK8);

Tomato allergens and antigenic determinants such as those from Lyc e 1 (e.g. SwissProt Accession No P13447);

Turnip allergens and antigenic determinants such as those from Bra r 2 (e.g. SwissProt Accession No P81729);

Wheat allergens and antigenic determinants such as those from agglutinins (e.g. SwissProt Accession Nos P10968, P02876 and P10969), alpha amylase and trypsin inhibitors (e.g. SwissProt Accession Nos P01084, P10846, P01083, P01085, P16852, P16159, P17314, P16851, P16850 and Q41540), gliadins (e.g. SwissProt Accession Nos P04728, P04726, P02863, Q41546, P04727, P04721, P04722, P04723, P04724, P04725, P18573, P02863, P21292, P08453, P06659, P04729, P04730, P08079, P02865, Q41548 and Q41543), phosphate isomerases (e.g. SwissProt Accession No Q9FS79), glutenins (e.g. SwissProt Accession Nos P08488, P10386, P10387, P02861, P02862, P08489, P10388, P10385, P16315, Q03872, Q41603, Q03871, Q41549, Q41550, Q41551, Q41552 and Q9S8D7), Tri a 2 (CAA10349), Tri a 3 (e.g. SwissProt Accession No Q41576) and profilins (e.g. SwissProt Accession Nos P49232, P49233 and P49234).

Nut Allergens

Nut allergens include all types of allergens found in nuts. Common examples include, for example albumins, profilins, vicilins, agglutinins, arachins, glycinins and profilins.

Any form of nut allergen or antigenic determinant or any polynucleotide coding for a nut allergen or antigenic determinant (including any biologically active fragment, derivative, homologue or variant) may be used in the present invention, including but not limited to polypeptide or polynucleotide sequences of the following:

allergens or antigenic determinants from peanut such as Ara h 1 (e.g. SwissProt Accession Nos P43238, P43237), Ara h 2 (e.g. ENTREZ Accession No AAK96887), Ara h 3 (e.g. SwissProt Accession No O82580), Ara h 4 (e.g. SwissProt Accession No Q9SQH7), Ara h 5 (e.g. SwissProt Accession No Q9SQI9), Ara h 6 (e.g. SwissProt Accession No Q9SQG5), Ara h 7 (e.g. SwissProt Accession No Q9SQH1);

allergens or antigenic determinants from brazil nut such as Ber e 1 (e.g. SwissProt Accession No P04403);

allergens or antigenic determinants from chestnut (e.g. Castanea sativa) such as Cas s 1 (e.g. SwissProt Accession No Q9S8Q4); and

allergens or antigenic determinants from hazel nut such as Cor a 1-5 (e.g. SwissProt Accession No P43216), Cor a 1 (e.g. SwissProt Accession Nos Q08407, Q39454, Q39453), Cor a 1.0401 (e.g. SwissProt Accession No Q9SWR4), Cor a 1.0402 (e.g. SwissProt Accession No Q9FPK4), Cor a 1.0403 (e.g. SwissProt Accession No Q9FPK3), Cor a 1.0404 (e.g. SwissProt Accession No Q9FPK2), Cor a 9 (e.g. ENTREZ Accession No AAL73404).

Animal Allergens

Animal allergens include all types of allergens generated by animals. Common examples include, for example lipocalins, serum albumins and protease inhibitors, which are commonly present, for example, in animal danders.

Any form of animal antigen or antigenic determinant or any polynucleotide coding for an animal antigen or antigenic determinant (including any biologically active fragment, derivative, homologue or variant) may be used, including but not limited to polypeptide or polynucleotide sequences of the following:

antigens or antigenic determinants from cat danders such as Fel d 1 (e.g. SwissProt Accession No P30440), Fel d 2 (e.g. SwissProt Accession No P49064) and Fel d 3 (e.g. Entrez Accession No AAL49391);

antigens or antigenic determinants from cow danders such as Bos d 2 (e.g. PIR Accession No B59225);

antigens or antigenic determinants from dog danders such as Can f 1 (e.g. SwissProt Accession No 018873), Can f 2 (e.g. SwissProt Accession No 018874) or Can f 3 (e.g. SwissProt Accession No P49822); and antigens or antigenic determinants from horse danders such as Equ c1 (e.g. SwissProt Accession No Q95182), Equ c 2.0101 (e.g. SwissProt Accession No P81216) and Equ c 2.0102 (e.g. SwissProt Accession No P81217).

Cockroach Allergens

Any form of cockroach antigen or antigenic determinant or any polynucleotide coding for a cockroach antigen or antigenic determinant (including any biologically active fragment, derivative, homologue or variant) may be used, such as allergens from Blatella and Periplanta, including but not limited to polypeptide or polynucleotide sequences of:

Blag 1.0101 (e.g. SwissProt Accession No Q9UAM5), Bla g 1.02 (e.g. SwissProt Accession No O96522), Bla g 2 (e.g. SwissProt Accession No P54958), Bla g 4 (e.g. SwissProt Accession No P54962), Bla g 5 (e.g. SwissProt Accession No O18598) Per a 1.0104 (e.g. SwissProt Accession No O18528), Per a 1.02 (e.g. SwissProt Accession No O18527), Per a 1.0101 (e.g. SwissProt Accession No Q9TZR6), Per a 3 (e.g. SwissProt Accession No Q25641), Per a 1.0102 (e.g. SwissProt Accession No O18535), Per a 1 (e.g. SwissProt Accession No O18530) and Per a 7 (e.g. SwissProt Accession No Q9UB83).

Venom Allergens

Venom allergens include all types of allergens found in venoms, especially insect venoms. Common types of venom allergen include, for example enzyme inhibitors such as melittin and venom enzymes such as phospholipases, hyaluronidases, and diphosphatases.

Any form of venom antigen or antigenic determinant or any polynucleotide coding for a venom antigen or antigenic determinant (including any biologically active fragment, derivative, homologue or variant) may be used, including but not limited to polypeptide or polynucleotide sequences of the following:

Bee venom allergens such as allergens from the honey bee and bumble bee, for example Api m 1 (e.g. SwissProt Accession No P00630), Api m 2 (e.g. SwissProt Accession No Q08169), Api m 3 (e.g. SwissProt Accession No P01501) and Bom t 1 (e.g. SwissProt Accession No P82971);

Hornet venom allergens from hornets such as, for example, the European Hornet, D. arenaria, D. maculata, Vespa crabro and Vespa mandarinia, for example Ves c 5.01 (e.g. SwissProt Accession No P35781), Ves c 5.02 (e.g. SwissProt Accession No P35782),

Dol a 5 (e.g. SwissProt Accession No Q05108), Dol m 1.01 (e.g. SwissProt Accession No Q06478), Dol m 1.02 (e.g. SwissProt Accession No P53357), Dol m 2 (e.g. SwissProt Accession No P49371), Dol m 5.01 (e.g. SwissProt Accession No P10736), Dol m 5.02 (e.g. SwissProt Accession No P10737), Vesp c 5.01 (e.g. SwissProt Accession No P35781), Vesp c 5.02 (e.g. SwissProt Accession No P35782), Vesp m 5 (e.g. SwissProt Accession No P81657);

Ant venom allergens from ants such as, for example, common ants and the fire ants S. invicta, S. richteri and S. geminata, for example Myr p 1 (e.g. SwissProt Accession No Q07932), Myr p 2 (e.g. SwissProt Accession No Q26464), Sol i 2 (e.g. SwissProt Accession No P35775), Sol i 3 (e.g. SwissProt Accession No P35778), Sol i 4 (e.g. SwissProt Accession No P35777), Sol j 4 (e.g. Entrez Accession No AAC97369), Sol r 2 (e.g. SwissProt Accession No P35776), Sol r 3 (e.g. SwissProt Accession No P35779), Sol g 4 (e.g. SwissProt Accession No Q9NH75).

Mosquito venom allergens from mosquitos such as Aedes aegypti, for example Aed a 1 (e.g. SwissProt Accession No P50635), Aed a 2 (e.g. SwissProt Accession No P18153) and Aed a 3 (e.g. SwissProt Accession No O01949).

Wasp venom allergens from wasps such as P. annularis, P. dominulus, P. exclamans and P. fascatus, such as Pol a 5 (e.g. SwissProt Accession No Q05109), Pol a 1 (e.g. SwissProt Accession No Q9U6W0) Pol a 2 (e.g. SwissProt Accession No Q9U6V9), Pol d 5 (e.g. SwissProt Accession No P81656), Pol e 5 (e.g. SwissProt Accession No P35759) and Pol f 5 (e.g. SwissProt Accession No P35780);

Yellow jacket venom allergens from yellow jackets such as V. flavopilosa, V. germanica, V. maculifrons, V. pensylvanica, V. squamosa, V. vidua and V. vulgaris, such as Ves f 5 (e.g. SwissProt Accession No P35783), Ves g 5 (e.g. SwissProt Accession No P35784), Ves m1 (e.g. SwissProt Accession No P51528), Ves m 5 (e.g. SwissProt Accession No P35760), Ves p 5 (e.g. SwissProt Accession No P35785), Ves s 5 (e.g. SwissProt Accession No P35786), Ves vi 5 (e.g. SwissProt Accession No P35787), Ves v 1 (e.g. SwissProt Accession No P49369), Ves v 2 (e.g. SwissProt Accession No P49370) and Ves v 5 (e.g. SwissProt Accession No Q05110).

Fungal Allergens

Fungal allergens include all types of allergens originating with fungi. Common examples include, for example, ribosomal proteins, heat shock proteins and enzymes (such as proteases, enolases, alcohol dehydrogenases and superoxide dismutases (SODs)). Fungi may include, for example, strains of Altemaria, Aspergillus, Candida, Cladosporium, Fusarium, Penicillium and Trichophyton.

Any form of fungal antigen or antigenic determinant or any polynucleotide coding for a fungal antigen or antigenic determinant (including any biologically active fragment, derivative, homologue or variant) may be used in the present invention, including but not limited to polypeptide or polynucleotide sequences of the following:

antigens or antigenic determinants from Altemaria alternata such as Alt a 1 (e.g. SwissProt Accession Nos P79085, Q00021), Alt a 2 (e.g. SwissProt Accession No O94095), Alt a 3 (e.g. SwissProt Accession No P78983), Alt a 6 (e.g. SwissProt Accession No P42037) Alt a 7 (e.g. SwissProt Accession No P42058), Alt a 10 (e.g. SwissProt Accession No P42041), Alt a 11 (e.g. SwissProt Accession No Q9HDT3), Alt a 12 (e.g. SwissProt Accession No P49148);

antigens or antigenic determinants from Aspergillus mitogillin such as Asp f 1 (e.g. SwissProt Accession Nos P04389, P82261, O60023, Q9P4F0), Asp f 2 (e.g. SwissProt Accession Nos P79017, P82262), Asp f 3 (e.g. SwissProt Accession Nos O43099, P82263, O43099), Asp f 4 (e.g. SwissProt Accession No O60024), Asp f 6 (e.g. SwissProt Accession No Q92450), Asp f 7 (e.g. SwissProt Accession No O42799), Asp f 8 (e.g. SwissProt Accession No Q9UUZ6), Asp f 9 (e.g. SwissProt Accession No O42800), Asp f 13 (e.g. SwissProt Accession No O60022), Asp 11 (e.g. SwissProt Accession No P82257), Asp f 11 (e.g. SwissProt Accession No Q9Y7F6), Asp 1 2 (e.g. SwissProt Accession No P82258), Asp 1 3 (e.g. SwissProt Accession No P82259) or Asp fl 1 (e.g. SwissProt Accession No Q9UVU3);

antigens or antigenic determinants from Candida such as Can a 1 (e.g. SwissProt Accession No P43067);

antigens or antigenic determinants from Cladosporium such as Cla h 6 (e.g. SwissProt Accession No P42040), Cla h 3 (e.g. SwissProt Accession No P40108), Cla h 4 (e.g. SwissProt Accession No P42039), Cla h 5 (e.g. SwissProt Accession No P42059) or Cla h 12 (e.g. SwissProt Accession No P50344);

antigens or antigenic determinants from Penicillium citrinum such as Pen c 19 (e.g. SwissProt Accession No Q92260) or Pen c 2 (e.g. SwissProt Accession No Q9Y755);

antigens or antigenic determinants from Penicillium notatum such as Pen n 13 (e.g. PIR Accession No JC7208);

antigens or antigenic determinants from Penicillium oxalicum such as Pen o 18 (e.g. ENTREZ Accession No AAG44478);

antigens or antigenic determinants from Trichophyton such as Tri r 4 (e.g. SwissProt Accession No Q9UW98) or Tri r 2 (e.g. SwissProt Accession No Q9UW97).

Drug Allergens

Drugs or drug-like agents capable of causing allergic reactions (drug allergens) include for example:

Antibiotics such as penicillins, sulphonamides, chloramphenicol, cephalosporins, neomycin, streptomycin, bacitracin, clindamycin, dapsone, cephalosporins and vancomycin; cardiovascular agents such as ACE inhibitors, quinidine, amiodarone and methyldopa; anaesthetic drugs and muscle relaxants such as thiopentone and halothane; analgesic agents, for example morphine derivatives such as morphine, pethidine and codeine; anti-inflammatory drugs such as diclofenac, ibuprofen and indomethacin; cancer chemotherapy drugs such as cisplatin, cyclophosphamide, methotrexate, bleomycin and cytarabine; antiseptics such as chlorhexidine, iodine and mercurochrome; solvents such as cremophor; vaccines such as tetanus toxoid and diphtheria vaccine; preservatives such as parabens, sulphites and benzalkonium chlorides; biological therapeutics such as erythropoietins (EPO), insulins, blood factors such as Factor VIII, therapeutic antibodies (e.g. anti-TNF antibodies) and therapeutic enzymes (e.g. chymopapain and streptokinase); dyes such as erythrosine and tartrazine; diagnostic agents such as fluoroscein and iodine contrast reagents; hormones such as ACTH, calcitonin, glucocorticoids, and insulins; antivenoms; serum albumins such as human serum albumin; and allergy immunotherapy vaccines.

It will be appreciated that combinations of such allergens and antigenic determinants and/or polynucleotide sequences coding for them may also be used as appropriate.

Pathogen Antigens

In an alternative embodiment, a polynucleotide coding for an inhibitor of Notch signalling, such as a Notch receptor antagonist, may be administered with a polynucleotide coding for a pathogen antigen or antigenic determinant, to upregulate the immune response to the antigen or antigenic determinant and thereby provide a prophylactic or therapeutic vaccine effect. Pathogen antigens include but are not limited to the following:

Sequences coding for viral antigens or antigenic determinants may be derived, for example, from:

Cytomegalovirus (especially Human, such as gB or derivatives thereof); Epstein Barr virus (such as gp350); flaviviruses (e.g. Yellow Fever Virus, Dengue Virus, Tick-borne encephalitis virus, Japanese Encephalitis Virus); hepatitis virus such as hepatitis B virus (for example Hepatitis B Surface antigen such as the PreS1, PreS2 and S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992,10, 915-920) or purified or recombinant proteins thereof, such as NP, NA, HA, or M proteins); measles virus; mumps virus; parainfluenza virus; rabies virus; Respiratory Syncytial virus (such as F and G proteins); rotavirus (including live attenuated viruses); smallpox virus; Varicella Zoster Virus (such as gpI, II and IE63); and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Sequences coding for bacterial antigens or antigenic determinants may be derived, for example, from:

Bacillus spp., including B. anthracis (e.g. botulinum toxin); Bordetella spp, including B. pertussis (for example pertactin, pertussis toxin, filamenteous hemagglutinin, adenylate cyclase, fimbriae); Borrelia spp., including B. burgdorferi (e.g. OspA, OspC, DbpA, DbpB), B. garinii (e.g. OspA, OspC, DbpA, DbpB), B. afzelii (e.g. OspA, OspC, DbpA, DbpB), B. andersonii (e.g. OspA, OspC, DbpA, DbpB), B. hermsii; Campylobacter spp, including C. jejuni (for example toxins, adhesins and invasins) and C. coli; Chlamydia spp., including C. trachomatis (e.g. MOMP, heparin-binding proteins), C. pneumonie (e.g. MOMP, heparin-binding proteins), C. psittaci; Clostridium spp., including C. tetani (such as tetanus toxin), C. botulinum (for example botulinum toxin), C. difficile (e.g. clostridium toxins A or B); Corynebacterium spp., including C. diphtheriae (e.g. diphtheria toxin); Ehrlichia spp., including E. equi and the agent of the Human Granulocytic Ehrlichiosis; Rickettsia spp, including R. rickettsii; Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (e.g. PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa; Legionella spp, including L. pneumophila; Leptospira spp., including L. interrogans; Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example WO97/41731)); Mycobacterium spp., including M. tuberculosis (for example ESAT6, Antigen 85A, -B or -C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (e.g. capsular polysaccharides and conjugates thereof, PsaA, PspA, streptolysin, choline-binding proteins) and the protein antigen Pneumolysin (Biochem Biophys Acta, 1989,67,1007; Rubins et al., Microbial Pathogenesis, 25,337-342), and mutant detoxified derivatives thereof (see for example WO 90/06951; WO 99/03884); Treponema spp., including T. pallidum (e.g. the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Sequences coding for parasitic/fungal antigens or antigenic determinants may be derived, for example, from:

Babesia spp., including B. microti; Candida spp., including C. albicans; Cryptococcus spp., including C. neoformans; Entamoeba spp., including E. histolytica; Giardia spp., including; G. lamblia; Leshmania spp., including L. major; Plasmodium. faciparum (MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, Pfs27/25, Pfs16, Pfs48/45, Pfs230 and their analogues in Plasmodium spp.); Pneumocystis spp., including P.; carinii; Schisostoma spp., including S. mansoni; Trichomonas spp., including T. vaginalis; Toxoplasma spp., including T. gondii (for example SAG2, SAG3, Tg34); Trypanosoma spp., including T. cruzi.

Tumour Antigens

In a further embodiment, a polynucleotide coding for an inhibitor of Notch signalling, such as a Notch receptor antagonist, may be administered with a polynucleotide coding for a cancer or tumour antigen or antigenic determinant, to upregulate the immune response to the antigen or antigenic determinant and thereby provide a tumour vaccine effect.

The term “cancer antigen or antigenic determinant” or “tumour antigen or antigenic determinant” as used herein preferably means an antigen or antigenic determinant which is present on (or associated with) a cancer cell and not typically on normal cells, or an antigen or antigenic determinant which is present on cancer cells in greater amounts than on normal (non-cancer) cells, or an antigen or antigenic determinant which is present on cancer cells in a different form than that found on normal (non-cancer) cells.

Cancer antigens include, for example (but without limitation):

beta chain of human chorionic gonadotropin (hCG beta) antigen, carcinoembryonic antigen, EGFRvIII antigen, Globo H antigen, GM2 antigen, GP100 antigen, HER2/neu antigen, KSA antigen, Le (y) antigen, MUC1 antigen, MAGE 1 antigen, MAGE 2 antigen, MUC2 antigen, MUC3 antigen, MUC4 antigen, MUC5AC antigen, MUC5B antigen, MUC7 antigen, PSA antigen, PSCA antigen, PSMA antigen,

Thompson-Friedenreich antigen (TF), Tn antigen, sTn antigen, TRP 1 antigen, TRP 2 antigen, tumor-specific immunoglobulin variable region and tyrosinase antigen.

### EXAMPLES

Various preferred features and embodiments of the present invention will now be described in more detail by way of non-limiting examples.

### Example 1

### Construction of pBUD Dual Expression Plasmids

(a) Ovalbumin (OVA)

1. Chicken ovalbumin cDNA was cloned from OVA in pBluescript (+) (supplied by N. Shastri of Univ. California, Berkley (USA), Clone #NSO446; GenBank accession #J00895) by PCR using Pfu Turbo polymerase (Stratagene, La Jolla, Calif., US) and the following oligonucleotide primers:—

OVA.1:

(SEQ ID NO: 85)

CAA AAG ACA ACT CAA GCT TCA CCA TGG GCT CCA TCG
OVA.2:

(SEQ ID NO: 86)

CAG CTT TCT TCG GAT CCA GGG GAA ACA CAT CTG CCA
AAG

PCR conditions were: 94° C., 3 min; 94° C., 50 sec; 65° C., 50 sec; 72° C., 1.5 min, for 18 cycles; then 72° C., 10 min.

The PCR product was excised from 1% agarose in 1×TAE buffer (0.04 M Tris-Acetate, 0.001 M EDTA, pH 8.0) and DNA purified using a Qiagen gel extraction kit according to the manufacturer's instructions (Qiagen, Valencia, Calif., US). The resulting ovalbumin cDNA was restricted with HindIII and BamHI and purified using a QIAquick PCR purification kit according to the manufacturer's instructions.

pBUDce4.1 (Invitrogen) was restricted with HindIII and BamHI then treated with shrimp alkaline phosphatase (Roche) prior to purification on 0.8% agarose in 1×TAE buffer using a Qiagen gel extraction kit.

The HindIII, BamHI trated ovalbumin cDNA was ligated into the HindIII, BamHI treated pBUDce4.1 using T4 ligase and transformed into TOP10 cells. Cloning sites sequence was verified. The resulting plasmid was designated pBUD.OVA.

(b) DerP1

pBud.Derp1: the mature peptide of Derp1 (nt344-nt1009, 665 bp fragment; see GenBank Accession Number Y00641) was cloned into the SalI-BamHI site of pBudCE4.1 (InVitrogen, UK) by directional PCR from plasmid pThioHis/proDerp1 (kindly donated by Dr Kalsheker, Edinburgh University Medical School).

Derp1 forward primer:

(SEQ ID NO: 87)

5′-ATTAAGTCGACATCCACCATGGAAACTAACGCC-3′
Derp1 reverse primer:

(SEQ ID NO: 88)

5′-GGATTCGGATCCGAGAATGACAACATATGGATA-3′

PCR conditions were 1 cycle at 95° C. for 1′30″; 15 cycles of 95° C./45″-65° C./45″-72° C./3′ and one cycle at 72° C. for 5′. The correct sequence of the insert was verified.

(c) human Jagged-1 (JAG-1)

2. pBUDce4.1 (InVitrogen), pBUD.DerP1 (from (b) above) and pBUD.OVA (from (a) above) were each restricted with PmeI and BglII, treated with shrimp alkaline phosphatase (Roche) and then gel purified on 1% agarose in 1×TAE using Qiagen gel extraction kit.

3. A full length human Jagged-1 (JAG-1) cDNA (GenBank Accession No AF028593) was cloned by PCR from a placental cDNA library made from placental polyA+ (Clontech) and inserted into pCDNA3.1 (Invitrogen) to give pCDNA3.1-JAG-1V5HIS. The sequence was then excised from pCDNA3.1-JAG-1V5HIS using PmeI and SalI. The JAG-1 DNA was then gel purified on 1% agarose in 1×TAE using a Qiagen gel extraction kit.

4. JAG-1 cDNA (PmeI, SalI) (from step 3) was ligated into pBUDce4.1, pBUD.DerP1 or pBUD.OVA (PmeI, BglII) (from steps 1 and 2) using T4 ligase and in each case transformed into TOP10 cells. Clones sequences were verified. The resulting plasmids were designated pBUD.JAG-1, pBUD.JAG-1.DerP1 and pBUD.JAG-1.OVA.

(d) Human Delta-1 (DLL-1)

5. A full length human Delta-1 (DLL-1) cDNA (GenBank Accession No AF003522) was cloned by PCR from a placental cDNA library made from placental polyA+(Clontech) and inserted into pCDNA3.1 (Invitrogen) to give pCDNA3.1-DLL-1V5HIS.

pCDNA3.1-DLL-1V5HIS was restricted with HindIII. The 5′-overhangs were ‘filled-in’ using Klenow fragment polymerase and the DNA was purified using a QIAquick PCR purification kit. DLL-1 cDNA was excised using PmeI and gel purified on 0.8% agarose in 1×TAE using a Qiagen gel extraction kit.

6. pBUDce4.1 or pBUD.OVA were each restricted with NotI. The 5′-overhangs were ‘filled-in’ using Klenow fragment polymerase and the DNA was purified using a QIAquick PCR purification kit. The DNA was then restricted using PmeI and gel purified on 0.8% agarose in 1×TAE using a Qiagen gel extraction kit, prior to treatment with shrimp alkaline phosphatase (Roche).

7. pBUD.DerP1 was restricted with NotI. The 5′-overhangs were ‘filled-in’ using Klenow fragment polymerase and the DNA was purified using a QIAquick PCR purification kit. DerP1 cDNA was excised using PmeI and gel purified on 0.8% agarose in 1×TAE using a Qiagen gel extraction kit, prior to treatment with shrimp alkaline phosphatase (Roche).

8. DLL-1 cDNA (HindIII, PmeI) (from step 5) was ligated into pBUDce4.1 (NotI, PmeI) (from step 6) or pBUD.DerP1 (NotI, PmeI) (from step 7) using T4 ligase. Clones sequences were verified. The resulting plasmids were designated: pBUD.DLL-1 and pBUD.DLL-1.DerP1.

9. DLL-1 cDNA (HindIII, PmeI) (from step 5) was ligated into pBUD.OVA (NotI, PmeI) (from step 6) using T4 ligase and transformed into TOP10 cells. Cloning sites sequence was verified. The resulting plasmid was designated: pBUD.DLL-1.OVA.

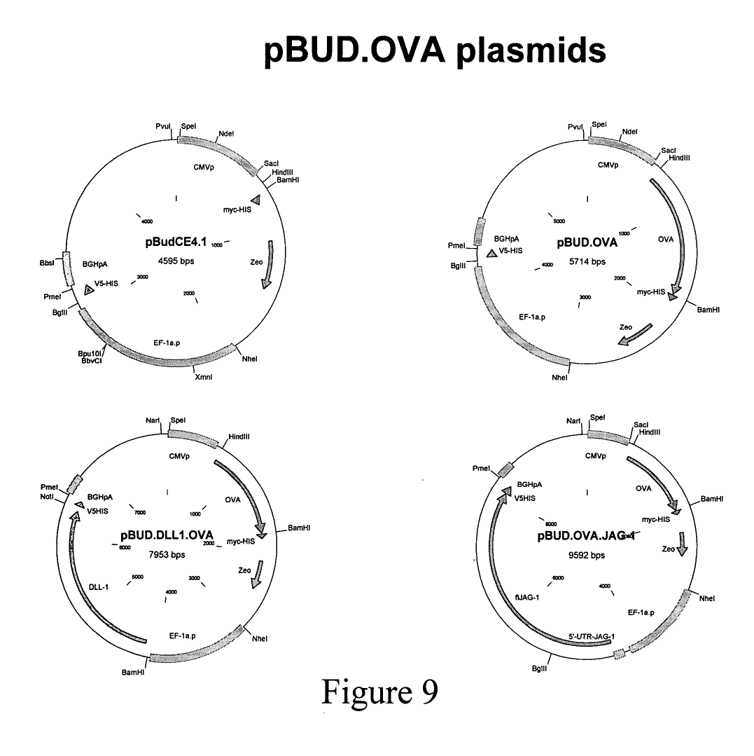

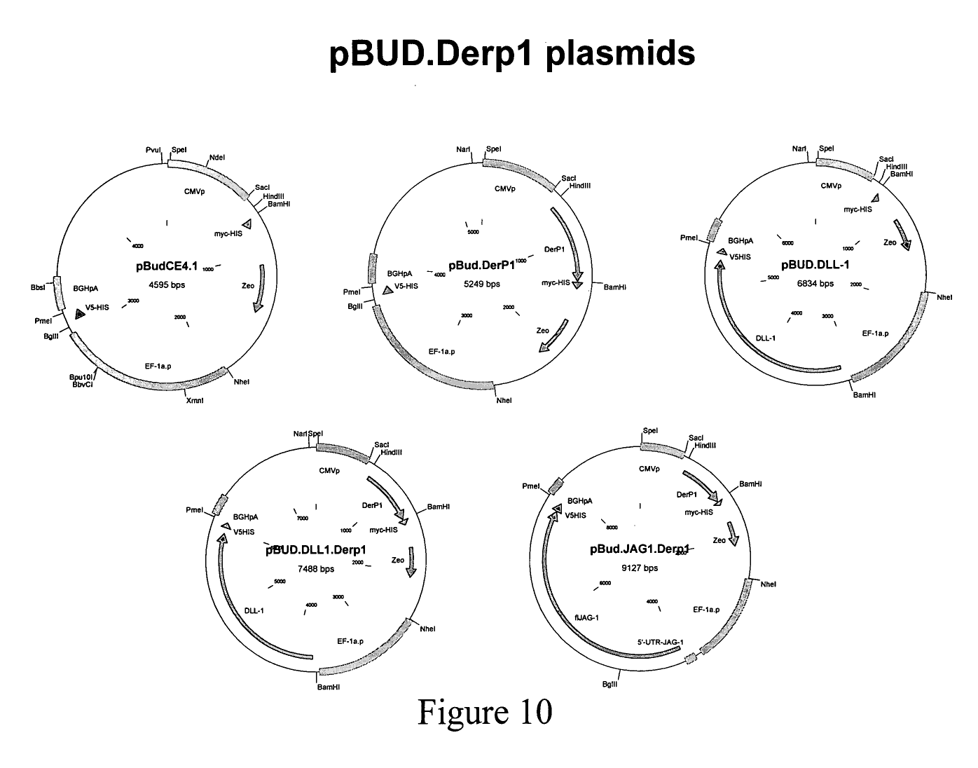

Maps of relevant plasmids are shown in FIGS. 9 and 10.

### Example 2

### CMV Dual Expression Plasmids (pJV Vectors)

Construction of CMV Dual Expression and Control Plasmids:

1. Full length human JAG-1 and DLL-1 were subjected to PCR to introduce a 5′-NheI site and 3′-stop codon and BamHI site. PCR was conducted with Pfu turbo using the following primers:

NheJAG-1:

(SEQ ID NO: 89)

GGC GCA GCG ATG GCT AGC CCA CGG ACG CGC GGC
BamJAG-1:

(SEQ ID NO: 90)

CTG CAG AGG ATC CCT ATA CGA TGT ACT CCA TTC GG
NheDLL-1:

(SEQ ID NO: 91)

CTT CAC CAT GGC TAG CCG GTG CGC GCT GG
BamDLL-1:

(SEQ ID NO: 92)

CTG CAG AGG ATC CTT ACA CCT CAG TTG CTA TGA CGC
ACT CAT

PCR conditions were 95° C., 3 min; 95° C., 1 min; 65° C., 1 min; 72° C., 5 min (JAG-1) or 4 min (DLL-1), for 18 cycles; then 72° C., 10 min.

2. DerP1 (mature form) was subjected to PCR to introduce a 5′-NheI site and a 3′-stop codon and BamHI site. PCR was conducted with Pfu turbo using the following primers:

NheDerP1:

(SEQ ID NO: 93)

CAT CCA CCA TGG CTA GCA ACG CCT GCA GTA TCA ATG
BamDerP1:

(SEQ ID NO: 94)

TTG TTC GGA TCC TTA GAG AAT GAC AAC ATA TGG

PCR conditions were 95° C., 3 min; 95° C., 1 min; 65° C., 1 min; 72° C., 2 min for 18 cycles; then 72° C., 10 min.

3. A donor expression plasmid (pJV donor) with a hCMV promoter (supplied by Powderject, Vaccines, Madison, US) was cut with NheI and BglII then treated with Shrimp alkaline phosphatase (Roche).

4. An acceptor expression plasmid (pJV acceptor) with a hCMV promoter (supplied by Powderject Vaccines) was cut with NheI and BglII then treated with Shrimp alkaline phosphatase (Roche).

5. The resulting DNA was gel purified on 0.8% agarose in 1×TAE and Qiagen gel purification kit.

6. The JAG-1, DLL-1 and DerP1 PCR products were ligated into pCRBluntII-TOPO using the Blunt TOPO PCR Cloning Kit (Invitrogen). 2 μl each ligation mix was added to a vial each of chemically competent One-Shot TOP10 cells. Incubated on ice for 30 minutes and heat-shocked at 42° C. for 35 seconds before cooling on ice for a further 2 minutes. 250 μl SOC media was added to the transformation and the sample incubated at 37° C. for 1 hour at 225 rpm. 20 and 100 μl each transformation reaction was spread onto LB/KANA plates and the plates incubated at 37° C. overnight. Individual colonies were picked into 10 ml LB/KANA broth and grown at 37° C. and 225 rpm 37° C. overnight. DNA was purified using the QIAGEN miniprep kit, according to the manufacturer's instructions. Sequences were verified.

7. The JAG-1, DLL-1 and DerP1 DNAs were then excised from pCRIIblunt with NheI and BamHI. The resulting fragments were gel purified on 1% agarose in 1×TAE and a Qiagen gel purification kit.

8. The JAG-1 and DLL-1 DNAs from step (7) were ligated into the acceptor plasmid from step (4) using T4 ligase and transformed into TOP10 cells. Cloning sites sequences were verified.

9. The JAG-1 and DLL-1 DNAs in the acceptor plasmids were linearised with MfeI prior to partial PstI restriction. The DerP1 DNA in donor plasmid was linearised with EcoRI prior to partial PstI restriction. The resulting products were resolved on 0.8% agarose gels with 1×TAE. 5940 bp DLL-1, 7417 bp JAG-1 and 1.3 kb DerP1 products were excised and purified using a Qiagen gel purification kit.

10. The DerP1 DNA in the donor (EcoRI to PstI) fragment was ligated into JAG-1 or DLL-1 in acceptor plasmid (MfeI to PstI) using T4 ligase and transformed into TOP10 cells. Cloning sites sequences were verified.

11. All plasmids were then transformed into electrocompetent competent E. coli DH5α cells by electroporation. 45 μl of cells were diluted 1:1 with 10% glycerol and chilled in an electroporation cuvette. 1 μl DNA was added to the cells before electroporation at 25 μF and 2.5 kV. Electroporated cells were transferred to 1 ml 2YT broth and incubated at 37° C., 250 rpm for 1 hour. Cells were pelleted by centrifugation at 4.5 k rpm for 2 minutes before resuspension in 250 μl LB/KANA broth and spreading of 20 or 100 μl onto LB/KANA plates. Plates were incubated at 37° C. overnight.

12. 5′-phosphates were added to adaptor primer oligonucleotides with 5′-NheI and 3′-BamHI sites using T4 polynucleotide kinase. The phosphorylated adaptors were ligated into dephosphorylated PJVdonor (NheI to BglII) DNA from (3) to give expression plasmids coding variously for JAG-1, DLL-1 and/or DerP1.

The resulting plasmids were transformed into TOP10 cells and positive clones were identified by restriction enzyme analysis.

13. The NheI/BamHI adapted donor plasmid was restricted with EcoRI and PstI and the resulting DNA was purified on 0.8% agarose in 1×TAE using a Qiagen gel extraction kit.

14. The acceptor plasmid was restricted with MfeI and PstI, then treated with shrimp alkaline phosphatase (Roche). The resulting DNA was gel purified from 0.8% in 1×TAE agarose using a Qiagen gel extraction kit.

15. The donor plasmid (NheI/BamHI adapted), EcoRI to PstI from (13) was ligated into the phosphatase-treated acceptor (MfeI to PstI) from (14) and the resulting dual expression plasmid was transformed into TOP10 cells. Cloning sites sequences were verified.

DerP1 Single Expression Vector (Control):—

16. The donor plasmid containing DerP1 sequence (EcoRI to PstI) from (9) was ligated into acceptor plasmid (MfeI to PstI) from (14). Transformed into TOP10 cells. Cloning sites sequences were verified.

17. The resulting control plasmids were subsequently transformed into Electrocompetent DH5α cells, as previously described (Step 11).

### Example 3

### Plasmid Preparation

(a) Plasmid Purification:

Cells from overnight LB/KANA 50 cultures of the relevant plasmids (50 or 500 ml) were pelleted by centrifugation. DNA was purified using Qiagen EndoFree Maxi or Mega Kits respectively, according to the manufacturer's instructions. The only exception to this method being that the cell lysate was filtered through a Whatman paper filter rather than the QIAfilter Mega Cartridge for the EndoFree Mega Kit method.

(b) Plasmid Identification:

Before particle/tube coating, the identity of all plasmids was verified by restriction enzyme digestion:

1 μg each plasmid was digested with 0.5 μl of the required enzyme in a total volume of 10 or 20 μl for 2 hours at 37° C. Bands were separated by electrophoresis at 100 V through a 1% agarose gel in 1×TBE buffer (0.089 M Tris Base, 0.089 M Boric Acid, 0.002 M EDTA, pH 8.3).

(c) Endotoxin Screening:

5 μl plasmid DNA was diluted to a final volume of 50 μl with BioWhittaker LAL water. Endotoxin screening was carried out using the BioWhittaker QCL-1000 Chromogenic LAL test, according to the manufacturer's instructions. Plasmids were only used for bullet preparation if the endotoxin level was found to be below 0.05 EU/μg DNA.

### Example 4

### Protein Expression

(a) Transient Transfection:

To detect the expression of hDLL1, hJAG1, Derp1 and OVA from the plasmids prepared as above, 5×10⁶ COS-1 cells were plated on day −1 in 175 cm² tissue culture flasks (NUNC). On day 0, cells were transfected with 20 μg of the relevant expression plasmid DNA using Lipofectin (Life Technologies, UK) or Lipofectamine 2000 (Invitrogen) at a 1:5 ratio DNA:transfection reagent. Transfections were diluted to a final volume of 8 ml in Opti-MEM Reduced Serum Media (Invitrogen). Cells were incubated in the DNA-Reagent complexes overnight at 37° C. in a CO₂ incubator before the transfection media was replaced with 20 ml D-MEM containing 10% FCS on day 2. Cell were incubated at 37° C. in a CO₂ incubator for a further 24 hours before removal of the media on day 3. The media was centrifuged at 1200 rpm for 4 minutes to remove cellular debris, whereas the cells were trypsinized to allow them to be transferred to a 1.5 ml tube. Cells were lysed and total proteins extracted with 300 μl high salt lysis buffer (500 mM NaCl, 1% NP-40, 50 mM Tris pH 8) containing a cocktail of protease inhibitors (Complete, Roche). An equal volume of 2×SDS PAGE Loading buffer was added to the lysate.

(b) Ovalbumin Concentration:

The ovalbumin gene contains an internal signal sequence, causing the expressed protein to be secreted to the media. In order to be able to screen for ovalbumin production by western blot analysis the protein needed to be concentrated by bead pull-down using the following method:

100 μl Ni-NTA beads (Qiagen), pre-equilibrated in PBS, were incubated with 16 ml D-MEM supernatant harvested from the transfected cells by rotation at 4° C. for 1-2 hours. Beads were collected by centrifugation at 2500 rpm, 4° C. for 10 minutes before washing with 10 ml PBS. Beads were resuspended in 100 μl 2×SDS loading buffer.

(c) Western Blot Analysis of Protein Expression:

Total proteins were separated by SDS-polyacrylamide gel electrophoresis through a 12% acrylamide gel at 200 V for 40 minutes before transfer to a Hybond-C membrane (Amersham) at 10 V, 200 mA for 60 minutes. Membranes were blocked with 5% skimmed milk in TBS-Tween 20 (TBST) at 4° C. overnight; washed 3 times (5 minutes each) with TBST and incubated at 4° C. in the relevant primary antibody (see table) in block buffer overnight (or for 1 hour). Blots were washed 3 times (5 minutes each) in TBST before incubation with the relevant secondary antibody (see table), diluted in TBST, for 1 hour. After washing 3 times (5 minutes each) in TBST, protein was detected using a chemiluminescent kit (ECL Plus, Amersham Pharmacia Biotech) according to the manufacturer's instructions.

| Protein | Primary Ab | Dilution | Secondary Ab | Dilution |
|---|---|---|---|---|
| Ovalbumin | α-OvalbuminG1 | 1:10000 | α rabbit HRP | 1:10000 |
| | (Abcam) | | | |
| Derp1 | α-Derp1 | 1:500 | α mouse HRP | 1:10000 |
| | (Lorantis tail | | | |
| | bleed 5) | | | |
| His-tagged | αV5-HRP | 1:5000 | — | — |
| hDLL-1 | (Invitrogen) | | | |
| His-tagged | αV5-HRP | 1:5000 | — | — |
| hJAG-1 | (Invitrogen) | | | |
| Untagged | T20 | 1:500 | α goat HRP | 1:10000 |
| hDLL-1 | (Santa Cruz) | | | |
| Untagged | C20 | 1:750 | α goat HRP | 1:10000 |
| hJAG-1 | (Santa Cruz) | 1:4 | α mouse HRP | 1:10000 |
| | or/TS115H | | | |
| | (Lorantis) | | | |

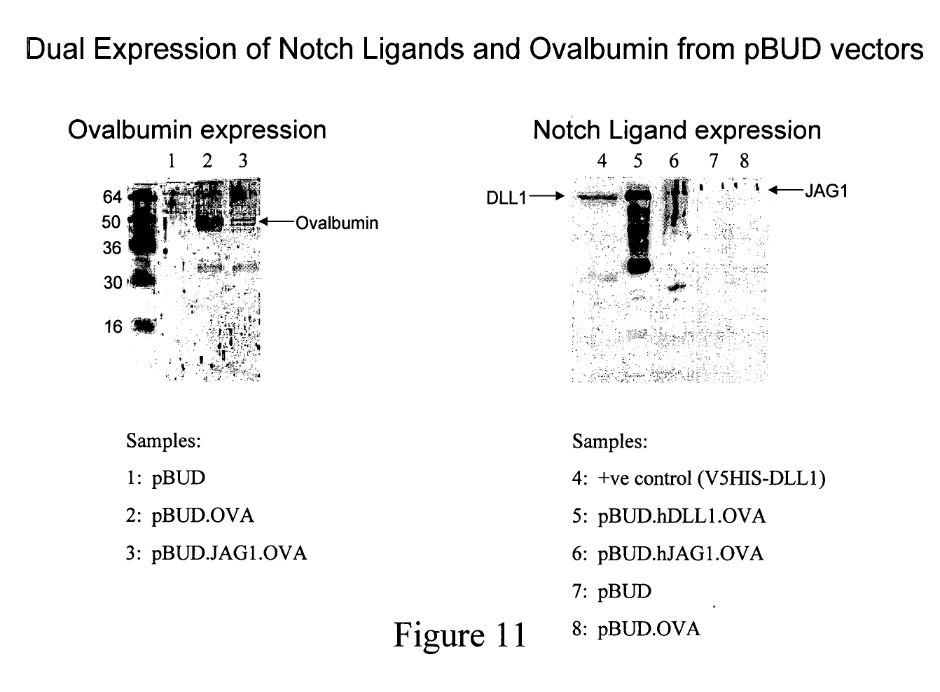

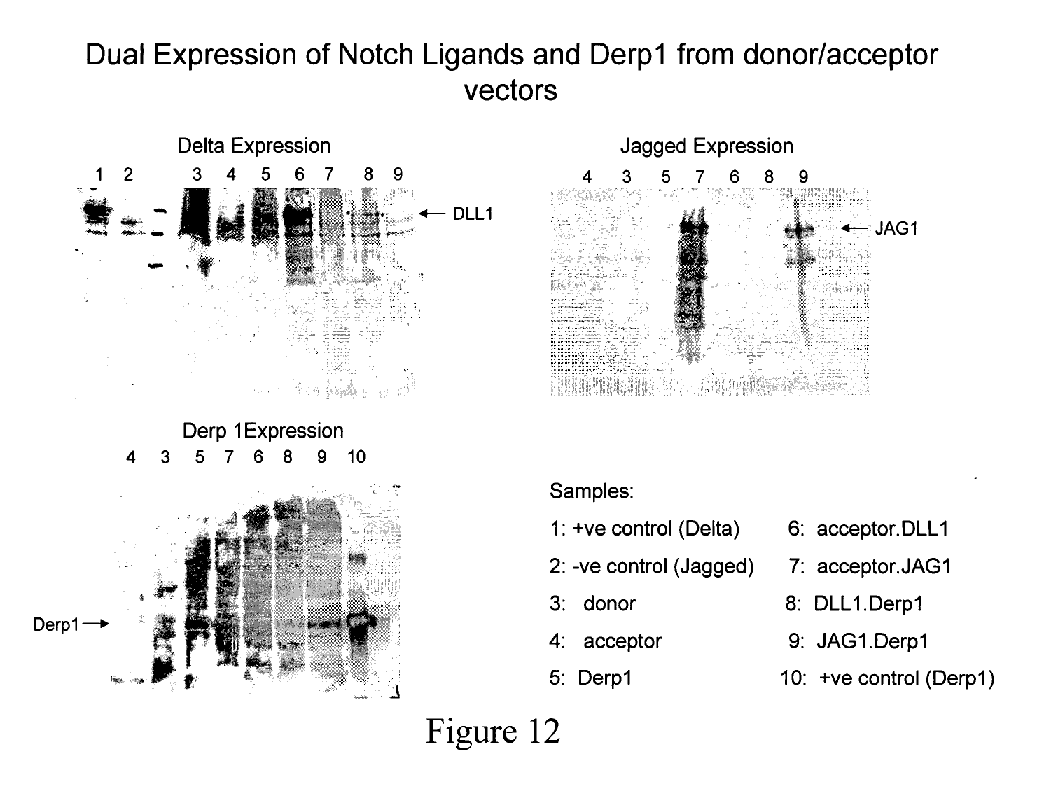

Results (blots) are shown in FIGS. 11 (pBUD vectors) and 12 (donor/acceptor vectors).

### Example 5

### In Vitro Assay of Surface-Expressed Notch Ligands

CHO—N2 Reporter Assay:

A flask of subconfluent N27#11 cells (for preparation of N27#11 see PCT Patent Publication WO 03/012441, Lorantis, Example 8) were trypsinised, spun down, resupsended in fresh complete DMEM+10% HI FCS, counted and adjusted to 2×10⁵ cells/ml. A 96-well plate of cells was set up with 100 μl of this cell suspension per well (i.e. 2×10⁴ cells per well) and placed in a CO₂ incubator for at least one hour to allow the cells to adhere.

COS-1 cells (transfected in each case with one of the above ligated DNA vaccine vectors) were trypsinised, spun down, resuspended in fresh complete DMEM+10% HI FCS and counted. The cell count was adjusted to a stock concentration of 5×10⁵ cells/ml, before being serially diluted two-fold using complete DMEM+10% HI FCS, four times. 100 μl of the stock or dilutions was added to wells of the N27#11 containing plate. CHO and CHO-hDelta1 co-culture controls were set up over the same range of cell concentrations as the transfected COS-1 cells. Plates were placed back in a CO₂ incubator overnight before performing a Steady-Glo luciferase assay (by the method described in WO 03/012441 (Lorantis)).

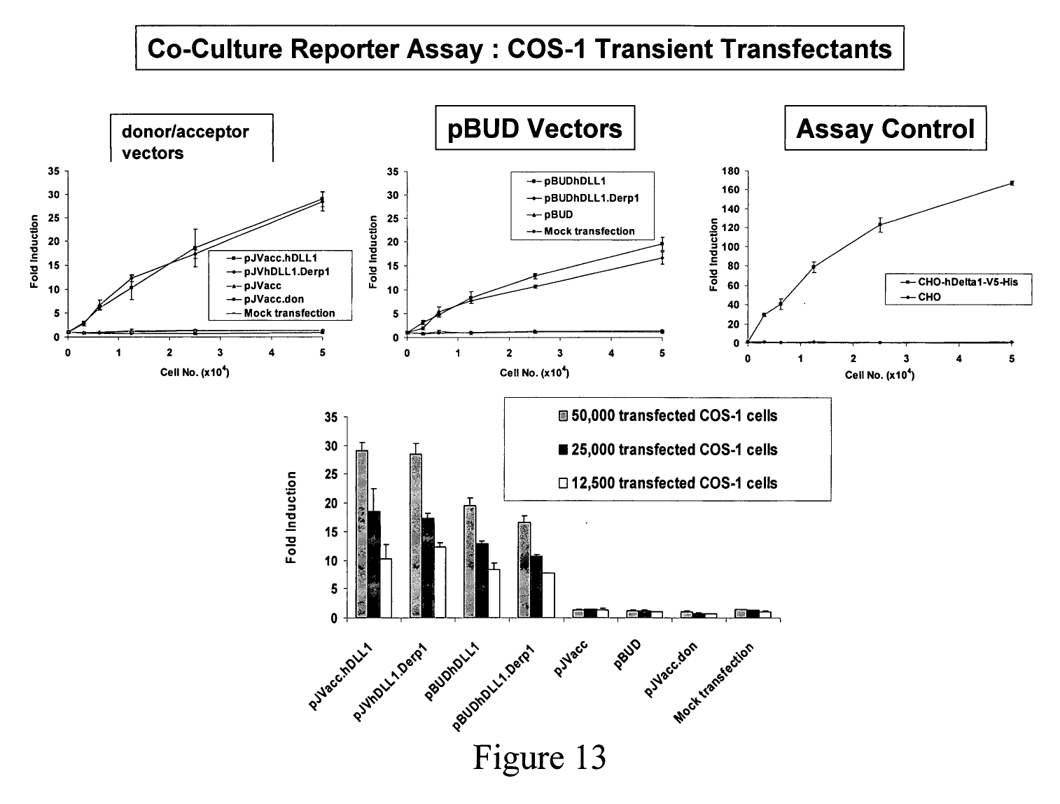

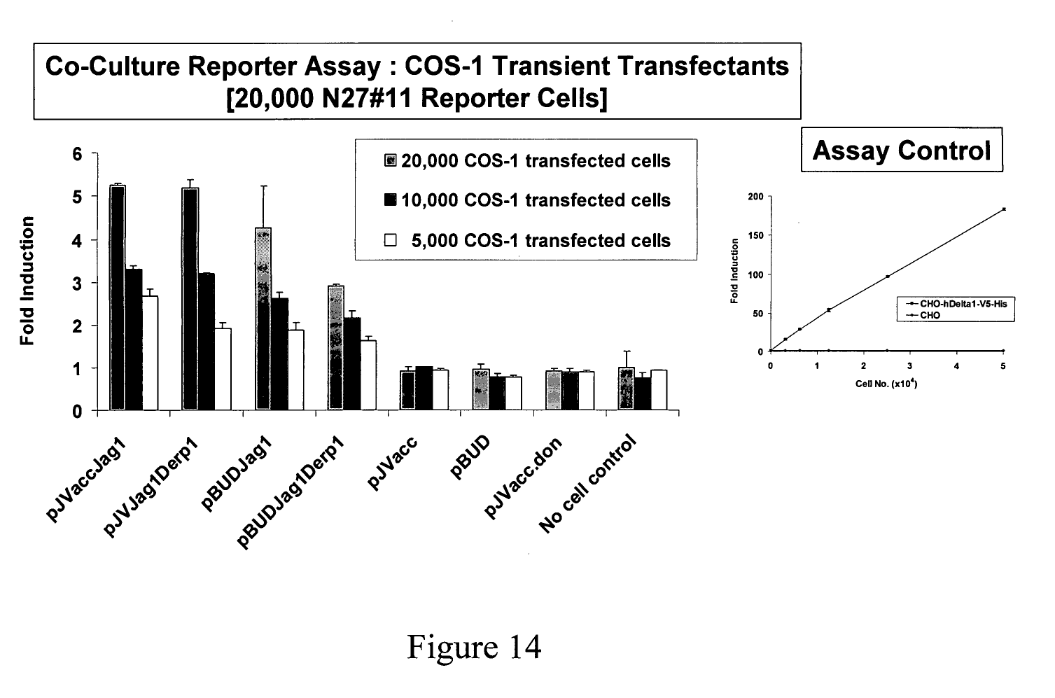

Results are shown in FIGS. 13 and 14.

### Example 6

### Gold Particle and Tube Preparation

(a) Preparation of Gold Particles and Coating onto Tubes:

25 mg gold microparticles (supplied by Powderject Vaccines, also available from Degussa) in a 1.5 ml tube was resuspended in 100 μl 50 mM spermidine (free-base, Sigma) before brief vortexing and sonicatation. Endotoxin free plasmid (<0.05 EU/μl) was added to a concentration of 2 μg per mg gold before drop wise addition of 100 μl 10% CaCl₂ solution (American Pharmaceutical Partners Inc). Samples were incubated at room temperature for 10′ prior to centrifugation for 15 seconds at room temperature. DNA/gold pellet was washed 3 times in 100% ethanol before resuspension in 100% ethanol to a concentration of 8 mg gold per ml ethanol. PVP (360 K) was added to a final concentration of 0.05 μg per ml. The DNA/gold slurry was briefly mixed by vortexing and sonication before loading into a slowly rotating, pre-purged (set to 0.5 bar on bottle regulator) 70 cm piece of Tefzel™ tubing (DuPont, US) mounted in a tube-turning device. The tube-turner speed was then altered to SPIN with the N₂ flow-rate set to 0.8 l/min during coating of the tube with DNA/gold particles. Tubing was dried under N₂ at a flow-rate of 0.1-0.2/min for 60 minutes before cutting into 0.5″ cartridges. Sections of tubing were incubated at RT overnight with desiccant before storage at 4° C.

(b) Restriction Enzyme Verification and Quantification of DNA:

DNA from 2 sections of tube was eluted into 60 μl endotoxin-free TE buffer by vortexing the tube sections in a 1.5 ml tube for 1 minute and centrifugation for 5 minutes to pellet the gold. 4 μl eluted DNA was cut with 0.5 μl EcoRI in a total volume of 10 μl for 2 hours at 37° C. to linearize the plasmid. DNA bands were separated by electrophoresis through a 1% agarose gel at 100 V in 1×TBE buffer for 1-2 hours. High DNA mass ladder was run alongside the digested DNA in order to allow identification of the plasmid and approximate quantification of the DNA loading rate. If required more DNA was digested with specific enzymes to further verify the plasmid identity.

### Example 7

(a) DNA Treatment and Basic Immunisation Protocol:

6-8 week old C57Bl/6 female mice received on two sites of their shaved abdomens DNA-coated gold microparticles delivered by needleless injection gene gun (Serial Number XR-1, supplied by Powderject Vaccines, Inc; Madison, Wis., US) from particle-coated tube sections from Example 6 above. Particles used had been coated variously with expression plasmids coding for Derp1 alone; both Derp1 and hDelta1; or vector alone (empty plasmid control). Each experimental group included 7-8 mice.

House Dust Mite (HDM) extract was prepared as follows: 80 ml of crude dust mite extract/powder was dissolved in 200 ml of sterile PBS, rotated at RT for 2 hours, centrifuged at 45000 rpm/10 min twice, supernatant filtered through 0.45 micron filter, concentrated in dialysis tube, using solid PEG, dialysed into sterile PBS, filtered through 0.2 micron filter, concentration established by Bradford and aliquots freezed.

Four weeks after the last injection the mice were immunised subcutaneously at the base of the tail with 50 μg/1001 μl of House Dust Mite (HDM) extract in Complete Freund's Adjuvant (CFA, Pierce) (1:1). After 2 weeks, to assess immune priming, mice were challenged in the right ear with 20 μg/20 μl HDM extract. Ear increases (difference between injected and non-injected ear) were measured with a digital caliper at different time points up to seven days after ear challenge.

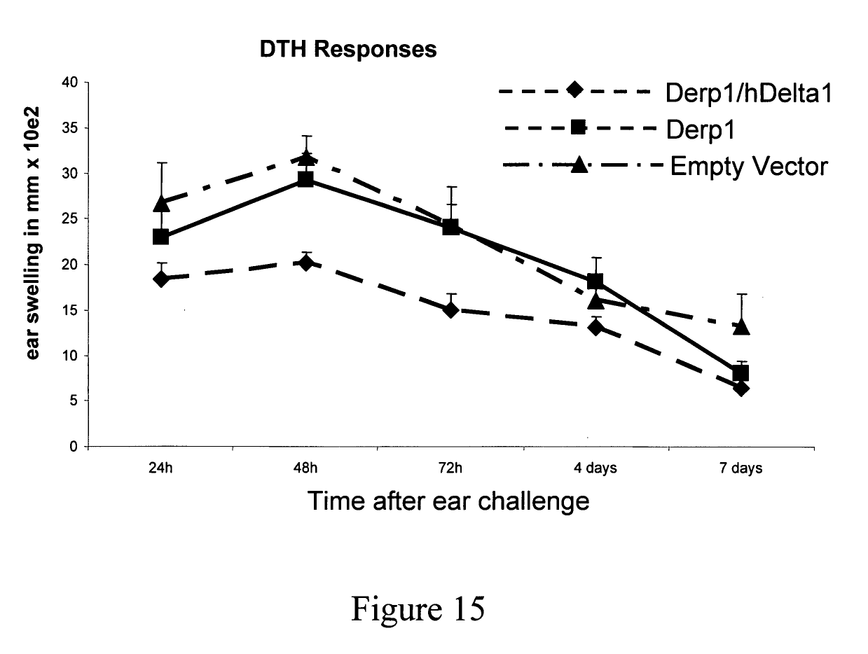

Results are shown in FIG. 15

Three weeks after ear injection the experiment was terminated and the mice sacrificed for ex vivo analysis: sera were collected by cardiac puncture and spleens removed for cell culture.

(b) Serum Antibody ELISA:

Ninety-six well plates were coated with 1 μg of HDM extract in binding buffer (50 mM Carbonate-Bicarbonate buffer, pH 9.6) and incubated overnight at 4 C.

Plates were washed in PBS/0.05% Tween buffer and blocked with 200 μl of 2% BSA-PBS/0.1% Tween-20 for 2 hours at room temperature.

After a washing step duplicate serial dilution of sample serum was added and incubated for 2 hours at room temperature. A biotinylated secondary antibody (IgG1 or IgG2a antibody (cross-reactive with IgG2c), Pharmingen) at concentration 2 μg/ml was added after a washing step for 1 hour at room temperature.

After a final wash 50 μg/well of Streptavidin-HRP (1:200 dilution in blocking buffer) was added for 30 minutes in the dark. Plates were developed by the addition of 100 μl of substrate (TMB, R & D Systems, UK) for 5-10 minutes. The reaction was stopped with 50 μl/well of 1 M H₂SO₄. The plates were read at λ450. A standard curve was included for each cytokine in order to quantify cytokine measures.

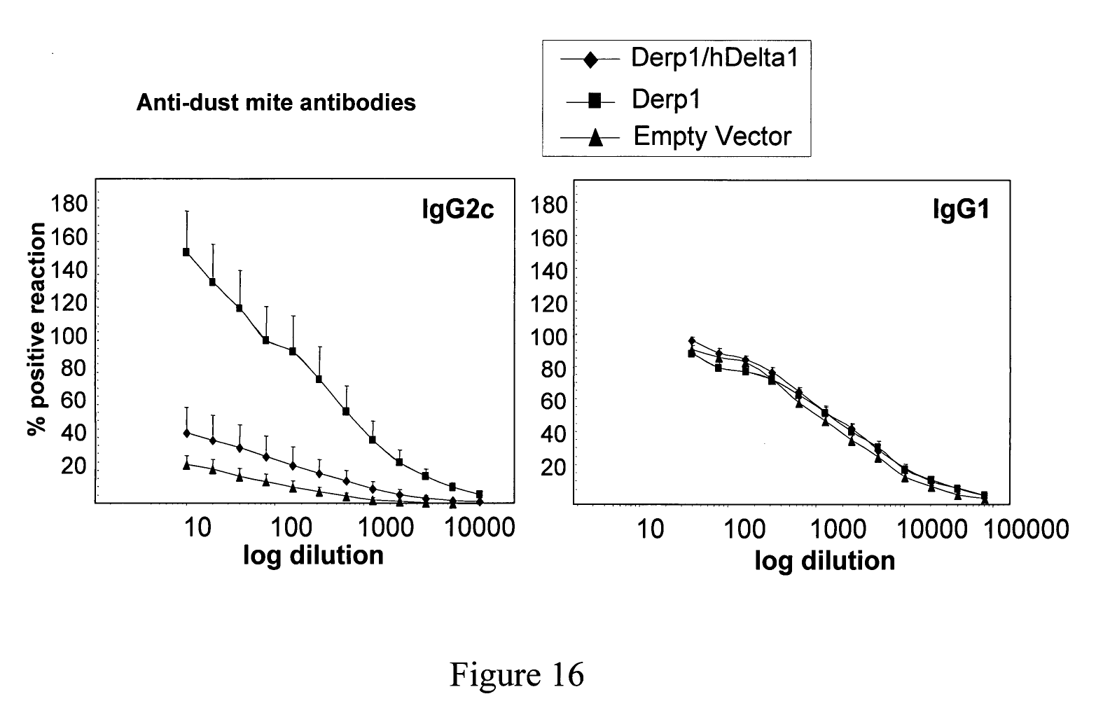

Results are shown in FIG. 16

(c) Cell Culture and Cytokine ELISA:

Spleens were removed asceptically and single cell suspension was prepared. 5×10e6 cells from individual mice were cultured in 24 well plates with 50 μg/ml of HDM. Supernatants were collected at 72 hours for cytokine analysis.

Ninety-six-well NUNC Maxisorp plate were coated with 100 ul/well capture antibody (R & D Systems, UK) in PBS (3 μg/ml for IL10, 4 μg/ml for IL13 and 4 μg/ml for IFNγ). The plates were incubated overnight at room temperature. After washing with PBS-0.05% Tween 20 the plates were blocked for 90 minutes with PBS/10% FBS. Diluted supernatants were added and incubated at room temperature for 90 minutes. Plates were washed and 100 μl/well of detection antibody (1 ng/ml for IL 10 and 2 ng/ml for IL 13 and IFNγ, R & D Systems, UK) was incubated for 90 minutes at room temperature. After a washing step, 100 ul/well of Streptavidin-HRP (diluted 1:200 in PBS/10% FBS) was added for 30 minutes at room temperature in the dark. After a final wash, 100 μl of substrate (TMB, R & D Systems, UK) was added and 30 minutes afterwards the reaction was stopped with 50 μl/well of 1 M H₂SO₄. The plates were read at λ450. A standard curve was included for each cytokine in order to quantify cytokine measures.

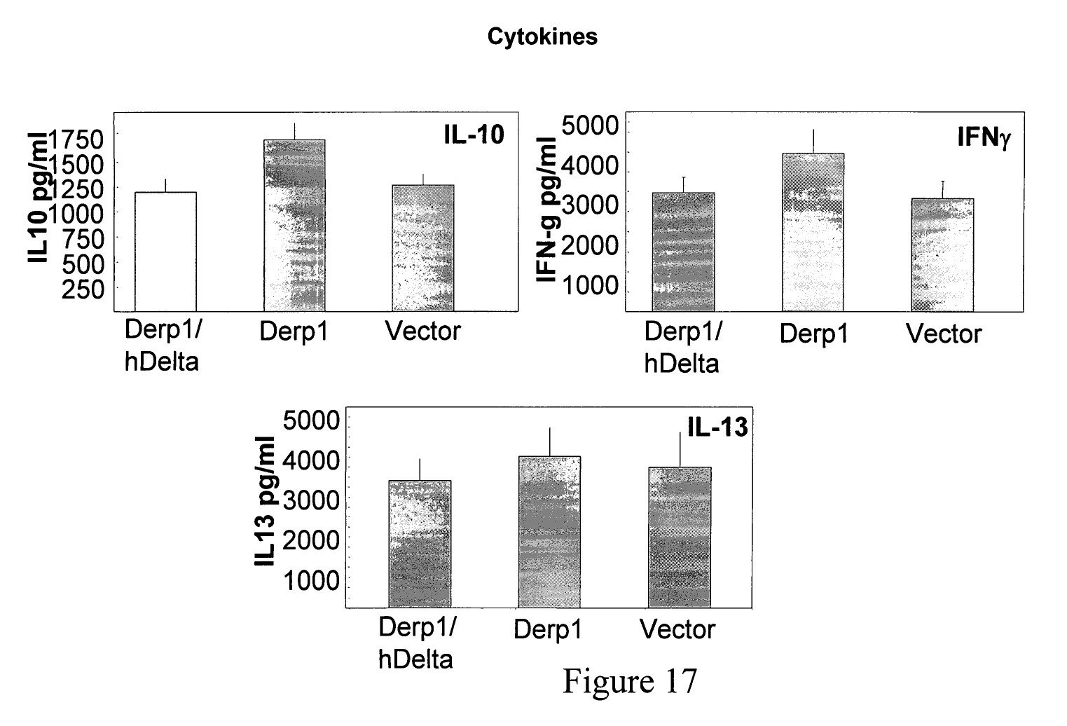

Results are shown in FIG. 17

### Example 8

(a) DNA Treatment and Basic Immunisation Protocol:

6-8 week old C57Bl/6 female mice received on two sites of their shaved abdomens DNA-coated gold microparticles delivered by gene gun (Serial Number XR-1, supplied by Powderject, Madison, US) from particle-coated tube sections from Example 6 above. Particles used had been coated variously with expression plasmids coding for Derp1 alone; both Derp1 and hDelta1; or vector alone (empty plasmid control). Each experimental group included 7-8 mice.

Seven weeks after the last injection the mice were immunised subcutaneously at the base of the tail with 50 μg/1001 μl of House Dust Mite (HDM) extract in Complete Freund's Adjuvant (CFA, Pierce) (1:1). After 2 weeks, to assess immune priming, mice were challenged in the right ear with 20 μg/20 μl HDM. Ear increases (difference between injected and non-injected ear) were measured with a digital caliper at different time points up to seven days after ear challenge.

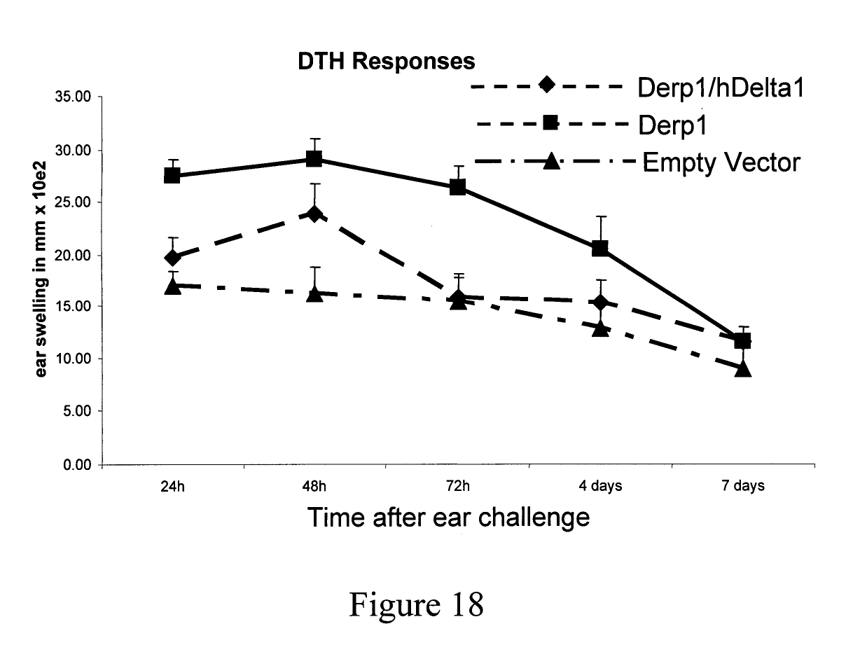

Results are shown in FIG. 18.

Three weeks after ear injection the experiment was terminated and the mice sacrificed for ex vivo analysis: sera were collected by cardiac puncture and spleens removed for cell culture.

(b) Serum Antibody ELISA:

Ninety-six well plates were coated with 1 μg of HDM extract in binding buffer (50 mM Carbonate-Bicarbonate buffer, pH 9.6) and incubated overnight at 4 C.

Plates were washed in PBS/0.05% Tween buffer and blocked with 200 μl of 2% BSA-PBS/0.1% Tween-20 for 2 hours at RT.

After a washing step duplicate serial dilution of sample serum was added and incubated for 2 hours at RT. A biotinylated secondary antibody (IgG1 or IgG2a antibody (cross-reactive with IgG2c), Pharmingen) at concentration 2 μg/ml was added after a washing step for 1 hour at RT.

After a final wash 50 μg/well of Streptavidin-HRP (1:200 dilution in blocking buffer) was added for 30 minutes in the dark. Plates were developed by the addition of 100 μl of substrate (TMB, R & D Systems, UK) for 5-10 minutes. The reaction was stopped with 50 μl/well of 1 M H₂SO₄. The plates were read at λ450. A standard curve was included for each cytokine in order to quantify cytokine measures.

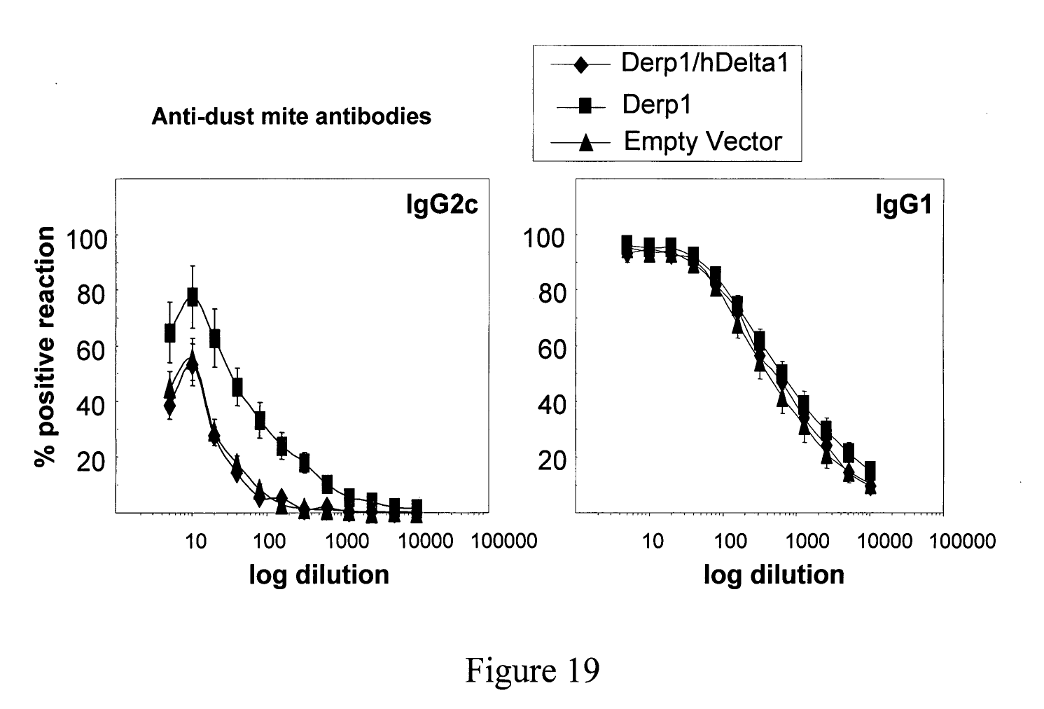

Results are shown in FIG. 19.

(c) Cell Culture and Cytokine ELISA:

Spleens were removed asceptically and single cell suspension was prepared. 5×10e6 cells from individual mice were cultured in 24 well plates with 50 μg/ml of HDM. Supernatants were collected at 72 hours for cytokine analysis.

Ninety-six-well NUNC Maxisorp plate were coated with 100 ul/well capture antibody (R & D Systems, UK) in PBS (3 μg/ml for IL10, 4 μg/ml for IL13 and 4 μg/ml for IFNγ). The plates were incubated overnight at room temperature (RT). After washing with PBS-0.05% Tween 20 the plates were blocked for 90 minutes with PBS/10% FBS. Diluted supernatants were added and incubated at RT for 90 minutes. Plates were washed and 100 μl/well of detection antibody (1 ng/ml for IL 10 and 2 ng/ml for IL 13 and IFNγ, R & D Systems, UK) was incubated for 90 minutes at RT. After a washing step, 100 ul/well of Streptavidin-HRP (diluted 1:200 in PBS/10% FBS) was added for 30 minutes at RT in the dark. After a final wash, 100 μl of substrate (TMB, R & D Systems, UK) was added and 30 minutes afterwards the reaction was stopped with 50 μl/well of 1 M H₂SO₄. The plates were read at λ450. A standard curve was included for each cytokine in order to quantify cytokine measures.

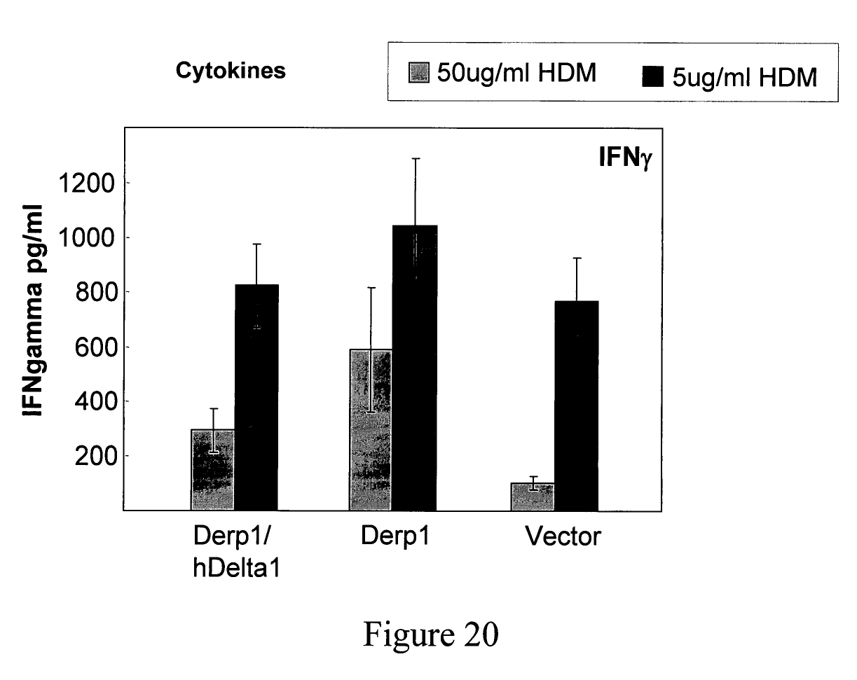

Results are shown in FIG. 20.

### Example 9

(a) DNA Treatment and Basic Immunisation Protocol:

6-8 week old C57Bl/6 female mice received on two sites of their shaved abdomens DNA-coated gold microparticles delivered by needleless injection gene gun (Serial Number XR-1, supplied by Powderject, Madison, US) from particle-coated tube sections from Example 6 above. Particles used had been coated variously with expression plasmids coding for Derp1 alone; both Derp1 and hDelta1; or vector alone (empty plasmid control). Each experimental group included 7-8 mice.

Four weeks after the last injection the mice were immunised subcutaneously at the base of the tail with 50 μg/100 μl of House Dust Mite (HDM) extract in Complete Freund's Adjuvant (CFA, Pierce) (1:1). After 2 weeks, to assess immune priming, mice were challenged in the right ear with 20 μg/2011 HDM. Ear increases (difference between injected and non-injected ear) were measured with a digital caliper at different time points up to seven days after ear challenge.

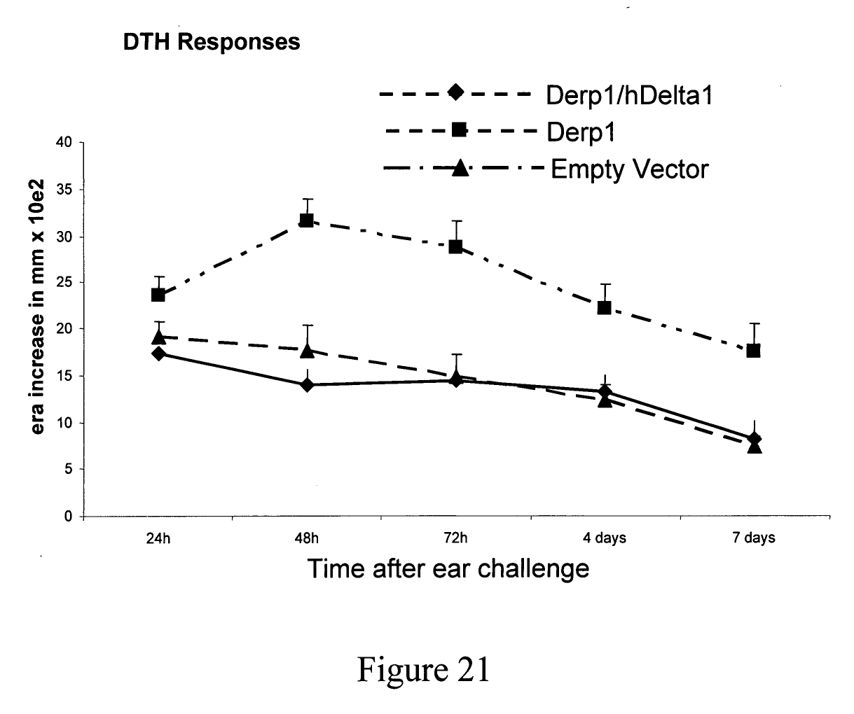

Results are shown in FIG. 21.

Three weeks after ear injection the experiment was terminated and the mice sacrificed for ex vivo analysis: sera were collected by cardiac puncture and spleens removed for cell culture.

(b) Serum Antibody ELISA:

Ninety-six well plates were coated with 1 μg of HDM extract in binding buffer (50 mM Carbonate-Bicarbonate buffer, pH 9.6) and incubated overnight at 4 C.

Plates were washed in PBS/0.05% Tween buffer and blocked with 200 μl of 2% BSA-PBS/0.1% Tween-20 for 2 hours at RT.

After a washing step duplicate serial dilution of sample serum was added and incubated for 2 hours at RT. A biotinylated secondary antibody (IgG1 or IgG2a antibody (cross-reactive with IgG2c), Pharmingen) at concentration 2 μg/ml was added after a washing step for 1 hour at RT. After a final wash 50 μg/well of Streptavidin-HRP (1:200 dilution in blocking buffer) was added for 30 minutes in the dark. Plates were developed by the addition of 100 μl of substrate (TMB, R & D Systems, UK) for 5-10 minutes. The reaction was stopped with 50 μl/well of 1 M H₂SO₄. The plates were read at λ450. A standard curve was included for each cytokine in order to quantify cytokine measures.

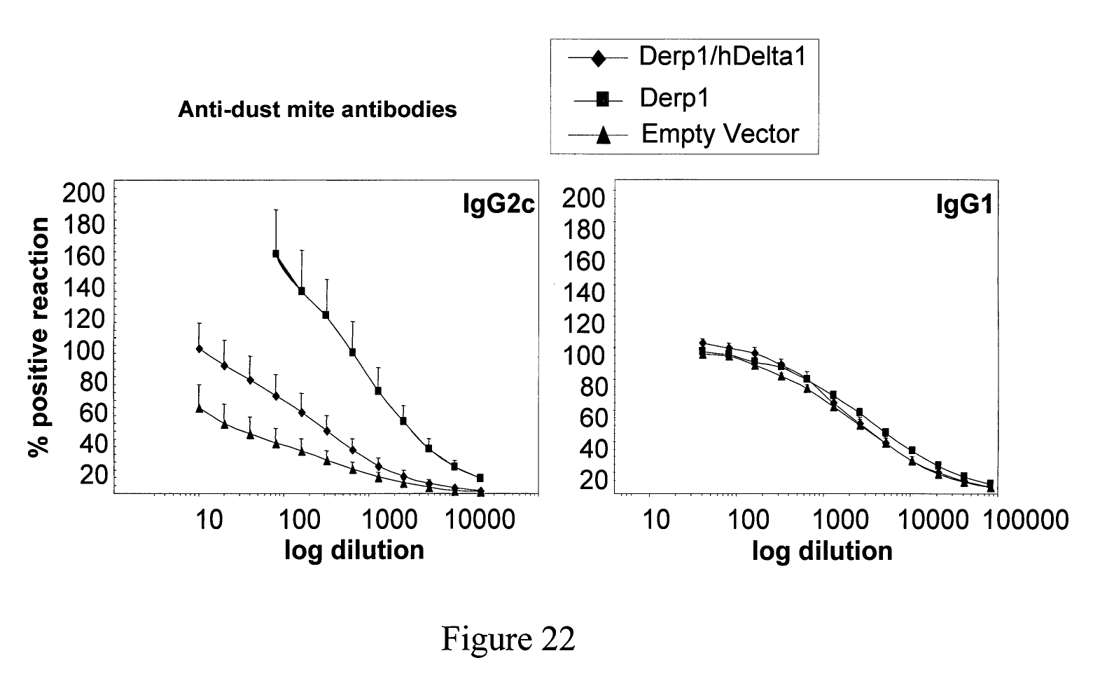

Results are shown in FIG. 22.

(c) Cell Culture and Cytokine ELISA:

Spleens were removed asceptically and single cell suspension was prepared. 5×10e6 cells from individual mice were cultured in 24 well plates with 50 μg/ml of HDM. Supernatants were collected at 72 hours for cytokine analysis.

Ninety-six-well NUNC Maxisorp plate were coated with 100 ul/well capture antibody (R & D Systems, UK) in PBS (3 μg/ml for IL10, 4 μg/ml for IL13 and 4 μg/ml for IFNγ). For TNFα a kit provided by R & D Systems was used in according to manufacturer's instructions. The plates were incubated overnight at room temperature (RT). After washing with PBS-0.05% Tween 20 the plates were blocked for 90 minutes with PBS/10% FBS. Diluted supernatants were added and incubated at RT for 90 minutes. Plates were washed and 100 μl/well of detection antibody (1 ng/ml for IL 10 and 2 ng/ml for IL 13 and IFNγ, R & D Systems, UK) was incubated for 90 minutes at RT. After a washing step, 100 ul/well of Streptavidin-HRP (diluted 1:200 in PBS/10% FBS) was added for 30 minutes at RT in the dark. After a final wash, 100 μl of substrate (TMB, R & D Systems, UK) was added and 30 minutes afterwards the reaction was stopped with 50 μl/well of 1 M H₂SO₄. The plates were read at λ450. A standard curve was included for each cytokine in order to quantify cytokine measures.

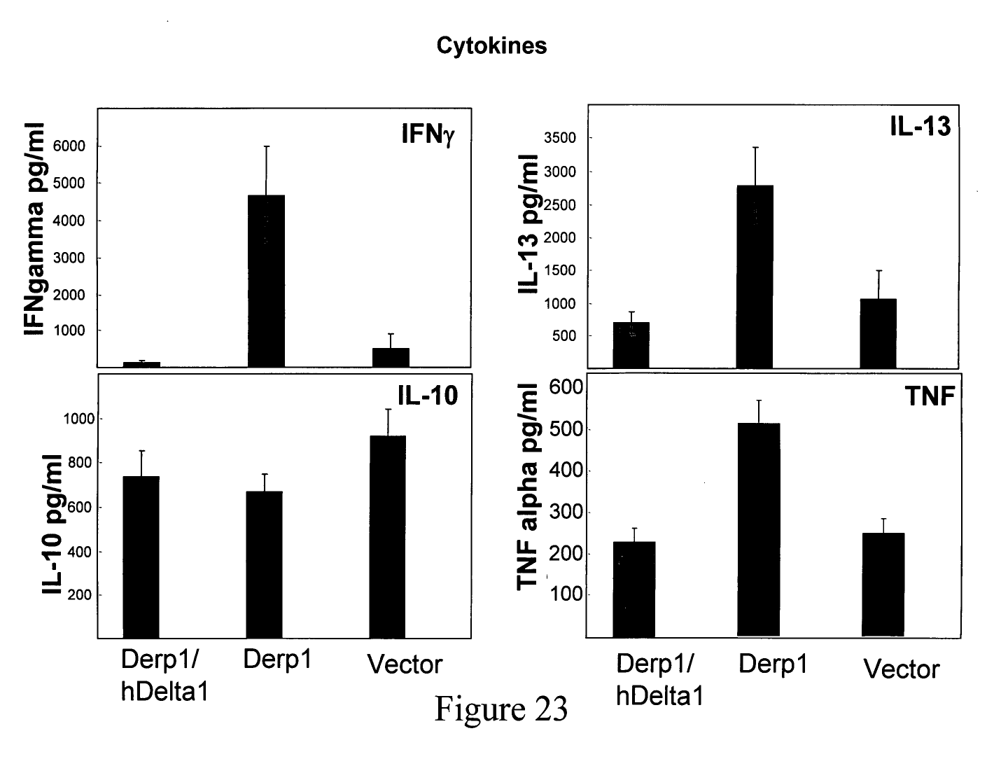

Results are shown in FIG. 23.

### Example 10

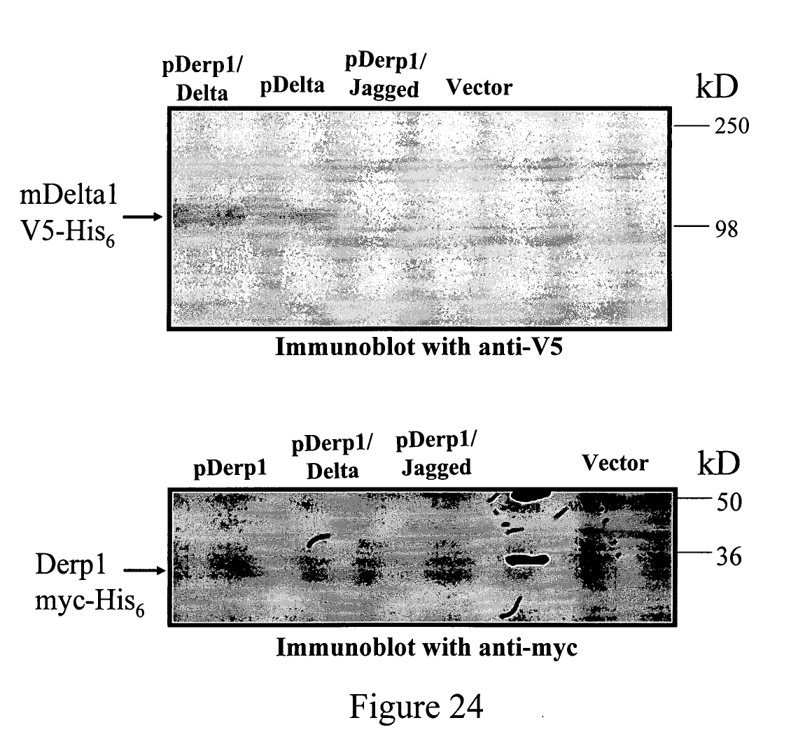

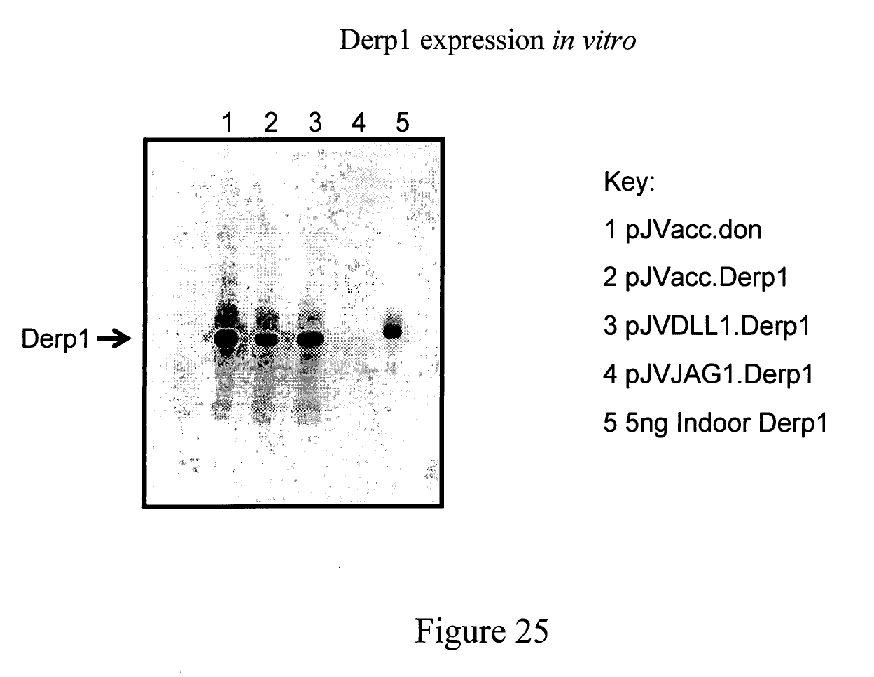

In vitro mouse or human Delta1 and Der p I expression (procedures generally as described in Example 3) from various vectors as used in the foregoing Examples was measured by Western blot. Results are shown in FIGS. 24 and 25.

### Example 11

(a) DNA Treatment and Basic Immunisation Protocol:

6-8 week old C57Bl/6 female mice received on two sites of their shaved abdomens DNA-coated gold microparticles delivered by gene gun (Serial Number XR-1, supplied by Powderject, Madison, US) from particle-coated tube sections prepared as described in Example 6 above. Particles used had been coated variously with expression plasmids coding for Derp1 alone; both Derp1 and hDelta1; both Derp1 and hJagged1; or vector alone (empty plasmid control). Gene gun treatments were repeated after 7 days and after 14 days. Each experimental group included 7-8 mice.

Approximately four weeks after the last injection to assess immune priming, mice were challenged in the right ear with 20 μg/20 μl Derp1. Ear increases (difference between injected and non-injected ear) were measured with a digital caliper at different time points up to seven days after ear challenge.

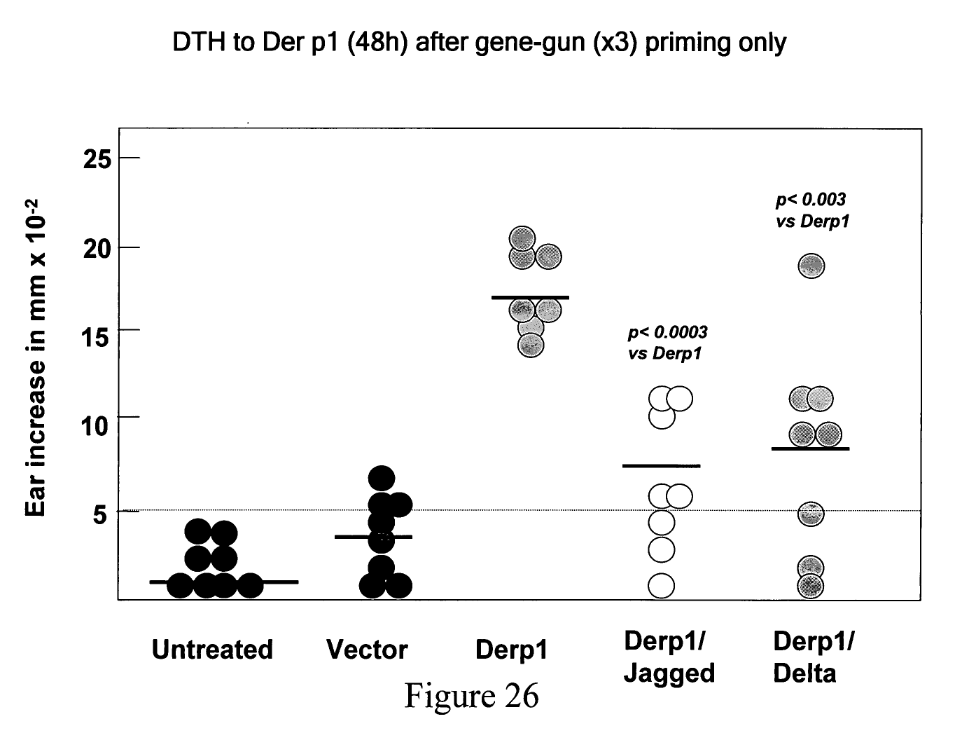

Results are shown in FIG. 26.

### Example 12

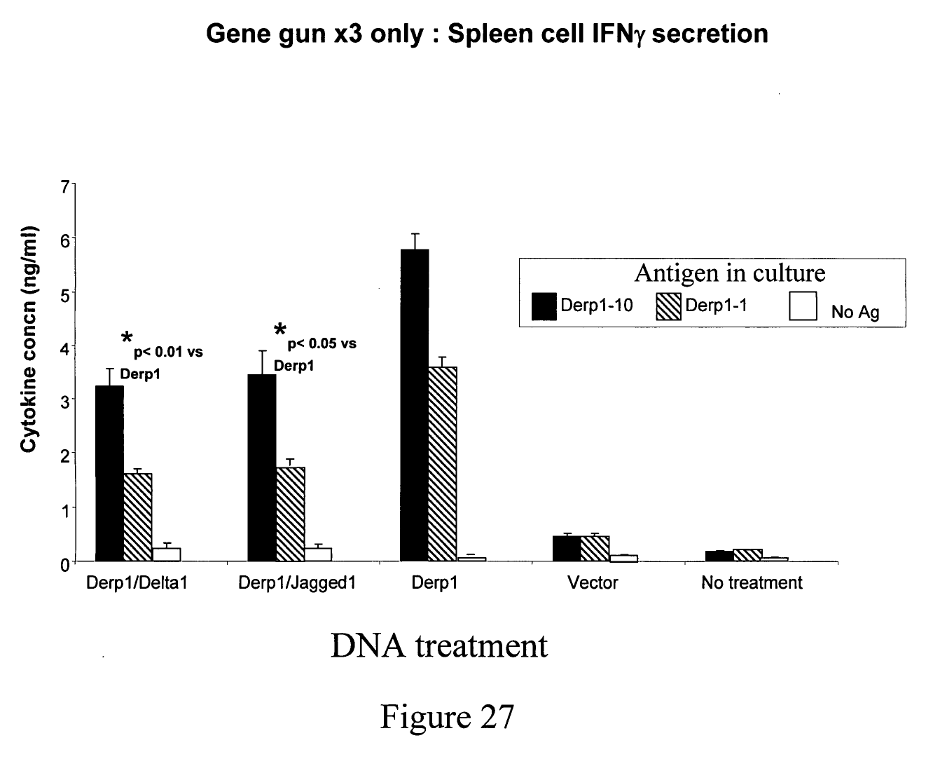

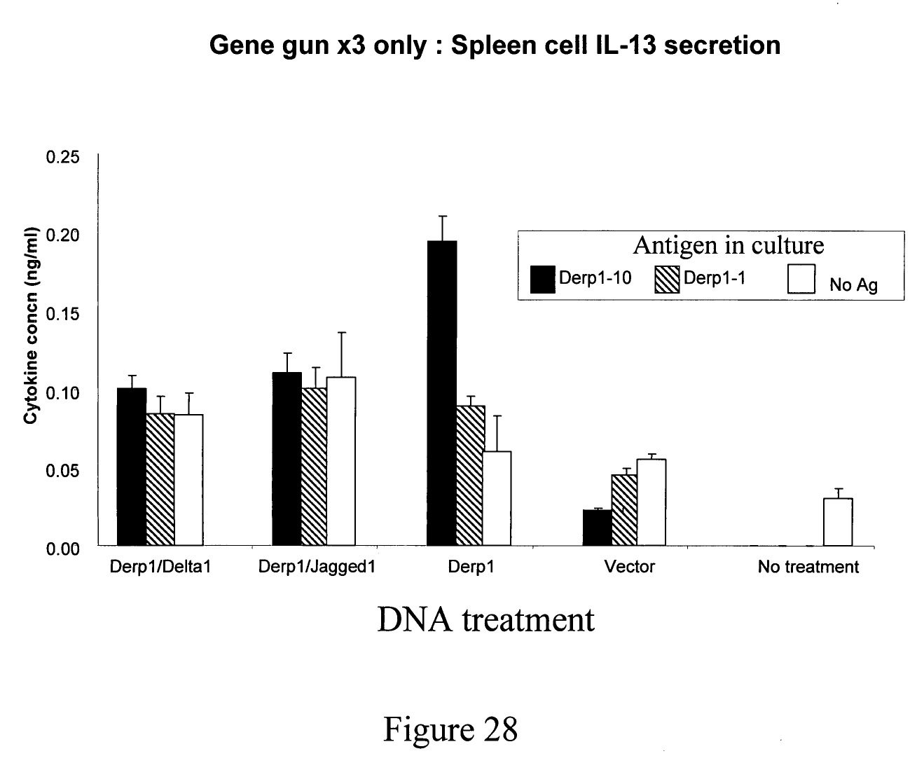

The procedure of Example 11 was repeated, but without the DTH step. Spleen cells were removed and cultured, and Der p 1 was added to the cultures at 10, 1 or 0 μg/ml. IFN gamma and IL-13 secretion was measured by ELISA. Results are shown in FIGS. 27 and 28 respectively.

### Example 13

### Prophylactic Gene Gun Treatment

6-8 week old C57Bl/6 female mice received on two sites of their shaved abdomens DNA-coated gold microparticles delivered by gene gun (Serial Number XR-1, supplied by Powderject, Madison, US) from particle-coated tube sections prepared as described in Example 6 above. Particles used had been coated variously with expression plasmids coding for Derp1 alone; both Derp1 and hDelta1; or vector alone (empty plasmid control). Each experimental group included 7-8 mice.

Approximately four weeks after the last injection the mice were immunised subcutaneously at the base of the tail with 100 μg/100 μl of a Derp1 dominant epitope spanning the region 110-131 of the protein in Complete Freund's Adjuvant (CFA, Pierce) (1:1). After 2 weeks, to assess immune priming, mice were challenged in the right ear with 20 μg/20 μl of peptide. Ear increases (difference between injected and non-injected ear) were measured with a digital caliper at different time points up to seven days after ear challenge.

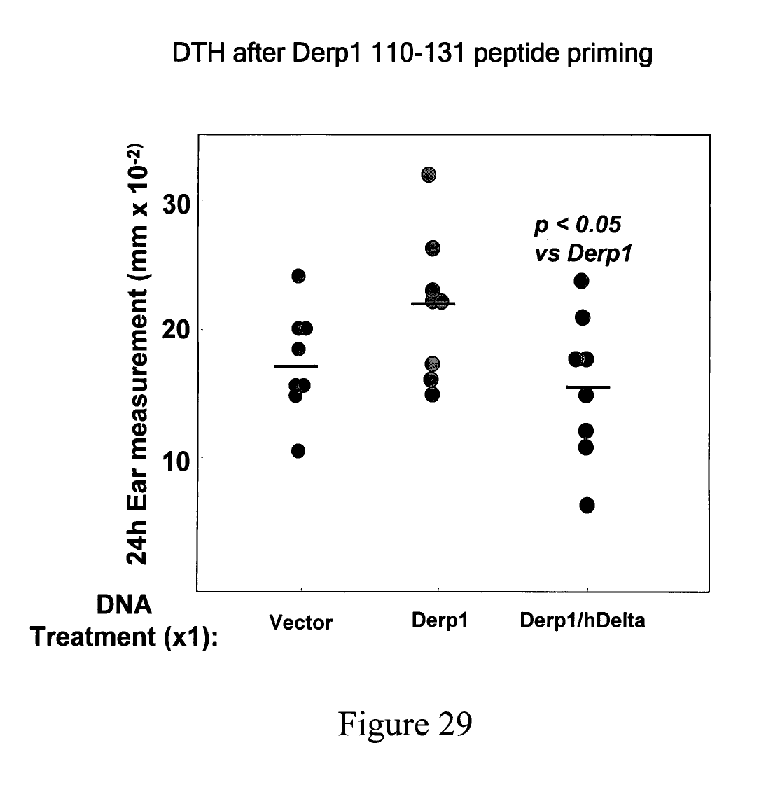

Results are shown in FIG. 29.

### Example 14

The procedure of Example 11 was repeated but without the DTH step. Approximately four weeks after the last injection the mice were immunised subcutaneously at the base of the tail with 50 μg/100 μl of House Dust Mite (HDM) extract in Complete Freund's Adjuvant (CFA, Pierce) (1:1). After 2 weeks, to assess immune priming, mice were challenged in the right ear with 20 μg/20 μl HDM. Ear increases (difference between injected and non-injected ear) were measured with a digital caliper at different time points up to seven days after ear challenge.

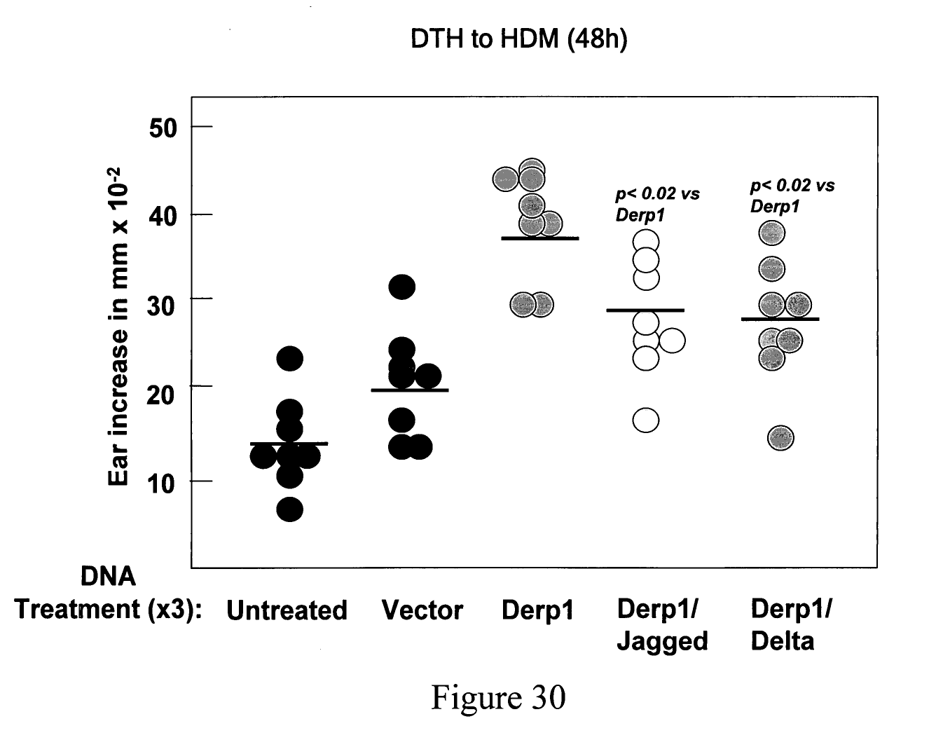

DTH results are shown in FIG. 30.

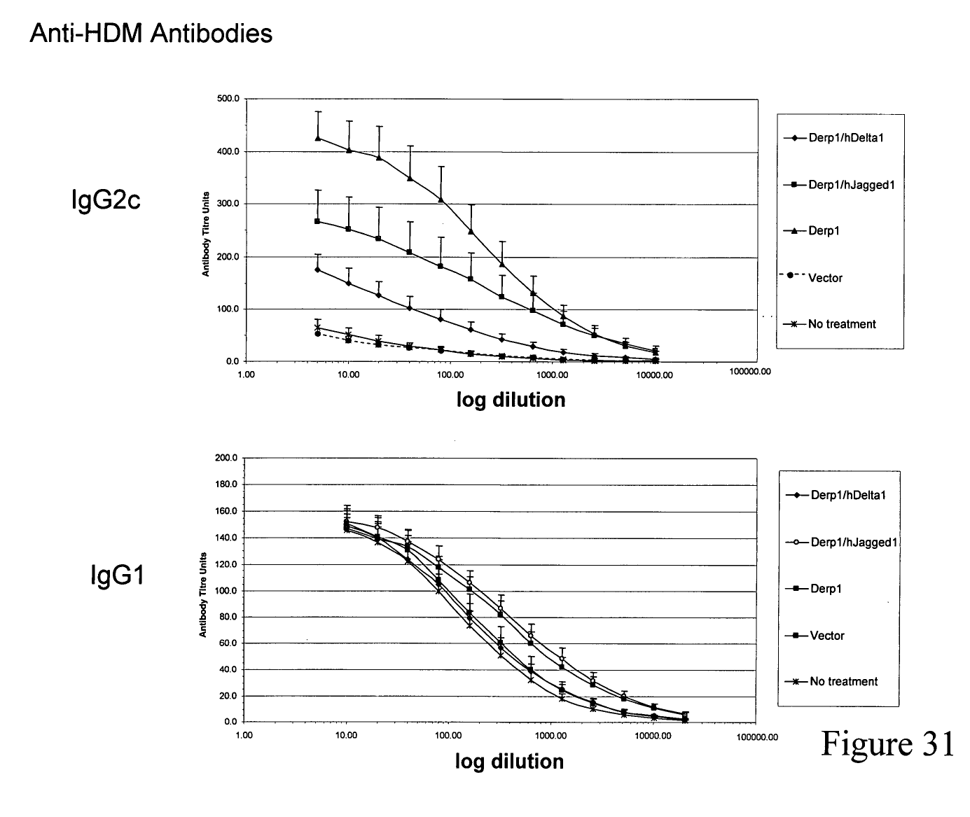

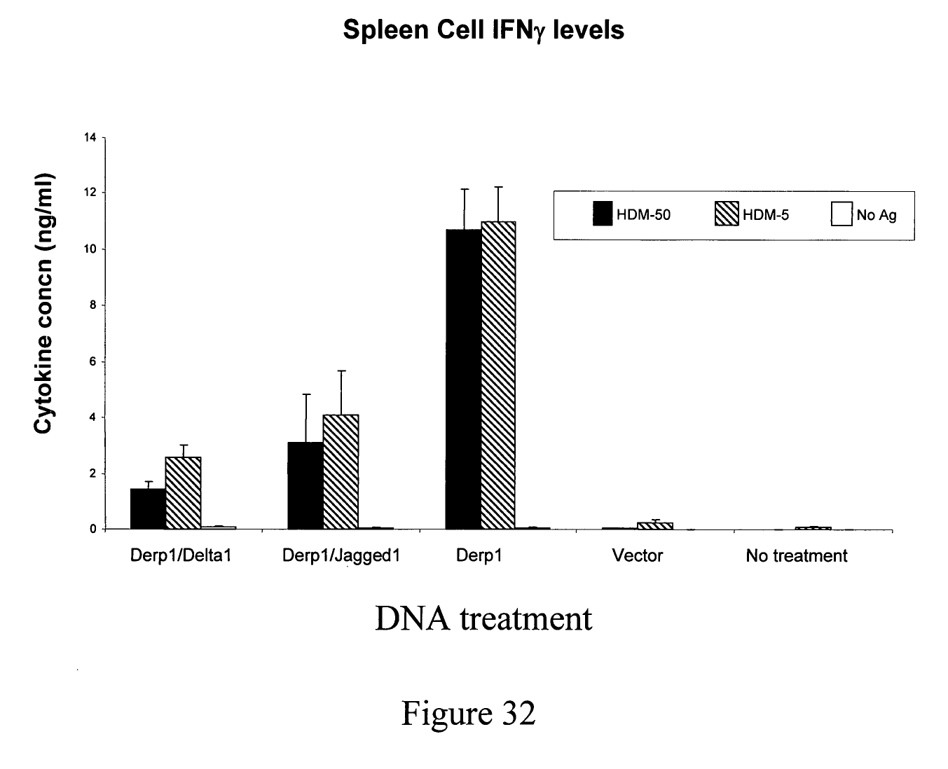

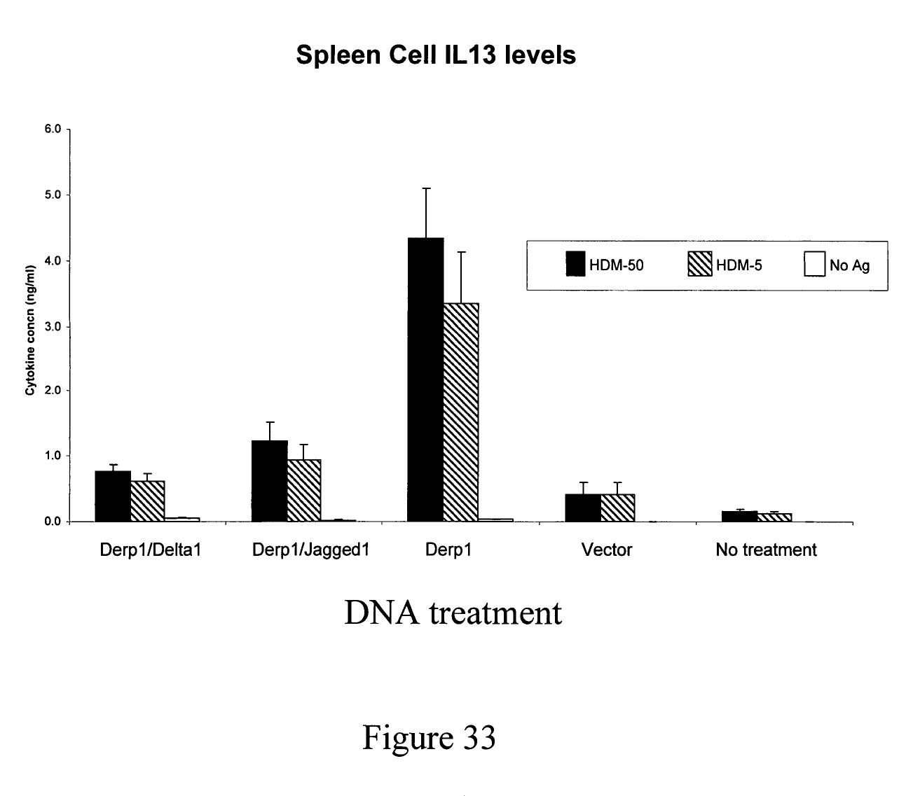

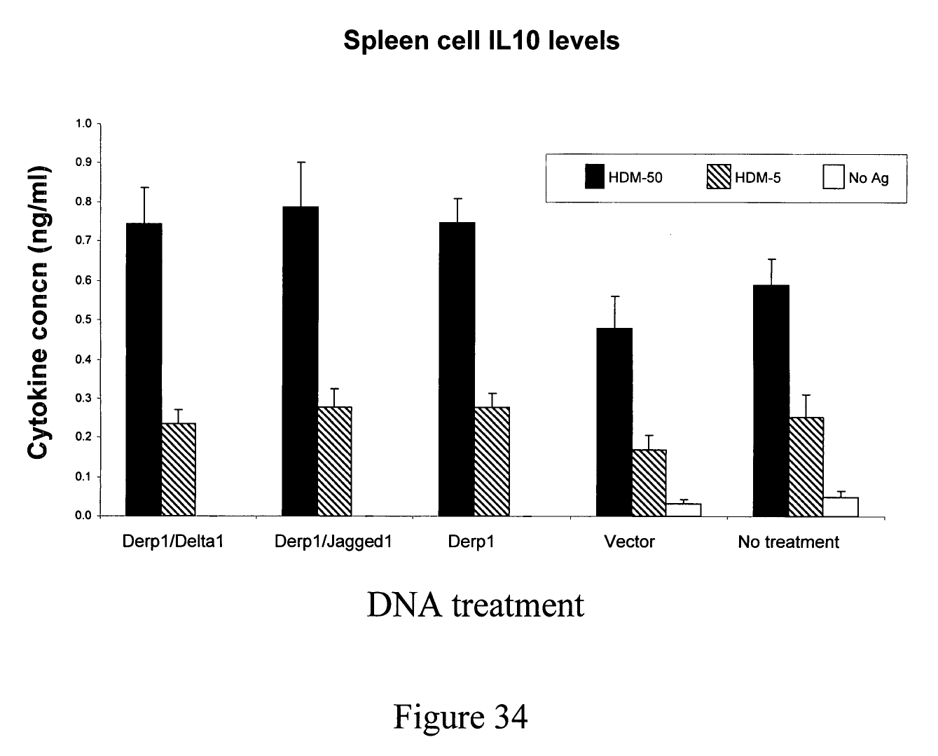

Three weeks after ear injection the experiment was terminated and the mice sacrificed for ex vivo analysis: sera were collected by cardiac puncture and spleens removed for cell culture. Serum antibody and cytokine (IFNg, IL-13 and IL-10) ELISAs were performed generally by the procedures described in Examples 7(b) and 7(c). Results are shown in FIGS. 31 to 34 respectively.

### Example 15

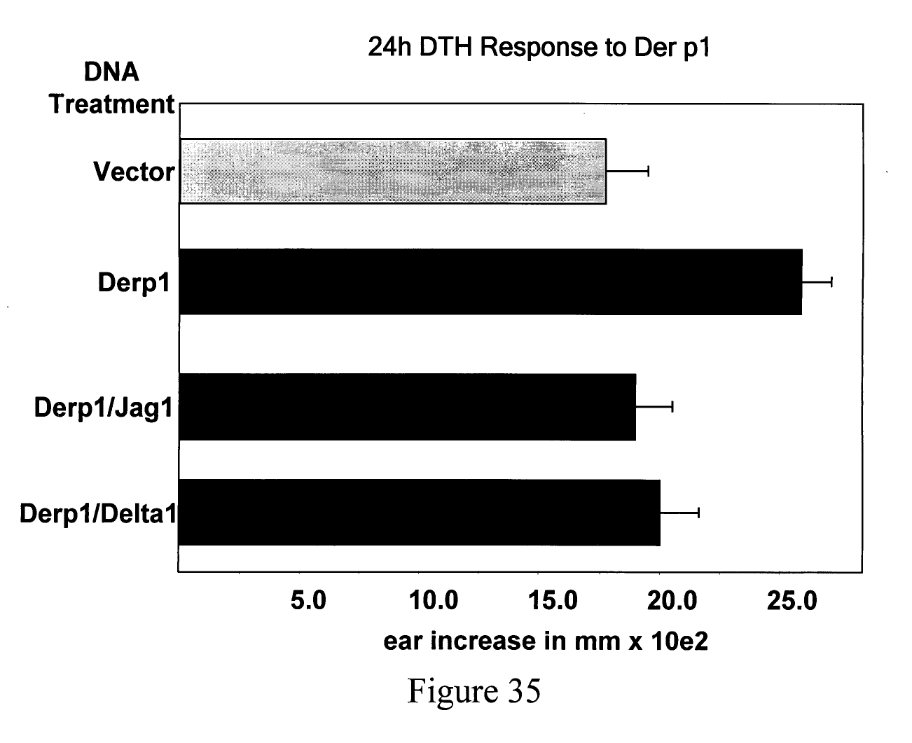

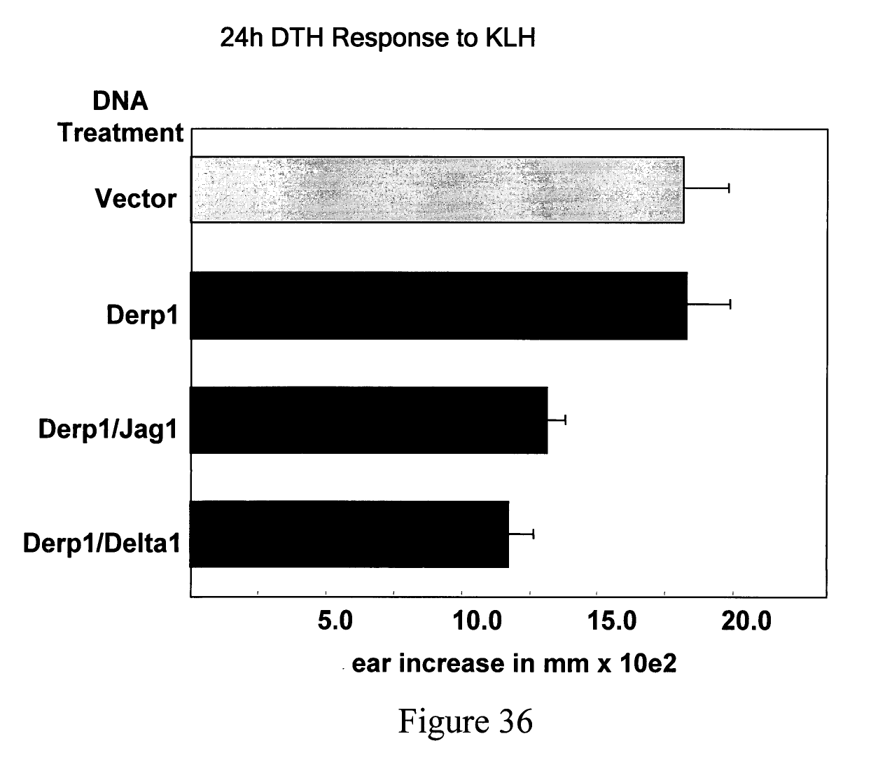

The procedure of Example 14 was repeated with the modification that 4 weeks after the last gen gun treatment one group was primed with a mixture of Der p 1 (25 μg) and KLH (5 ng) in CFA. 24 hour Der p 1 DTH results are shown in FIG. 35; and 24 hour DTH results for KLH are shown in FIG. 36.

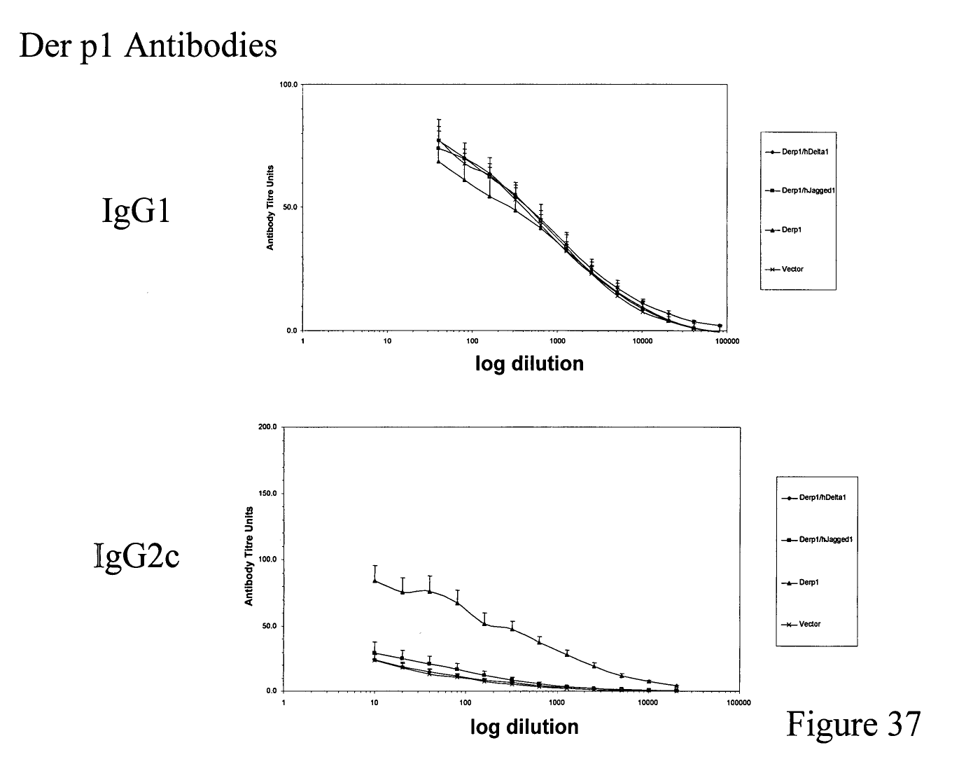

Three weeks after ear injection the experiment was terminated and the mice sacrificed for ex vivo analysis: sera were collected by cardiac puncture and spleens removed for cell culture. Serum antibody ELISAs were performed generally by the procedure described in Example 7(b). Results are shown in FIG. 37.

### Example 16

### Therapeutic Gene Gun Treatment (Der p 1 Pre-Priming)

6-8 week old C57Bl/6 female mice were immunised subcutaneously at the base of the tail with 50 μg/100 μl of House Dust Mite (HDM) extract in Complete Freund's Adjuvant (CFA, Pierce) (1:1).

14 days later, mice received on two sites of their shaved abdomens DNA-coated gold microparticles delivered by gene gun (Serial Number XR-1, supplied by Powderject, Madison, US) from particle-coated tube sections prepared as described in Example 6 above. Particles used had been coated variously with expression plasmids coding for Derp1 alone; both Derp1 and hDelta1; both Derp1 and hJagged1; or vector alone (empty plasmid control). Each experimental group included 7-8 mice.

After 4 weeks, to assess immune priming, mice were challenged in the right ear with 20 μg/20 μl HDM extract. Ear increases (difference between injected and non-injected ear) were measured with a digital caliper at different time points up to seventeen days after ear challenge.

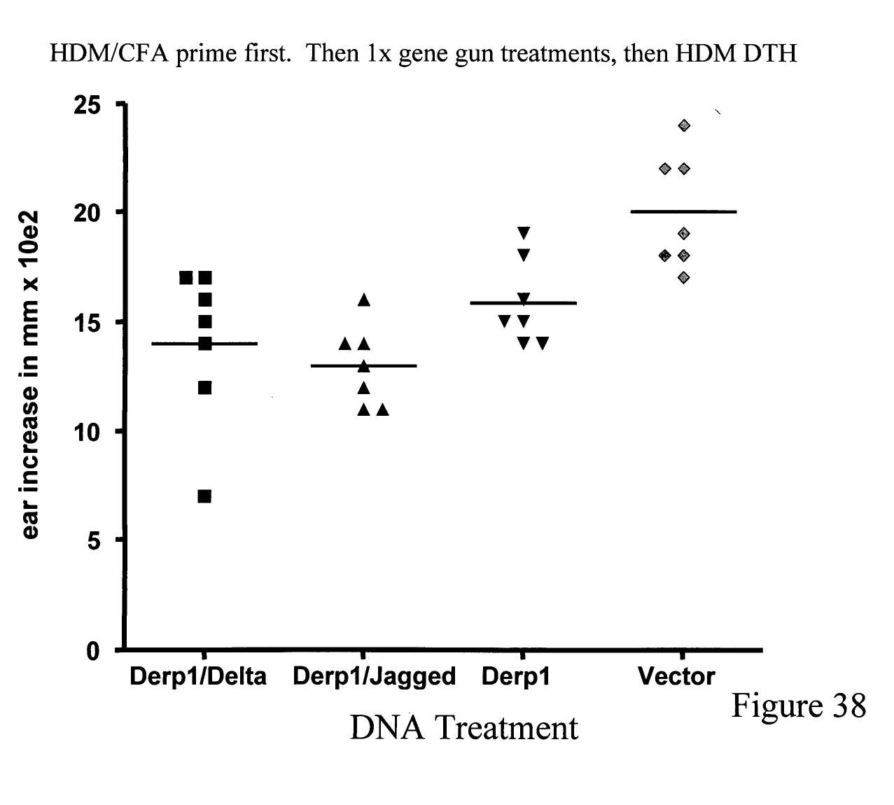

Results are shown in FIG. 38.

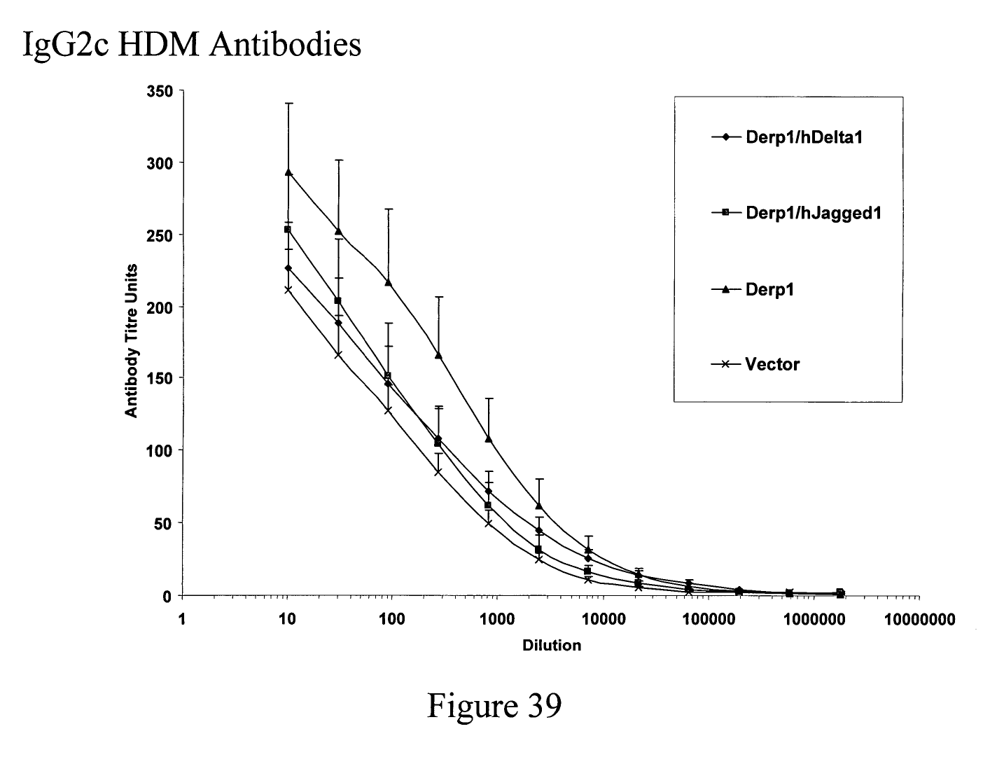

Three weeks after ear injection the experiment was terminated and the mice sacrificed for ex vivo analysis: sera were collected by cardiac puncture and spleens removed for cell culture. Serum antibody ELISAs were performed generally by the procedure described in Example 7(b). Results are shown in FIG. 39.

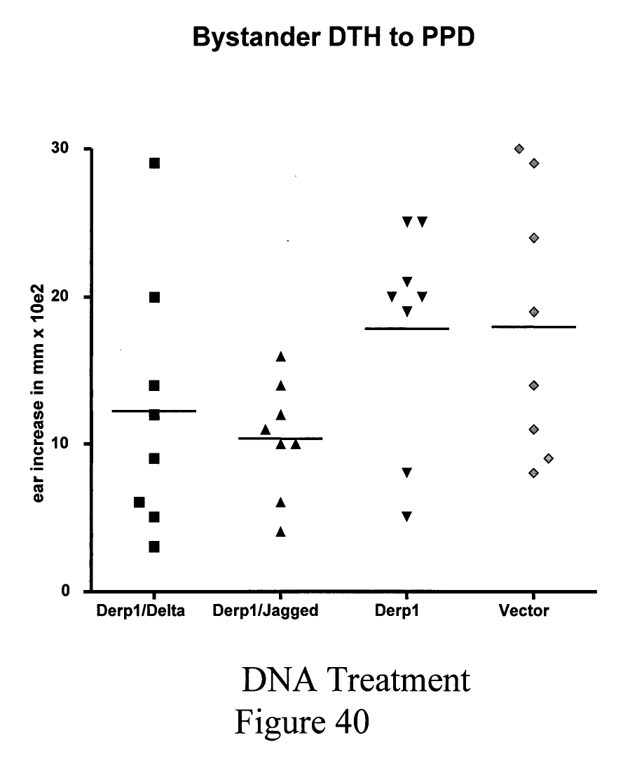

Two weeks later a bystander response to PPD was measured by challenging the left ear with 20 μg/20 μl of PPD. Results are shown in FIG. 40.

### Example 17

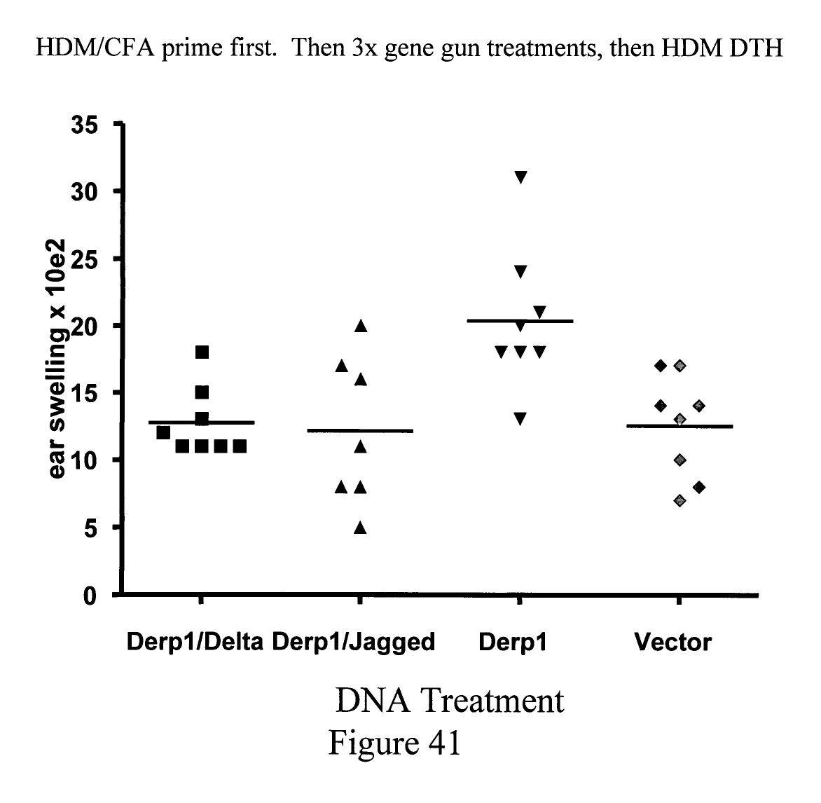

The procedure of Example 16 was repeated, with the modification that mice received 3 separate Gene gun treatments at 7 day intervals to give a total of three treatments. DTH results are shown in FIG. 41.

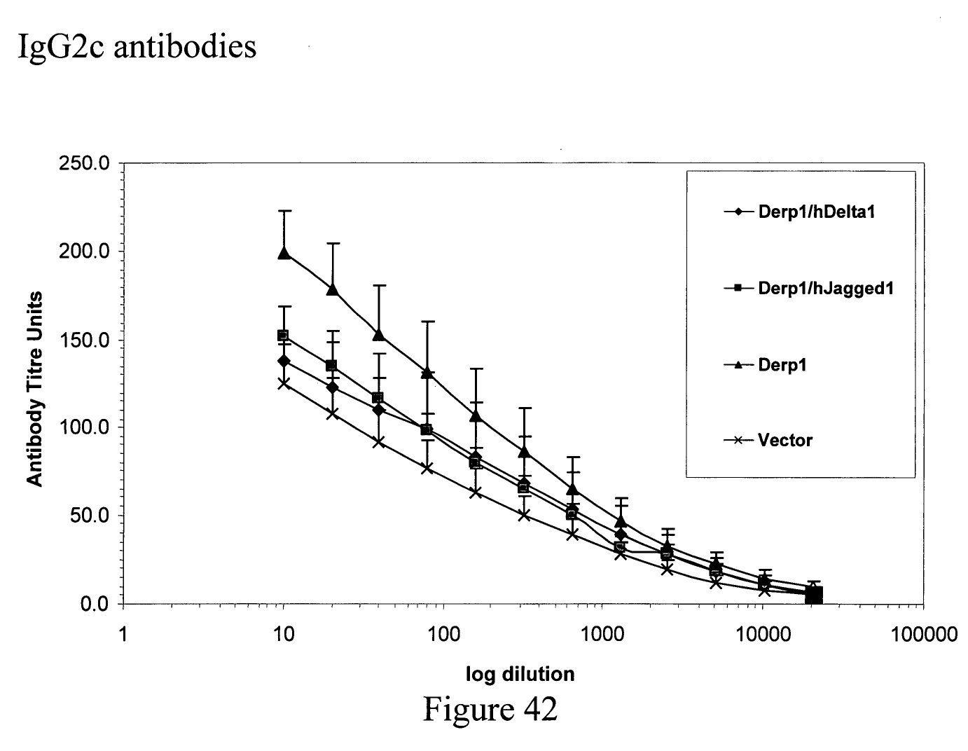

Three weeks after ear injection the experiment was terminated and the mice sacrificed for ex vivo analysis: sera were collected by cardiac puncture and spleens removed for cell culture. Serum antibody ELISAs were performed generally by the procedure described in Example 7(b). Results are shown in FIG. 42.

### Example 18

6-8 week old C57Bl/6 female mice were pre-primed with HDM 50 ug/Titermax Gold (1:1) 100 ul i.p/mouse. Priming was repeated 7 days later.

14 days later, mice received received on two sites of their shaved abdomens DNA-coated gold microparticles delivered by gene gun (Serial Number XR-1, supplied by Powderject, Madison, US) from particle-coated tube sections prepared as described in Example 6 above. Particles used had been coated variously with expression plasmids coding for Derp1 alone; both Derp1 and hDelta1; both Derp1 and hJagged1; or vector alone (empty plasmid control). Each experimental group included 7-8 mice.

Approximately 30 days later, mice received HDM extract 20 ug/20 ul intranasal in PBS (3.2 mg/ml batch).

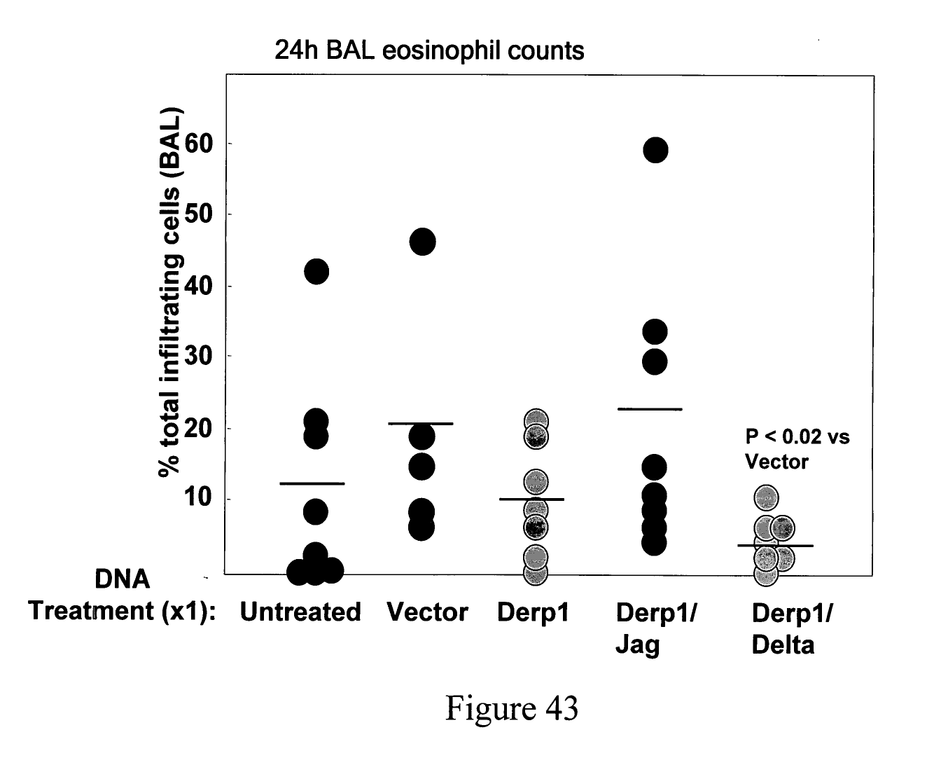

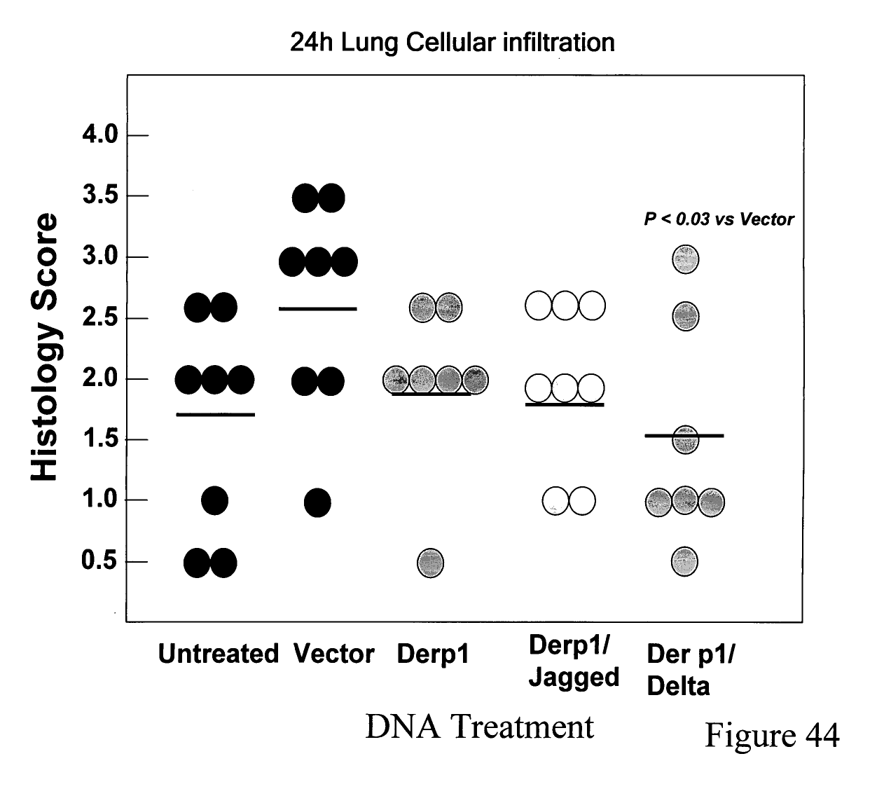

1 day later mice were sacrificed for bronchioalveolar lavage (BAL) eosinophil counts and lung histology (measurement of lung cellular infiltration). BAL lavage was collected by washing incannulated lungs 3-5 times with 1 ml of complete medium. After total cell counting, differential counts were performed on cytospin preparation (5×10⁵ cells) stained with Kwik Diff Solution Stain Kit (Thermo Shandon). Results are shown in FIGS. 43 and 44 respectively.

Lung cellular infiltration was assessed on paraffin-embedded lungs stained with standard H&E methods. Three lung sections at different levels were scored blindly. For each level the score goes from 1 to 5 (maximum score is 15).

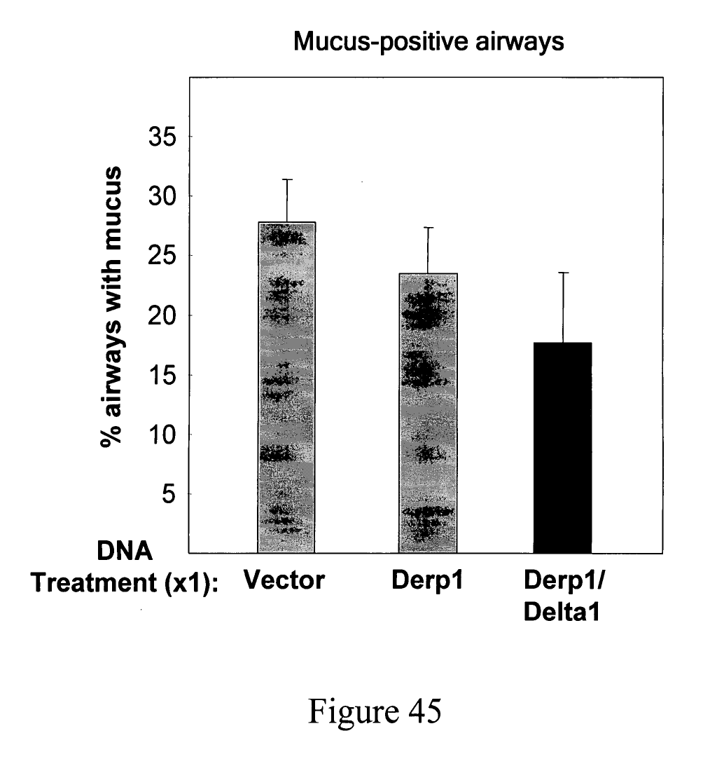

Mucous positive airways were also assessed staining the lung sections with AB-PAS (Alcian Blue-Periodic Acid Schiff), results are shown in FIG. 45. The results are expressed as percentage of mucous positive airways over the total airways number.

### Example 19

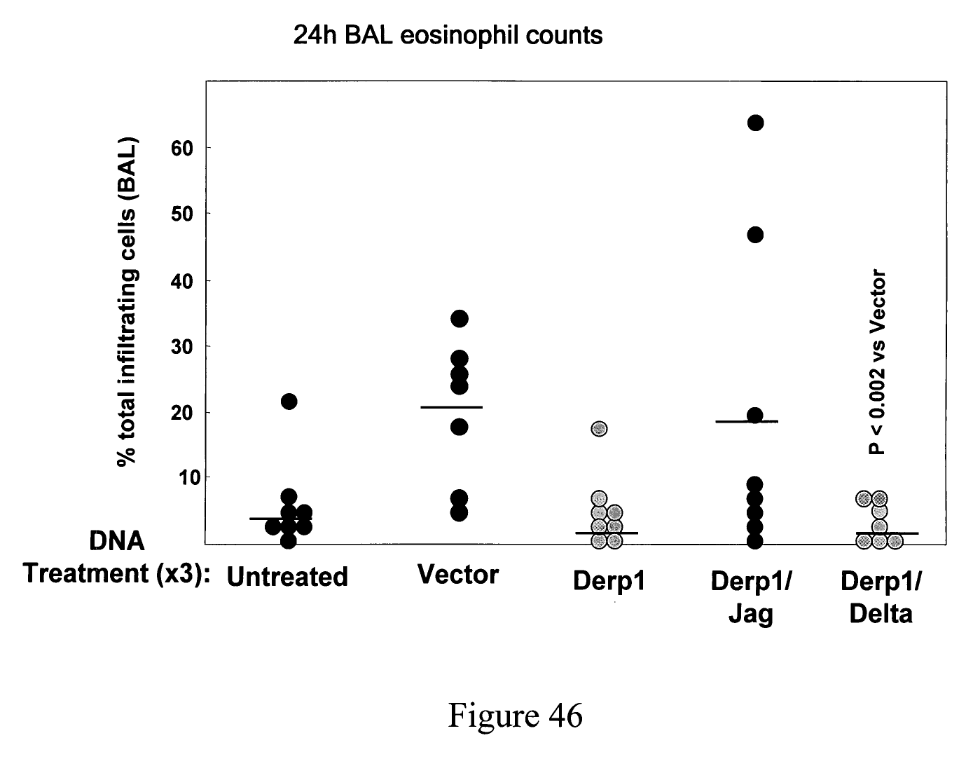

The procedure of Example 18 was repeated, with the modification that mice received 3 separate Gene gun treatments at 7 day intervals to give a total of three treatments. Eosinophil counts are shown in FIG. 46.

### Example 20

6-8 week old C57Bl/6 female mice were pre-primed with HDM 50 ug/Alum (3:1) 100 ul i.p/mouse. Priming was repeated 7 days later.

14 days later, mice received received on two sites of their shaved abdomens DNA-coated gold microparticles delivered by gene gun (Serial Number XR-1, supplied by Powderject, Madison, US) from particle-coated tube sections prepared as described in Example 6 above. Particles used had been coated variously with expression plasmids coding for Derp1 alone; both Derp1 and hDelta1; hDelta1 alone; or vector alone (empty plasmid control). Each experimental group included 7-8 mice.

Approximately 30 days later, mice received HDM extract 20 ug/20 ul intratracheally in PBS.

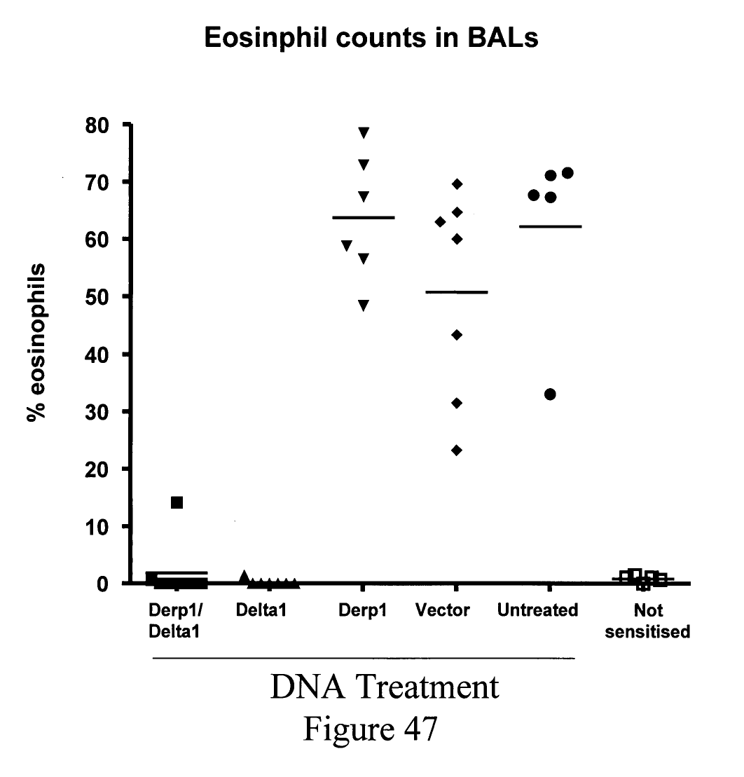

3 days later mice were sacrificed for bronchioalveolar lavage (BAL) eosinophil counts. BAL lavage was collected by washing incannulated lungs 3-5 times with 1 ml of complete medium. After total cell counting, differential counts were performed on cytospin preparation (5×10⁵ cells) stained with Kwik Diff Solution Stain Kit (Thermo Shandon). Results are shown in FIG. 47.

### REFERENCES (INCORPORATED HEREIN BY REFERENCE)

Artavanis-Tsakonas S, et al. (1995) Science 268:225-232.
Artavanis-Tsakonas S, et al. (1999) Science 284:770-776.
Brucker K, et al. (2000) Nature 406:411-415.
Camilli et al. (1994) Proc Natl Acad Sci USA 91:2634-2638.
Caux C, et al. (1992) Nature 360: 258-261.
Chee M. et al. (1996) Science 274:601-614.
Dkuz et al. (1997) Cell 90: 271-280.
Hemmati-Brivanlou and Melton (1997) Cell 88:13-17.
Hicks C, et al. (2000) Nat. Cell. Biol. 2:515-520.
Hoyne G. F. et al (1999) Int Arch Allergy Immunol 118:122-124.
Hoyne et al. (2000) Immunology 100:281-288.
Hoyne G. F. et al (2000) Intl Immunol 12:177-185.
Hoyne, G. et al. (2001) Immunological Reviews 182:215-227.
Hsieh et al. (1996) Molecular & Cell Biology 16(3):952-959.
Iemura et al. (1998) PNAS 95:9337-9345.
Irvine K D (1999) Curr. Opin. Genet. Devel. 9:434-441.
Ju B J, et al. (2000) Nature 405:191-195.
Kusano and Raab-Truab (2001) J Virol 75(1):384-395.
Leimeister C. et al. (1999) Mech Dev 85(1-2):173-7.
L1 et al. (1998) Immunity 8(1):43-55.
Lieber, T. et al. (1993) Genes Dev 7(10):1949-65.
Lu, F. M. et al. (1996) Proc Natl Acad Sci 93(11):5663-7.
Matsuno K, et al. (1998) Nat. Genet. 19:74-78.
Matsuno, K. et al. (1995) Development 121(8):2633-44.
McGuinness T. Et al (1996) Genomics 35(3):473-85.
Medhzhitov et al. (1997) Nature 388:394-397.
Moloney D J, et al. (2000) Nature 406:369-375.
Munro S, Freeman M. (2000) Curr. Biol. 10:813-820.
Ordentlich et al. (1998) Mol. Cell. Biol. 18:2230-2239.
Osborne B, Miele L. (1999) Immunity 11:653-663.
Panin V M, et al. (1997) Nature 387:908-912.
Sallusto F and Lanzavecchia A (1994) J. Exp. Med. 179: 1109-1118.
Sasai et al. (1994) Cell 79:779-790.
Schroeter, E. H. et al. (1998) Nature 393(6683):382-6.
Strobl et al. (2000) J Virol 74(4):1727-35.
Struhl G, Adachi A. Cell 93(4):649-60.
Takebayashi K. et al. (1994) J Biol Chem 269(7):150-6.
Tamura K, et al. (1995) Curr. Biol. 5:1416-1423.
Valenzuela et al. (1995) J. Neurosci. 15:6077-6084.
Weinmaster G. (2000) Curr. Opin. Genet. Dev. 10:363-369.
Wilson and Hemmati-Brivanlou (1997) Neuron 18:699-710.
Zhao et al. (1995) J. Immunol. 155:3904-3911.

Various modifications and variations of the described methods and system of the invention will be apparent to those skilled in the art without departing from the scope and spirit of the invention. Although the invention has been described in connection with specific preferred embodiments, it should be understood that the invention as described should not be unduly limited to such specific embodiments. Indeed, various modifications of the described modes for carrying out the invention which are obvious to those skilled in chemistry, biology or related fields are intended to be within the scope of the following claims.

1. A particle capable of being inserted into or taken up by a cell comprising:
i) a polynucleotide coding for a modulator of Notch signalling;
ii) a polynucleotide coding for an antigen or antigenic determinant thereof; and
iii) optionally, a matrix, carrier or substrate wherein one or both polynucleotides are borne within or on the surface of the matrix, carrier or substrate.

2. The particle as claimed in claim 1 in the form of a microparticle.

3. (canceled)

4. The particle as claimed in claim 1, wherein the cell is an immune cell.

5. The particle as claimed in claim 4, wherein the immune cell is an antigen presenting cell.

6. (canceled)

7. (canceled)

8. The particle as claimed in claim 1, which is capable of being inserted into a cell by a ballistic/biolistic delivery method.

9. (canceled)

10. The particle as claimed in claim 1, which is capable of being taken up by a cell by endocytosis or phagocytosis.

11. (canceled)

12. (canceled)

13. The particle as claimed in claim 1, wherein the polynucleotide coding for a modulator of Notch signalling codes for an activator of Notch signalling.

14. The particle as claimed in claim 1, wherein the polynucleotide coding for a modulator of Notch signalling codes for an inhibitor of Notch signalling.

15. (canceled)

16. (canceled)

17. The particle as claimed in claim 1, wherein the polynucleotide coding for the modulator of Notch signalling codes for:
(i) a polypeptide comprising a Notch ligand or an active fragment, derivative, homologue, analogue or allelic variant thereof;
(ii) a fusion protein comprising a segment of a Notch ligand extracellular domain and an immunoglobulin F_{c} segment:
(iii) a protein or polypeptide comprising a Notch ligand DSL domain, at least one EGF-like domain, and optionally a membrane-binding or transmembrane domain;
(iv) Notch intracellular domain (Notch IC) or a fragment, derivative, homologue, analogue or allelic variant thereof; or
(v) a dominant negative version of a Notch signalling repressor.

18. The particle as claimed in claim 17, wherein the Notch ligand is Delta or Serrate/Jagged protein or a fragment, derivative, homologue, analogue or allelic variant thereof.

19-25. (canceled)

26. The particle as claimed in claim 1, wherein the polynucleotide coding for the modulator of Notch signalling codes for a polypeptide capable of upregulating the expression or activity of a Notch ligand or a downstream component of the Notch signalling pathway.

27. The particle as claimed in claim 1, wherein the antigen or antigenic determinant is:
(i) an allergen or antigenic determinant thereof;
(ii) an autoantigen or antigenic determinant thereof;
(iii) an MHC or transplant antigen or antigenic determinant thereof;
(iv) a pathogen antigen or antigenic determinant thereof; or
(v) a tumour antigen or antigenic determinant thereof.

28-31. (canceled)

32. The particle as claimed in claim 1 comprising a liposomal structure.

33. The particle as claimed in claim 1, which is suitable for transdermal, intradermal, or mucosal delivery to a subject or administration to a subject by means of a needleless syringe or biolistic delivery device.

34. (canceled)

35. The particle as claimed in claim 1 having a size of from about 0.005 to about 500 micrometres.

36. (canceled)

37. (canceled)

38. The particle as claimed in claim 1 comprising a carrier particle.

39. The particle as claimed in claim 38, wherein the carrier particle is a metal particle.

40. The particle as claimed in claim 39, wherein the carrier particle is selected from tungsten, gold, platinum and iridium particles.

41. (canceled)

42. The particle as claimed in claim 1 comprising a polymeric matrix or carrier or a lipid matrix or carrier.

43. (canceled)

44. The particle as claimed in claim 42, wherein the lipid is selected from the group consisting of a cationic lipid, an anionic lipid, and a zwitterionic lipid.

45. The particle as claimed in claim 42, wherein the lipid comprises cetyltrimethylammonium.

46. The particle as claimed in claim 42, wherein the lipid comprises a phospholipid.

47. (canceled)

48. The particle as claimed in claim 1, wherein one or both of the polynucleotides comprises an expression control sequence operatively linked to a coding sequence.

49. The particle as claimed in claim 48, wherein one or both of the polynucleotides is present in an expression vector.

50. (canceled)

51. (canceled)

52. The particle as claimed in claim 1 further comprising a targeting molecule.

53. The particle as claimed in claim 1 further comprising a stabilizer.

54. The particle as claimed in claim 1, wherein one or both of the polynucleotides codes for a trafficking sequence selected from the group consisting of a sequence which trafficks to endoplasmic reticulum, a sequence which trafficks to a lysosome, a sequence which trafficks to an endosome, a sequence which trafficks to an intracellular vesicle, and a sequence which trafficks to the nucleus.

55-59. (canceled)

60. The A particle as claimed in claim 1, wherein at least about 10% of the polynucleotide, by weight, comprises supercoiled DNA molecules.

61. (canceled)

62. (canceled)

63. A composition comprising the particle as claimed in claim 1.

64. A method for preparing the particle as claimed in claim 1 comprising combining (in any order):
i) a polynucleotide coding for a modulator of Notch signalling;
ii) a polynucleotide coding for an antigen or antigenic determinant thereof; and optionally a substrate, matrix or carrier.

65. A method for modulating an immune response to an antigen in a subject by administering the particle as claimed in claim 1 to a subject in need thereof.

66. (canceled)

67. (canceled)

68. A method for modifying an immune response to an antigen or antigenic determinant in a subject by causing to be inserted into or taken up by a cell in the subject:
i) a polynucleotide coding for a modulator of Notch signalling; and
ii) a polynucleotide coding for the antigen or antigenic determinant thereof;
whereby expression of the polynucleotides in the cell modifies an immune response to the antigen or antigenic determinant in the subject.

69. The method as claimed in claim 68, wherein the antigen or antigenic determinant is an allergen, autoantigen, tumour antigen or pathogen antigen or an antigenic determinant thereof.

70. The method as claimed in claim 68, wherein the polynucleotides are administered in or on a particle or particles.

71. The method as claimed in claim 70, wherein the polynucleotide coding for a modulator of Notch signalling and the polynucleotide coding for the antigen or antigenic determinant thereof are administered in or on the same or separate particles.

72. (canceled)

73. The method as claimed in claim 68, wherein the particle is administered directly into skin or muscle tissue, to mucosal tissue, by inhalation, or topically.

74-76. (canceled)

77. A particle acceleration device suitable for use for biolistic delivery, wherein said device is loaded with particles as claimed in claim 1.

78. A polynucleotide conjugate comprising first and second polynucleotide sequences, wherein the first sequence is a polynucleotide for Notch signaling modulation or codes for a polypeptide for Notch signalling modulation and the second sequence encodes an antigen or antigenic determinant.

79. The polynucleotide conjugate as claimed in claim 78 in the form of a vector.

80. (canceled)

81. (canceled)

82. The polynucleotide conjugate as claimed in claim 78, wherein the first polynucleotide sequence codes for:
(i) a Notch ligand or a fragment, derivative, homologue, analogue or allelic variant thereof;
(ii) a protein or polypeptide which comprises a Notch ligand DSL domain and at least one Notch ligand EGF-like domain;
(iii) a protein or polypeptide which comprises a Notch ligand DSL domain, at least two Notch ligand EGF-like domains, and optionally a membrane binding or transmembrane domain.

83. The polynucleotide conjugate as claimed in claim 82, wherein the Notch ligand is Delta or Serrate/Jagged protein or a fragment, derivative, homologue, analogue or allelic variant thereof.

84. (canceled)

85. (canceled)

86. The polynucleotide conjugate as claimed in claim 78, wherein the first and second sequences are operably linked to one or more promoters.

87. The polynucleotide conjugate as claimed in claim 86, wherein the first and second sequences are operably linked to one or more enhancers.

88. The polynucleotide conjugate as claimed in claim 78, wherein the first sequence is operably linked to a first promoter and the second sequence is operably linked to a second promoter.

89. The polynucleotide conjugate as claimed in claim 78, wherein the first and second sequences are operably linked to one or more polyadenylation sequences.

90. The polynucleotide conjugate as claimed in claim 78 for expression in mammalian cells.

91. The polynucleotide conjugate as claimed in claim 78 comprising a selection marker.

92-94. (canceled)

95. A vaccine composition comprising the particle as claimed in claim 1.


SEQUENCE LISTING (95 sequences)

SEQ ID NO: 1 - protein, 310 aa, Homo sapiens
GVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDEC

SEQ ID NO: 2 - protein, 63 aa, Drosophila sp.
WKTNKSESQYTSLEYDFRVTCDLNYYGSGCAKFCRPRDDSFGHSTCSETGEIICLTGWQGDYC

SEQ ID NO: 3 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 4 - protein, 63 aa, Mus sp.
WSQDLHSSGRTDLRYSYRFVCDEHYYGEGCSVFCRPRDDAFGHFTCGDRGEKMCDPGWKGQYC

SEQ ID NO: 5 - protein, 63 aa, Rattus sp.
WSQDLHSSGRTDLRYSYRFVCDEHYYGEGCSVFCRPRDDAFGHFTCGERGEKMCDPGWKGQYC

SEQ ID NO: 6 - protein, 63 aa, Mus sp.
WRTDEQNDTLTRLSYSYRVICSDNYYGESCSRLCKKRDDHFGHYECQPDGSLSCLPGWTGKYC

SEQ ID NO: 7 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 8 - protein, 63 aa, Rattus sp.
WQTLKQNTGIAHFEYQIRVTCDDHYYGFGCNKFCRPRDDFFGHYACDQNGNKTCMEGWMGPEC

SEQ ID NO: 9 - protein, 63 aa, Mus sp.
WQTLKQNTGIAHFEYQIRVTCDDHYYGFGCNKFCRPRDDFFGHYACDQNGNKTCMEGWMGPDC

SEQ ID NO: 10 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 11 - protein, 63 aa, Gallus sp.
WQTLKHNTGAAHFEYQIRVTCAEHYYGFGCNKFCRPRDDFFTHHTCDQNGNKTCLEGWTGPEC

SEQ ID NO: 12 - protein, 63 aa, Gallus sp.
WKTLQFNGPVANFEVQIRVKCDENYYSALCNKFCGPRDDFVGHYTCDQNGNKACMEGWMGEEC

SEQ ID NO: 13 - protein, 63 aa, Mus sp.
WKSLHFSGHVAHLELQIRVRCDENYYSATCNKFCRPRNDFFGHYTCDQYGNKACMDGWMGKEC

SEQ ID NO: 14 - protein, 63 aa, Homo sapiens; identical to SEQ ID NO: 13

SEQ ID NO: 15 - protein, 63 aa, Rattus sp.; identical to SEQ ID NO: 13

SEQ ID NO: 16 - protein, 63 aa, Homo sapiens; identical to SEQ ID NO: 13

SEQ ID NO: 17 - protein, 63 aa, Drosophila sp.
WKTLDHIGRNARITYRVRVQCAVTYYNTTCTTFCRPRDDQFGHYACGSEGQKLCLNGWQGVNC

SEQ ID NO: 18 - DNA, 3162 bp, Homo sapiens
gaattccattttaagttatacaaaactgattaccataagtgcggtcgactgcttttatttttacgttgtgtgtgttggaaaaatgctaaaacatcagtctacaattctatatattgttattaaagattaatccaaccagcaacccaaggacatataagcgatttccactattgcatcagagcactcggcaggaaaggcctagccacggggaacattagaagctacagaagcattgcagagaagagaagatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccacggctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaggaattc

SEQ ID NO: 19 - DNA, 2058 bp, Homo sapiens
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 20 - DNA, 5590 bp, Homo sapiens
ctgcggccggcccgcgagctaggctgggtttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtatggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattacgagaacaagaactccaaaatgtctaaaataaggacacacaattctgaagtagaagaggacgacatggacaaacaccagcagaaagcccggtttgccaagcagccggcgtatacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttatttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaattttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatctgactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaac

SEQ ID NO: 21 - protein, 43 aa, Artificial Sequence
  Description of Artificial Sequence Synthetic consensus amino acid sequence
CXXXXXXXXCXXXCXXXXXXXXXXXCXXXXXXXCXXXXXXXXC

SEQ ID NO: 22 - protein, 43 aa, Artificial Sequence; identical to SEQ ID NO: 21
  Description of Artificial Sequence Synthetic consensus amino acid sequence

SEQ ID NO: 23 - protein, 43 aa, Artificial Sequence
  Description of Artificial Sequence Synthetic consensus amino acid sequence
CXXXYYXXXCXXXCRPRBDXFGHXXCXXXGXXXCXXGWXGXXC

SEQ ID NO: 24 - protein, 175 aa, Artificial Sequence
  Description of Artificial Sequence Synthetic EGF-like domain sequence
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX

SEQ ID NO: 25 - protein, 15 aa, Homo sapiens
LNSKIAFKIVSQEPA

SEQ ID NO: 26 - protein, 15 aa, Homo sapiens
TPMFLLSRNTGEVRT

SEQ ID NO: 27 - protein, 277 aa, Homo sapiens [sequence omitted]

SEQ ID NO: 28 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 29 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 30 - protein, 626 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESERRLGSERRLLGLRGEPPELDLSYSHSDLGKRPTKDSYTLTEELAEYAEIRVK

SEQ ID NO: 31 - protein, 23 aa, Homo sapiens
GRTQDENPVVHFFKNIVTPRTPP

SEQ ID NO: 32 - protein, 22 aa, Homo sapiens
AVYVYIYFNTWTTCQFIAFPFK

SEQ ID NO: 33 - protein, 21 aa, Homo sapiens
SQRHGSKYLATASTMDHARHG

SEQ ID NO: 34 - protein, 20 aa, Homo sapiens
RDTGILDSIGRFFGGDRGAP

SEQ ID NO: 35 - protein, 20 aa, Homo sapiens
QKSHGRTQDENPVVHFFKNI

SEQ ID NO: 36 - protein, 14 aa, Homo sapiens
DENPVVHFFKNIVT

SEQ ID NO: 37 - protein, 15 aa, Homo sapiens
ENPVVHFFKNIVTPR

SEQ ID NO: 38 - protein, 13 aa, Homo sapiens
HFFKNIVTPRTPP

SEQ ID NO: 39 - protein, 14 aa, Homo sapiens
KGFKGVDAQGTLSK

SEQ ID NO: 40 - protein, 20 aa, Homo sapiens
VDAQGTLSKIFKLGGRDSRS

SEQ ID NO: 41 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 42 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 43 - protein, 979 aa, Homo sapiens
MRRPRRPGGLGGSGGLRLLLCLLLLSSRPGGCSAVSAHGCLFDRRLCSHLEVCIQDGLFGQCQVGVGQARPLLQVTSPVLQRLQGVLRQLMSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQKPLSLAAGVKLLEILAEHVHMSSGSFINISVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 44 - protein, 622 aa, Homo sapiens
MSMRSPISAQLALDGVGTMVNCTIKSEEKKEPCHEAPQGSATAAEPQPGDPARASQDSADPQAPAQGNFRGSWDCSSPEGNGSPEPKRPGASEAASGSQEKLDFNRNLKEVVPAIEKLLSSDWKERFLGRNSMEAKDVKGTQESLAEKELQLLVMIHQLSTLRDQLLTAHSEQKNMAAMLFEKQQQQMELARQQQEQIAKQQQQLIQQQHKINLLQQQIQQVNMPYVMIPAFPPSHQPLPVTPDSQLALPIQPIPCKPVEYPLQLLHSPPAPVVKRPGAMATHHPLQEPSQPLNLTAKPKAPELPNTSSSPSLKMSSCVPRPPSHGGPTRDLQSSPPSLPLGFLGEGDAVTKAIQDARQLLHSHSGALDGSPNTPFRKDLISLDSSPAKERLEDGCVHPLEEAMLSCDMDGSRHFPESRNSSHIKRPMNAFMVWAKDERRKILQAFPDMHNSSISKILGSRWKSMTNQEKQPYYEEQARLSRQHLEKYPDYKYKPRPKRTCIVEGKRLRVGEYKALMRTRRQDARQSYVIPPQAGQVQMSSSDVLYPRAAGMPLAQPLVEHYVPRSLDPNMPVIVNTCSLREEGEGTDDRHSVADGEMYRYSEDEDSEGEEKSDGELVVLTD

SEQ ID NO: 45 - protein, 483 aa, Homo sapiens [sequence omitted]

SEQ ID NO: 46 - protein, 471 aa, Homo sapiens
MSHHPSGLRAGFSSTSYRRTFGPPPSLSPGAFSYSSSSRFSSSRLLGSASPSSSVRLGSFRSPRAGAGALLRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYESIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLNIKTTVPEVEPPQDSHSRKTVLIKTIETRNGEQVVTESQKEQRSELDKSSAHSY

SEQ ID NO: 47 - protein, 306 aa, Homo sapiens
DCQAKSTPVIVSATTKKGLSSDLEGEKTTSLKWKSDEVDEQVACQEVKVSVAIEEDLEPENGILELETKSSKLVQNIIQTAVDQFVRTEETATEMLTSELQTQAHMIKADSQDAGQETEKEGEEPQASAQDETPITSAKEESESTAVGQAHSDISKDMSEASEKTMTVEVEGSTVNDQQLEEVVLPSEEEGGGAGTKSVPEDDGHALLAERIEKSLVEPKEDEKGDDVDDPENQNSALADTDASGGLTKESPDTNGPKQKEKEDAQEVELQEGKVHSESDKAITPQAQEELQKQERESAKSELTES

SEQ ID NO: 48 - protein, 520 aa, Homo sapiens
MHGGQGPLLLLLLLAVCLGGTQRNLRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACSISVTYFPFDWQNCSLIFQSQTYSTNEIDLQLSQEDGQTIEWIFIDPEAFTENGEWAIQHRPAKMLLDPAAPAQEAGHQKVVFYLLIQRKPLFYVINIIAPCVLISSVAILIHFLPAKAGGQKCTVAINVLLAQTVFLFLVAKKVPETSQAVPLISKYLTFLLVVTILIVVNAVVVLNVSLRSPHTHSMARGVRKVFLRLLPQLLRMHVRPLAPAAVQDTQSRLQNGSSGWSITTGEEVALCLPRSELLFQQWQRQGLVAAALEKLEKGPELGLSQFCGSLKQAAPAIQACVEACNLIACARHQQSHFDNGNEEWFLVGRVLDRVCFLAMLSLFICGTAGIFLMAHYNRVPALPFPGDPRPYLPSPD

SEQ ID NO: 49 - protein, 457 aa, Homo sapiens [sequence omitted]

SEQ ID NO: 50 - protein, 36 aa, Homo sapiens
TVGLQLIQLINVDEVNQIVTTNVRLKQQWVDYNLKW

SEQ ID NO: 51 - protein, 20 aa, Homo sapiens
QIVTTNVRLKQQWVDYNLKW

SEQ ID NO: 52 - protein, 7 aa, Homo sapiens
QWVDYNL

SEQ ID NO: 53 - protein, 18 aa, Homo sapiens
GGVKKIHIPSEKIWRPDL

SEQ ID NO: 54 - protein, 12 aa, Homo sapiens
AIVKFTKVLLQY

SEQ ID NO: 55 - protein, 20 aa, Homo sapiens
WTPPAIFKSYCEIIVTHFPF

SEQ ID NO: 56 - protein, 13 aa, Homo sapiens
MKLGTWTYDGSVV

SEQ ID NO: 57 - protein, 13 aa, Homo sapiens
MKLGIWTYDGSVV

SEQ ID NO: 58 - protein, 9 aa, Homo sapiens
WTYDGSVVA

SEQ ID NO: 59 - protein, 17 aa, Homo sapiens
SCCPDTPYLDITYHFVM

SEQ ID NO: 60 - protein, 18 aa, Homo sapiens
DTPYLDITYHFVMQRLPL

SEQ ID NO: 61 - protein, 21 aa, Homo sapiens
FIVNVIIPCLLFSFLTGLVFY

SEQ ID NO: 62 - protein, 13 aa, Homo sapiens
LLVIVELIPSTSS

SEQ ID NO: 63 - protein, 19 aa, Homo sapiens
STHVMPNWVRKVFIDTIPN

SEQ ID NO: 64 - protein, 18 aa, Homo sapiens
NWVRKVFIDTIPNIMFFS

SEQ ID NO: 65 - protein, 18 aa, Homo sapiens
IPNIMFFSTMKRPSREKQ

SEQ ID NO: 66 - protein, 16 aa, Homo sapiens
AAAEWKYVAMVMDHIL

SEQ ID NO: 67 - protein, 19 aa, Homo sapiens
IIGTLAVFAGRLIELNQQG

SEQ ID NO: 68 - protein, 20 aa, Homo sapiens
GQTIEWIFIDPEAFTENGEW

SEQ ID NO: 69 - protein, 20 aa, Homo sapiens
MAHYNRVPALPFPGDPRPYL

SEQ ID NO: 70 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 71 - protein, 732 aa, Homo sapiens
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISLQFFLPFSLGKEDGSGDRGDGPFRLGGHGPSFPLKGITEQQKEGLEIVKMVMISLEGEDGLDEIYSFSESLRKLCVFKKIERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI

SEQ ID NO: 72 - protein, 408 aa, Homo sapiens
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 73 - protein, 475 aa, Homo sapiens [sequence omitted]

SEQ ID NO: 74 - protein, 525 aa, Homo sapiens
MEESVNQMQPLNEKQIANSQDGYVWQVTDMNRLHRFLCFGSEGGTYYIKEQKLGLENAEALIRLIEDGRGCEVIQEIKSFSQEGRTTKQEPMLFALAICSQCSDISTKQAAFKAVSEVCRIPTHLFTFIQFKKDLKESMKCGMWGRALRKAIADWYNEKGGMALALAVTKYKQRNGWSHKDLLRLSHLKPSSEGLAIVTKYITKGWKEVHELYKEKALSVETEKLLKYLEAVEKVKRTRDELEVIHLIEEHRLVREHLLTNHLKSKEVWKALLQEMPLTALLRNLGKMTANSVLEPGNSEVSLVCEKLCNEKLLKKARIHPFHILIALETYKTGHGLRGKLKWRPDEEILKALDAAFYKTFKTVEPTGKRFLLAVDVSASMNQRVLGSILNASTVAAAMCMVVTRTEKDSYVVAFSDEMVPCPVTTDMTLQQVLMAMSQIPAGGTDCSLPMIWAQKTNTPADVFIVFTDNETFAGGVHPAIALREYRKKMDIPAKLIVCGMTSNGFTIADPDDRALQNTLLNKSF

SEQ ID NO: 75 - protein, 764 aa, Homo sapiens [sequence omitted]

SEQ ID NO: 76 - protein, 933 aa, Homo sapiens [sequence omitted]

SEQ ID NO: 77 - protein, 263 aa, Lolium perenne
MASSSSVLLVVALFAVFLGSAHGIAKVPPGPNITAEYGDKWLDAKSTWYGKPTGAGPKDNGGACGYKNVDKAPFNGMTGCGNTPIFKDGRGCGSCFEIKCTKPESCSGEAVTVTITDDNEEPIAPYHFDLSGHAFGSMAKKGEEQNVRSAGELELQFRRVKCKYPDDTKPTFHVEKASNPNYLAILVKYVDGDGDVVAVDIKEKGKDKWTELKESWGAVWRIDTPDKLTGPFTVRYTTEGGTKSEFEDVIPEGWKADTSYSAK

SEQ ID NO: 78 - protein, 263 aa, Phleum pratense
MASSSSVLLVVVLFAVFLGSAYGIPKVPPGPNITATYGDKWLDAKSTWYGKPTGAGPKDNGGACGYKDVDKPPFSGMTGCGNTPIFKSGRGCGSCFEIKCTKPEACSGEPVVVHITDDNEEPIAPYHFDLSGHAFGAMAKKGDEQKLRSAGELELQFRRVKCKYPEGTKVTFHVEKGSNPNYLALLVKYVNGDGDVVAVDIKEKGKDKWIELKESWGAIWRIDTPDKLTGPFTVRYTTEGGTKTEAEDVIPEGWKADTSYESK

SEQ ID NO: 79 - protein, 160 aa, Betula pendula
MGVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 80 - protein, 396 aa, Ambrosia artemisiifolia
MGIKHCCYILYFTLALVTLLQPVRSAEDLQEILPVNETRRLTTSGAYNIIDGCWRGKADWAENRKALADCAQGFGKGTVGGKDGDIYTVTSELDDDVANPKEGTLRFGAAQNRPLWIIFERDMVIRLDKEMVVNSDKTIDGRGAKVEIINAGFTLNGVKNVIIHNINMHDVKVNPGGLIKSNDGPAAPRAGSDGDAISISGSSQIWIDHCSLSKSVDGLVDAKLGTTRLTVSNSLFTQHQFVLLFGAGDENIEDRGMLATVAFNTFTDNVDQRMPRCRHGFFQVVNNNYDKWGSYAIGGSASPTILSQGNRFCAPDERSKKNVLGRHGEAAAESMKWNWRTNKDVLENGAIFVASGVDPVLTPEQSAGMIPAEPGESALSLTSSAGVLSCQPGAPC

SEQ ID NO: 81 - protein, 79 aa, Brassica napus
MADAEHERIFKKFDTDGDGKISAAELEEALKKLGSVTPDDVTRMMAKIDTDGDGNISFQEFTEFASANPGLMKDVAKVF

SEQ ID NO: 82 - protein, 320 aa, Dermatophagoides pteronyssinus
MKIVLAIASLLALSAVYARPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 83 - protein, 321 aa, Dermatophagoides farinae
MKFVLAIASLLVLSTVYARPASIKTFEEFKKAFNKNYATVEEEEVARKNFLESLKYVEANKGAINHLSDLSLDEFKNRYLMSAEAFEQLKTQFDLNAETSACRINSVNVPSELDLRSLRTVTPIRMQGGCGSCWAFSGVAATESAYLAYRNTSLDLSEQELVDCASQHGCHGDTIPRGIEYIQQNGVVEERSYPYVAREQRCRRPNSQHYGISNYCQIYPPDVKQIREALTQTHTAIAVIIGIKDLRAFQHYDGRTIIQHDNGYQPNYHAVNIVGYGSTQGDDYWIVRNSWDTTWGDSGYGYFQAGNNLMMIEQYPYVVIM

SEQ ID NO: 84 - protein, 13 aa, Homo sapiens
VHFFKNIVTPRTP

SEQ ID NO: 85 - DNA, 36 bp, Artificial Sequence
  Description of Artificial Sequence Synthetic primer
caaaagacaactcaagcttcaccatgggctccatcg

SEQ ID NO: 86 - DNA, 39 bp, Artificial Sequence
  Description of Artificial Sequence Synthetic primer
cagctttcttcggatccaggggaaacacatctgccaaag

SEQ ID NO: 87 - DNA, 33 bp, Artificial Sequence
  Description of Artificial Sequence Synthetic primer
attaagtcgacatccaccatggaaactaacgcc

SEQ ID NO: 88 - DNA, 33 bp, Artificial Sequence
  Description of Artificial Sequence Synthetic primer
ggattcggatccgagaatgacaacatatggata

SEQ ID NO: 89 - DNA, 33 bp, Artificial Sequence
  Description of Artificial Sequence Synthetic primer
ggcgcagcgatggctagcccacggacgcgcggc

SEQ ID NO: 90 - DNA, 35 bp, Artificial Sequence
  Description of Artificial Sequence Synthetic primer
ctgcagaggatccctatacgatgtactccattcgg

SEQ ID NO: 91 - DNA, 29 bp, Artificial Sequence
  Description of Artificial Sequence Synthetic primer
cttcaccatggctagccggtgcgcgctgg

SEQ ID NO: 92 - DNA, 44 bp, Artificial Sequence
  Description of Artificial Sequence Synthetic primer
ctgcagaggatccttacacctcagttgctatgacgcactcatcc

SEQ ID NO: 93 - DNA, 36 bp, Artificial Sequence
  Description of Artificial Sequence Synthetic primer
catccaccatggctagcaacgcctgcagtatcaatg

SEQ ID NO: 94 - DNA, 33 bp, Artificial Sequence
  Description of Artificial Sequence Synthetic primer
ttgttcggatccttagagaatgacaacatatgg

SEQ ID NO: 95 - protein, 7 aa, Homo sapiens
FKNIVTP